(19) United States
(12) Patent Application Publication
(10) Pub. No.: US 2025/0152724 A1
(43) Pub. Date: May 15, 2025

(54) ANTIBODY CONJUGATES SPECIFIC FOR MUCIN-1 AND METHODS OF USE THEREOF

(71) Applicant: R.P. Scherer Technologies, LLC, Carson City, NV (US)

(72) Inventors: David RABUKA, Kensington, CA (US); Penelope M. DRAKE, Castro Valley, CA (US); Yun Cheol KIM, Walnut Creek, CA (US); Robyn M. BARFIELD, Emeryville, CA (US); Maxine BAUZON, Hercules, CA (US); Ayodele OGUNKOYA, Emeryville, CA (US); Stepan CHUPRAKOV, Emeryville, CA (US)

(21) Appl. No.: 18/291,900

(22) PCT Filed: Oct. 28, 2022
(86) PCT No.: PCT/US2022/038904

§ 371 Date: Jan. 24, 2024

Related U.S. Application Data
(60) Provisional application No. 63/344,932, filed on May 23, 2022.
(60) Provisional application No. 63/322,914, filed on Mar. 23, 2022.

(51) Int. Cl. A61K 47/68; A61P 35/00; C07K 16/30

(52) U.S. Cl. CPC A61K 47/68031; A61K 47/68037; A61K 47/6851; A61K 47/6889; A61P 35/00; C07K 16/3092

(57) ABSTRACT

The present disclosure provides antibody conjugates (e.g., antibody-drug conjugates (ADCs)) specific for MUC1. The disclosure also encompasses methods of production of such conjugates, as well as methods of using the same. Also provided are compositions that include the ADC of the present disclosure, including in some instances, pharmaceutical compositions. In certain aspects, provided are methods of using the ADC that include administering to an individual having a cell proliferative disorder a therapeutically effective amount of the ADC of the present disclosure.

### CROSS-REFERENCE TO RELATED APPLICATIONS

This application claims the benefit of priority to U.S. Provisional Application No. 63/227,666, filed Jul. 30, 2021, U.S. Provisional Application No. 63/322,914, filed Mar. 23, 2022, and U.S. Provisional Application No. 63/344,932, filed May 23, 2022, the disclosures of each of which are incorporated herein by reference.

### INCORPORATION-BY-REFERENCE OF MATERIAL ELECTRONICALLY SUBMITTED

A Sequence Listing is provided herewith as a Sequence Listing XML, “RDWD-045WO_SEQ_LIST” created on Oct. 13, 2022 and having a size of 164 KB. The contents of the Sequence Listing XML are incorporated by reference herein in their entirety.

### Introduction

The field of protein-small molecule therapeutic conjugates has advanced greatly, providing a number of clinically beneficial drugs with the promise of providing more in the years to come. Protein-conjugate therapeutics can provide several advantages, due to, for example, specificity, multiplicity of functions and relatively low off-target activity, resulting in fewer side effects. Chemical modification of proteins may extend these advantages by rendering them more potent, stable, or multimodal.

A number of standard chemical transformations are commonly used to create and manipulate post-translational modifications on proteins. There are a number of methods where one is able to modify the side chains of certain amino acids selectively. For example, carboxylic acid side chains (aspartate and glutamate) may be targeted by initial activation with a water-soluble carbodiimide reagent and subsequent reaction with an amine. Similarly, lysine can be targeted through the use of activated esters or isothiocyanates, and cysteine thiols can be targeted with maleimides and α-halo-carbonyls.

One significant obstacle to the creation of a chemically altered protein therapeutic or reagent is the production of the protein in a biologically active, homogenous form. Conjugation of a drug or detectable label to a polypeptide can be difficult to control, resulting in a heterogeneous mixture of conjugates that differ in the number of drug molecules attached and in the position of chemical conjugation. In some instances, it may be desirable to control the site of conjugation and/or the drug or detectable label conjugated to the polypeptide using the tools of synthetic organic chemistry to direct the precise and selective formation of chemical bonds on a polypeptide.

Mucin-1 (also referred to as Mucin 1 or MUC1) is a member of the mucin family. Mucins are O-glycosylated proteins that play an essential role in forming protective mucous barriers on epithelial surfaces. MUC1 is expressed on the apical surface of epithelial cells that line the mucosal surfaces of many different tissues including lung, breast, stomach and pancreas. This protein is proteolytically cleaved into alpha and beta subunits that form a heterodimeric complex. The N-terminal alpha subunit functions in cell-adhesion and the C-terminal beta subunit is involved in cell signaling. Overexpression, aberrant intracellular localization, and changes in glycosylation of this protein have been associated with carcinomas.

There is a need in the art for safe and effective agents that target MUC1 for the treatment of MUC1-associated conditions, such as cancer.

### SUMMARY

The present disclosure provides antibody conjugates (e.g., antibody-drug conjugates (ADCs)) specific for MUC1. The disclosure also encompasses methods of production of such conjugates, as well as methods of using the same. Embodiments of each are described in more detail in the sections below. Also provided are compositions that include the ADC of the present disclosure, including in some instances, pharmaceutical compositions. In certain aspects, provided are methods of using the ADC that include administering to an individual having a cell proliferative disorder a therapeutically effective amount of the ADC of the present disclosure.

### BRIEF DESCRIPTION OF THE FIGURES

FIG. 1 shows that anti-MUC1 antibodies, MUC1 gB06, MUC1 G12, and MUC1 H02 are more than 99%, more than 99%, and more than 98% monomeric, respectively, as determined by size exclusion chromatography (SEC).

FIGS. 2A-2C show that anti-MUC1 antibodies, MUC1 gB06, MUC1 G12, and MUC1 H02 bind to recombinant 20mer MUC1 glycosylated-biotin but not to recombinant 60mer MUC1 non-glycosylated-biotin or to a decoy peptide as assessed by ELISA.

FIGS. 3A-3B show level of binding by the anti-MUC1 antibodies, MUC1 gB06, MUC1 G12, and MUC1 H02 to uncoated streptavidin or Maxisorp plate.

FIG. 4 shows superimposed histograms showing the binding of the indicated antibodies to the named cell lines, tested in triplicates.

FIG. 5 shows staggered histograms showing the binding of the indicated antibodies to the named cell lines.

FIG. 6 shows the melting temperature of CH2 and Fab regions of the B06, G12, and H02 anti-MUC1 antibodies as determined by differential scanning fluorimetry.

FIG. 7. Aldehyde-tagged antibody production and ADC generation using HIPS-mediated conjugation. (A) The formylglycine recognition sequence (CXPXR) is genetically encoded into the antibody. (B) Co-translationally formylglycine-generating enzyme converts the cysteine within the recognition sequence to a formylglycine residue containing an aldehyde functional group that can be specifically conjugated with (C) the Hydrazino-iso-Pictet-Spengler (HIPS) conjugation element.

FIG. 8. CT-tagged B06 antibody conjugated to RED-601 yields a DAR (drug antibody ratio) of 1.85 as determined by HIC.

FIG. 9. CT-tagged B06 antibody conjugated to RED-601 is 99.3% monomeric as determined by SEC.

FIG. 10. CT-tagged G12 antibody conjugated to RED-601 yields a DAR of 1.89 as determined by HIC.

FIG. 11. CT-tagged G12 antibody conjugated to RED-601 is 99.9% monomeric as determined by SEC.

FIG. 12. CT-tagged H02 antibody conjugated to RED-601 yields a DAR of 1.90 as determined by HIC.

FIG. 13. CT-tagged H02 antibody conjugated to RED-601 is 99.1% monomeric as determined by SEC.

FIG. 14. In vitro potency against T47D cells of Monomethyl auristatin E (MMAE)-conjugated anti-MUC1 ADCs made from the B06, G12, or H02 variant antibodies. Free MMAE is included as a benchmark for potency of the payload.

FIG. 15. In vivo efficacy against a T47D xenograft of MMAE-conjugated anti-MUC1ADCs−B06 RED-601 and H02 RED-601—carrying an MMAE payload. n=10 mice/group; dosing is indicated by arrows.

FIG. 16. Representative data showing B06 ADC binding to primary human ovarian adenocarcinomas. Four ovarian adenocarcinoma specimens were reacted with B06 ADC (top row) or isotype control ADC (bottom row). Brown color indicates ADC binding.

FIG. 17. Representative data showing B06 ADC binding to primary human lung tumors. Four lung cancer specimens were reacted with B06 ADC (top row) or isotype control ADC (bottom row). Adenocarcinoma two left columns; squamous cell carcinoma two right columns. Brown color indicates ADC binding.

FIG. 18. Representative data showing B06 ADC binding to primary human breast tumors. Four breast ductal carcinoma specimens were reacted with B06 ADC (top row) or isotype control ADC (bottom row). Brown color indicates ADC binding.

FIG. 19. B06 ADC binds strongly to patient-derived xenograft (PDX) tumor models. Four Charles River Laboratories PDX specimens were reacted with B06 ADC (top row) or isotype control ADC (bottom row). Brown color indicates ADC binding. Tumor origin from left to right: gastric, breast, lung, gastric.

FIG. 20. Structure for RED-601, a linker-payload conjugated to the anti-MUC1 antibodies (see Compound 8 in Example 2).

FIG. 21A depicts a site map showing possible modification sites for generation of an aldehyde tagged Ig polypeptide. The upper sequence is the amino acid sequence of the conserved region of an IgG1 light chain polypeptide (SEQ ID NO:74) and shows possible modification sites in an Ig light chain; the lower sequence is the amino acid sequence of the conserved region of an Ig heavy chain polypeptide (SEQ ID NO:75) (GenBank Accession No. AAG00909) and shows possible modification sites in an Ig heavy chain. The heavy and light chain numbering is based on the full-length heavy and light chains.

FIGS. 21B-21C depicts an alignment of Homo sapiens immunoglobulin heavy chain constant regions for IgG1 (SEQ ID NO:47; GenBank P01857.1), IgG2 (SEQ ID NO:48; GenBank P01859.2), IgG3 (SEQ ID NO:49; GenBank P01860.2), IgG4 (SEQ ID NO:50; GenBank AAB59394.1), and IgA (SEQ ID NO:51; GenBank AAAT74070), showing modification sites at which aldehyde tags can be provided in an immunoglobulin heavy chain. The heavy and light chain numbering is based on the full heavy and light chains.

FIG. 21D depicts an alignment of immunoglobulin light chain constant regions, showing modification sites at which aldehyde tags can be provided in an immunoglobulin light chain. Seq1=Homo sapiens kappa light chain constant region; GenBank CAA75031.1; SEQ ID NO:52. Seq2=Homo sapiens kappa light chain constant region; GenBank BAC0168.1; SEQ ID NO:53. Seq3=Homo sapiens lambda light chain constant region; GenBank CAA75033; SEQ ID NO:54. Seq4=Mus musculus light chain constant region; GenBank AAB09710.1; SEQ ID NO:55. Seq5=Rattus norvegicus light chain constant region; GenBank AAD10133; SEQ ID NO:56.

FIG. 22. Binding of anti-MUC1 affinity-matured variant B06 and comparator antibodies, PankoMab and 1B2, to 20mer MUC1 glycosylated-biotin peptide as assessed by ELISA.

FIG. 23. Binding of anti-MUC1 affinity-matured variants G12 and H02 to 20mer MUC1 glycosylated-biotin peptide as assessed by ELISA.

FIG. 24. Binding of anti-MUC1 affinity-matured variant B06 and comparator antibodies, PankoMab and 1B2, to 60mer MUC1 non-glycosylated-biotin peptide as assessed by ELISA.

FIG. 25. Binding of anti-MUC1 affinity-matured variants G12 and H02 to 60mer MUC1 non-glycosylated-biotin peptide as assessed by ELISA.

FIG. 26. Binding of anti-MUC1 affinity-matured variants and parental antibody to antigen-positive T47D cells or antigen-negative HEK cells as assessed by flow cytometry.

FIG. 27. In vitro potency against UACC-812 cells of maytansine or monomethyl auristatin E (MMAE)-conjugated anti-MUC1 ADCs made from the B06 or H02 variant antibodies. Free maytansine was included as a benchmark for potency of the payload.

FIG. 28. Single-tagged B06 antibody conjugated at 91N to a branched MMAE linker-payload was 96.4% monomeric as determined by SEC.

FIG. 29. Single-tagged B06 antibody conjugated at 91N to Compound 8 yields a DAR of 1.78 as determined by HIC.

FIG. 30. Single-tagged B06 antibody conjugated at 91N to Compound 8 is 96.2% monomeric as determined by SEC.

FIG. 31. Single-tagged B06 antibody conjugated at 91N to Compound 21 yields a DAR of 3.74 as determined by PLRP.

FIG. 32. Single-tagged B06 antibody conjugated at 91N to Compound 21 is 95.9% monomeric as determined by SEC.

FIG. 33. Double-tagged B06 antibody conjugated to Compound 21 yields a DAR of 7.47 as determined by PLRP.

FIG. 34. Double-tagged B06 antibody conjugated to Compound 21 is 96.7% monomeric as determined by SEC.

### DEFINITIONS

“Alkyl” refers to monovalent saturated aliphatic hydrocarbyl groups having from 1 to 10 carbon atoms and such as 1 to 6 carbon atoms, or 1 to 5, or 1 to 4, or 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain (except the C₁ carbon atom) have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C(O)—, —C(O)NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl,” “alkylaminoalkenyl,” and “alkylaminoalkynyl” refers to the groups R′NHR″—where R′ is alkyl group as defined herein and R″ is alkylene, alkenylene or alkynylene group as defined herein.

The term “alkaryl” or “aralkyl” refers to the groups -alkylene-aryl and -substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, and —SO₂-heteroaryl.

“Alkynyloxy” refers to the group —O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

“Acylamino” refers to the groups —NR²⁰C(O)alkyl, —NR²⁰C(O)substituted alkyl, N R²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR⁵¹R⁵², wherein R⁵¹ and R⁵² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵¹ and R⁵² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR⁵¹C(O)NR⁵²R⁵³ where R⁵¹, R⁵², and R⁵³ are independently selected from hydrogen, alkyl, aryl or cycloalkyl, or where two R groups are joined to form a heterocyclyl group.

The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aminosulfonyl” refers to the group —SO₂NR⁵¹R⁵², wherein R⁵¹ and R⁵² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R⁵¹ and R⁵² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR⁵¹SO₂R⁵², wherein R⁵¹ and R⁵² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵¹ and R⁵² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O— alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O— substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and preferably from 1 to 2 double bonds.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic. To satisfy valence requirements, any heteroatoms in such heteroaryl rings may or may not be bonded to H or a substituent group, e.g., an alkyl group or other substituent as described herein. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from nitrogen, sulfur, or oxygen, where, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties. To satisfy valence requirements, any heteroatoms in such heterocyclic rings may or may not be bonded to one or more H or one or more substituent group(s), e.g., an alkyl group or other substituent as described herein.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Sulfonyloxy” refers to the group —OSO₂-alkyl, OSO₂-substituted alkyl, OSO₂-alkenyl, OSO₂-substituted alkenyl, OSO₂-cycloalkyl, OSO₂-substituted cycloalkyl, OSO₂-cycloalkenyl, OSO₂-substituted cylcoalkenyl, OSO₂-aryl, OSO₂-substituted aryl, OSO₂-heteroaryl, OSO₂-substituted heteroaryl, OSO₂-heterocyclic, and OSO₂ substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (═S).

“Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻ M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻ M⁺, —P(O)(OR⁷⁰) 2, —C(O)R⁷⁰, —C(S)R⁷⁰, —C(NR⁷⁰)R⁷⁰, —C(O)O⁻ M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of 0, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca^{2+]}_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

The terms “antibodies” and “immunoglobulin” include antibodies or immunoglobulins of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the term are Fab′, Fv, F(ab′)₂, and or other antibody fragments that retain specific binding to antigen, and monoclonal antibodies. An antibody may be monovalent or bivalent. “Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH₁) of the heavy chain. Fab fragments differ from Fab′ fragments by the addition of a few residues at the carboxyl terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. In some aspects, the Fv polypeptide further comprises a polypeptide linker between the VH and VL domains, which enables the sFv to form the desired structure for antigen binding.

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites.

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. A subject anti-MUC1 antibody binds specifically to an epitope within a MUC1 polypeptide, e.g., a human MUC1 polypeptide, for example, a glycosylated MUC1 or a fragment thereof. Non-specific binding would refer to binding with an affinity of less than about 10⁻⁷ M, e.g., binding with an affinity of 10⁻⁶ M, 10⁻¹ M, 10⁻⁴ M, etc.

The term “specifically binds” in the context of an antibody and an antigen means that the antibody binds to or associates with the antigen with an affinity or Kₐ (that is, an equilibrium association constant of a particular binding interaction with units of 1/M) of, for example, greater than or equal to about 10⁵ M⁻¹.

“High affinity” binding refers to binding with a Kₐ of at least 10⁷ M⁻¹, at least 10⁸ M⁻¹, at least 10⁹ M⁻¹, at least 10¹⁰ M⁻¹ at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10⁻⁵ M to 10⁻¹³ M, or less). In some embodiments, specific binding means the antibody binds to the antigen with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the antibody for an antigen can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

As used herein, the term “CDR” or “complementarity determining region” is intended to mean the non-contiguous antigen combining sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1
CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

Throughout the present disclosure, the numbering of the residues in an immunoglobulin heavy chain and in an immunoglobulin light chain is that as in Kabat et al., Sequences of Proteins of Immunological Interest, 5th Ed. Public Health Service, National Institutes of Health, Bethesda, Md. (1991), expressly incorporated herein by reference.

As used herein, the term “framework” when used in reference to an antibody variable region is intended to mean all amino acid residues outside the CDR regions within the variable region of an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by the CDRs.

A “parent Ig polypeptide” is a polypeptide comprising an amino acid sequence which lacks an aldehyde-tagged constant region as described herein. The parent polypeptide may comprise a native sequence constant region, or may comprise a constant region with pre-existing amino acid sequence modifications (such as additions, deletions and/or substitutions).

In the context of an Ig polypeptide, the term “constant region” is well understood in the art, and refers to a C-terminal region of an Ig heavy chain, or an Ig light chain. An Ig heavy chain constant region includes CH1, CH2, and CH3 domains (and CH4 domains, where the heavy chain is μ or an ε heavy chain). In a native Ig heavy chain, the CH1, CH2, CH3 (and, if present, CH4) domains begin immediately after (C-terminal to) the heavy chain variable (VH) region, and are each from about 100 amino acids to about 130 amino acids in length. In a native Ig light chain, the constant region begins immediately after (C-terminal to) the light chain variable (VL) region, and is about 100 amino acids to 120 amino acids in length.

An “epitope” is a site on an antigen (e.g., a site on MUC1) to which an antibody binds. Epitopes can be formed both from contiguous amino acids or noncontiguous amino acids juxtaposed by folding (e.g., tertiary folding) of a protein. Epitopes formed from contiguous amino acids are typically retained on exposure to denaturing solvents whereas epitopes formed by folding are typically lost on treatment with denaturing solvents. An epitope typically includes at least 3, and more usually, at least 5 or 8-10 amino acids in a linear or spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996). Several commercial laboratories offer epitope mapping services. Epitopes bound by an antibody immunoreactive with a membrane associated antigen can reside on the surface of the cell (e.g., in the extracellular region of a transmembrane protein), so that such epitopes are considered cell-surface accessible, solvent accessible, and/or cell-surface exposed.

By “genetically-encodable” as used in reference to an amino acid sequence of polypeptide, peptide or protein means that the amino acid sequence is composed of amino acid residues that are capable of production by transcription and translation of a nucleic acid encoding the amino acid sequence, where transcription and/or translation may occur in a cell or in a cell-free in vitro transcription/translation system.

The term “control sequences” refers to DNA sequences that facilitate expression of an operably linked coding sequence in a particular expression system, e.g., mammalian cell, bacterial cell, cell-free synthesis, etc. The control sequences that are suitable for prokaryote systems, for example, include a promoter, optionally an operator sequence, and a ribosome binding site. Eukaryotic cell systems may utilize promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate the initiation of translation. Generally, “operably linked” means that the DNA sequences being linked are contiguous, and, in the case of a secretory leader, contiguous and in reading frame. Linking is accomplished by ligation or through amplification reactions. Synthetic oligonucleotide adaptors or linkers may be used for linking sequences in accordance with conventional practice.

The term “expression cassette” as used herein refers to a segment of nucleic acid, usually DNA, that can be inserted into a nucleic acid (e.g., by use of restriction sites compatible with ligation into a construct of interest or by homologous recombination into a construct of interest or into a host cell genome). In general, the nucleic acid segment comprises a polynucleotide that encodes a polypeptide of interest, and the cassette and restriction sites are designed to facilitate insertion of the cassette in the proper reading frame for transcription and translation. Expression cassettes can also comprise elements that facilitate expression of a polynucleotide encoding a polypeptide of interest in a host cell, e.g., a mammalian host cell. These elements may include, but are not limited to: a promoter, a minimal promoter, an enhancer, a response element, a terminator sequence, a polyadenylation sequence, and the like.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In some embodiments, the antibody will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated antibody includes the antibody in situ within recombinant cells since at least one component of the antibody's natural environment will not be present. In some instances, isolated antibody will be prepared by at least one purification step.

The term “natural antibody” refers to an antibody in which the heavy and light chains of the antibody have been made and paired by the immune system of a multi-cellular organism. Spleen, lymph nodes, bone marrow and serum are examples of tissues that produce natural antibodies. For example, the antibodies produced by the antibody producing cells isolated from a first animal immunized with an antigen are natural antibodies.

The term “humanized antibody” or “humanized immunoglobulin” refers to a non-human (e.g., mouse or rabbit) antibody containing one or more amino acids (in a framework region, a constant region or a CDR, for example) that have been substituted with a correspondingly positioned amino acid from a human antibody. In general, humanized antibodies produce a reduced immune response in a human host, as compared to a non-humanized version of the same antibody. Antibodies can be humanized using a variety of techniques known in the art including, for example, CDR-grafting, veneering or resurfacing, chain shuffling, and the like. In certain embodiments, framework substitutions are identified by modeling of the interactions of the CDR and framework residues to identify framework residues important for antigen binding and sequence comparison to identify unusual framework residues at particular positions. Accordingly, the antibodies described above may be humanized using methods that are well known in the art.

In certain embodiments, the antibody molecules disclosed herein include a heavy chain comprising a variable heavy chain region as provided herein and a human IgG1 constant region having the amino acid sequence sequence set forth in UniProt: P01857-1, version 1. In certain embodiments, the antibody molecules disclosed herein include a light chain comprising a variable light chain region as provided herein and a human light chain constant region. In certain embodiments, the human light chain constant region is a human kappa light chain constant region having the amino acid set forth in UniProtKB/Swiss-Prot: P01834.2. In certain embodiments, the human IgG1 heavy chain constant region present in the subject antibodies may include mutations, e.g., substitutions to modulate Fc function. For example, the LALAPG effector function mutations (L234A, L235A, and P329G) or the N297A mutation may be introduced to reduce antibody dependent cellular cytotoxicity (ADCC). The numbering of the substitutions is based on the EU numbering system. The “EU numbering system” or “EU index” is generally used when referring to a residue in an immunoglobulin heavy chain constant region (e.g., the EU index reported in Kabat et al., Sequences of Proteins of Immunological Interest, 5th Ed. Public Health Service, National Institutes of Health, Bethesda, MD. (1991)). The “EU index as in Kabat” refers to the residue numbering of the human IgG 1 EU antibody.

The term “chimeric antibodies” refer to antibodies whose light and heavy chain genes have been constructed, typically by genetic engineering, from antibody variable and constant region genes belonging to different species. For example, the variable segments of the genes from a mouse monoclonal antibody may be joined to human constant segments, such as gamma 1 and gamma 3. An example of a therapeutic chimeric antibody is a hybrid protein composed of the variable or antigen-binding domain from a mouse antibody and the constant or effector domain from a human antibody, although domains from other mammalian species may be used.

The terms “polypeptide,” “peptide,” and “protein” are used interchangeably herein to refer to a polymeric form of amino acids of any length. Unless specifically indicated otherwise, “polypeptide,” “peptide,” and “protein” can include genetically coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, proteins which contain at least one N-terminal methionine residue (e.g., to facilitate production in a recombinant host cell); immunologically tagged proteins; and the like. In the context of an antibody, it is clear that a chain or a domain comprises a polypeptide.

“Native amino acid sequence” or “parent amino acid sequence” are used interchangeably herein to refer to the amino acid sequence of a polypeptide prior to modification to include a modified amino acid residue.

The terms “amino acid analog,” “unnatural amino acid,” and the like may be used interchangeably, and include amino acid-like compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or combinations thereof. For example, amino acid analogs may include α-hydroxy acids, and α-amino acids, and the like.

The terms “amino acid side chain” or “side chain of an amino acid” and the like may be used to refer to the substituent attached to the α-carbon of an amino acid residue, including natural amino acids, unnatural amino acids, and amino acid analogs. An amino acid side chain can also include an amino acid side chain as described in the context of the modified amino acids and/or conjugates described herein.

The term “conjugated” generally refers to a chemical linkage, either covalent or non-covalent, usually covalent, that proximally associates one molecule of interest with a second molecule of interest. In some embodiments, the agent is selected from a half-life extending moiety, a labeling agent, and a therapeutic agent. For half-life extension, for example, the antibodies of the present disclosure can optionally be modified to provide for improved pharmacokinetic profile (e.g., by PEGylation, hyperglycosylation, and the like). Modifications that can enhance serum half-life are of interest.

The term “carbohydrate” and the like may be used to refer to monomers units and/or polymers of monosaccharides, disaccharides, oligosaccharides, and polysaccharides. The term sugar may be used to refer to the smaller carbohydrates, such as monosaccharides, disaccharides. The term “carbohydrate derivative” includes compounds where one or more functional groups of a carbohydrate of interest are substituted (replaced by any convenient substituent), modified (converted to another group using any convenient chemistry) or absent (e.g., eliminated or replaced by H). A variety of carbohydrates and carbohydrate derivatives are available and may be adapted for use in the subject compounds and conjugates.

As used herein the term “isolated” is meant to describe a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, at least 75% free, at least 80% free, at least 85% free, at least 90% free, at least 95% free, at least 98% free, or more than 98% free, from other components with which it is naturally associated.

The term “physiological conditions” is meant to encompass those conditions compatible with living cells, e.g., predominantly aqueous conditions of a temperature, pH, salinity, etc. that are compatible with living cells.

By “reactive partner” is meant a molecule or molecular moiety that specifically reacts with another reactive partner to produce a reaction product. Exemplary reactive partners include a cysteine or serine of a sulfatase motif and Formylglycine Generating Enzyme (FGE), which react to form a reaction product of a converted aldehyde tag containing a formylglycine (fGly) in lieu of cysteine or serine in the motif. Other exemplary reactive partners include an aldehyde of an fGly residue of a converted aldehyde tag (e.g., a reactive aldehyde group) and an “aldehyde-reactive reactive partner,” which comprises an aldehyde-reactive group and a moiety of interest, and which reacts to form a reaction product of a polypeptide having the moiety of interest conjugated to the polypeptide through the fGly residue.

“N-terminus” refers to the terminal amino acid residue of a polypeptide having a free amine group, which amine group in non-N-terminus amino acid residues normally forms part of the covalent backbone of the polypeptide.

“C-terminus” refers to the terminal amino acid residue of a polypeptide having a free carboxyl group, which carboxyl group in non-C-terminus amino acid residues normally forms part of the covalent backbone of the polypeptide.

By “internal site” as used in referenced to a polypeptide or an amino acid sequence of a polypeptide means a region of the polypeptide that is not at the N-terminus or at the C-terminus.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, e.g., arresting its development; and (c) relieving the disease, e.g., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” refers to the amount of a subject anti-MUC1 Ab that, when administered to a mammal or other subject for treating a disease, is sufficient to effect such treatment for the disease. The “therapeutically effective amount” will vary depending on the anti-MUC1 Ab, the disease and its severity and the age, weight, etc., of the subject to be treated.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an antibody” includes a plurality of such antibodies and reference to “the CDR” includes reference to one or more CDRs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides antibody conjugates (e.g., antibody-drug conjugates (ADCs)) specific for MUC1. The disclosure also encompasses methods of production of such conjugates, as well as methods of using the same. Embodiments of each are described in more detail in the sections below. Also provided are compositions that include the ADC of the present disclosure, including in some instances, pharmaceutical compositions. In certain aspects, provided are methods of using the ADC that include administering to an individual having a cell proliferative disorder a therapeutically effective amount of the ADC of the present disclosure.

### MUC1 Antibodies and Antibody-Drug Conjugates Thereof

As summarized above, the present disclosure provides conjugates (e.g., antibody-drug conjugates (ADCs)) of antibodies specific for MUC1. In addition, the present disclosure provides anti-MUC1 antibodies comprising a fGly residue.

### Antibody-Drug Conjugates

The present disclosure provides a conjugate, e.g., an antibody-drug conjugate (ADC) of antibodies specific for MUC1. By “conjugate” is meant a polypeptide (e.g., an antibody) is covalently attached to a moiety of interest (e.g., a drug or active agent). For example, an antibody-drug conjugate according to the present disclosure includes one or more drugs or active agents covalently attached to an antibody. In certain embodiments, the polypeptide (e.g., antibody) and the one or more drugs or active agents are bound to each other through one or more functional groups and covalent bonds. For example, the one or more functional groups and covalent bonds can include a linker, such as a cleavable linker, as described herein.

In certain embodiments, the conjugate is a polypeptide conjugate, which includes a polypeptide (e.g., an antibody) conjugated to one or more other moieties. In certain embodiments, the one or more moieties conjugated to the polypeptide can each independently be any of a variety of moieties of interest such as, but not limited to, a drug, an active agent, a detectable label, a water-soluble polymer, or a moiety for immobilization of the polypeptide to a membrane or a surface. In certain embodiments, the conjugate is a drug conjugate, where a polypeptide is an antibody, thus providing an antibody-drug conjugate. For instance, the conjugate can be a drug conjugate, where a polypeptide is conjugated to one or more drugs or active agents. Various types of drugs and active agents may be used in the conjugates and are described in more detail below.

The one or more drugs or active agents can be conjugated to the polypeptide (e.g., antibody) at any desired site of the polypeptide. Thus, the present disclosure provides, for example, a polypeptide having a drug or active agent conjugated at a site at or near the C-terminus of the polypeptide. Other examples include a polypeptide having a drug or active agent conjugated at a position at or near the N-terminus of the polypeptide. Examples also include a polypeptide having a drug or active agent conjugated at a position between the C-terminus and the N-terminus of the polypeptide (e.g., at an internal site of the polypeptide). Combinations of the above are also possible where the polypeptide is conjugated to two or more drugs or active agents.

In certain embodiments, a conjugate of the present disclosure includes one or more drugs or active agents conjugated to an amino acid residue of a polypeptide at the α-carbon of an amino acid residue. Stated another way, a conjugate includes a polypeptide where the side chain of one or more amino acid residues in the polypeptide has been modified and attached to one or more drugs or active agents (e.g., attached to one or more drugs or active agents through a linker as described herein). For example, a conjugate includes a polypeptide where the α-carbon of one or more amino acid residues in the polypeptide has been modified and attached to one or more drugs or active agents (e.g., attached to one or more drugs or active agents through a linker as described herein).

Embodiments of the present disclosure include conjugates where a polypeptide is conjugated to one or more moieties, such as 2 moieties, 3 moieties, 4 moieties, 5 moieties, 6 moieties, 7 moieties, 8 moieties, 9 moieties, or 10 or more moieties. The moieties may be conjugated to the polypeptide at one or more sites in the polypeptide. For example, one or more moieties may be conjugated to a single amino acid residue of the polypeptide. In some cases, one moiety is conjugated to an amino acid residue of the polypeptide. In other embodiments, two moieties may be conjugated to the same amino acid residue of the polypeptide. In other embodiments, a first moiety is conjugated to a first amino acid residue of the polypeptide and a second moiety is conjugated to a second amino acid residue of the polypeptide. Combinations of the above are also possible, for example where a polypeptide is conjugated to a first moiety at a first amino acid residue and conjugated to two other moieties at a second amino acid residue. Other combinations are also possible, such as, but not limited to, a polypeptide conjugated to first and second moieties at a first amino acid residue and conjugated to third and fourth moieties at a second amino acid residue, etc.

The one or more amino acid residues of the polypeptide that are conjugated to the one or more moieties of interest may be naturally occurring amino acids, unnatural amino acids, or combinations thereof. For instance, the conjugate may include one or more drugs or active agents conjugated to a naturally occurring amino acid residue of the polypeptide. In other instances, the conjugate may include one or more drugs or active agents conjugated to an unnatural amino acid residue of the polypeptide. One or more drugs or active agents may be conjugated to the polypeptide at a single natural or unnatural amino acid residue as described herein. One or more natural or unnatural amino acid residues in the polypeptide may be conjugated to the moiety or moieties as described herein. For example, two (or more) amino acid residues (e.g., natural or unnatural amino acid residues) in the polypeptide may each be conjugated to one or two moieties, such that multiple sites in the polypeptide are conjugated to the moieties of interest.

In certain embodiments, the polypeptide (e.g., antibody) and the moiety of interest (e.g., drug or active agent) are conjugated through a conjugation moiety. For example, the polypeptide and the moiety of interest may each be bound (e.g., covalently bonded) to the conjugation moiety, thus indirectly binding the polypeptide and the moiety of interest together through the conjugation moiety. In some cases, the conjugation moiety includes a hydrazinyl-indolyl or a hydrazinyl-pyrrolo-pyridinyl compound, or a derivative of a hydrazinyl-indolyl or a hydrazinyl-pyrrolo-pyridinyl compound. For instance, a general scheme for coupling a moiety of interest to a polypeptide through a hydrazinyl-indolyl or a hydrazinyl-pyrrolo-pyridinyl conjugation moiety is shown in the general reaction scheme below. Hydrazinyl-indolyl and hydrazinyl-pyrrolo-pyridinyl conjugation moiety are also referred to herein as a hydrazino-iso-Pictet-Spengler (HIPS) conjugation moiety and an aza-hydrazino-iso-Pictet-Spengler (azaHIPS) conjugation moiety, respectively.

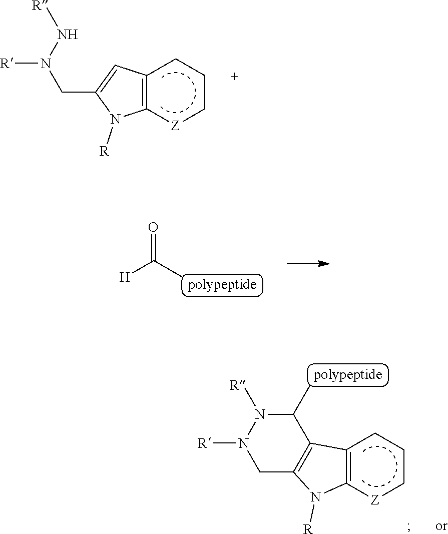

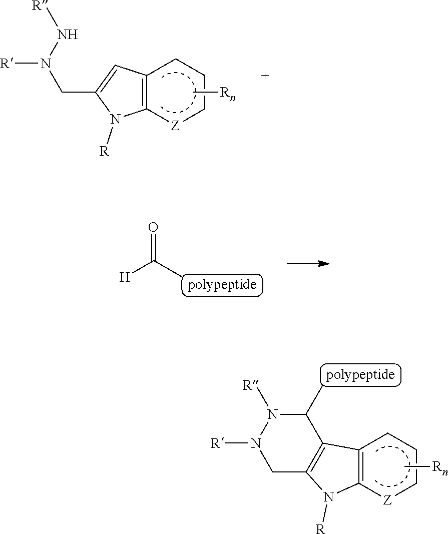

In the reaction scheme above, each R includes the moiety of interest (e.g., a drug or active agent) that is conjugated to the polypeptide (e.g., conjugated to the polypeptide through a cleavable linker as described herein), where n is an integer from 1 to 4. As shown in the reaction scheme above, a polypeptide that includes a 2-formylglycine residue (fGly) is reacted with a drug or active agent that has been modified to include a conjugation moiety (e.g., a hydrazinyl-indolyl or a hydrazinyl-pyrrolo-pyridinyl conjugation moiety) to produce a polypeptide conjugate attached to the conjugation moiety, thus attaching the drug or active agent to the polypeptide through the conjugation moiety.

As described herein, the moiety can be any of a variety of moieties such as, but not limited to, chemical entities, such as detectable labels, or drugs or active agents. R′ and R″ may each independently be any desired substituent, such as, but not limited to, hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. Z may be CR⁶¹, NR⁶², N, O or S, where R⁶¹ and R⁶² are each independently selected from any of the substituents described for R′ and R″ above.

Other hydrazinyl-indolyl or hydrazinyl-pyrrolo-pyridinyl coupling moieties are also possible, as shown in the conjugates and compounds described herein. For example, the hydrazinyl-indolyl or hydrazinyl-pyrrolo-pyridinyl coupling moieties may be attached (e.g., covalently attached) to a linker. As such, embodiments of the present disclosure include a hydrazinyl-indolyl or hydrazinyl-pyrrolo-pyridinyl conjugation moiety attached to a drug or active agent through a linker. Various embodiments of the linker that may couple the hydrazinyl-indolyl or hydrazinyl-pyrrolo-pyridinyl conjugation moiety to the drug or active agent are described in detail herein. For example, in some instances, the linker is a cleavable linker, as described herein.

Additional hydrazinyl-indolyl or hydrazinyl-pyrrolo-pyridinyl conjugation moieties are also possible, as shown in the conjugates and compounds described herein. For example, the hydrazinyl-indolyl or hydrazinyl-pyrrolo-pyridinyl conjugation moieties may be attached (e.g., covalently attached) to two or more linkers. As such, embodiments of the present disclosure include a hydrazinyl-indolyl or hydrazinyl-pyrrolo-pyridinyl conjugation moiety attached to two or more drugs or active agents each through a corresponding linker. Thus, conjugates of the present disclosure may include two or more linkers, where each linker attaches a corresponding drug or active agent to the hydrazinyl-indolyl or hydrazinyl-pyrrolo-pyridinyl conjugation moiety. Accordingly, the hydrazinyl-indolyl or hydrazinyl-pyrrolo-pyridinyl conjugation moiety and two or more linkers may be viewed overall as a “branched linker”, where the hydrazinyl-indolyl or hydrazinyl-pyrrolo-pyridinyl conjugation moiety is attached to two of more “branches”, where each branch includes a linker attached to a drug or active agent.

Combinations of the same of different payloads may be conjugated to the poypeptide through the branched linker. In certain embodiments, the two payloads (e.g., drugs, active agents or detectable labels) attached to the branched linker are the same payload (e.g., drug, active agent or detectable label). For example, a first branch of a branched linker may be attached to a payload (e.g., drug, active agent or detectable label) and a second branch of the branched linker may be attached to the same payload (e.g., drug, active agent or detectable label) as the first branch.

In other embodiments, the two payloads (e.g., drugs, active agents or detectable labels) attached to the branched linker are different payloads (e.g., drugs, active agents or detectable labels). For example, a first branch of a branched linker may be attached to a first payload (e.g., a first drug, active agent or detectable label) and a second branch of the branched linker may be attached to a second payload (e.g., a second drug, active agent or detectable label) different from the first payload (e.g., the first drug, active agent or detectable label) attached to the first branch.

In certain embodiments, the polypeptide (e.g., antibody) may be conjugated to one or more moieties of interest, where one or more amino acid residues of the polypeptide are modified before conjugation to the moiety of interest. Modification of one or more amino acid residues of the polypeptide may produce a polypeptide that contains one or more reactive groups suitable for conjugation to the moiety of interest. In some cases, the polypeptide may include one or more modified amino acid residues to provide one or more reactive groups suitable for conjugation to the moiety of interest (e.g., one or more moieties that includes a conjugation moiety, such as a hydrazinyl-indolyl or a hydrazinyl-pyrrolo-pyridinyl conjugation moiety as described above). For example, an amino acid of the polypeptide may be modified to include a reactive aldehyde group (e.g., a reactive aldehyde). A reactive aldehyde may be included in an “aldehyde tag” or “ald-tag”, which as used herein refers to an amino acid sequence derived from a sulfatase motif (e.g., L(C/S)TPSR (SEQ ID NO:5)) that has been converted by action of a formylglycine generating enzyme (FGE) to contain a 2-formylglycine residue (referred to herein as “fGly”). The fGly residue generated by an FGE may also be referred to as a “formylglycine”. Stated differently, the term “aldehyde tag” is used herein to refer to an amino acid sequence that includes a “converted” sulfatase motif (e.g., a sulfatase motif in which a cysteine or serine residue has been converted to fGly by action of an FGE, e.g., L(fGly)TPSR (SEQ ID NO:24)). A converted sulfatase motif may be produced from an amino acid sequence that includes an “unconverted” sulfatase motif (e.g., a sulfatase motif in which the cysteine or serine residue has not been converted to fGly by an FGE, but is capable of being converted, e.g., an unconverted sulfatase motif with the sequence: L(C/S)TPSR). By “conversion” as used in the context of action of a formylglycine generating enzyme (FGE) on a sulfatase motif refers to biochemical modification of a cysteine or serine residue in a sulfatase motif to a formylglycine (fGly) residue (e.g., Cys to fGly, or Ser to fGly). Additional aspects of aldehyde tags and uses thereof in site-specific protein modification are described in U.S. Pat. Nos. 7,985,783 and 8,729,232, the disclosures of each of which are incorporated herein by reference.

In some cases, to produce the conjugate, the polypeptide containing the fGly residue may be conjugated to the moiety of interest by reaction of the fGly with a compound (e.g., a compound containing a hydrazinyl-indolyl or a hydrazinyl-pyrrolo-pyridinyl conjugation moiety, as described above). For example, an fGly-containing polypeptide may be contacted with a reactive partner-containing drug under conditions suitable to provide for conjugation of the drug to the polypeptide. In some instances, the reactive partner-containing drug may include a hydrazinyl-indolyl or a hydrazinyl-pyrrolo-pyridinyl conjugation moiety as described above. For example, a drug or active agent may be modified to include a hydrazinyl-indolyl or a hydrazinyl-pyrrolo-pyridinyl conjugation moiety. In some cases, the drug or active agent is attached to a hydrazinyl-indolyl or a hydrazinyl-pyrrolo-pyridinyl, such as covalently attached to a hydrazinyl-indolyl or a hydrazinyl-pyrrolo-pyridinyl through a linker, such as a linker as described in detail herein.

In certain embodiments, a conjugate of the present disclosure includes a polypeptide (e.g., an antibody) having at least one amino acid residue that has been attached to one or more moieties of interest (e.g., drugs or active agents). In order to make the conjugate, an amino acid residue of the polypeptide may be modified and then coupled to one or more drugs or active agents attached to a hydrazinyl-indolyl or a hydrazinyl-pyrrolo-pyridinyl conjugation moiety as described above. In certain embodiments, an amino acid residue of the polypeptide (e.g., antibody) is a cysteine or serine residue that is modified to an fGly residue, as described above. In certain embodiments, the modified amino acid residue (e.g., fGly residue) is conjugated to a drug or active agent containing a hydrazinyl-indolyl or a hydrazinyl-pyrrolo-pyridinyl conjugation moiety as described above to provide a conjugate of the present disclosure where the one or more drugs or active agents are conjugated to the polypeptide through the hydrazinyl-indolyl or hydrazinyl-pyrrolo-pyridinyl conjugation moiety. As used herein, the term fGly′ refers to the modified amino acid residue of the polypeptide (e.g., antibody) that is coupled to the moiety of interest (e.g., a drug or active agent).

In certain embodiments, the conjugate includes a polypeptide (e.g., an antibody) having at least one amino acid residue attached to a linker as described herein, which in turn is attached to one or more drugs or active agents. For instance, the conjugate may include a polypeptide (e.g., an antibody) having at least one amino acid residue (fGly′) that is conjugated to the one or more moieties of interest (e.g., one or more drugs or active agents) as described above.

Aspects of the present disclosure include a conjugate of formula (I):

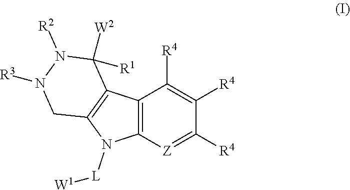

wherein
Z is CR⁴ or N;
R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L is a linker;
W¹ is a drug; and
W² is an anti-MUC1 antibody.

In certain embodiments, Z is CR⁴ or N. In certain embodiments, Z is CR⁴. In certain embodiments, Z is N.

In certain embodiments, R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl.

In certain embodiments, R¹ is hydrogen. In certain embodiments, R¹ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹ is methyl. In certain embodiments, R¹ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹ is alkynyl or substituted alkynyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl.

In certain embodiments, R² is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R² is hydrogen. In certain embodiments, R² is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R² is methyl. In certain embodiments, R² is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R² is alkynyl or substituted alkynyl. In certain embodiments, R² is alkoxy or substituted alkoxy. In certain embodiments, R² is amino or substituted amino. In certain embodiments, R² is carboxyl or carboxyl ester. In certain embodiments, R² is acyl or acyloxy. In certain embodiments, R² is acyl amino or amino acyl. In certain embodiments, R² is alkylamide or substituted alkylamide. In certain embodiments, R² is sulfonyl. In certain embodiments, R² is thioalkoxy or substituted thioalkoxy. In certain embodiments, R² is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R² is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R² is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R² is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R³ is hydrogen. In certain embodiments, R³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³ is methyl. In certain embodiments, R³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³ is alkynyl or substituted alkynyl. In certain embodiments, R³ is alkoxy or substituted alkoxy. In certain embodiments, R³ is amino or substituted amino. In certain embodiments, R³ is carboxyl or carboxyl ester. In certain embodiments, R³ is acyl or acyloxy. In certain embodiments, R³ is acyl amino or amino acyl. In certain embodiments, R³ is alkylamide or substituted alkylamide. In certain embodiments, R³ is sulfonyl. In certain embodiments, R³ is thioalkoxy or substituted thioalkoxy. In certain embodiments, R³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R² and R³ are optionally cyclically linked to form a 5 or 6-membered heterocyclyl. In certain embodiments, R² and R³ are cyclically linked to form a 5 or 6-membered heterocyclyl. In certain embodiments, R² and R³ are cyclically linked to form a 5-membered heterocyclyl. In certain embodiments, R² and R³ are cyclically linked to form a 6-membered heterocyclyl.

In certain embodiments, each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

The various possibilities for each R⁴ are described in more detail as follows. In certain embodiments, R⁴ is hydrogen. In certain embodiments, each R⁴ is hydrogen. In certain embodiments, R⁴ is halogen, such as F, Cl, Br or I. In certain embodiments, R⁴ is F. In certain embodiments, R⁴ is Cl. In certain embodiments, R⁴ is Br. In certain embodiments, R⁴ is I. In certain embodiments, R⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R⁴ is methyl. In certain embodiments, R⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R⁴ is alkynyl or substituted alkynyl. In certain embodiments, R⁴ is alkoxy or substituted alkoxy. In certain embodiments, R⁴ is amino or substituted amino. In certain embodiments, R⁴ is carboxyl or carboxyl ester. In certain embodiments, R⁴ is acyl or acyloxy. In certain embodiments, R⁴ is acyl amino or amino acyl. In certain embodiments, R⁴ is alkylamide or substituted alkylamide. In certain embodiments, R⁴ is sulfonyl. In certain embodiments, R⁴ is thioalkoxy or substituted thioalkoxy. In certain embodiments, R⁴ is aryl or substituted aryl, such as C₅₋₈ aryl or C₅₋₈ substituted aryl, such as a C₅ aryl or C₅ substituted aryl, or a C₆ aryl or C₆ substituted aryl (e.g., phenyl or substituted phenyl). In certain embodiments, R⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R⁴ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, W¹ is a drug. Further description of the drug is found in the disclosure herein.

In certain embodiments, W² is an anti-MUC1 antibody. In certain embodiments, W² comprises one or more fGly′ residues as described herein. In certain embodiments, the anti-MUC1 antibody is attached to the rest of the conjugate through an fGly′ residue as described herein. Further description of anti-MUC1 antibodies that find use in the subject conjugates is found in the disclosure herein.

In certain embodiments, the compounds of formula (I) include a linker, L. The linker may be utilized to bind the conjugation moiety (e.g., a hydrazinyl-indolyl or a hydrazinyl-pyrrolo-pyridinyl conjugation moiety) to one or more moieties of interest. The linker may be bound (e.g., covalently bonded) to the conjugation moiety (e.g., as described herein) at any convenient position. For example, the linker may attach a hydrazinyl-indolyl or a hydrazinyl-pyrrolo-pyridinyl conjugation moiety to a drug. The hydrazinyl-indolyl or hydrazinyl-pyrrolo-pyridinyl coupling moiety may be used to conjugate the linker (and thus the drug) to a polypeptide, such as an anti-MUC1 antibody. For example, the conjugation moiety may be used to conjugate the linker (and thus the drug) to a modified amino acid residue of the polypeptide, such as an fGly residue of an anti-MUC1 antibody.

In certain embodiments, L attaches the conjugation moiety to W¹, and thus the conjugation moiety is indirectly bonded to W¹ through the linker L. As described above, W¹ is a drug, and thus L attaches the conjugation moiety to a drug, e.g., the conjugation moiety is indirectly bonded to the drug through the linker, L.

Any convenient linker may be utilized in the subject conjugates. In certain embodiments, L includes a group selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In certain embodiments, L includes an alkyl or substituted alkyl group. In certain embodiments, L includes an alkenyl or substituted alkenyl group. In certain embodiments, L includes an alkynyl or substituted alkynyl group. In certain embodiments, L includes an alkoxy or substituted alkoxy group. In certain embodiments, L includes an amino or substituted amino group. In certain embodiments, L includes a carboxyl or carboxyl ester group. In certain embodiments, L includes an acyl amino group. In certain embodiments, L includes an alkylamide or substituted alkylamide group. In certain embodiments, L includes an aryl or substituted aryl group. In certain embodiments, L includes a heteroaryl or substituted heteroaryl group. In certain embodiments, L includes a cycloalkyl or substituted cycloalkyl group. In certain embodiments, L includes a heterocyclyl or substituted heterocyclyl group.

In certain embodiments, L includes a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In certain embodiments, the polymer is a polyalkylene glycol. In certain embodiments, the polymer is a polyethylene glycol. Other linkers are also possible, as shown in the conjugates and compounds described in more detail below.

In some embodiments, L is a linker described by the formula:

-(L¹)ₐ-(L²)_{b}-(L³)_{c}-(L⁴)_{d}-(L⁵)ₑ-(L⁶)_{f}-,

wherein L¹, L², L³, L⁴, L⁵ and L⁶ are each independently a linker subunit, and a, b, c, d, e and f are each independently 0 or 1, wherein the sum of a, b, c, d, e and f is 1 to 6.

In certain embodiments, the sum of a, b, c, d, e and f is 1. In certain embodiments, the sum of a, b, c, d, e and f is 2. In certain embodiments, the sum of a, b, c, d, e and f is 3. In certain embodiments, the sum of a, b, c, d, e and f is 4. In certain embodiments, the sum of a, b, c, d, e and f is 5. In certain embodiments, the sum of a, b, c, d, e and f is 6. In certain embodiments, a, b, c, d, e and f are each 1. In certain embodiments, a, b, c, d and e are each 1 and f is 0. In certain embodiments, a, b, c and d are each 1 and e and f are each 0. In certain embodiments, a, b, and c are each 1 and d, e and f are each 0. In certain embodiments, a and b are each 1 and c, d, e and f are each 0. In certain embodiments, a is 1 and b, c, d, e and f are each 0.

In certain embodiments, the linker subunit L¹ is attached to the hydrazinyl-indolyl or the hydrazinyl-pyrrolo-pyridinyl conjugation moiety (e.g., as shown in formula (I) above). In certain embodiments, the linker subunit L², if present, is attached to drug. In certain embodiments, the linker subunit L³, if present, is attached to the drug. In certain embodiments, the linker subunit L⁴, if present, is attached to the drug. In certain embodiments, the linker subunit L⁵, if present, is attached to the drug. In certain embodiments, the linker subunit L⁶, if present, is attached to the drug.

Any convenient linker subunits may be utilized in the linker L. Linker subunits of interest include, but are not limited to, units of polymers such as polyethylene glycols, polyethylenes and polyacrylates, amino acid residue(s), carbohydrate-based polymers or carbohydrate residues and derivatives thereof, polynucleotides, alkyl groups, aryl groups, heterocyclic groups, combinations thereof, and substituted versions thereof. In some embodiments, each of L¹, L², L³, L⁴, L⁵ and L⁶ (if present) comprise one or more groups independently selected from a polyethylene glycol, a modified polyethylene glycol, an amino acid residue, an alkyl group, a substituted alkyl, an aryl group, a substituted aryl group, and a diamine (e.g., a linking group that includes an alkylene diamine).

In some embodiments, L¹ (if present) comprises a polyethylene glycol, a modified polyethylene glycol, an amino acid residue, an alkyl group, a substituted alkyl, an aryl group, a substituted aryl group, or a diamine. In some embodiments, L¹ comprises a polyethylene glycol. In some embodiments, L¹ comprises a modified polyethylene glycol. In some embodiments, L¹ comprises an amino acid residue. In some embodiments, L¹ comprises an alkyl group or a substituted alkyl. In some embodiments, L¹ comprises an aryl group or a substituted aryl group. In some embodiments, L¹ comprises a diamine (e.g., a linking group comprising an alkylene diamine).

In some embodiments, L² (if present) comprises a polyethylene glycol, a modified polyethylene glycol, an amino acid residue, an alkyl group, a substituted alkyl, an aryl group, a substituted aryl group, or a diamine. In some embodiments, L² comprises a polyethylene glycol. In some embodiments, L² comprises a modified polyethylene glycol. In some embodiments, L² comprises an amino acid residue. In some embodiments, L² comprises an alkyl group or a substituted alkyl. In some embodiments, L² comprises an aryl group or a substituted aryl group. In some embodiments, L² comprises a diamine (e.g., a linking group comprising an alkylene diamine).

In some embodiments, L³ (if present) comprises a polyethylene glycol, a modified polyethylene glycol, an amino acid residue, an alkyl group, a substituted alkyl, an aryl group, a substituted aryl group, or a diamine. In some embodiments, L³ comprises a polyethylene glycol. In some embodiments, L³ comprises a modified polyethylene glycol. In some embodiments, L³ comprises an amino acid residue. In some embodiments, L³ comprises an alkyl group or a substituted alkyl. In some embodiments, L³ comprises an aryl group or a substituted aryl group. In some embodiments, L³ comprises a diamine (e.g., a linking group comprising an alkylene diamine).

In some embodiments, L⁴ (if present) comprises a polyethylene glycol, a modified polyethylene glycol, an amino acid residue, an alkyl group, a substituted alkyl, an aryl group, a substituted aryl group, or a diamine. In some embodiments, L⁴ comprises a polyethylene glycol. In some embodiments, L⁴ comprises a modified polyethylene glycol. In some embodiments, L⁴ comprises an amino acid residue. In some embodiments, L⁴ comprises an alkyl group or a substituted alkyl. In some embodiments, L⁴ comprises an aryl group or a substituted aryl group. In some embodiments, L⁴ comprises a diamine (e.g., a linking group comprising an alkylene diamine).

In some embodiments, L⁵ (if present) comprises a polyethylene glycol, a modified polyethylene glycol, an amino acid residue, an alkyl group, a substituted alkyl, an aryl group, a substituted aryl group, or a diamine. In some embodiments, L⁵ comprises a polyethylene glycol. In some embodiments, L⁵ comprises a modified polyethylene glycol. In some embodiments, L⁵ comprises an amino acid residue. In some embodiments, L⁵ comprises an alkyl group or a substituted alkyl. In some embodiments, L⁵ comprises an aryl group or a substituted aryl group. In some embodiments, L⁵ comprises a diamine (e.g., a linking group comprising an alkylene diamine).

In some embodiments, L⁶ (if present) comprises a polyethylene glycol, a modified polyethylene glycol, an amino acid residue, an alkyl group, a substituted alkyl, an aryl group, a substituted aryl group, or a diamine. In some embodiments, L⁶ comprises a polyethylene glycol. In some embodiments, L⁶ comprises a modified polyethylene glycol. In some embodiments, L⁶ comprises an amino acid residue. In some embodiments, L⁶ comprises an alkyl group or a substituted alkyl. In some embodiments, L⁶ comprises an aryl group or a substituted aryl group. In some embodiments, L⁶ comprises a diamine (e.g., a linking group comprising an alkylene diamine).

In some embodiments, L is a linker comprising -(L¹)ₐ-(L²)_{b}-(L³)_{c}-(L⁴)_{d}-(L⁵)ₑ-(L⁶)_{f}-, where:
-(L¹)ₐ- is -(T¹-V¹)ₐ-;
-(L²)_{b}- is -(T²-V²)_{b}-;
-(L³)_{c}- is -(T³-V³)_{c}-;
-(L⁴)_{d}- is -(T⁴-V⁴)_{d}-;
-(L⁵)ₑ- is -(T⁵-V⁵)ₑ-; and
-(L⁶)_{f}- is -(T⁶-V⁶)_{f}-,
wherein T¹, T², T³, T⁴, T⁵ and T⁶, if present, are tether groups;
V¹, V², V³, V⁴, V⁵ and V⁶, if present, are covalent bonds or linking functional groups; and
a, b, c, d, e and f are each independently 0 or 1, wherein the sum of a, b, c, d, e and f is 1 to 6.

As described above, in certain embodiments, L¹ is attached to the hydrazinyl-indolyl or the hydrazinyl-pyrrolo-pyridinyl conjugation moiety (e.g., as shown in formula (I) above). As such, in certain embodiments, T¹ is attached to the hydrazinyl-indolyl or the hydrazinyl-pyrrolo-pyridinyl conjugation moiety (e.g., as shown in formula (I) above). In certain embodiments, V¹ is attached to the drug. In certain embodiments, L², if present, is attached to the drug. As such, in certain embodiments, T², if present, is attached to the drug, or V², if present, is attached to the drug. In certain embodiments, L³, if present, is attached to the drug. As such, in certain embodiments, T³, if present, is attached to the drug, or V³, if present, is attached to the drug. In certain embodiments, L⁴, if present, is attached to the drug. As such, in certain embodiments, T⁴, if present, is attached to the drug, or V⁴, if present, is attached to the drug. In certain embodiments, L⁵, if present, is attached to the drug. As such, in certain embodiments, T⁵, if present, is attached to the drug, or V⁵, if present, is attached to the drug. In certain embodiments, L⁶, if present, is attached to the drug. As such, in certain embodiments, T⁶, if present, is attached to the drug, or V⁶, if present, is attached to the drug.

Regarding the tether groups, T¹, T², T³, T⁴, T⁵ and T⁶, any convenient tether groups may be utilized in the subject linkers. In some embodiments, T¹, T², T³, T⁴, T⁵ and T⁶ each comprise one or more groups independently selected from a covalent bond, a (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, where each w is an integer from 1 to 20, each n is an integer from 1 to 30, each p is an integer from 1 to 20, and each m is an integer from 1 to 12.

In certain embodiments, the tether group (e.g., T¹, T², T³, T⁴, T⁵ and/or T⁶) includes a (C₁-C₁₂)alkyl or a substituted (C₁-C₁₂)alkyl. In certain embodiments, (C₁-C₁₂)alkyl is a straight chain or branched alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, (C₁-C₁₂)alkyl may be an alkyl or substituted alkyl, such as C₁-C₁₂ alkyl, or C₁-C₁₀ alkyl, or C₁-C₆ alkyl, or C₁-C₃ alkyl. In some instances, (C₁-C₁₂)alkyl is a C₂-alkyl. For example, (C₁-C₁₂)alkyl may be an alkylene or substituted alkylene, such as C₁-C₁₂ alkylene, or C₁-C₁₀ alkylene, or C₁-C₆ alkylene, or C₁-C₃ alkylene. In some instances, (C₁-C₁₂)alkyl is a C₂-alkylene (e.g., CH₂CH₂).

In certain embodiments, substituted (C₁-C₁₂)alkyl is a straight chain or branched substituted alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, substituted (C₁-C₁₂)alkyl may be a substituted alkyl, such as substituted C₁-C₁₂ alkyl, or substituted C₁-C₁₀ alkyl, or substituted C₁-C₆ alkyl, or substituted C₁-C₃ alkyl. In some instances, substituted (C₁-C₁₂)alkyl is a substituted C₂-alkyl. For example, substituted (C₁-C₁₂)alkyl may be a substituted alkylene, such as substituted C₁-C₁₂ alkylene, or substituted C₁-C₁₀ alkylene, or substituted C₁-C₆ alkylene, or substituted C₁-C₃ alkylene. In some instances, substituted (C₁-C₁₂)alkyl is a substituted C₂-alkylene.

In certain embodiments, the tether group (e.g., T¹, T², T³, T⁴, T⁵ and/or T⁶) includes an aryl, substituted aryl, heteroaryl, substituted heteroaryl, cycloalkyl, substituted cycloalkyl, heterocyclyl, or substituted heterocyclyl. In some instances, the tether group (e.g., T¹, T², T³, T⁴, T⁵ and T⁶) includes an aryl or substituted aryl. For example, the aryl can be phenyl. In some cases, the substituted aryl is a substituted phenyl. The substituted phenyl can be substituted with one or more substituents selected from (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl. In some instances, the substituted aryl is a substituted phenyl, where the substituent includes a cleavable moiety as described herein (e.g., an enzymatically cleavable moiety, such as a glycoside or glycoside derivative).

In some instances, the tether group (e.g., T¹, T², T³, T⁴, T⁵ and/or T⁶) includes a heteroaryl or substituted heteroaryl. In some instances, the tether group (e.g., T¹, T², T³, T⁴, T⁵ and T⁶) includes a cycloalkyl or substituted cycloalkyl. In some instances, the tether group (e.g., T¹, T², T³, T⁴, T⁵ and T⁶) includes a heterocyclyl or substituted heterocyclyl. In some instances, the substituent on the substituted heteroaryl, substituted cycloalkyl or substituted heterocyclyl includes a cleavable moiety as described herein (e.g., an enzymatically cleavable moiety, such as a glycoside or glycoside derivative).

In certain embodiments, the tether group (e.g., T¹, T², T³, T⁴, T⁵ and/or T⁶) includes an ethylene diamine (EDA) moiety, e.g., an EDA containing tether group. In certain embodiments, (EDA)_{w} includes one or more EDA moieties, such as where w is an integer from 1 to 50, such as from 1 to 40, from 1 to 30, from 1 to 20, from 1 to 12 or from 1 to 6, such as 1, 2, 3, 4, 5 or 6). The linked ethylene diamine (EDA) moieties may optionally be substituted at one or more convenient positions with any convenient substituents, e.g., with an alkyl, a substituted alkyl, an acyl, a substituted acyl, an aryl or a substituted aryl. In certain embodiments, the EDA moiety is described by the structure:

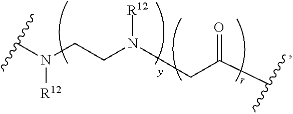

where y is an integer from 1 to 6, or is 0 or 1, and each R¹² is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, y is 1, 2, 3, 4, 5 or 6. In certain embodiments, y is 1 and r is 0. In certain embodiments, y is 1 and r is 1. In certain embodiments, y is 2 and r is 0. In certain embodiments, y is 2 and r is 1. In certain embodiments, each R¹² is independently selected from hydrogen, an alkyl, a substituted alkyl, an aryl and a substituted aryl. In certain embodiments, any two adjacent R¹² groups of the EDA may be cyclically linked, e.g., to form a piperazinyl ring. In certain embodiments, y is 1 and the two adjacent R¹² groups are an alkyl group, cyclically linked to form a piperazinyl ring. In certain embodiments, y is 1 and the adjacent R¹² groups are selected from hydrogen, an alkyl (e.g., methyl) and a substituted alkyl (e.g., lower alkyl-OH, such as ethyl-OH or propyl-OH).

In certain embodiments, the tether group (e.g., T¹, T², T³, T⁴, T⁵ and/or T⁶) includes a 4-amino-piperidine (4AP) moiety (also referred to herein as piperidin-4-amino, P4A). The 4AP moiety may optionally be substituted at one or more convenient positions with any convenient substituents, e.g., with an alkyl, a substituted alkyl, a polyethylene glycol moiety, an acyl, a substituted acyl, an aryl or a substituted aryl. In certain embodiments, the 4AP moiety is described by the structure:

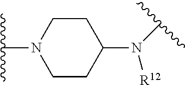

where R¹² is selected from hydrogen, alkyl, substituted alkyl, a polyethylene glycol moiety (e.g., a polyethylene glycol or a modified polyethylene glycol), alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹² is a polyethylene glycol moiety. In certain embodiments, R¹² is a carboxy modified polyethylene glycol.

In certain embodiments, R¹² includes a polyethylene glycol moiety described by the formula: (PEG)ₖ, which may be represented by the structure:

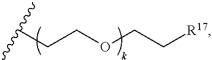

where k is an integer from 1 to 20, such as from 1 to 18, or from 1 to 16, or from 1 to 14, or from 1 to 12, or from 1 to 10, or from 1 to 8, or from 1 to 6, or from 1 to 4, or 1 or 2, such as 1, 2, 3, 4, 5, 6, 7, 8, 9, 10, 11, 12, 13, 14, 15, 16, 17, 18, 19 or 20. In some instances, k is 2. In certain embodiments, R¹⁷ is selected from OH, COOH, or COOR, where R is selected from alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R¹⁷ is COOH.

In certain embodiments, a tether group (e.g., T¹, T², T³, T⁴, T⁵ and/or T⁶) includes (PEG)ₙ, where (PEG)ₙ is a polyethylene glycol or a modified polyethylene glycol linking unit. In certain embodiments, (PEG))ₙ is described by the structure:

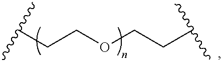

where n is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In some instances, n is 2. In some instances, n is 3. In some instances, n is 6. In some instances, n is 12.

In certain embodiments, a tether group (e.g., T¹, T², T³, T⁴, T⁵ and/or T⁶) includes (AA)ₚ, where AA is an amino acid residue. Any convenient amino acids may be utilized. Amino acids of interest include but are not limited to, L- and D-amino acids, naturally occurring amino acids such as any of the 20 primary alpha-amino acids and beta-alanine, non-naturally occurring amino acids (e.g., amino acid analogs), such as a non-naturally occurring alpha-amino acid or a non-naturally occurring beta-amino acid, etc. In certain embodiments, p is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In certain embodiments, p is 1. In certain embodiments, p is 2.

In certain embodiments, a tether group (e.g., T¹, T², T³, T⁴, T⁵ and/or T⁶) includes an amino acid analog. Amino acid analogs include compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or combinations thereof. For example, amino acid analogs may include α-hydroxy acids, and α-amino acids, and the like. Examples of amino acid analogs include, but are not limited to, sulfoalanine, and the like.

In certain embodiments, a tether group (e.g., T¹, T², T³, T⁴, T⁵ and/or T⁶) includes a moiety described by the formula —(CR¹³OH)ₘ—, where m is 0 or n is an integer from 1 to 50, such as from 1 to 40, from 1 to 30, from 1 to 20, from 1 to 12 or from 1 to 6, such as 1, 2, 3, 4, 5, 6, 7, 8, 9, 10, 11 or 12. In certain embodiments, m is 1. In certain embodiments, m is 2. In certain embodiments, R¹³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹³ is hydrogen. In certain embodiments, R¹³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹³ is alkynyl or substituted alkynyl. In certain embodiments, R¹³ is alkoxy or substituted alkoxy. In certain embodiments, R¹³ is amino or substituted amino. In certain embodiments, R¹³ is carboxyl or carboxyl ester. In certain embodiments, R¹³ is acyl or acyloxy. In certain embodiments, R¹³ is acyl amino or amino acyl. In certain embodiments, R¹³ is alkylamide or substituted alkylamide. In certain embodiments, R¹³ is sulfonyl. In certain embodiments, R¹³ is thioalkoxy or substituted thioalkoxy. In certain embodiments, R¹³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R¹³ is selected from hydrogen, alkyl, substituted alkyl, aryl, and substituted aryl. In these embodiments, alkyl, substituted alkyl, aryl, and substituted aryl are as described above for R¹³.

In certain embodiments, a tether group (e.g., T¹, T², T³, T⁴, T⁵ and/or T⁶) includes a meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), or para-hydroxy-phenyl (PHP).

In some embodiments, a tether includes a MABO group described by the following structure:

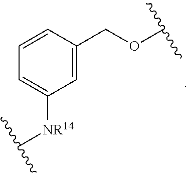

In some embodiments, a tether includes a MABC group described by the following structure:

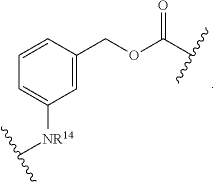

In some embodiments, a tether includes a PABO group described by the following structure:

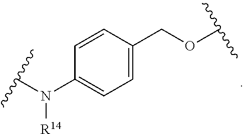

In some embodiments, a tether includes a PABC group described by the following structure:

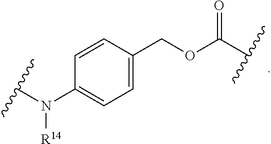

In some embodiments, a tether includes a PAB group described by the following structure:

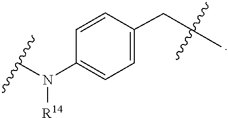

In some embodiments, a tether includes a PABA group described by the following structure:

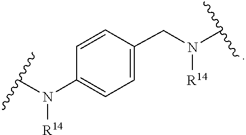

In some embodiments, a tether includes a PAP group described by the following structure:

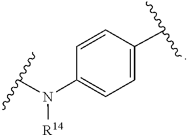

In some embodiments, a tether includes a PHP group described by the following structure:

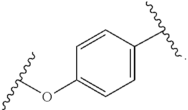 [SMILES: *Oc1ccc(*)cc1]

In certain embodiments, each R¹⁴ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, R¹⁴ is hydrogen. In certain embodiments, each R¹⁴ is hydrogen. In certain embodiments, R¹⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹⁴ is alkynyl or substituted alkynyl. In certain embodiments, R¹⁴ is alkoxy or substituted alkoxy. In certain embodiments, R¹⁴ is amino or substituted amino. In certain embodiments, R¹⁴ is carboxyl or carboxyl ester. In certain embodiments, R¹⁴ is acyl or acyloxy. In certain embodiments, R¹⁴ is acyl amino or amino acyl. In certain embodiments, R¹⁴ is alkylamide or substituted alkylamide. In certain embodiments, R¹⁴ is sulfonyl. In certain embodiments, R¹⁴ is thioalkoxy or substituted thioalkoxy. In certain embodiments, R¹⁴ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹⁴ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In some embodiments of the MABO, MABC, PABO, PABC, PAB, PABA, PAP, and PHP tether structures shown above, the phenyl ring may be substituted with one or more additional groups selected from halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments of the linker L, one or more of the tether groups T¹, T², T³, T⁴, T⁵ or T⁶ is each optionally substituted with a glycoside or glycoside derivative. In certain embodiments, the glycoside or glycoside derivative is selected from a glucuronide, a galactoside, a glucoside, a mannoside, a fucoside, O-GlcNAc, and O-GalNAc.

In certain embodiments, the MABO, MABC, PABO, PABC, PAB, PABA, PAP, and PHP tether structures shown above may be substituted with one or more additional groups selected from a glycoside and a glycoside derivative. For example, in some embodiments of the MABO, MABC, PABO, PABC, PAB, PABA, PAP, and PHP tether structures shown above, the phenyl ring may be substituted with one or more additional groups selected from a glycoside and a glycoside derivative. In certain embodiments, the glycoside or glycoside derivative is selected from a glucuronide, a galactoside, a glucoside, a mannoside, a fucoside, O-GlcNAc, and O-GalNAc.

For example, in some embodiments, the glycoside or glycoside derivative can be selected from the following structures:

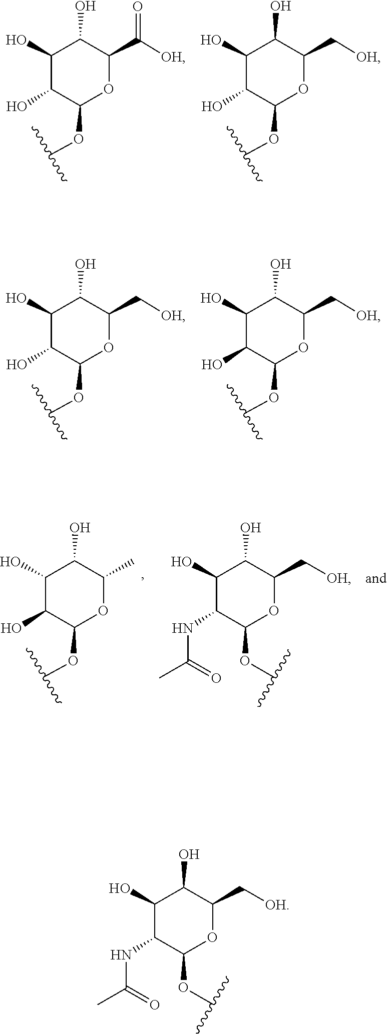

Regarding the linking functional groups, V¹, V², V³, V⁴, V⁵ and V⁶, any convenient linking functional groups may be utilized in the linker L. Linking functional groups of interest include, but are not limited to, amino, carbonyl, amido, oxycarbonyl, carboxy, sulfonyl, sulfoxide, sulfonylamino, aminosulfonyl, thio, oxy, phospho, phosphoramidate, thiophosphoraidate, and the like. In some embodiments, V¹, V², V³, V⁴, V⁵ and V⁶ are each independently selected from a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, where q is an integer from 1 to 6. In certain embodiments, q is an integer from 1 to 6 (e.g., 1, 2, 3, 4, 5 or 6). In certain embodiments, q is 1. In certain embodiments, q is 2. In certain embodiments, q is 3. In certain embodiments, q is 4. In certain embodiments, q is 5. In certain embodiments, q is 6.

In some embodiments, each R¹⁵ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, R¹⁵ is hydrogen. In certain embodiments, each R¹⁵ is hydrogen. In certain embodiments, R¹⁵ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹⁵ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹⁵ is alkynyl or substituted alkynyl. In certain embodiments, R¹⁵ is alkoxy or substituted alkoxy. In certain embodiments, R¹⁵ is amino or substituted amino. In certain embodiments, R¹⁵ is carboxyl or carboxyl ester. In certain embodiments, R¹⁵ is acyl or acyloxy. In certain embodiments, R¹⁵ is acyl amino or amino acyl. In certain embodiments, R¹⁵ is alkylamide or substituted alkylamide. In certain embodiments, R¹⁵ is sulfonyl. In certain embodiments, R¹⁵ is thioalkoxy or substituted thioalkoxy. In certain embodiments, R¹⁵ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹⁵ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹⁵ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹⁵ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl. In these embodiments, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl are as described above for R¹⁵.

In certain embodiments, the tether group includes an acetal group, a disulfide, a hydrazine, or an ester. In some embodiments, the tether group includes an acetal group. In some embodiments, the tether group includes a hydrazine. In some embodiments, the tether group includes a disulfide. In some embodiments, the tether group includes an ester.

As described above, in some embodiments, L is a linker comprising -(T¹-V¹)ₐ-(T²-V²)_{b}-(T³-V³)_{c}-(T⁴-V⁴)_{d}-(T⁵-V⁵)ₑ-(T⁶-V⁶)_{f}-, where a, b, c, d, e and f are each independently 0 or 1, where the sum of a, b, c, d, e and f is 1 to 6.

In some embodiments, in the linker L:
T¹ is selected from a (C₁-C₁₂)alkyl and a substituted (C₁-C₁₂)alkyl;
T², T³, T⁴, T⁵ and T⁶ are each independently selected from (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, 4-amino-piperidine (4AP), MABO, MABC, PABO, PABC, PAB, PABA, PAP, PHP, an acetal group, a disulfide, a hydrazine, and an ester; and
V¹, V², V³, V⁴, V⁵ and V⁶ are each independently selected from a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6;
wherein:
(PEG)ₙ is

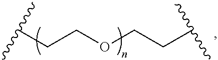

where n is an integer from 1 to 30;
EDA is an ethylene diamine moiety having the following structure:

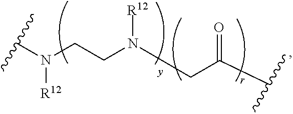

where y is an integer from 1 to 6 and r is 0 or 1;
4-amino-piperidine (4AP) is

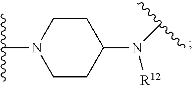

AA is an amino acid residue, where p is an integer from 1 to 20; and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

In certain embodiments, T¹, T², T³, T⁴, T⁵ and T⁶ and V¹, V², V³, V⁴, V⁵ and V⁶ are selected from the following:
wherein:
   T¹ is (C₁-C₁₂)alkyl and V¹ is —CO—;
   T² is an amino acid analog and V² is —NH—;
   T³ is (PEG)ₙ and V³ is —CO—;
   T⁴ is AA and V⁴ is absent;
   T⁵ is PABC and V⁵ is absent; and
   f is 0; or
wherein:
   T¹ is (C₁-C₁₂)alkyl and V¹ is —CO—;
   T² is 4AP and V² is —CO—;
   T³ is (C₁-C₁₂)alkyl and V³ is —CO—;
   d, e and f are each 0.

For example, in certain embodiments, the conjugate of formula (I) has a structure selected from the following:

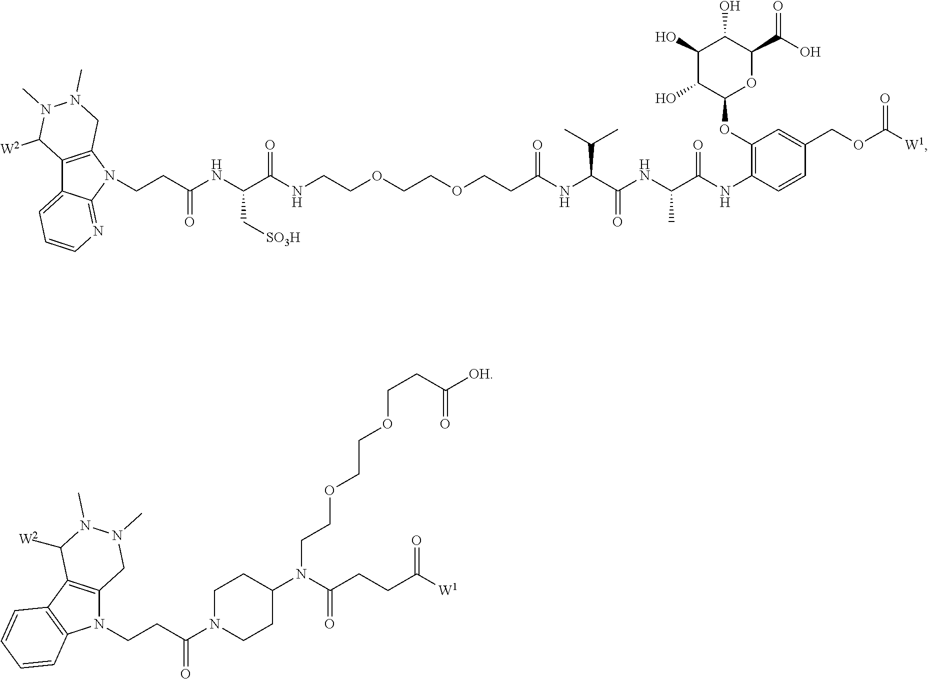

In certain embodiments, the left-hand side of the linker structure is attached to the hydrazinyl-indolyl or the hydrazinyl-pyrrolo-pyridinyl conjugation moiety, and the right-hand side of the linker structure is attached to the drug W¹, e.g., as shown above.

In certain embodiments, the conjugate is an antibody-drug conjugate where the antibody and the drug are linked together by a linker (e.g., L), as described above. In some instances, the linker is a cleavable linker. A cleavable linker is a linker that includes one or more cleavable moieties, where the cleavable moiety includes one or more bonds that can dissociate under certain conditions, thus separating the cleavable linker into two or more separatable portions. For example, the cleavable moiety may include one or more covalent bonds, which under certain conditions, can dissociate or break apart to separate the cleavable linker into two or more portions. As such a cleavable linker can be included in an antibody-drug conjugate, such that under appropriate conditions, the cleavable linker is cleaved to separate or release the drug from the antibody at a desired target site of action for the drug.

In some instances, the cleavable linker includes two cleavable moieties, such as a first cleavable moiety and a second cleavable moiety. The cleavable moieties can be configured such that cleavage of both cleavable moieties is needed in order to separate or release the drug from the antibody at a desired target site of action for the drug. For example, cleavage of the cleavable linker can be achieved by initially cleaving one of the two cleavable moieties and then cleaving the other of the two cleavable moieties. In certain embodiments, the cleavable linker includes a first cleavable moiety and a second cleavable moiety that hinders cleavage of the first cleavable moiety. By “hinders cleavage” is meant that the presence of an uncleaved second cleavable moiety reduces the likelihood or substantially inhibits the cleavage of the first cleavable moiety, thus substantially reducing the amount or preventing the cleavage of the cleavable linker. For instance, the presence of uncleaved second cleavable moiety can hinder cleavage of the first cleavable moiety. The hinderance of cleavage of the first cleavable moiety by the presence of the second cleavable moiety, in turn, substantially reduces the amount or prevents the release of the drug from the antibody. For example, the premature release of the drug from the antibody can be substantially reduced or prevented until the antibody-drug conjugate is at or near the desired target site of action for the drug.

In some cases, since the second cleavable moiety hinders cleavage of the first cleavable moiety, cleavage of the cleavable linker can be achieved by initially cleaving the second cleavable moiety and then cleaving the first cleavable moiety. Cleavage of the second cleavable moiety can reduce or eliminate the hinderance on the cleavage of the first cleavable moiety, thus allowing the first cleavable moiety to be cleaved. Cleavage of the first cleavable moiety can result in the cleavable linker dissociating or separating into two or more portions as described above to release the drug from the antibody-drug conjugate. In some instances, cleavage of the first cleavable moiety does not substantially occur in the presence of an uncleaved second cleavable moiety. By substantially is meant that about 10% or less cleavage of the first cleavable moiety occurs in the presence of an uncleaved second cleavable moiety, such as about 9% or less, or about 8% or less, or about 7% or less, or about 6% or less, or about 5% or less, or about 4% or less, or about 3% or less, or about 2% or less, or about 1% or less, or about 0.5% or less, or about 0.1% or less cleavage of the first cleavable moiety occurs in the presence of an uncleaved second cleavable moiety.

Stated another way, the second cleavable moiety can protect the first cleavable moiety from cleavage. For instance, the presence of uncleaved second cleavable moiety can protect the first cleavable moiety from cleavage, and thus substantially reduce or prevent premature release of the drug from the antibody until the antibody-drug conjugate is at or near the desired target site of action for the drug. As such, cleavage of the second cleavable moiety exposes the first cleavable moiety (e.g., deprotects the first cleavable moiety), thus allowing the first cleavable moiety to be cleaved, which results in cleavage of the cleavable linker, which, in turn, separates or releases the drug from the antibody at a desired target site of action for the drug as described above. In certain instances, cleavage of the second cleavable moiety exposes the first cleavable moiety to subsequent cleavage, but cleavage of the second cleavable moiety does not in and of itself result in cleavage of the cleavable linker (e.g., cleavage of the first cleavable moiety is still needed in order to cleave the cleavable linker).

The cleavable moieties included in the cleavable linker may each be an enzymatically cleavable moiety. For example, the first cleavable moiety can be a first enzymatically cleavable moiety and the second cleavable moiety can be a second enzymatically cleavable moiety. An enzymatically cleavable moiety is a cleavable moiety that can be separated into two or more portions as described above through the enzymatic action of an enzyme. The enzymatically cleavable moiety can be any cleavable moiety that can be cleaved through the enzymatic action of an enzyme, such as, but not limited to, a peptide, a glycoside, and the like. In some instances, the enzyme that cleaves the enzymatically cleavable moiety is present at a desired target site of action, such as the desired target site of action of the drug that is to be released from the antibody-drug conjugate. In some cases, the enzyme that cleaves the enzymatically cleavable moiety is not present in a significant amount in other areas, such as in whole blood, plasma or serum. As such, the cleavage of an enzymatically cleavable moiety can be controlled such that substantial cleavage occurs at the desired site of action, whereas cleavage does not significantly occur in other areas or before the antibody-drug conjugate reaches the desired site of action.

For example, as described herein, antibody-drug conjugates of the present disclosure can be used for the treatment of cancer, such as for the delivery of a cancer therapeutic drug to a desired site of action where the cancer cells are present. In some cases, enzymes, such as the protease enzyme cathepsin B, can be a biomarker for cancer that is overexpressed in cancer cells. The overexpression, and thus localization, of certain enzymes in cancer can be used in the context of the enzymatically cleavable moieties included in the cleavable linkers of the antibody-drug conjugates of the present disclosure to specifically release the drug at the desired site of action (e.g., the site of the cancer (and overexpressed enzyme)). Thus, in some embodiments, the enzymatically cleavable moiety is a cleavable moiety (e.g., a peptide) that can be cleaved by an enzyme that is overexpressed in cancer cells. For instance, the enzyme can be the protease enzyme cathepsin B. As such, in some instances, the enzymatically cleavable moiety is a cleavable moiety (e.g., a peptide) that can be cleaved by a protease enzyme, such as cathepsin B.

In certain embodiments, the enzymatically cleavable moiety is a peptide. The peptide can be any peptide suitable for use in the cleavable linker and that can be cleaved through the enzymatic action of an enzyme. Non-limiting examples of peptides that can be used as an enzymatically cleavable moiety include, for example, Val-Ala, Phe-Lys, and the like. For example, the first cleavable moiety described above (e.g., the cleavable moiety protected from premature cleavage by the second cleavable moiety) can include a peptide. The presence of uncleaved second cleavable moiety can protect the first cleavable moiety (peptide) from cleavage by a protease enzyme (e.g., cathepsin B), and thus substantially reduce or prevent premature release of the drug from the antibody until the antibody-drug conjugate is at or near the desired target site of action for the drug. In some instances, one of the amino acid residues of the peptide that comprises the first cleavable moiety is linked to or includes a substituent, where the substituent comprises the second cleavable moiety. In some instances, the second cleavable moiety includes a glycoside.

In some embodiments, the enzymatically cleavable moiety is sugar moiety, such as a glycoside (or glyosyl). In some cases, the glycoside can facilitate an increase in the hydrophilicity of the cleavable linker as compared to a cleavable linker that does not include the glycoside. The glycoside can be any glycoside or glycoside derivative suitable for use in the cleavable linker and that can be cleaved through the enzymatic action of an enzyme. For example, the second cleavable moiety (e.g., the cleavable moiety that protects the first cleavable moiety from premature cleavage) can be a glycoside. For instance, in some embodiments, the first cleavable moiety includes a peptide and the second cleavable moiety includes a glycoside. In certain embodiments, the second cleavable moiety is a glycoside or glycoside derivative selected from a glucuronide, a galactoside, a glucoside, a mannoside, a fucoside, O-GlcNAc, and O-GalNAc. In some instances, the second cleavable moiety is a glucuronide. In some instances, the second cleavable moiety is a galactoside. In some instances, the second cleavable moiety is a glucoside. In some instances, the second cleavable moiety is a mannoside. In some instances, the second cleavable moiety is a fucoside. In some instances, the second cleavable moiety is O-GlcNAc. In some instances, the second cleavable moiety is O-GalNAc.

The glycoside can be attached (e.g., covalently bonded) to the cleavable linker through a glycosidic bond. The glycosidic bond can link the glycoside to the cleavable linker through various types of bonds, such as, but not limited to, an O-glycosidic bond (an O-glycoside), an N-glycosidic bond (a glycosylamine), an S-glycosidic bond (a thioglycoside), or C-glycosidic bond (a C-glycoside or C-glycosyl). In some instances, the glycosidic bond is an O-glycosidic bond (an O-glycoside). In some cases, the glycoside can be cleaved from the cleavable linker it is attached to by an enzyme (e.g., through enzymatically-mediated hydrolysis of the glycosidic bond). A glycoside can be removed or cleaved from the cleavable linker by any convenient enzyme that is able to carry out the cleavage (hydrolysis) of the glycosidic bond that attaches the glycoside to the cleavable linker. An example of an enzyme that can be used to mediate the cleavage (hydrolysis) of the glycosidic bond that attaches the glycoside to the cleavable linker is a glucuronidase, a glycosidase, such as a galactosidase, a glucosidase, a mannosidase, a fucosidase, and the like. Other suitable enzymes may also be used to mediate the cleavage (hydrolysis) of the glycosidic bond that attaches the glycoside to the cleavable linker. In some cases, the enzyme used to mediate the cleavage (hydrolysis) of the glycosidic bond that attaches the glycoside to the cleavable linker is found at or near the desired site of action for the drug of the antibody-drug conjugate. For instance, the enzyme can be a lysosomal enzyme, such as a lysosomal glycosidase, found in cells at or near the desired site of action for the drug of the antibody-drug conjugate. In some cases, the enzyme is an enzyme found at or near the target site where the enzyme that mediates cleavage of the first cleavable moiety is found.

In certain embodiments, the conjugate of formula (I) has a structure selected from the following:

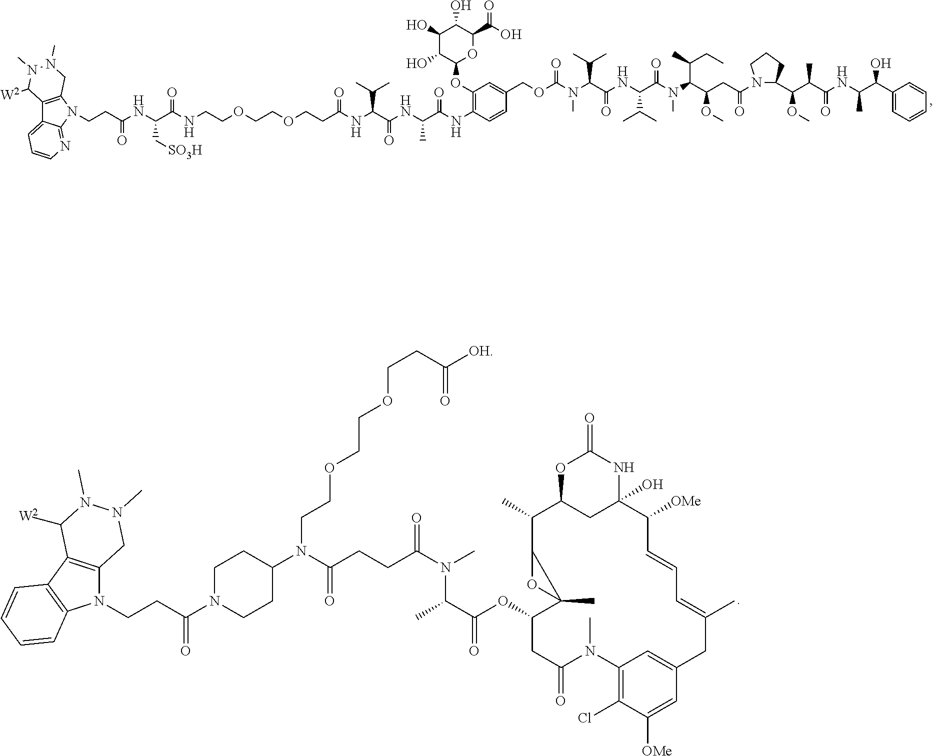

Any of the chemical entities, drugs, linkers and coupling moieties set forth in the description and structures above may be adapted for use in the subject conjugates.

Additional disclosure related to hydrazinyl-indolyl and hydrazinyl-pyrrolo-pyridinyl compounds and methods for producing a conjugate is found in U.S. Pat. Nos. 9,310,374 and 9,493,413, the disclosures of each of which are incorporated herein by reference. Additional disclosure related to cleavable linkers is found in U.S. Provisional Application No. 63/214,525, filed Jun. 24, 2021, the disclosure of which is incorporated herein by reference.

Aspects of the present disclosure include a conjugate of formula (II):

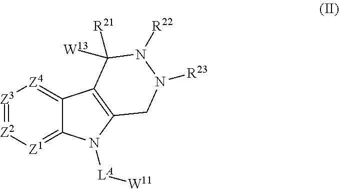

wherein:
Z¹, Z², Z³ and Z⁴ are each independently selected from CR²⁴, N and C-L^{B}-W¹², wherein at least one Z¹, Z², Z³ and Z⁴ is C-L^{B}-W¹²;
R²¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R²² and R²³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R²² and R²³ are optionally cyclically linked to form a 5 or 6-membered heterocyclyl;
each R²⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L^{A} is a first linker;
L^{B} is a second linker;
W¹¹ is a first drug;
W¹² is a second drug; and
W¹³ is a polypeptide.

The substituents related to conjugates of formula (II) are described in more detail below.

In certain embodiments, Z¹, Z², Z³ and Z⁴ are each independently selected from CR²⁴, N and C-L^{B}-W¹², wherein at least one Z¹, Z², Z³ and Z⁴ is C-L^{B}-W¹². In certain embodiments, Z¹ is CR²⁴. In certain embodiments, Z¹ is N. In certain embodiments, Z¹ is C-L^{B}-W¹². In certain embodiments, Z² is CR²⁴. In certain embodiments, Z² is N. In certain embodiments, Z² is C-L^{B}-W¹². In certain embodiments, Z³ is CR²⁴. In certain embodiments, Z³ is N. In certain embodiments, Z³ is C-L^{B}-W¹². In certain embodiments, Z⁴ is CR²⁴. In certain embodiments, Z⁴ is N. In certain embodiments, Z⁴ is C-L^{B}-W¹².

Combinations of various Z¹, Z², Z³ and Z⁴ are possible. For example, in some instances, Z¹ is C-L^{B}-W¹², Z² is CR²⁴, Z³ is CR²⁴, and Z⁴ is CR²⁴. In some instances, Z¹ is CR²⁴, Z² is C-L^{B}-W¹², Z³ is CR²⁴, and Z⁴ is CR²⁴. In some instances, Z¹ is CR²⁴, Z² is CR²⁴, Z³ is C-L^{B}-W¹², and Z⁴ is CR²⁴. In some instances, Z¹ is CR²⁴, Z² is CR²⁴, Z³ is CR²⁴, and Z⁴ is C-L^{B}-W¹².

In certain embodiments, R²¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, substituted heterocyclyl. In certain embodiments, R²¹ is hydrogen. In certain embodiments, R²¹ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R²¹ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R²¹ is alkynyl or substituted alkynyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R²¹ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R²¹ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R²¹ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R²¹ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R²² and R²³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R²² and R²³ are optionally cyclically linked to form a 5 or 6-membered heterocyclyl.

In certain embodiments, R²² is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R²² is hydrogen. In certain embodiments, R²² is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R²² is methyl. In certain embodiments, R²² is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R²² is alkynyl or substituted alkynyl. In certain embodiments, R²² is alkoxy or substituted alkoxy. In certain embodiments, R²² is amino or substituted amino. In certain embodiments, R²² is carboxyl or carboxyl ester. In certain embodiments, R²² is acyl or acyloxy. In certain embodiments, R²² is acyl amino or amino acyl. In certain embodiments, R²² is alkylamide or substituted alkylamide. In certain embodiments, R²² is sulfonyl. In certain embodiments, R²² is thioalkoxy or substituted thioalkoxy. In certain embodiments, R²² is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R²² is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R²² is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R²² is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R²³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R²³ is hydrogen. In certain embodiments, R²³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R²³ is methyl. In certain embodiments, R²³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R²³ is alkynyl or substituted alkynyl. In certain embodiments, R²³ is alkoxy or substituted alkoxy. In certain embodiments, R²³ is amino or substituted amino. In certain embodiments, R²³ is carboxyl or carboxyl ester. In certain embodiments, R²³ is acyl or acyloxy. In certain embodiments, R²³ is acyl amino or amino acyl. In certain embodiments, R²³ is alkylamide or substituted alkylamide. In certain embodiments, R²³ is sulfonyl. In certain embodiments, R²³ is thioalkoxy or substituted thioalkoxy. In certain embodiments, R²³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R²³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R²³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R²³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiment, both R²² and R²³ are methyl.

In certain embodiments, R²² and R²³ are optionally cyclically linked to form a 5 or 6-membered heterocyclyl. In certain embodiments, R²² and R²³ are cyclically linked to form a 5 or 6-membered heterocyclyl. In certain embodiments, R²² and R²³ are cyclically linked to form a 5-membered heterocyclyl. In certain embodiments, R²² and R²³ are cyclically linked to form a 6-membered heterocyclyl.

In certain embodiments, each R²⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

The various possibilities for each R²⁴ are described in more detail as follows. In certain embodiments, R²⁴ is hydrogen. In certain embodiments, each R²⁴ is hydrogen. In certain embodiments, R²⁴ is halogen, such as F, Cl, Br or I. In certain embodiments, R²⁴ is F. In certain embodiments, R²⁴ is Cl. In certain embodiments, R²⁴ is Br. In certain embodiments, R²⁴ is I. In certain embodiments, R²⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R²⁴ is methyl. In certain embodiments, R²⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R²⁴ is alkynyl or substituted alkynyl. In certain embodiments, R²⁴ is alkoxy or substituted alkoxy. In certain embodiments, R²⁴ is amino or substituted amino. In certain embodiments, R²⁴ is carboxyl or carboxyl ester. In certain embodiments, R²⁴ is acyl or acyloxy. In certain embodiments, R²⁴ is acyl amino or amino acyl. In certain embodiments, R²⁴ is alkylamide or substituted alkylamide. In certain embodiments, R²⁴ is sulfonyl. In certain embodiments, R²⁴ is thioalkoxy or substituted thioalkoxy. In certain embodiments, R²⁴ is aryl or substituted aryl, such as C₅₋₈ aryl or C₅₋₈ substituted aryl, such as a C₅ aryl or C₅ substituted aryl, or a C₆ aryl or C₆ substituted aryl (e.g., phenyl or substituted phenyl). In certain embodiments, R²⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R²⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R²⁴ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, L^{A} is a first linker. Examples of linkers that can be used in the conjugates of the present disclosure are described in more detail below.

In certain embodiments, L^{B} is a second linker. Examples of linkers that can be used in the conjugates of the present disclosure are described in more detail below.

In certain embodiments, W¹¹ is a first drug (or a first active agent). Examples of drugs and active agents that can be used in the conjugates of the present disclosure are described in more detail below.

In certain embodiments, W¹¹ is a second drug (or a second active agent).

Examples of drugs and active agents that can be used in the conjugates of the present disclosure are described in more detail below.

In certain embodiments, W¹³ is a polypeptide (e.g., an antibody). In certain embodiments, W¹³ comprises one or more fGly′ residues as described herein. In certain embodiments, the polypeptide is attached to the rest of the conjugate through an fGly′ residue as described herein. Examples of polypeptides and antibodies that can be used in the conjugates of the present disclosure are described in more detail below.

In certain embodiments, the conjugate of formula (II) includes a first linker, L^{A}. The first linker, L^{A}, may be utilized to bind a first moiety of interest (e.g., a first drug or active agent) to a polypeptide (e.g., an antibody) through a conjugation moiety. The first linker, L^{A}, may be bound (e.g., covalently bonded) to the conjugation moiety (e.g., as described herein). For example, the first linker, L^{A}, may attach a hydrazinyl-indolyl or a hydrazinyl-pyrrolo-pyridinyl conjugation moiety to a first drug. The hydrazinyl-indolyl or hydrazinyl-pyrrolo-pyridinyl conjugation moiety may be used to conjugate the first linker, L^{A}, (and thus the first drug) to a polypeptide, such as an antibody.

For example, as shown in formula (II) above, L^{A} is attached to W¹³ through a conjugation moiety, and thus W¹³ is indirectly bonded to the linker L^{A} through the hydrazinyl-indolyl or a hydrazinyl-pyrrolo-pyridinyl conjugation moiety. As described above, W¹³ is a polypeptide (e.g., an antibody), and thus L^{A} is attached through the hydrazinyl-indolyl or a hydrazinyl-pyrrolo-pyridinyl conjugation moiety to the polypeptide (antibody), e.g., the linker L^{A} is indirectly bonded to the polypeptide (antibody) through the hydrazinyl-indolyl or a hydrazinyl-pyrrolo-pyridinyl conjugation moiety.

Any convenient linker may be utilized for the first linker L^{A} in the subject conjugates and compounds. In certain embodiments, the first linker L^{A} may include a group selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In certain embodiments, the first linker L^{A} may include an alkyl or substituted alkyl group. In certain embodiments, the first linker L^{A} may include an alkenyl or substituted alkenyl group. In certain embodiments, the first linker L^{A} may include an alkynyl or substituted alkynyl group. In certain embodiments, the first linker L^{A} may include an alkoxy or substituted alkoxy group. In certain embodiments, the first linker L^{A} may include an amino or substituted amino group. In certain embodiments, the first linker L^{A} may include a carboxyl or carboxyl ester group. In certain embodiments, the first linker L^{A} may include an acyl amino group. In certain embodiments, the first linker L^{A} may include an alkylamide or substituted alkylamide group. In certain embodiments, the first linker L^{A} may include an aryl or substituted aryl group. In certain embodiments, the first linker L^{A} may include a heteroaryl or substituted heteroaryl group. In certain embodiments, the first linker L^{A} may include a cycloalkyl or substituted cycloalkyl group. In certain embodiments, the first linker L^{A} may include a heterocyclyl or substituted heterocyclyl group.

In certain embodiments, the first linker L^{A} may include a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In certain embodiments, the polymer is a polyalkylene glycol. In certain embodiments, the polymer is a polyethylene glycol. Other linkers are also possible, as shown in the conjugates and compounds described in more detail below.

In some embodiments, L^{A} is a first linker described by the formula:

-(L¹)ₐ-(L²)_{b}-(L³)_{c}-(L⁴)_{d}-(L⁵)ₑ-(L⁶)_{f}-,
wherein L¹, L², L³, L⁴, L⁵ and L⁶ are each independently a linker subunit, and a, b, c, d, e and f are each independently 0 or 1.

In certain embodiments, the sum of a, b, c, d, e and f is 0 to 6. In certain embodiments, the sum of a, b, c, d, e and f is 0. In certain embodiments, the sum of a, b, c, d, e and f is 1. In certain embodiments, the sum of a, b, c, d, e and f is 2. In certain embodiments, the sum of a, b, c, d, e and f is 3. In certain embodiments, the sum of a, b, c, d, e and f is 4. In certain embodiments, the sum of a, b, c, d, e and f is 5. In certain embodiments, the sum of a, b, c, d, e and f is 6. In certain embodiments, a, b, c, d, e and f are each 1. In certain embodiments, a, b, c, d and e are each 1 and f is 0. In certain embodiments, a, b, c and d are each 1 and e and f are each 0. In certain embodiments, a, b, and c are each 1 and d, e and f are each 0. In certain embodiments, a and b are each 1 and c, d, e and f are each 0. In certain embodiments, a is 1 and b, c, d, e and f are each 0.

In certain embodiments, the linker subunit L¹ is attached to the hydrazinyl-indolyl or a hydrazinyl-pyrrolo-pyridinyl conjugation moiety (e.g., as shown in formula (I) above). In certain embodiments, the linker subunit L², if present, is attached to the first drug or active agent W¹¹. In certain embodiments, the linker subunit L³, if present, is attached to the first drug or active agent W¹¹. In certain embodiments, the linker subunit L⁴, if present, is attached to the first drug or active agent W¹¹. In certain embodiments, the linker subunit L⁵, if present, is attached to the first drug or active agent W¹¹. In certain embodiments, the linker subunit L⁶, if present, is attached to the first drug or active agent W¹¹.

Any convenient linker subunits may be utilized in the first linker L^{A}. Linker subunits of interest include, but are not limited to, units of polymers such as polyethylene glycols, polyethylenes and polyacrylates, amino acid residue(s), carbohydrate-based polymers or carbohydrate residues and derivatives thereof, polynucleotides, alkyl groups, aryl groups, heterocyclic groups, combinations thereof, and substituted versions thereof. In some embodiments, each of L¹, L², L³, L⁴, L⁵ and L⁶ (if present) comprise one or more groups independently selected from a polyethylene glycol, a modified polyethylene glycol, an amino acid residue, an alkyl group, a substituted alkyl, an aryl group, a substituted aryl group, and a diamine (e.g., a linking group that includes an alkylene diamine).

In some embodiments, L¹ (if present) comprises a polyethylene glycol, a modified polyethylene glycol, an amino acid residue, an alkyl group, a substituted alkyl, an aryl group, a substituted aryl group, or a diamine. In some embodiments, L¹ comprises a polyethylene glycol. In some embodiments, L¹ comprises a modified polyethylene glycol. In some embodiments, L¹ comprises an amino acid residue. In some embodiments, L¹ comprises an alkyl group or a substituted alkyl. In some embodiments, L¹ comprises an aryl group or a substituted aryl group. In some embodiments, L¹ comprises a diamine (e.g., a linking group comprising an alkylene diamine).

In some embodiments, L² (if present) comprises a polyethylene glycol, a modified polyethylene glycol, an amino acid residue, an alkyl group, a substituted alkyl, an aryl group, a substituted aryl group, or a diamine. In some embodiments, L² comprises a polyethylene glycol. In some embodiments, L² comprises a modified polyethylene glycol. In some embodiments, L² comprises an amino acid residue. In some embodiments, L² comprises an alkyl group or a substituted alkyl. In some embodiments, L² comprises an aryl group or a substituted aryl group. In some embodiments, L² comprises a diamine (e.g., a linking group comprising an alkylene diamine).

In some embodiments, L³ (if present) comprises a polyethylene glycol, a modified polyethylene glycol, an amino acid residue, an alkyl group, a substituted alkyl, an aryl group, a substituted aryl group, or a diamine. In some embodiments, L³ comprises a polyethylene glycol. In some embodiments, L³ comprises a modified polyethylene glycol. In some embodiments, L³ comprises an amino acid residue. In some embodiments, L³ comprises an alkyl group or a substituted alkyl. In some embodiments, L³ comprises an aryl group or a substituted aryl group. In some embodiments, L³ comprises a diamine (e.g., a linking group comprising an alkylene diamine).

In some embodiments, L⁴ (if present) comprises a polyethylene glycol, a modified polyethylene glycol, an amino acid residue, an alkyl group, a substituted alkyl, an aryl group, a substituted aryl group, or a diamine. In some embodiments, L⁴ comprises a polyethylene glycol. In some embodiments, L⁴ comprises a modified polyethylene glycol. In some embodiments, L⁴ comprises an amino acid residue. In some embodiments, L⁴ comprises an alkyl group or a substituted alkyl. In some embodiments, L⁴ comprises an aryl group or a substituted aryl group. In some embodiments, L⁴ comprises a diamine (e.g., a linking group comprising an alkylene diamine).

In some embodiments, L⁵ (if present) comprises a polyethylene glycol, a modified polyethylene glycol, an amino acid residue, an alkyl group, a substituted alkyl, an aryl group, a substituted aryl group, or a diamine. In some embodiments, L⁵ comprises a polyethylene glycol. In some embodiments, L⁵ comprises a modified polyethylene glycol. In some embodiments, L⁵ comprises an amino acid residue. In some embodiments, L⁵ comprises an alkyl group or a substituted alkyl. In some embodiments, L⁵ comprises an aryl group or a substituted aryl group. In some embodiments, L⁵ comprises a diamine (e.g., a linking group comprising an alkylene diamine).

In some embodiments, L⁶ (if present) comprises a polyethylene glycol, a modified polyethylene glycol, an amino acid residue, an alkyl group, a substituted alkyl, an aryl group, a substituted aryl group, or a diamine. In some embodiments, L⁶ comprises a polyethylene glycol. In some embodiments, L⁶ comprises a modified polyethylene glycol. In some embodiments, L⁶ comprises an amino acid residue. In some embodiments, L⁶ comprises an alkyl group or a substituted alkyl. In some embodiments, L⁶ comprises an aryl group or a substituted aryl group. In some embodiments, L⁶ comprises a diamine (e.g., a linking group comprising an alkylene diamine).

In some embodiments, L^{A} is a first linker comprising -(L¹)ₐ-(L²)_{b}-(L³)_{c}-(L⁴)_{d}-(L⁵)ₑ-(L⁶)_{f}-, where:
-(L¹)ₐ- is -(T¹I-V¹)ₐ-;
-(L²)_{b}- is -(T²-V²)_{b}-;
-(L³)_{c}- is -(T³-V³)_{c}-;
-(L⁴)_{d}- is -(T⁴-V⁴)_{d}-;
-(L⁵)ₑ- is -(T⁵-V⁵)ₑ-; and
-(L⁶)_{f}- is -(T⁶-V⁶)_{f}-,
wherein T, T², T³, T⁴, T⁵ and T⁶, if present, are tether groups;
V¹, V², V³, V⁴, V⁵ and V⁶, if present, are covalent bonds or linking functional groups; and
a, b, c, d, e and f are each independently 0 or 1.

In certain embodiments, the sum of a, b, c, d, e and f is 0 to 6. In certain embodiments, the sum of a, b, c, d, e and f is 0. In certain embodiments, the sum of a, b, c, d, e and f is 1. In certain embodiments, the sum of a, b, c, d, e and f is 2. In certain embodiments, the sum of a, b, c, d, e and f is 3. In certain embodiments, the sum of a, b, c, d, e and f is 4. In certain embodiments, the sum of a, b, c, d, e and f is 5. In certain embodiments, the sum of a, b, c, d, e and f is 6. In certain embodiments, a, b, c, d, e and f are each 1. In certain embodiments, a, b, c, d and e are each 1 and f is 0. In certain embodiments, a, b, c and d are each 1 and e and f are each 0. In certain embodiments, a, b, and c are each 1 and d, e and f are each 0. In certain embodiments, a and b are each 1 and c, d, e and f are each 0. In certain embodiments, a is 1 and b, c, d, e and f are each 0.

As described above, in certain embodiments, L¹ is attached to the hydrazinyl-indolyl or a hydrazinyl-pyrrolo-pyridinyl conjugation moiety (e.g., as shown in formula (II) above). As such, in certain embodiments, T¹ is attached to the hydrazinyl-indolyl or a hydrazinyl-pyrrolo-pyridinyl conjugation moiety (e.g., as shown in formula (II) above). In certain embodiments, V¹ is attached to the first drug or active agent. In certain embodiments, L², if present, is attached to the first drug or active agent. As such, in certain embodiments, T², if present, is attached to the first drug or active agent, or V², if present, is attached to the first drug or active agent. In certain embodiments, L³, if present, is attached to the first drug or active agent. As such, in certain embodiments, T³, if present, is attached to the first drug or active agent, or V³, if present, is attached to the first drug or active agent. In certain embodiments, L⁴, if present, is attached to the first drug or active agent. As such, in certain embodiments, T⁴, if present, is attached to the first drug or active agent, or V⁴, if present, is attached to the first drug or active agent. In certain embodiments, L⁵, if present, is attached to the first drug or active agent. As such, in certain embodiments, T⁵, if present, is attached to the first drug or active agent, or V⁵, if present, is attached to the first drug or active agent. In certain embodiments, L⁶, if present, is attached to the first drug or active agent. As such, in certain embodiments, T⁶, if present, is attached to the first drug or active agent, or V⁶, if present, is attached to the first drug or active agent.

In certain embodiments, the conjugate of formula (II) includes a second linker, L^{B}. The second linker, L^{B}, may be utilized to bind a second moiety of interest (e.g., a second drug or active agent) to a polypeptide (e.g., an antibody) through a conjugation moiety. The second linker, L^{B}, may be bound (e.g., covalently bonded) to the conjugation moiety (e.g., as described herein). For example, the second linker, L^{B}, may attach a hydrazinyl-indolyl or a hydrazinyl-pyrrolo-pyridinyl conjugation moiety to a second drug. The hydrazinyl-indolyl or hydrazinyl-pyrrolo-pyridinyl conjugation moiety may be used to conjugate the second linker, L^{B}, (and thus the second drug) to a polypeptide, such as an antibody.

For example, as shown in formula (II) above, L^{B} is attached to W¹³ through a conjugation moiety, and thus W¹³ is indirectly bonded to the second linker L^{B} through the hydrazinyl-indolyl or a hydrazinyl-pyrrolo-pyridinyl conjugation moiety. As described above, W¹³ is a polypeptide (e.g., an antibody), and thus L^{B} is attached through the hydrazinyl-indolyl or a hydrazinyl-pyrrolo-pyridinyl conjugation moiety to the polypeptide (antibody), e.g., the linker L^{B} is indirectly bonded to the polypeptide (antibody) through the hydrazinyl-indolyl or a hydrazinyl-pyrrolo-pyridinyl conjugation moiety.

Any convenient linker may be utilized for the second linker L^{B} in the subject conjugates and compounds. In certain embodiments, the second linker L^{B} may include a group selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In certain embodiments, the second linker L^{B} may include an alkyl or substituted alkyl group. In certain embodiments, the second linker L^{B} may include an alkenyl or substituted alkenyl group. In certain embodiments, the second linker L^{B} may include an alkynyl or substituted alkynyl group. In certain embodiments, the second linker L^{B} may include an alkoxy or substituted alkoxy group. In certain embodiments, the second linker L^{B} may include an amino or substituted amino group. In certain embodiments, the second linker L^{B} may include a carboxyl or carboxyl ester group. In certain embodiments, the second linker L^{B} may include an acyl amino group. In certain embodiments, the second linker L^{B} may include an alkylamide or substituted alkylamide group. In certain embodiments, the second linker L^{B} may include an aryl or substituted aryl group. In certain embodiments, the second linker L^{B} may include a heteroaryl or substituted heteroaryl group. In certain embodiments, the second linker L^{B} may include a cycloalkyl or substituted cycloalkyl group. In certain embodiments, the second linker L^{B} may include a heterocyclyl or substituted heterocyclyl group.

In certain embodiments, the second linker L^{B} may include a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In certain embodiments, the polymer is a polyalkylene glycol. In certain embodiments, the polymer is a polyethylene glycol. Other linkers are also possible, as shown in the conjugates and compounds described in more detail below.

In some embodiments, L^{B} is a second linker described by the formula:

-(L⁷)_{g}-(L⁸)ₕ-(L⁹)ᵢ-(L¹⁰)ⱼ-(L¹¹)ₖ-(L¹²)ₗ-(L¹³)ₘ,
wherein L⁷, L⁸, L⁹, L¹⁰, L¹¹, L¹² and L¹³ are each independently a linker subunit, and g, h, i, j, k, l and m are each independently 0 or 1.

In certain embodiments, the sum of g, h, i, j, k, l and m is 0 to 7. In certain embodiments, the sum of g, h, i, j, k, l and m is 0. In certain embodiments, the sum of g, h, i, j, k, 1 and m is 1. In certain embodiments, the sum of g, h, i, j, k, l and m is 2. In certain embodiments, the sum of g, h, i, j, k, l and m is 3. In certain embodiments, the sum of g, h, i, j, k, l and m is 4. In certain embodiments, the sum of g, h, i, j, k, l and m is 5. In certain embodiments, the sum of g, h, i, j, k, l and m is 6. In certain embodiments, the sum of g, h, i, j, k, l and m is 7. In certain embodiments, g, h, i, j, k, l and m are each 1. In certain embodiments, g, h, i, j, k and 1 are each 1 and m is 0. In certain embodiments, g, h, i, j and k are each 1 and 1 and m are each 0. In certain embodiments, g, h, i and j are each 1 and k, l and m are each 0. In certain embodiments, g, h, and i are each 1 and j, k, l and m are each 0. In certain embodiments, g and h are each 1 and i, j, k, 1 and m are each 0. In certain embodiments, g is 1 and h, i, j, k, l and m are each 0. In certain embodiments, g, h, i, j, k, l and m are each 0.

In certain embodiments, the linker subunit L⁷ is attached to the hydrazinyl-indolyl or a hydrazinyl-pyrrolo-pyridinyl conjugation moiety (e.g., as shown in formula (II) above). In certain embodiments, the linker subunit L⁸, if present, is attached to the second drug or active agent W¹². In certain embodiments, the linker subunit L⁹, if present, is attached to the second drug or active agent W¹². In certain embodiments, the linker subunit L¹⁰, if present, is attached to the second drug or active agent W¹². In certain embodiments, the linker subunit L¹¹, if present, is attached to the second drug or active agent W¹². In certain embodiments, the linker subunit L¹², if present, is attached to the second drug or active agent W¹². In certain embodiments, the linker subunit L¹³, if present, is attached to the second drug or active agent W¹².

Any convenient linker subunits may be utilized in the second linker L^{B}. Linker subunits of interest include, but are not limited to, units of polymers such as polyethylene glycols, polyethylenes and polyacrylates, amino acid residue(s), carbohydrate-based polymers or carbohydrate residues and derivatives thereof, polynucleotides, alkyl groups, aryl groups, heterocyclic groups, combinations thereof, and substituted versions thereof. In some embodiments, each of L⁷, L⁸, L⁹, L¹⁰, L¹¹, L¹² and L¹³ (if present) comprise one or more groups independently selected from a polyethylene glycol, a modified polyethylene glycol, an amino acid residue, an alkyl group, a substituted alkyl, an aryl group, a substituted aryl group, and a diamine (e.g., a linking group that includes an alkylene diamine).

In some embodiments, L⁷ (if present) comprises a polyethylene glycol, a modified polyethylene glycol, an amino acid residue, an alkyl group, a substituted alkyl, an aryl group, a substituted aryl group, or a diamine. In some embodiments, L⁷ comprises a polyethylene glycol. In some embodiments, L⁷ comprises a modified polyethylene glycol. In some embodiments, L⁷ comprises an amino acid residue. In some embodiments, L⁷ comprises an alkyl group or a substituted alkyl. In some embodiments, L⁷ comprises an aryl group or a substituted aryl group. In some embodiments, L⁷ comprises a diamine (e.g., a linking group comprising an alkylene diamine).

In some embodiments, L⁸ (if present) comprises a polyethylene glycol, a modified polyethylene glycol, an amino acid residue, an alkyl group, a substituted alkyl, an aryl group, a substituted aryl group, or a diamine. In some embodiments, L⁸ comprises a polyethylene glycol. In some embodiments, L⁸ comprises a modified polyethylene glycol. In some embodiments, L⁸ comprises an amino acid residue. In some embodiments, L⁸ comprises an alkyl group or a substituted alkyl. In some embodiments, L⁸ comprises an aryl group or a substituted aryl group. In some embodiments, L⁸ comprises a diamine (e.g., a linking group comprising an alkylene diamine).

In some embodiments, L⁹ (if present) comprises a polyethylene glycol, a modified polyethylene glycol, an amino acid residue, an alkyl group, a substituted alkyl, an aryl group, a substituted aryl group, or a diamine. In some embodiments, L⁹ comprises a polyethylene glycol. In some embodiments, L⁹ comprises a modified polyethylene glycol. In some embodiments, L⁹ comprises an amino acid residue. In some embodiments, L⁹ comprises an alkyl group or a substituted alkyl. In some embodiments, L⁹ comprises an aryl group or a substituted aryl group. In some embodiments, L⁹ comprises a diamine (e.g., a linking group comprising an alkylene diamine).

In some embodiments, L¹⁰ (if present) comprises a polyethylene glycol, a modified polyethylene glycol, an amino acid residue, an alkyl group, a substituted alkyl, an aryl group, a substituted aryl group, or a diamine. In some embodiments, L¹⁰ comprises a polyethylene glycol. In some embodiments, L¹⁰ comprises a modified polyethylene glycol. In some embodiments, L¹⁰ comprises an amino acid residue. In some embodiments, L¹⁰ comprises an alkyl group or a substituted alkyl. In some embodiments, L¹⁰ comprises an aryl group or a substituted aryl group. In some embodiments, L¹⁰ comprises a diamine (e.g., a linking group comprising an alkylene diamine).

In some embodiments, L¹¹ (if present) comprises a polyethylene glycol, a modified polyethylene glycol, an amino acid residue, an alkyl group, a substituted alkyl, an aryl group, a substituted aryl group, or a diamine. In some embodiments, L¹¹ comprises a polyethylene glycol. In some embodiments, L¹¹ comprises a modified polyethylene glycol. In some embodiments, L¹¹ comprises an amino acid residue. In some embodiments, L¹¹ comprises an alkyl group or a substituted alkyl. In some embodiments, L¹¹ comprises an aryl group or a substituted aryl group. In some embodiments, L¹¹ comprises a diamine (e.g., a linking group comprising an alkylene diamine).

In some embodiments, L¹² (if present) comprises a polyethylene glycol, a modified polyethylene glycol, an amino acid residue, an alkyl group, a substituted alkyl, an aryl group, a substituted aryl group, or a diamine. In some embodiments, L¹² comprises a polyethylene glycol. In some embodiments, L¹² comprises a modified polyethylene glycol. In some embodiments, L¹² comprises an amino acid residue. In some embodiments, L¹² comprises an alkyl group or a substituted alkyl. In some embodiments, L¹² comprises an aryl group or a substituted aryl group. In some embodiments, L¹² comprises a diamine (e.g., a linking group comprising an alkylene diamine).

In some embodiments, L¹³ (if present) comprises a polyethylene glycol, a modified polyethylene glycol, an amino acid residue, an alkyl group, a substituted alkyl, an aryl group, a substituted aryl group, or a diamine. In some embodiments, L¹³ comprises a polyethylene glycol. In some embodiments, L¹³ comprises a modified polyethylene glycol. In some embodiments, L¹³ comprises an amino acid residue. In some embodiments, L¹³ comprises an alkyl group or a substituted alkyl. In some embodiments, L¹³ comprises an aryl group or a substituted aryl group. In some embodiments, L¹³ comprises a diamine (e.g., a linking group comprising an alkylene diamine).

In some embodiments, L^{B} is a second linker comprising -(L⁷)_{g}-(L⁸)ₕ-(L⁹)ᵢ-(L¹⁰)ⱼ-(L¹¹)ₖ-(L¹²)ₗ-(L¹³)ₘ, where:
-(L⁷)_{g}- is -(T⁷-V⁷)_{g}-;
-(L⁸)ₕ- is -(T⁸-V⁸)ₕ-;
-(L⁹)ᵢ- is -(T⁹-V⁹)ᵢ-;
-(L¹⁰)ⱼ- is -(T¹⁰-V¹⁰)ⱼ-;
-(L¹¹)ₖ- is -(T¹¹-V¹¹)ₖ-;
-(L¹²)₁- is -(T¹²-V¹²)ₗ-; and
-(L¹³)ₘ- is -(T¹³-V¹³)ₘ-,
wherein T⁷, T⁸, T⁹, T¹⁰, T¹¹, T¹² and T¹³, if present, are tether groups;
V⁷, V⁸, V⁹, V¹⁰, V¹¹, V¹² and V¹³, if present, are covalent bonds or linking functional groups; and
g, h, i, j, k, l and m are each independently 0 or 1.

In certain embodiments, the sum of g, h, i, j, k, l and m is 0 to 7. In certain embodiments, the sum of g, h, i, j, k, l and m is 0. In certain embodiments, the sum of g, h, i, j, k, l and m is 1. In certain embodiments, the sum of g, h, i, j, k, l and m is 2. In certain embodiments, the sum of g, h, i, j, k, l and m is 3. In certain embodiments, the sum of g, h, i, j, k, l and m is 4. In certain embodiments, the sum of g, h, i, j, k, l and m is 5. In certain embodiments, the sum of g, h, i, j, k, l and m is 6. In certain embodiments, the sum of g, h, i, j, k, l and m is 7. In certain embodiments, g, h, i, j, k, l and m are each 1. In certain embodiments, g, h, i, j, k and l are each 1 and m is 0. In certain embodiments, g, h, i, j and k are each 1 and 1 and m are each 0. In certain embodiments, g, h, i and j are each 1 and k, l and m are each 0. In certain embodiments, g, h, and i are each 1 and j, k, l and m are each 0. In certain embodiments, g and h are each 1 and i, j, k, 1 and m are each 0. In certain embodiments, g is 1 and h, i, j, k, l and m are each 0. In certain embodiments, g, h, i, j, k, l and m are each 0.

As described above, in certain embodiments, L⁷ is attached to the hydrazinyl-indolyl or a hydrazinyl-pyrrolo-pyridinyl conjugation moiety (e.g., as shown in formula (II) above). As such, in certain embodiments, T⁷ is attached to the hydrazinyl-indolyl or a hydrazinyl-pyrrolo-pyridinyl conjugation moiety (e.g., as shown in formula (II) above). In certain embodiments, V⁷ is attached to the second drug or active agent. In certain embodiments, L⁸, if present, is attached to the second drug or active agent. As such, in certain embodiments, T⁸, if present, is attached to the second drug or active agent, or V⁸, if present, is attached to the second drug or active agent. In certain embodiments, L⁹, if present, is attached to the second drug or active agent. As such, in certain embodiments, T⁹, if present, is attached to the second drug or active agent, or V⁹, if present, is attached to the second drug or active agent. In certain embodiments, L¹⁰, if present, is attached to the second drug or active agent. As such, in certain embodiments, T¹⁰, if present, is attached to the second drug or active agent, or V10⁴, if present, is attached to the second drug or active agent. In certain embodiments, L¹¹, if present, is attached to the second drug or active agent. As such, in certain embodiments, T¹¹, if present, is attached to the second drug or active agent, or V¹¹, if present, is attached to the second drug or active agent. In certain embodiments, L¹², if present, is attached to the second drug or active agent. As such, in certain embodiments, T¹², if present, is attached to the second drug or active agent, or V¹², if present, is attached to the second drug or active agent. In certain embodiments, L¹³, if present, is attached to the second drug or active agent. As such, in certain embodiments, T¹³, if present, is attached to the second drug or active agent, or V¹³, if present, is attached to the second drug or active agent.

Regarding the tether groups, T¹, T², T³, T⁴, T⁵, T⁶, T⁷, T⁸, T⁹, T¹⁰, T¹¹, T¹² and T¹³, any convenient tether groups may be utilized in the subject linkers. In some embodiments, T¹, T², T³, T⁴, T⁵, T⁶, T⁷, T⁸, T⁹, T¹⁰, T¹¹, T¹² and T¹³ each comprise one or more groups independently selected from a covalent bond, a (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, where each w is an integer from 1 to 20, each n is an integer from 1 to 30, each p is an integer from 1 to 20, and each x is an integer from 1 to 12.

In certain embodiments, the tether group (e.g., T¹, T², T³, T⁴, T⁵, T⁶, T⁷, T⁸, T⁹, T¹⁰, T¹¹, T¹² and/or T¹³) includes a (C₁-C₁₂)alkyl or a substituted (C₁-C₁₂)alkyl. In certain embodiments, (C₁-C₁₂)alkyl is a straight chain or branched alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, (C₁-C₁₂)alkyl may be an alkyl or substituted alkyl, such as C₁-C₁₂ alkyl, or C₁-C₁₀ alkyl, or C₁-C₆ alkyl, or C₁-C₃ alkyl. In some instances, (C₁-C₁₂)alkyl is a C₂-alkyl. For example, (C₁-C₁₂)alkyl may be an alkylene or substituted alkylene, such as C₁-C₁₂ alkylene, or C₁-C₁₀ alkylene, or C₁-C₆ alkylene, or C₁-C₃ alkylene. In some instances, (C₁-C₁₂)alkyl is a C₁-alkylene (e.g., CH₂). In some instances, (C₁-C₁₂)alkyl is a C₂-alkylene (e.g., CH₂CH₂). In some instances, (C₁-C₁₂)alkyl is a C₃-alkylene (e.g., CH₂CH₂CH₂).

In certain embodiments, substituted (C₁-C₁₂)alkyl is a straight chain or branched substituted alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, substituted (C₁-C₁₂)alkyl may be a substituted alkyl, such as substituted C₁-C₁₂ alkyl, or substituted C₁-C₁₀ alkyl, or substituted C₁-C₆ alkyl, or substituted C₁-C₃ alkyl. In some instances, substituted (C₁-C₁₂)alkyl is a substituted C₂-alkyl. For example, substituted (C₁-C₁₂)alkyl may be a substituted alkylene, such as substituted C₁-C₁₂ alkylene, or substituted C₁-C₁₀ alkylene, or substituted C₁-C₆ alkylene, or substituted C₁-C₃ alkylene. In some instances, substituted (C₁-C₁₂)alkyl is a substituted C₁-alkylene (e.g., C₁-alkylene substituted with —SO₃H). In some instances, substituted (C₁-C₁₂)alkyl is a substituted C₂-alkylene. In some instances, substituted (C₁-C₁₂)alkyl is a substituted C₃-alkylene. For example, substituted (C₁-C₁₂)alkyl may include C₁-C₁₂ alkylene (e.g., C₃-alkylene or C₅-alkylene) substituted with a (PEG)ₖ group as described herein (e.g., —CONH(PEG)ₖ, such as —CONH(PEG)₃ or —CONH(PEG)₅; or —NHCO(PEG)ₖ, such as —NHCO(PEG)₇), or may include C₁-C₁₂ alkylene (e.g., C₃-alkylene) substituted with a —CONHCH₂CH₂SO₃H group, or may include C₁-C₁₂ alkylene (e.g., C₅-alkylene) substituted with a —NHCOCH₂SO₃H group.

In certain embodiments, the tether group (e.g., T¹, T², T³, T⁴, T⁵, T⁶, T⁷, T⁸, T⁹, T¹⁰, T¹¹, T¹² and/or T¹³) includes an aryl, substituted aryl, heteroaryl, substituted heteroaryl, cycloalkyl, substituted cycloalkyl, heterocyclyl, or substituted heterocyclyl. In some instances, the tether group (e.g., T¹, T², T³, T⁴, T⁵, T⁶, T⁷, T⁸, T⁹, T¹⁰, T¹H, T¹² and/or T¹³) includes an aryl or substituted aryl. For example, the aryl can be phenyl. In some cases, the substituted aryl is a substituted phenyl. The substituted phenyl can be substituted with one or more substituents selected from (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl. In some instances, the substituted aryl is a substituted phenyl, where the substituent includes a cleavable moiety as described herein (e.g., an enzymatically cleavable moiety, such as a glycoside or glycoside derivative).

In some instances, the tether group (e.g., T¹, T², T³, T⁴, T⁵, T⁶, T⁷, T⁸, T⁹, T¹⁰, T¹¹, T¹² and/or T¹³) includes a heteroaryl or substituted heteroaryl, such triazolyl (e.g., 1,2,3-triazolyl). In some instances, the tether group (e.g., T¹, T², T³, T⁴, T⁵, T⁶, T⁷, T⁸, T⁹, T¹⁰, T¹¹, T¹² and/or T¹³) includes a cycloalkyl or substituted cycloalkyl. In some instances, the tether group (e.g., T¹, T², T³, T⁴, T⁵, T⁶, T⁷, T⁸, T⁹, T¹⁰, T¹¹, T¹² and/or T¹³) includes a heterocyclyl or substituted heterocyclyl. In some instances, the substituent on the substituted heteroaryl, substituted cycloalkyl or substituted heterocyclyl includes a cleavable moiety as described herein (e.g., an enzymatically cleavable moiety, such as a glycoside or glycoside derivative).

In certain embodiments, the tether group (e.g., T¹, T², T³, T⁴, T⁵, T⁶, T⁷, T⁸, T⁹, T¹⁰, T¹¹, T¹² and/or T¹³) includes an ethylene diamine (EDA) moiety, e.g., an EDA containing tether group. In certain embodiments, (EDA)_{w} includes one or more EDA moieties, such as where w is an integer from 1 to 50, such as from 1 to 40, from 1 to 30, from 1 to 20, from 1 to 12 or from 1 to 6, such as 1, 2, 3, 4, 5 or 6). The linked ethylene diamine (EDA) moieties may optionally be substituted at one or more convenient positions with any convenient substituents, e.g., with an alkyl, a substituted alkyl, an acyl, a substituted acyl, an aryl or a substituted aryl. In certain embodiments, the EDA moiety is described by the structure:

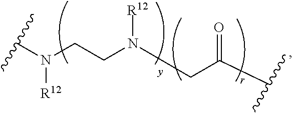

where y is an integer from 1 to 6, or is 0 or 1, and each R¹² is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, y is 1, 2, 3, 4, 5 or 6. In certain embodiments, y is 1 and r is 0. In certain embodiments, y is 1 and r is 1. In certain embodiments, y is 2 and r is 0. In certain embodiments, y is 2 and r is 1. In certain embodiments, each R¹² is independently selected from hydrogen, an alkyl, a substituted alkyl, an aryl and a substituted aryl. In certain embodiments, any two adjacent R¹² groups of the EDA may be cyclically linked, e.g., to form a piperazinyl ring. In certain embodiments, y is 1 and the two adjacent R¹² groups are an alkyl group, cyclically linked to form a piperazinyl ring. In certain embodiments, y is 1 and the adjacent R¹² groups are selected from hydrogen, an alkyl (e.g., methyl) and a substituted alkyl (e.g., lower alkyl-OH, such as ethyl-OH or propyl-OH).

In certain embodiments, the tether group (e.g., T, T², T³, T⁴, T⁵, T⁶, T⁷, T, T⁹, T¹⁰, T¹¹, T¹² and/or T¹³) includes a 4-amino-piperidine (4AP) moiety (also referred to herein as piperidin-4-amino, P4A). The 4AP moiety may optionally be substituted at one or more convenient positions with any convenient substituents, e.g., with an alkyl, a substituted alkyl, a polyethylene glycol moiety, an acyl, a substituted acyl, an aryl or a substituted aryl. In certain embodiments, the 4AP moiety is described by the structure:

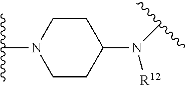

where R¹² is selected from hydrogen, alkyl, substituted alkyl, a polyethylene glycol moiety (e.g., a polyethylene glycol or a modified polyethylene glycol), alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹² is a polyethylene glycol moiety. In certain embodiments, R¹² is a carboxy modified polyethylene glycol.

In certain embodiments, R¹² includes a polyethylene glycol moiety described by the formula: (PEG)ₖ, which may be represented by the structure:

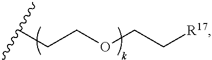

where k is an integer from 1 to 20, such as from 1 to 18, or from 1 to 16, or from 1 to 14, or from 1 to 12, or from 1 to 10, or from 1 to 8, or from 1 to 6, or from 1 to 4, or 1 or 2, such as 1, 2, 3, 4, 5, 6, 7, 8, 9, 10, 11, 12, 13, 14, 15, 16, 17, 18, 19 or 20. In some instances, k is 2. In certain embodiments, R¹⁷ is selected from OH, COOH, OR, or COOR, where R is selected from alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R¹⁷ is COOH. In certain embodiments, R¹⁷ is OH. In certain embodiments, R¹⁷ is OCH₃.

In certain embodiments, a tether group (e.g., T¹, T², T³, T⁴, T⁵, T⁶, T⁷, T⁸, T⁹, T¹⁰, T¹¹, T¹² and/or T¹³) includes (PEG)ₙ, where (PEG)ₙ is a polyethylene glycol or a modified polyethylene glycol linking unit. In certain embodiments, (PEG)ₙ is described by the structure:

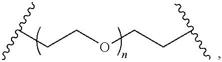

where n is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In some instances, n is 2. In some instances, n is 3. In some instances, n is 6. In some instances, n is 12.

In certain embodiments, a tether group (e.g., T¹, T², T³, T⁴, T⁵, T⁶, T⁷, T⁸, T⁹, T¹⁰, T¹¹, T¹² and/or T¹³) includes (AA)ₚ, where AA is an amino acid residue. Any convenient amino acids may be utilized. Amino acids of interest include but are not limited to, L- and D-amino acids, naturally occurring amino acids such as any of the 20 primary alpha-amino acids and beta-alanine, non-naturally occurring amino acids (e.g., amino acid analogs), such as a non-naturally occurring alpha-amino acid or a non-naturally occurring beta-amino acid, etc. In certain embodiments, p is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In certain embodiments, p is 1. In certain embodiments, p is 2.

In certain embodiments, a tether group (e.g., T¹, T², T³, T⁴, T⁵, T⁶, T⁷, T⁸, T⁹, T¹⁰, T¹¹, T¹² and/or T¹³) includes an amino acid analog. Amino acid analogs include compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or combinations thereof. For example, amino acid analogs may include α-hydroxy acids, and α-amino acids, and the like. Examples of amino acid analogs include, but are not limited to, sulfoalanine, and the like.

In certain embodiments, a tether group (e.g., T, T², T³, T⁴, T⁵, T⁶, T⁷, T, T⁹, T¹⁰, T¹¹, T¹² and/or T¹³) includes a moiety described by the formula —(CR¹³OH)ₓ—, where x is 0 or x is an integer from 1 to 50, such as from 1 to 40, from 1 to 30, from 1 to 20, from 1 to 12 or from 1 to 6, such as 1, 2, 3, 4, 5, 6, 7, 8, 9, 10, 11 or 12. In certain embodiments, x is 1. In certain embodiments, x is 2. In certain embodiments, R¹³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹³ is hydrogen. In certain embodiments, R¹³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹³ is alkynyl or substituted alkynyl. In certain embodiments, R¹³ is alkoxy or substituted alkoxy. In certain embodiments, R¹³ is amino or substituted amino. In certain embodiments, R¹³ is carboxyl or carboxyl ester. In certain embodiments, R¹³ is acyl or acyloxy. In certain embodiments, R¹³ is acyl amino or amino acyl. In certain embodiments, R¹³ is alkylamide or substituted alkylamide. In certain embodiments, R¹³ is sulfonyl. In certain embodiments, R¹³ is thioalkoxy or substituted thioalkoxy. In certain embodiments, R¹³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R¹³ is selected from hydrogen, alkyl, substituted alkyl, aryl, and substituted aryl. In these embodiments, alkyl, substituted alkyl, aryl, and substituted aryl are as described above for R¹³.

In certain embodiments, the tether group (e.g., T¹, T², T³, T⁴, T⁵, T⁶, T⁷, T, T⁹, T¹⁰, T¹¹, T¹² and/or T¹³) includes an acetal group, a disulfide, a hydrazine, or an ester. In some embodiments, the tether group includes an acetal group. In some embodiments, the tether group includes a hydrazine. In some embodiments, the tether group includes a disulfide. In some embodiments, the tether group includes an ester.

In certain embodiments, a tether group (e.g., T¹, T², T³, T⁴, T⁵, T⁶, T⁷, T, T⁹, T¹⁰, T¹¹, T¹² and/or T¹³) includes a meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), or para-hydroxy-phenyl (PHP).

In some embodiments, a tether group includes a MABO group described by the following structure:

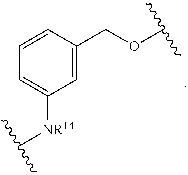

In some embodiments, a tether group includes a MABC group described by the following structure:

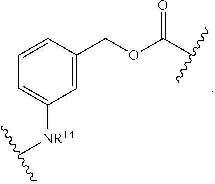

In some embodiments, a tether group includes a PABO group described by the following structure:

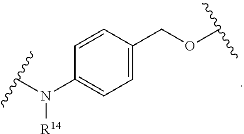

In some embodiments, a tether group includes a PABC group described by the following structure:

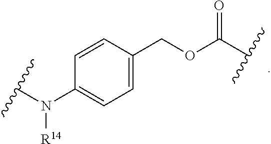

In some embodiments, a tether group includes a PAB group described by the following structure:

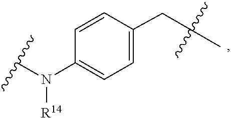

In some embodiments, a tether group includes a PABA group described by the following structure:

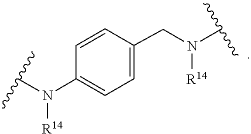

In some embodiments, a tether group includes a PAP group described by the following structure:

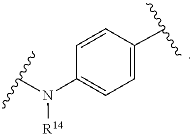

In some embodiments, a tether group includes a PHP group described by the following structure:

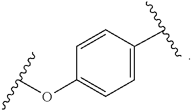 [SMILES: *Oc1ccc(*)cc1]

In certain embodiments, each R¹⁴ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, R¹⁴ is hydrogen. In certain embodiments, each R¹⁴ is hydrogen. In certain embodiments, R¹⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹⁴ is alkynyl or substituted alkynyl. In certain embodiments, R¹⁴ is alkoxy or substituted alkoxy. In certain embodiments, R¹⁴ is amino or substituted amino. In certain embodiments, R¹⁴ is carboxyl or carboxyl ester. In certain embodiments, R¹⁴ is acyl or acyloxy. In certain embodiments, R¹⁴ is acyl amino or amino acyl. In certain embodiments, R¹⁴ is alkylamide or substituted alkylamide. In certain embodiments, R¹⁴ is sulfonyl. In certain embodiments, R¹⁴ is thioalkoxy or substituted thioalkoxy. In certain embodiments, R¹⁴ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹⁴ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In some embodiments of the MABO, MABC, PABO, PABC, PAB, PABA, PAP, and PHP tether structures shown above, the phenyl ring may be substituted with one or more additional groups selected from halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, one or more of the tether groups T¹, T², T³, T⁴, T⁵, T⁶, T⁷, T⁸, T⁹, T¹⁰, T¹¹, T¹² and/or T¹³ is each optionally substituted with a glycoside or glycoside derivative. For example, in some instances, T¹, T², T³, T⁴, T⁵ and T⁶ are each optionally substituted with a glycoside. In some instances, T⁷, T⁸, T⁹, T¹⁰, T¹¹, T¹² and T¹³ are each optionally substituted with a glycoside. In certain embodiments, the glycoside or glycoside derivative is selected from a glucuronide, a galactoside, a glucoside, a mannoside, a fucoside, O-GlcNAc, and O-GalNAc.

In certain embodiments, the MABO, MABC, PABO, PABC, PAB, PABA, PAP, and PHP tether structures shown above may be substituted with an one or more additional groups selected from a glycoside and a glycoside derivative. For example, in some embodiments of the MABO, MABC, PABO, PABC, PAB, PABA, PAP, and PHP tether structures shown above, the phenyl ring may be substituted with one or more additional groups selected from a glycoside and a glycoside derivative. In certain embodiments, the glycoside or glycoside derivative is selected from a glucuronide, a galactoside, a glucoside, a mannoside, a fucoside, O-GlcNAc, and O-GalNAc.

For example, in some embodiments, the glycoside or glycoside derivative can be selected from the following structures:

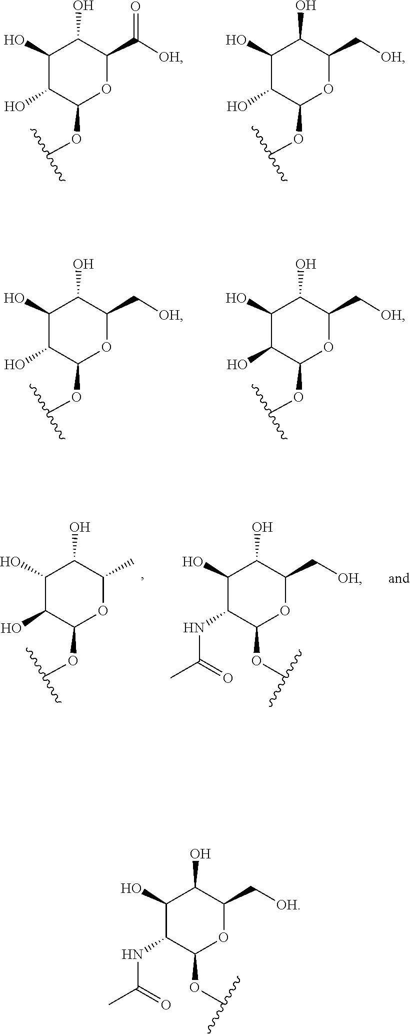

Regarding the linking functional groups, V¹, V², V³, V⁴, V⁵, V⁶, V⁷, V⁸, V⁹, V¹⁰, V¹¹, V¹² and V¹³ any convenient linking functional groups may be utilized in the subject linkers. Linking functional groups of interest include, but are not limited to, amino, carbonyl, amido, oxycarbonyl, carboxy, sulfonyl, sulfoxide, sulfonylamino, aminosulfonyl, thio, oxy, phospho, phosphoramidate, thiophosphoraidate, and the like. In some embodiments, V¹, V², V³, V⁴, V⁵, V⁶, V⁷, V⁸, V⁹, V¹⁰, V¹¹, V¹² and V¹³ are each independently selected from a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)——SO₂—, —SO₂NR¹⁵—, —NR¹⁵SO₂— and —P(O)OH—, where q is an integer from 1 to 6. In certain embodiments, q is an integer from 1 to 6 (e.g., 1, 2, 3, 4, 5 or 6). In certain embodiments, q is 1. In certain embodiments, q is 2. In certain embodiments, q is 3. In certain embodiments, q is 4. In certain embodiments, q is 5. In certain embodiments, q is 6.

In some embodiments, each R¹⁵ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, R¹⁵ is hydrogen. In certain embodiments, each R¹⁵ is hydrogen. In certain embodiments, R¹⁵ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹⁵ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹⁵ is alkynyl or substituted alkynyl. In certain embodiments, R¹⁵ is alkoxy or substituted alkoxy. In certain embodiments, R¹⁵ is amino or substituted amino. In certain embodiments, R¹⁵ is carboxyl or carboxyl ester. In certain embodiments, R¹⁵ is acyl or acyloxy. In certain embodiments, R¹⁵ is acyl amino or amino acyl. In certain embodiments, R¹⁵ is alkylamide or substituted alkylamide. In certain embodiments, R¹⁵ is sulfonyl. In certain embodiments, R¹⁵ is thioalkoxy or substituted thioalkoxy. In certain embodiments, R¹⁵ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹⁵ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹⁵ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹⁵ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl. In these embodiments, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl are as described above for R¹⁵.

As described above, in some embodiments, L^{A} is a first linker comprising -(T¹-V¹)ₐ-(T²-V²)_{b}-(T³-V³)_{c}-(T⁴-V⁴)_{d}-(T⁵-V⁵)ₑ-(T⁶-V⁶)_{f}-, where a, b, c, d, e and f are each independently 0 or 1.

In some embodiments, in the first linker L^{A}:
T¹ is selected from a (C₁-C₁₂)alkyl and a substituted (C₁-C₁₂)alkyl;
T², T³, T⁴, T⁵ and T⁶ are each independently selected from (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PHP, an acetal group, a disulfide, a hydrazine, and an ester; and
V¹, V², V³, V⁴, V⁵ and V⁶ are each independently selected from a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6;
   wherein:
(PEG)ₙ is

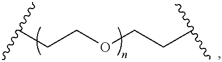

where n is an integer from 1 to 30;
EDA is an ethylene diamine moiety having the following structure:

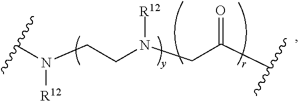

where y is an integer from 1 to 6 and r is 0 or 1;
4-amino-piperidine (4AP) is

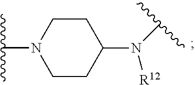

AA is an amino acid residue, where p is an integer from 1 to 20; and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

In certain embodiments, T¹, T², T³, T⁴, T⁵ and T⁶ and V¹, V², V³, V⁴, V⁵ and V⁶ are selected from the following:
wherein:
   T¹ is (C₁-C₁₂)alkyl and V¹ is —CONH—;
   T² is substituted (C₁-C₁₂)alkyl and V² is —CO—;
   T³ is AA and V³ is absent;
   T⁴ is PABC and V⁴ is absent; and
   e and f are each 0.

In certain embodiments, the left-hand side of the above linker structure for the first linker L^{A} is attached to the hydrazinyl-indolyl or a hydrazinyl-pyrrolo-pyridinyl conjugation moiety, and the right-hand side of the above linker structure for the first linker L^{A} is attached to the first drug or active agent.

As described above, in some embodiments, L^{B} is a second linker comprising -(T⁷-V⁷)_{g}-(T⁸-V⁸)ₕ-(T⁹-V⁹)ᵢ-(T¹⁰-V¹⁰)ⱼ-(T¹¹-V¹¹)ₖ-(T¹²-V¹²)ₗ(T¹³-V¹³)ₘ, where g, h, i, j, k, l and m are each independently 0 or 1.

In some embodiments, in the second linker L^{B}:
T⁷ is selected from a (C₁-C₁₂)alkyl and a substituted (C₁-C₁₂)alkyl;
T⁸, T⁹, T¹⁰, T¹¹, T¹² and T¹³ are each independently selected from (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PHP, an acetal group, a disulfide, a hydrazine, and an ester; and
V⁷, V⁸, V⁹, V¹⁰, V¹¹, V¹² and V¹³ are each independently selected from a covalent bond, —CO—, —NR¹⁵—, —NR⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6; wherein:
(PEG)ₙ is

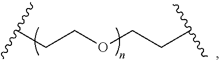

where n is an integer from 1 to 30;
EDA is an ethylene diamine moiety having the following structure:

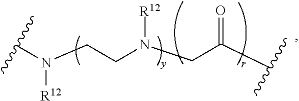

where y is an integer from 1 to 6 and r is 0 or 1;
4-amino-piperidine (4AP) is

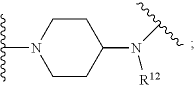

AA is an amino acid residue, where p is an integer from 1 to 20; and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

Any convenient tether groups may be utilized for T⁷, T⁸, T⁹, T¹⁰, T¹¹, T¹² and T¹³. For example, any of the tether groups described above in relation to T¹, T², T³, T⁴, T⁵ and T⁶ may be used for the tether groups T⁷, T⁸, T⁹, T¹⁰, T¹¹, T¹² and T¹³.

Any convenient linking functional groups may be utilized for V⁷, V⁸, V⁹, V¹⁰, V¹¹, V¹² and V¹³. For example, any of the linking functional groups described above in relation to V¹, V², V³, V⁴, V⁵ and V⁶ may be used for the linking functional groups V⁷, V⁸, V⁹, V¹⁰, V¹¹, V¹² and V¹³.

In certain embodiments, each R¹³ is independently selected from hydrogen, alkyl, substituted alkyl, aryl, and substituted aryl. In these embodiments, alkyl, substituted alkyl, aryl, and substituted aryl are as described above for R¹³.

In certain embodiments, each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl. In these embodiments, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl are as described above for R¹⁵. In these embodiments, various possible substituents are as described above for R¹⁵.

In certain embodiments of the second linker L^{B}, one or more of the tether groups T⁷, T⁸, T⁹, T¹⁰, T¹¹, T¹² and T¹³ is each optionally substituted with a glycoside or glycoside derivative. In certain embodiments, the glycoside or glycoside derivative is selected from a glucuronide, a galactoside, a glucoside, a mannoside, a fucoside, O-GlcNAc, and O-GalNAc.

In certain embodiments of the second linker L^{B}, the MABO, MABC, PABO, PABC, PAB, PABA, PAP, and PHP tether structures shown above may be substituted with an one or more additional groups selected from a glycoside and a glycoside derivative. For example, in some embodiments of the MABO, MABC, PABO, PABC, PAB, PABA, PAP, and PHP tether structures shown above, the phenyl ring may be substituted with one or more additional groups selected from a glycoside and a glycoside derivative. In certain embodiments, the glycoside or glycoside derivative is selected from a glucuronide, a galactoside, a glucoside, a mannoside, a fucoside, O-GlcNAc, and O-GalNAc.

In certain embodiments, T⁷, T⁸, T⁹, T¹⁰, T¹¹, T¹² and T¹³ and V⁷, V⁸, V⁹, V¹⁰, V¹¹, V¹² and V¹³ are selected from the following:
wherein:
   T⁷ is absent and V⁷ is —NHCO—;
   T⁸ is (C₁-C₁₂)alkyl and V⁸ is —CONH—;
   T⁹ is substituted (C₁-C₁₂)alkyl and V⁹ is —CO—;
   T¹⁰ is AA and V¹⁰ is absent;
   T¹¹ is PABC and V¹¹ is absent; and
   1 and m are each 0.

In certain embodiments, the left-hand side of the above linker structure for the second linker L^{B} is attached to the hydrazinyl-indolyl or a hydrazinyl-pyrrolo-pyridinyl conjugation moiety, and the right-hand side of the above linker structure for the second linker L^{B} is attached to the second drug or active agent.

In certain embodiments, the conjugate is an antibody-drug conjugate where the antibody and the drugs are linked together by linkers as described above. In some instances, the linker m(e.g., L^{A} and/or L^{B}) is a cleavable linker. A cleavable linker is a linker that includes one or more cleavable moieties, where the cleavable moiety includes one or more bonds that can dissociate under certain conditions, thus separating the cleavable linker into two or more separable portions. For example, the cleavable moiety may include one or more covalent bonds, which under certain conditions, can dissociate or break apart to separate the cleavable linker into two or more portions. As such the linkers that are included in an antibody-drug conjugate can be cleavable linkers, such that under appropriate conditions, the cleavable linker is cleaved to separate or release the drug from the antibody at a desired target site of action for the drug. In some instances, a cleavable linker includes two cleavable moieties, such as a first cleavable moiety and a second cleavable moiety. The cleavable moieties can be configured such that cleavage of both cleavable moieties is needed in order to separate or release the drug from the antibody at a desired target site of action for the drug. For example, cleavage of a cleavable linker can be achieved by initially cleaving one of the two cleavable moieties and then cleaving the other of the two cleavable moieties. In certain embodiments, a cleavable linker includes a first cleavable moiety and a second cleavable moiety that hinders cleavage of the first cleavable moiety. By “hinders cleavage” is meant that the presence of an uncleaved second cleavable moiety reduces the likelihood or substantially inhibits the cleavage of the first cleavable moiety, thus substantially reducing the amount or preventing the cleavage of the cleavable linker. For instance, the presence of uncleaved second cleavable moiety can hinder cleavage of the first cleavable moiety. The hinderance of cleavage of the first cleavable moiety by the presence of the second cleavable moiety, in turn, substantially reduces the amount or prevents the release of the drug from the antibody. For example, the premature release of the drug from the antibody can be substantially reduced or prevented until the antibody-drug conjugate is at or near the desired target site of action for the drug.

In some cases, since the second cleavable moiety hinders cleavage of the first cleavable moiety, cleavage of the cleavable linker can be achieved by initially cleaving the second cleavable moiety and then cleaving the first cleavable moiety. Cleavage of the second cleavable moiety can reduce or eliminate the hinderance on the cleavage of the first cleavable moiety, thus allowing the first cleavable moiety to be cleaved. Cleavage of the first cleavable moiety can result in the cleavable linker dissociating or separating into two or more portions as described above to release the drug from the antibody-drug conjugate. In some instances, cleavage of the first cleavable moiety does not substantially occur in the presence of an uncleaved second cleavable moiety. By substantially is meant that about 10% or less cleavage of the first cleavable moiety occurs in the presence of an uncleaved second cleavable moiety, such as about 9% or less, or about 8% or less, or about 7% or less, or about 6% or less, or about 5% or less, or about 4% or less, or about 3% or less, or about 2% or less, or about 1% or less, or about 0.5% or less, or about 0.1% or less cleavage of the first cleavable moiety occurs in the presence of an uncleaved second cleavable moiety.

Stated another way, the second cleavable moiety can protect the first cleavable moiety from cleavage. For instance, the presence of uncleaved second cleavable moiety can protect the first cleavable moiety from cleavage, and thus substantially reduce or prevent premature release of the drug from the antibody until the antibody-drug conjugate is at or near the desired target site of action for the drug. As such, cleavage of the second cleavable moiety exposes the first cleavable moiety (e.g., deprotects the first cleavable moiety), thus allowing the first cleavable moiety to be cleaved, which results in cleavage of the cleavable linker, which, in turn, separates or releases the drug from the antibody at a desired target site of action for the drug as described above. In certain instances, cleavage of the second cleavable moiety exposes the first cleavable moiety to subsequent cleavage, but cleavage of the second cleavable moiety does not in and of itself result in cleavage of the cleavable linker (i.e., cleavage of the first cleavable moiety is still needed in order to cleave the cleavable linker).

The cleavable moieties included in the cleavable linker may each be an enzymatically cleavable moiety. For example, the first cleavable moiety can be a first enzymatically cleavable moiety and the second cleavable moiety can be a second enzymatically cleavable moiety. An enzymatically cleavable moiety is a cleavable moiety that can be separated into two or more portions as described above through the enzymatic action of an enzyme. The enzymatically cleavable moiety can be any cleavable moiety that can be cleaved through the enzymatic action of an enzyme, such as, but not limited to, an ester, a peptide, a glycoside, and the like. In some instances, the enzyme that cleaves the enzymatically cleavable moiety is present at a desired target site of action, such as the desired target site of action of the drug that is to be released from the antibody-drug conjugate. In some cases, the enzyme that cleaves the enzymatically cleavable moiety is not present in a significant amount in other areas, such as in whole blood, plasma or serum. As such, the cleavage of an enzymatically cleavable moiety can be controlled such that substantial cleavage occurs at the desired site of action, whereas cleavage does not significantly occur in other areas or before the antibody-drug conjugate reaches the desired site of action.

For example, as described herein, antibody-drug conjugates of the present disclosure can be used for the treatment of cancer, such as for the delivery of a cancer therapeutic drug to a desired site of action where the cancer cells are present. In some cases, enzymes, such as an esterase that cleaves ester bonds or a glycosidase that cleaves glycosidic bonds, can be a biomarker for cancer that is overexpressed in cancer cells. The overexpression, and thus localization, of certain enzymes in cancer can be used in the context of the enzymatically cleavable moieties included in the cleavable linkers of the antibody-drug conjugates of the present disclosure to specifically release the drug at the desired site of action (i.e., the site of the cancer (and overexpressed enzyme)). Thus, in some embodiments, the enzymatically cleavable moiety is a cleavable moiety (e.g., an ester or a glycoside) that can be cleaved by an enzyme that is overexpressed in cancer cells. For instance, the enzyme can be an esterase. As such, in some instances, the enzymatically cleavable moiety is a cleavable moiety (e.g., an ester) that can be cleaved by an esterase enzyme. In some instances, the enzyme can be a glycosidase. As such, in some instances, the enzymatically cleavable moiety is a cleavable moiety (e.g., a glycoside or glycoside derivative) that can be cleaved by a glycosidase enzyme.

In certain embodiments, the enzymatically cleavable moiety is an ester bond. For example, the first cleavable moiety described above (i.e., the cleavable moiety protected from premature cleavage by the second cleavable moiety) can include an ester. The presence of uncleaved second cleavable moiety can protect the first cleavable moiety (ester) from cleavage by an esterase enzyme, and thus substantially reduce or prevent premature release of the drug from the antibody until the antibody-drug conjugate is at or near the desired target site of action for the drug. In some instances, a portion of the linker adjacent to the first cleavable moiety is linked to or includes a substituent, where the substituent comprises the second cleavable moiety. In some instances, the second cleavable moiety includes a glycoside or glycoside derivative.

In some embodiments, the enzymatically cleavable moiety is sugar moiety, such as a glycoside (or glyosyl) or glycoside derivative. In some cases, the glycoside or glycoside derivative can facilitate an increase in the hydrophilicity of the cleavable linker as compared to a cleavable linker that does not include the glycoside or glycoside derivative. The glycoside or glycoside derivative can be any glycoside or glycoside derivative suitable for use in the cleavable linker and that can be cleaved through the enzymatic action of an enzyme. For example, the second cleavable moiety (i.e., the cleavable moiety that protects the first cleavable moiety from premature cleavage) can be a glycoside or glycoside derivative. For instance, in some embodiments, the first cleavable moiety includes an ester and the second cleavable moiety includes a glycoside or glycoside derivative. In certain embodiments, the second cleavable moiety is a glycoside or glycoside derivative selected from a glucuronide, a galactoside, a glucoside, a mannoside, a fucoside, O-GlcNAc, and O-GalNAc. In some instances, the second cleavable moiety is a glucuronide. In some instances, the second cleavable moiety is a galactoside. In some instances, the second cleavable moiety is a glucoside. In some instances, the second cleavable moiety is a mannoside. In some instances, the second cleavable moiety is a fucoside. In some instances, the second cleavable moiety is O-GlcNAc. In some instances, the second cleavable moiety is O-GalNAc.

The glycoside or glycoside derivative can be attached (covalently bonded) to the cleavable linker through a glycosidic bond. The glycosidic bond can link the glycoside or glycoside derivative to the cleavable linker through various types of bonds, such as, but not limited to, an O-glycosidic bond (an O-glycoside), an N-glycosidic bond (a glycosylamine), an S-glycosidic bond (a thioglycoside), or C-glycosidic bond (a C-glycoside or C-glycosyl). In some instances, the glycosidic bond is an O-glycosidic bond (an O-glycoside). In some cases, the glycoside or glycoside derivative can be cleaved from the cleavable linker it is attached to by an enzyme (e.g., through enzymatically-mediated hydrolysis of the glycosidic bond). A glycoside or glycoside derivative can be removed or cleaved from the cleavable linker by any convenient enzyme that is able to carry out the cleavage (hydrolysis) of the glycosidic bond that attaches the glycoside or glycoside derivative to the cleavable linker. An example of an enzyme that can be used to mediate the cleavage (hydrolysis) of the glycosidic bond that attaches the glycoside or glycoside derivative to the cleavable linker is a glycosidase, such as a glucuronidase, a galactosidase, a glucosidase, a mannosidase, a fucosidase, and the like. Other suitable enzymes may also be used to mediate the cleavage (hydrolysis) of the glycosidic bond that attaches the glycoside or glycoside derivative to the cleavable linker. In some cases, the enzyme used to mediate the cleavage (hydrolysis) of the glycosidic bond that attaches the glycoside or glycoside derivative to the cleavable linker is found at or near the desired site of action for the drug of the antibody-drug conjugate. For instance, the enzyme can be a lysosomal enzyme, such as a lysosomal glycosidase, found in cells at or near the desired site of action for the drug of the antibody-drug conjugate. In some cases, the enzyme is an enzyme found at or near the target site where the enzyme that mediates cleavage of the first cleavable moiety is found.

Examples of conjugates according to the present disclosure include, but are not limited to, the following structure:

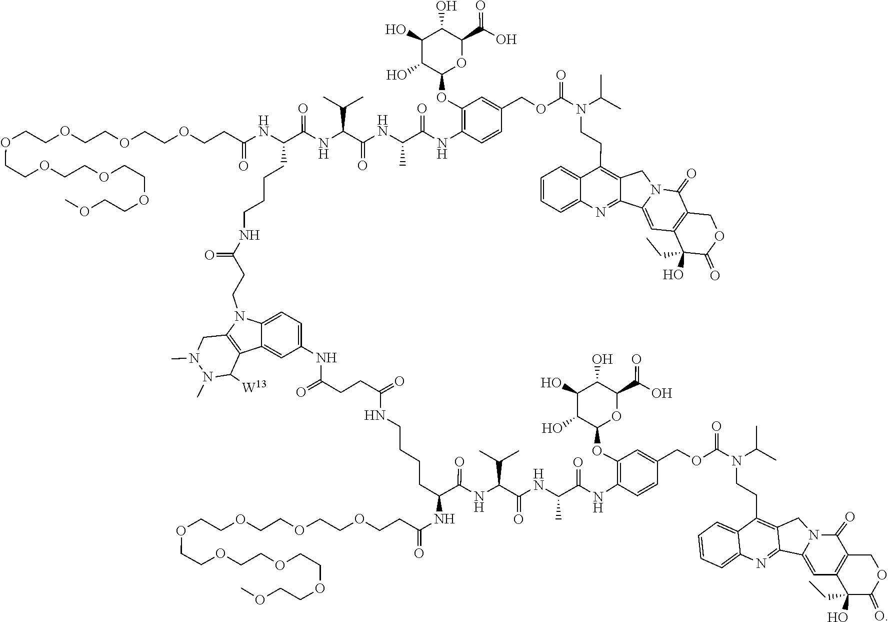

Any of the chemical entities, linkers and conjugation moieties set forth in the structures above may be adapted for use in the subject compounds and conjugates.

Additional disclosure related to hydrazinyl-indolyl and hydrazinyl-pyrrolo-pyridinyl compounds and methods for producing a conjugate is found in U.S. Pat. Nos. 9,310,374 and 9,493,413, the disclosures of each of which are incorporated herein by reference.

### Anti-MUC1 Antibodies

As noted above, a subject conjugate can comprise, as substituent W² an anti-MUC1 antibody, where the amino acid sequence of the anti-MUC1 antibody has been modified to include a 2-formylglycine (fGly) residue. As used herein, amino acids may be referred to by their standard name, their standard three letter abbreviation and/or their standard one letter abbreviation, such as: Alanine or Ala or A; Cysteine or Cys or C; Aspartic acid or Asp or D; Glutamic acid or Glu or E; Phenylalanine or Phe or F; Glycine or Gly or G; Histidine or His or H; Isoleucine or Ile or I; Lysine or Lys or K; Leucine or Len or L; Methionine or Met or M; Asparagine or Asn or N; Proline or Pro or P; Glutamine or Gln or Q; Arginine or Arg or R; Serine or Ser or S; Threonine or Thr or T; Valine or Val or V; Tryptophan or Trp or W; and Tyrosine or Tyr or Y.

According to some embodiments, an antibody of the present disclosure specifically binds to MUC1 and comprises:
a variable heavy chain (VH) chain comprising heavy chain CDRs1-3 (HCDRs1-3) of a VH chain having the sequence:
EVQLVQSGAEVKKPGATVKISCKVSGYTFTDHTMHWIKQRPGKGLEWM GYFYPRDDSTNYNEKFKGRVTLTADKSTDTAYMELSSLRSEDTAVYYCARGLRYALDY WGQGTLVTVSS (SEQ ID NO:1); and
a variable light chain (VL) chain comprising light chain CDRs1-3 (LCDRs1-3) of a VL chain having the sequence:

(SEQ ID NO: 2)
EIVLTQSPATLSLSPGERATLSCRASSSVSSSYLYWYQQKPGQAP
RLWIYGTSNLASGVPARFSGSGSGTDYTLTISSLEPEDAAVYYCH
QYAWSPPTFGQGTKLEIK;
(SEQ ID NO: 3)
EIVLTQSPATLSLSPGERATLSCRASSSVGSSNLYWYQQKPGQAP
RLWIYRSTKLASGVPARFSGSGSGTDYTLTISSLEPEDAAVYYCH
QYRWSPPTFGQGTKLEIK;
or
(SEQ ID NO: 4)
EIVLTQSPATLSLSPGERATLSCRASSSVSSSYLYWYQQKPGQAP
RLWIIGTSNLASGVPARFSGSGSGTDYTLTISSLEPEDAAVYYCH
QYSWSPPTFGQGTKLEIK.

The HCDRs1-3 and LCDRs1-3 may be as defined by Chothia, Kabat, or IMT nomenclature. The HCDRs1-3 of the anti-MUC1 antibodies disclosed herein as defined per the listed nomenclatures may be as follows:

TABLE 2

| Anti-MUC1 | | | |
|---|---|---|---|
| Antibody | Chothia | Kabat | IMGT |
| HCDR1 | GYTFTDH | DHTMH | GYTFTDHT |
| | (SEQ ID NO: 7) | (SEQ ID NO: 17) | (SEQ ID NO: 34) |
| HCDR2 | YPRDDS | YFYPRDDSTNYNEKFKG | FYPRDDST |
| | (SEQ ID NO: 8) | (SEQ ID NO: 18) | (SEQ ID NO: 44) |
| HCDR3 | GLRYALDY | GLRYALDY | ARGLRYALDY |
| | (SEQ ID NO: 9) | (SEQ ID NO: 9) | (SEQ ID NO: 45) |

The LCDRs 1-3 of the anti-MUC1 antibodies disclosed herein may be as defined per the nomenclatures listed in Tables 3-5.

TABLE 3

| Anti-MUC1 | | |
|---|---|---|
| Antibody | Chothia and Kabat | IMGT |
| LCDR1 | RASSSVSSSYLY | SSVSSSY |
| | (SEQ ID NO: 10) | (SEQ ID NO: 33) |
| LCDR2 | GTSNLAS | GT |
| | (SEQ ID NO: 11) | |
| LCDR3 | HQYAWSPPT | HQYAWSPPT |
| | (SEQ ID NO: 12) | (SEQ ID NO: 12) |

TABLE 4

| Anti-MUC1 | | |
|---|---|---|
| Antibody | Chothia and Kabat | IMGT |
| LCDR1 | RASSSVGSSNLY | SSVGSSN |
| | (SEQ ID NO: 13) | (SEQ ID NO: 46) |
| LCDR2 | RSTKLAS | RS |
| | (SEQ ID NO: 14) | |
| LCDR3 | HQYRWSPPT | HQYRWSPPT |
| | (SEQ ID NO: 15) | (SEQ ID NO: 15) |

TABLE 5

| Anti-MUC1 | | |
|---|---|---|
| Antibody | Chothia and Kabat | IMGT |
| LCDR1 | RASSSVSSSYLY | SSVSSSY |
| | (SEQ ID NO: 10) | (SEQ ID NO: 33) |
| LCDR2 | GTSNLAS | GT |
| | (SEQ ID NO: 11) | |
| LCDR3 | HQYSWSPPT | HQYSWSPPT |
| | (SEQ ID NO: 16) | (SEQ ID NO: 16) |

In certain embodiments, the VH chain of an anti-MUC1 antibody comprises the HCDRs1-3 as set forth herein and the VL chain of the anti-MUC1 antibody comprises LCDRs1-3, wherein

The LCDR1 comprises the amino acid sequence
(SEQ ID NO: 41)
RASSSVG/SSSYLY;
the LCDR2 comprises the amino acid sequence
(SEQ ID NO:42)
G/RT/SS/TN/KLAS;
the LCDR3 comprises the amino acid sequence
(SEQ ID NO: 43)
HQYA/R/SWSPPT, as per Kabat definition.

In certain embodiments, the VH chain of an anti-MUC1 antibody comprises the HCDRs1-3 as set forth herein and comprises an amino acid sequence having 80% or greater, 85% or greater, 90% or greater, 95% or greater, 99% or greater, or 100% sequence identity to the amino acid sequence set forth in SEQ ID NO:1. In certain embodiments, any amino acid differences between the VH chain of an anti-MUC1 antibody of the present disclosure and SEQ ID NO:1 may be limited to regions outside of the CDRs, e.g., in one or more of the framework regions (FR), e.g., FR1, FR2, FR3, and/or FR4.

In certain embodiments, the VL chain of an anti-MUC1 antibody comprises the LCDRs1-3 as set forth herein in Table 3 and comprises an amino acid sequence having 80% or greater, 85% or greater, 90% or greater, 95% or greater, 99% or greater, or 100% sequence identity to the amino acid sequence set forth in SEQ ID NO:2.

In certain embodiments, the VL chain of an anti-MUC1 antibody comprises the LCDRs1-3 as set forth herein in Table 4 and comprises an amino acid sequence having 80% or greater, 85% or greater, 90% or greater, 95% or greater, 99% or greater, or 100% sequence identity to the amino acid sequence set forth in SEQ ID NO:3.

In certain embodiments, the VL chain of an anti-MUC1 antibody comprises the LCDRs1-3 as set forth herein in Table 5 and comprises an amino acid sequence having 80% or greater, 85% or greater, 90% or greater, 95% or greater, 99% or greater, or 100% sequence identity to the amino acid sequence set forth in SEQ ID NO:4.

In certain embodiments, any amino acid differences between the VL chain of an anti-MUC1 antibody of the present disclosure and SEQ ID NO:2, 3, and 4 may be limited to regions outside of the CDRs, e.g., in one or more of the framework regions (FR), e.g., FR1, FR2, FR3, and/or FR4.

In certain embodiments, an anti-MUC1 antibody of the present disclosure can comprise: a) a heavy chain comprising a VH region having the amino acid sequence set forth in SEQ ID NO:1; and b) a light chain comprising the VL region having the amino acid sequence set forth in SEQ ID NO:2, 3, or 4.

In certain embodiments, an anti-MUC1 antibody of the present disclosure can comprise: a) a heavy chain comprising a VH region having the amino acid sequence set forth in SEQ ID NO:1 and a heavy chain constant region having the amino acid sequence set forth in any one of SEQ ID NOs: 57-73; and b) a light chain comprising the VL region having the amino acid sequence set forth in SEQ ID NO:2, 3, or 4.

In certain embodiments, an anti-MUC1 antibody of the present disclosure can comprise: a) a heavy chain comprising a VH region having the amino acid sequence set forth in SEQ ID NO:1 and a heavy chain constant region having the amino acid sequence set forth in any one of SEQ ID NOs: 57-73, wherein the C present in the sequence LCTPSR in the constant region is replaced by fGly; and b) a light chain comprising the VL region having the amino acid sequence set forth in SEQ ID NO:2, 3, or 4.

In certain embodiments, an anti-MUC1 antibody of the present disclosure can comprise: a) a heavy chain comprising a VH region having the amino acid sequence set forth in SEQ ID NO:1 and a heavy chain constant region having the amino acid sequence set forth in any one of SEQ ID NOs: 57-73, wherein the C present in the sequence LCTPSR in the constant region is replaced by fGly′, wherein fGly′ refers to the amino acid residue conjugated to a moiety of interest; and b) a light chain comprising the VL region having the amino acid sequence set forth in SEQ ID NO:2, 3, or 4.

In certain embodiments, an anti-MUC1 antibody of the present disclosure can comprise: a) a heavy chain comprising a VH region comprising an amino acid sequence at least 85% identical (e.g., at least 90%, at least 95%, at least 96%, at least 97%, at least 98%, or at least 99% identical) to the amino acid sequence set forth in SEQ ID NO:1 and a heavy chain constant region comprising an amino acid sequence at least 85% identical (e.g., at least 90%, at least 95%, at least 96%, at least 97%, at least 98%, or at least 99% identical to the amino acid sequence set forth in any one of SEQ ID NOs: 57-73, wherein the C present in the sequence LCTPSR in the constant region is replaced by fGly′, wherein fGly′ refers to the amino acid residue conjugated to a moiety of interest; and b) a light chain comprising a VL region comprising an amino acid sequence at least 85% identical (e.g., at least 90%, at least 95%, at least 96%, at least 97%, at least 98%, or at least 99% identical) to the amino acid sequence set forth in SEQ ID NO:2, 3, or 4.

The anti-MUC1 antibodies of the present disclosure may bind to MUC-1 with an EC50 of about 0.4-1 nM, e.g., 0.5-0.9 nM, 0.6-0.8 nM, or 0.65-0.75 nM as measured by ELISA. The concentration of an antibody that provides half maximal response (e.g., half of the maximum fluorescence intensity) is measured as the EC50. The MUC-1 may be the 20mer glycosylated MUC1 peptide as disclosed in Example 1.

The anti-MUC1 antibodies of the present disclosure may bind to 20mer MUC1 glycosylated peptide but not to recombinant 60mer MUC1 non-glycosylated peptide, as disclosed in Example 1.

The anti-MUC1 antibodies of the present disclosure may bind to cancerous tissue and may show no binding (e.g., insignificant binding as measured by immunohistochemistry or binding undetectable by immunohistochemistry) to normal tissue. For example, the anti-MUC1 antibodies described herein may bind to human gastric, breast, and/or lung tissue that have cancerous cells while showing no detectable binding to human gastric, breast, and/or lung tissue that do not have cancerous cells.

The antibodies find use in a variety of research, diagnostic, and therapeutic applications, including for performing any of the methods described in U.S. Patent Application Nos. US20120141375A1, US20160145343A1, the disclosure of which is incorporated herein by reference in its entirety for all purposes.

A subject antibody specifically binds a MUC1 polypeptide, where the epitope comprises amino acid residues within a human MUC1 antigen comprising the amino acid sequence set forth in SEQ ID NO:20:

(SEQ ID NO: 20)
MTPGTQSPFFLLLLLTVLTVVTGSGHASSTPGGEKETSATQRSSVPSSTEK
NAVSMTSSVLSSHSPGSGSSTTQGQDVTLAPATEPASGSAATWGQDVTSVPVTRPALGS
TTPPAHDVTSAPDNKPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTSAPDNRPALGS
TAPPVHNVTSASGSASGSASTLVHNGTSARATTTPASKSTPFSIPSHHSDTPTTLASHSTK
TDASSTHHSSVPPLTSSNHSTSPQLSTGVSFFFLSFHISNLQFNSSLEDPSTDYYQELQRDIS
EMFLQIYKQGGFLGLSNIKFRPGSVVVQLTLAFREGTINVHDVETQFNQYKTEAASRYN
LTISDVSVSDVPFPFSAQSGAGVPGWGIALLVLVCVLVALAIVYLIALAVCQCRRKNYG
QLDIFPARDTYHPMSEYPTYHTHGRYVPPSSTDRSPYEKVSAGNGGSSLSYTNPAVAAT
SANL

In certain embodiments, the MUC1 epitope bound by the anti-MUC1 antibodies disclosed herein is glycosylated. In certain embodiments, the MUC1 epitope bound by the anti-MUC1 antibodies disclosed herein is present on MUC1 expressed by epipulmonary adenocarcinoma cell lines and pulmonary epithelial cells.

A subject antibody exhibits high affinity binding to MUC1. For example, a subject antibody binds to MUC1 with an affinity of at least about 10⁻⁷ M, at least about 10⁻⁸ M, at least about 10⁻⁹ M, at least about 10⁻¹⁰ M, at least about 10⁻¹¹ M, or at least about 10⁻¹² M, or greater than 10⁻¹² M. A subject antibody binds to an epitope present on MUC1 with an affinity of from about 10⁻⁷ M to about 10⁻⁸ M, from about 10⁻⁸ M to about 10⁻⁹ M, from about 10⁻⁹ M to about 10⁻¹¹ M, from about 10⁻¹⁰ M to about 10⁻¹¹ M, or from about 10⁻¹¹ M to about 10⁻¹² M, or greater than 10⁻¹² M.

An anti-MUC1 antibody of the present disclosure can in some cases induce apoptosis in a cell that expresses MUC1 on its cell surface.

A “MUC1 antigen” or “MUC1 polypeptide” can comprises an amino acid sequence having at least about 75%, at least about 80%, at least about 90%, at least about 95%, at least about 98%, at least about 99%, or 100%, amino acid sequence identity to SEQ ID NO:20.

As used herein the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes; and numerous immunoglobulin variable region genes. Full-length immunoglobulin light chains (about 25 kD or 214 amino acids) are encoded by a variable region gene at the N-terminus (about 110 amino acids) and a kappa or lambda constant region at the C-terminus. Full-length immunoglobulin heavy chains (about 50 kD or 446 amino acids) are encoded by a variable region gene at the N-terminus (about 116 amino acids) and one of the other aforementioned constant region genes at the C-terminus, e.g., gamma (encoding about 330 amino acids). In some embodiments, a subject antibody comprises full-length immunoglobulin heavy chain and a full-length immunoglobulin light chain.

In some embodiments, a subject antibody does not comprise a full-length immunoglobulin heavy chain and a full-length immunoglobulin light chain, and instead comprises antigen-binding fragments of a full-length immunoglobulin heavy chain and a full-length immunoglobulin light chain. In some embodiments, the antigen-binding fragments are contained on separate polypeptide chains; in other embodiments, the antigen-binding fragments are contained within a single polypeptide chain. The term “antigen-binding fragment” refers to one or more fragments of a full-length antibody that are capable of specifically binding to MUC1, as described above. Examples of binding fragments include (i) a Fab fragment (a monovalent fragment consisting of the VL, VH, CL and CH₁ domains; (ii) a F(ab′)₂ fragment (a bivalent fragment comprising two Fab fragments linked by a disulfide bridge at the hinge region; (iii) a Fd fragment (consisting of the VH and CH₁ domains); (iv) a Fv fragment (consisting of the VH and VL domains of a single arm of an antibody); (v) a dAb fragment (consisting of the VH domain); (vi) an isolated CDR; (vii) a single chain Fv (scFv) (consisting of the VH and VL domains of a single arm of an antibody joined by a synthetic linker using recombinant means such that the VH and VL domains pair to form a monovalent molecule); (viii) diabodies (consisting of two scFvs in which the VH and VL domains are joined such that they do not pair to form a monovalent molecule; the VH of each one of the scFv pairs with the VL domain of the other scFv to form a bivalent molecule); (ix) bi-specific antibodies (consisting of at least two antigen binding regions, each region binding a different epitope). In some embodiments, a subject antibody fragment is a Fab fragment. In some embodiments, a subject antibody fragment is a single-chain antibody (scFv).

In some embodiments, a subject antibody is a recombinant or modified antibody, e.g., a chimeric, humanized, deimmunized or an in vitro generated antibody. The term “recombinant” or “modified” antibody as used herein is intended to include all antibodies that are prepared, expressed, created, or isolated by recombinant means, such as (i) antibodies expressed using a recombinant expression vector transfected into a host cell; (ii) antibodies isolated from a recombinant, combinatorial antibody library; (iii) antibodies isolated from an animal (e.g. a mouse) that is transgenic for human immunoglobulin genes; or (iv) antibodies prepared, expressed, created, or isolated by any other means that involves splicing of human immunoglobulin gene sequences to other DNA sequences. Such recombinant antibodies include humanized, CDR grafted, chimeric, deimmunized, and in vitro generated antibodies; and can optionally include constant regions derived from human germline immunoglobulin sequences.

Full length bispecific antibodies may be generated for example using Fab arm exchange (or half molecule exchange) between two monospecific bivalent antibodies by introducing substitutions at the heavy chain CH₃ interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH₃ domains. The heavy chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH₃ domains of the parent antibodies release and reform by dissociation-association. The CH₃ domains of the Fab arms may be engineered to favor heterodimerization over homodimerization. The resulting product is a bispecific antibody having two Fab arms or half molecules which each bind a distinct epitope.

The “knob-in-hole” strategy (see, e.g., PCT Intl. Publ. No. WO 2006/028936) may be used to generate full length bispecific antibodies. Briefly, selected amino acids forming the interface of the CH₃ domains in human IgG can be mutated at positions affecting CH₃ domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH₃ substitution pairs forming a knob and a hole are (expressed as modified position in the first CH₃ domain of the first heavy chain/modified position in the second CH₃ domain of the second heavy chain): T366Y/F405A, T366W/F405W, F405W/Y407A, T394W/Y407T, T3945/Y407A, T366W/T394S, F405W/T394S and T366W/T366S/L³⁶⁸A/Y407V.

Other strategies such as promoting heavy chain heterodimerization using electrostatic interactions by substituting positively charged residues at one CH₃ surface and negatively charged residues at a second CH₃ surface may be used, as described in US Pat. Publ. No. US2010/0015133; US Pat. Publ. No. US2009/0182127; US Pat. Publ. No. U82010/028637 or US Pat. Publ. No. US2011/0123532. In other strategies, heterodimerization may be promoted by following substitutions (expressed as modified position in the first CH₃ domain of the first heavy chain/modified position in the second CH₃ domain of the second heavy chain): L351 Y/F405A/Y407V/T394W, T366I/K392M/T394W/F405A/Y407V, T366L/K392M/T394W/F405A/Y407V, L351Y/Y407A/T366A/K409F, L351Y/Y407A/T366V/K409F, Y407A/T366A/K409F, or T350V/L351Y/F405A/Y407V, T350V/T366L/K392L/T394W as described in U.S. Pat. Pub. No. US2012/0149876 or U.S. Pat. Pub. No. US2013/0195849.

Also provided are single chain bispecific antibodies. In some embodiments, a single chain bispecific antibody of the present disclosure is a bispecific scFv. A subject antibody can be humanized. The constant region(s), if present, can also be substantially or entirely from a human immunoglobulin.

Methods of making humanized antibodies are known in the art. The substitution of mouse CDRs into a human variable domain framework can result in retention of their correct spatial orientation where, e.g., the human variable domain framework adopts the same or similar conformation to the mouse variable framework from which the CDRs originated. This can be achieved by obtaining the human variable domains from human antibodies whose framework sequences exhibit a high degree of sequence identity with the murine variable framework domains from which the CDRs were derived. The heavy and light chain variable framework regions can be derived from the same or different human antibody sequences. The human antibody sequences can be the sequences of naturally occurring human antibodies or can be consensus sequences of several human antibodies.

Having identified the complementarity determining regions of the murine donor immunoglobulin and appropriate human acceptor immunoglobulins, the next step is to determine which, if any, residues from these components should be substituted to optimize the properties of the resulting humanized antibody. In general, substitution of human amino acid residues with murine should be minimized, because introduction of murine residues increases the risk of the antibody eliciting a human-anti-mouse-antibody (HAMA) response in humans. Art-recognized methods of determining immune response can be performed to monitor a HAMA response in a particular patient or during clinical trials. Patients administered humanized antibodies can be given an immunogenicity assessment at the beginning and throughout the administration of said therapy. The HAMA response is measured, for example, by detecting antibodies to the humanized therapeutic reagent, in serum samples from the patient using a method known to one in the art, including surface plasmon resonance technology (BIACORE) and/or solid-phase ELISA analysis. In many embodiments, a subject humanized antibody does not substantially elicit a HAMA response in a human subject.

Certain amino acids from the human variable region framework residues are selected for substitution based on their possible influence on CDR conformation and/or binding to antigen. The unnatural juxtaposition of murine CDR regions with human variable framework region can result in unnatural conformational restraints, which, unless corrected by substitution of certain amino acid residues, lead to loss of binding affinity. The selection of amino acid residues for substitution can be determined, in part, by computer modeling. Computer hardware and software for producing three-dimensional images of immunoglobulin molecules are known in the art. In general, molecular models are produced starting from solved structures for immunoglobulin chains or domains thereof. The chains to be modeled are compared for amino acid sequence similarity with chains or domains of solved three-dimensional structures, and the chains or domains showing the greatest sequence similarity is/are selected as starting points for construction of the molecular model. Chains or domains sharing at least 50% sequence identity are selected for modeling, and preferably those sharing at least 60%, 70%, 80%, 90% sequence identity or more are selected for modeling. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

When framework residues, as defined by Kabat, supra, constitute structural loop residues as defined by Chothia, supra, the amino acids present in the mouse antibody may be selected for substitution into the humanized antibody. Residues which are “adjacent to a CDR region” include amino acid residues in positions immediately adjacent to one or more of the CDRs in the primary sequence of the humanized immunoglobulin chain, for example, in positions immediately adjacent to a CDR as defined by Kabat, or a CDR as defined by Chothia (See e.g., Chothia and Lesk JMB 196:901 (1987)). These amino acids are particularly likely to interact with the amino acids in the CDRs and, if chosen from the acceptor, to distort the donor CDRs and reduce affinity. Moreover, the adjacent amino acids may interact directly with the antigen (Amit et al., Science, 233:747 (1986)) and selecting these amino acids from the donor may be desirable to keep all the antigen contacts that provide affinity in the original antibody.

In some embodiments, a subject antibody comprises scFv multimers. For example, in some embodiments, a subject antibody is an scFv dimer (e.g., comprises two tandem scFv (scFv₂)), an scFv trimer (e.g., comprises three tandem scFv (scFv3)), an scFv tetramer (e.g., comprises four tandem scFv (scFv₄)), or is a multimer of more than four scFv (e.g., in tandem). The scFv monomers can be linked in tandem via linkers of from about 2 amino acids to about 10 amino acids in length, e.g., 2 aa, 3 aa, 4 aa, 5 aa, 6 aa, 7 aa, 8 aa, 9 aa, or 10 aa in length. Suitable linkers include, e.g., (Gly)ₓ, where x is an integer from 2 to 10, glycine-serine polymers, and the like.

In certain embodiments, the antibody is conjugated to the agent via a cleavable or a non-cleavable linker. Linkers suitable for use a subject antibody include “flexible linkers.” If present, the linker molecules are generally of sufficient length to permit some flexible movement between linked regions. The linker molecules are generally about 6-50 atoms long. The linker molecules may also be, for example, aryl acetylene, ethylene glycol oligomers containing 2-10 monomer units, diamines, diacids, amino acids, or combinations thereof. Other linker molecules which can bind to polypeptides may be used in light of this disclosure.

According to some embodiments, the linker is a chemically-labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell (e.g., a cancer cell). Chemically-labile linkers include, but are not limited to, hydrazone-based linkers, oxime-based linkers, carbonate-based linkers, ester-based linkers, etc. In certain embodiments, the linker is an enzyme-labile linker, such as an enzyme-labile linker that is stable in the bloodstream but undergoes enzymatic cleavage upon internalization into a target cell, e.g., by a lysosomal protease (such as cathepsin or plasmin) in a lysosome of the target cell (e.g., a cancer cell). Enzyme-labile linkers include, but are not limited to, linkers that include peptidic bonds, e.g., dipeptide-based linkers such as valine-citrulline (VC) linkers, such as a maleimidocaproyl-valine-citruline-p-aminobenzyl (MC-vc-PAB) linker, a valyl-alanyl-para-aminobenzyloxy (Val-Ala-PAB) linker, and the like.

In some embodiments, a subject antibody comprises a constant region of an immunoglobulin (e.g., an Fc region). The Fc region, if present, can be a human Fc region. If constant regions are present, the antibody can contain both light chain and heavy chain constant regions. The antibodies described herein include antibodies having all types of constant regions, including IgM, IgG, IgD, IgA and IgE, and any isotype, including IgG1, IgG2, IgG3 and IgG4. An example of a suitable heavy chain Fc region is a human isotype IgG1 Fc. Light chain constant regions can be lambda or kappa. A subject antibody (e.g., a subject humanized antibody) can comprise sequences from more than one class or isotype. Antibodies can be expressed as tetramers containing two light and two heavy chains, as separate heavy chains, light chains, as Fab, Fab′ F(ab′)₂, and Fv, or as single chain antibodies in which heavy and light chain variable domains are linked through a spacer.

In some embodiments, an anti-MUC1 antibody of the present disclosure may include one or more amino acid substitutions introduced in the Fc region. In some embodiments, the one or more of the amino acid substitutions may be at the positions 239, 298, 326, 330 and 332 in the Fc region. In some embodiments, an anti-MUC1 antibody of the present disclosure may include one or more of the following amino acid substitutions introduced in the Fc region: I332E; S239D/A330L/I332E; S239D/S298A/I332E; S239D/K326T/I332E; S239D/S298A/K326T/I332E; or S239D/A330L/I332E/D356E/L358M.

In some embodiments, a subject antibody comprises a free thiol (—SH) group at the carboxyl terminus, where the free thiol group can be used to attach the antibody to a second polypeptide (e.g., another antibody, including a subject antibody), a scaffold, a carrier, etc.

In some embodiments, a subject antibody comprises one or more non-naturally occurring amino acids. In some embodiments, the non-naturally encoded amino acid comprises a carbonyl group, an acetyl group, an aminooxy group, a hydrazine group, a hydrazide group, a semicarbazide group, an azide group, or an alkyne group. Inclusion of a non-naturally occurring amino acid can provide for linkage to a polymer, a second polypeptide, a scaffold, etc. Examples of such non-naturally-occurring amino acids include, but are not limited to, N-acetylglucosaminyl-L-serine, N-acetylglucosaminyl-L-threonine, and O-phosphotyrosine.

The present disclosure also provides anti-MUC1 antibodies having an attached moiety of interest, e.g., a detectable label, drug, half-life-extending moiety, and the like. Modification of antibodies can be accomplished by a variety of synthetic and/or recombinant methods. The moiety or moieties attached to an antibody can provide for one or more of a wide variety of functions or features. Exemplary moieties include detectable labels (e.g., dye labels (e.g., chromophores, fluorophores), biophysical probes (spin labels, nuclear magnetic resonance (NMR) probes), fluorescence Resonance Energy Transfer (FRET)-type labels (e.g., at least one member of a FRET pair, including at least one member of a fluorophore/quencher pair), Bioluminescence Resonance Energy Transfer (BRET)-type labels (e.g., at least one member of a BRET pair), immunodetectable tags (e.g., FLAG, His(6), and the like); water soluble polymers (e.g., PEGylation); purification tags (e.g., to facilitate isolation by affinity chromatography (e.g., attachment of a FLAG epitope; membrane localization domains (e.g., lipids or glycophosphatidylinositol (GPI)-type anchors); immobilization tags (e.g., to facilitate attachment of the polypeptide to a surface, including selective attachment); drugs (e.g., to facilitate drug targeting, e.g., through attachment of the drug to an antibody); and the like.

In some embodiments, a subject antibody is linked (e.g., covalently linked) to a polymer (e.g., a polymer other than a polypeptide). Suitable polymers include, e.g., biocompatible polymers, and water-soluble biocompatible polymers. Suitable polymers include synthetic polymers and naturally-occurring polymers. Suitable polymers include, e.g., substituted or unsubstituted straight or branched chain polyalkylene, polyalkenylene or polyoxyalkylene polymers or branched or unbranched polysaccharides, e.g., a homo- or hetero-polysaccharide. Suitable polymers include, e.g., ethylene vinyl alcohol copolymer (commonly known by the generic name EVOH or by the trade name EVAL); polybutylmethacrylate; poly(hydroxyvalerate); poly(L-lactic acid); polycaprolactone; poly(lactide-co-glycolide); poly(hydroxybutyrate); poly(hydroxybutyrate-co-valerate); polydioxanone; polyorthoester; polyanhydride; poly(glycolic acid); poly(D,L-lactic acid); poly(glycolic acid-co-trimethylene carbonate); polyphosphoester; polyphosphoester urethane; poly(amino acids); cyanoacrylates; poly(trimethylene carbonate); poly(iminocarbonate); copoly(ether-esters) (e.g., poly(ethylene oxide)-poly(lactic acid) (PEO/PLA) co-polymers); polyalkylene oxalates; polyphosphazenes; biomolecules, such as fibrin, fibrinogen, cellulose, starch, collagen and hyaluronic acid; polyurethanes; silicones; polyesters; polyolefins; polyisobutylene and ethylene-alphaolefin copolymers; acrylic polymers and copolymers; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; amorphous Teflon; poly(ethylene glycol); and carboxymethyl cellulose.

Suitable synthetic polymers include unsubstituted and substituted straight or branched chain poly(ethyleneglycol), poly(propyleneglycol) poly(vinylalcohol), and derivatives thereof, e.g., substituted poly(ethyleneglycol) such as methoxypoly(ethyleneglycol), and derivatives thereof. Suitable naturally-occurring polymers include, e.g., albumin, amylose, dextran, glycogen, and derivatives thereof.

Suitable polymers can have an average molecular weight in a range of from 500 Da to 50000 Da, e.g., from 5000 Da to 40000 Da, or from 25000 to 40000 Da. For example, in some embodiments, where a subject antibody comprises a poly(ethylene glycol) (PEG) or methoxypoly(ethyleneglycol) polymer, the PEG or methoxypoly(ethyleneglycol) polymer can have a molecular weight in a range of from about 0.5 kiloDaltons (kDa) to 1 kDa, from about 1 kDa to 5 kDa, from 5 kDa to 10 kDa, from 10 kDa to 25 kDa, from 25 kDa to 40 kDa, or from 40 kDa to 60 kDa.

In some embodiments, a subject antibody is covalently linked to a PEG polymer. In some embodiments, a subject scFv multimer is covalently linked to a PEG polymer. PEG suitable for conjugation to a protein is generally soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons. The PEG conjugated to the subject antibody can be linear or branched. Branched PEG derivatives include star-PEG's and multi-armed PEG's.

A subject antibody can be glycosylated, e.g., a subject antibody can comprise a covalently linked carbohydrate or polysaccharide moiety. Glycosylation of antibodies is typically either N-linked or O-linked. Addition of glycosylation sites to an antibody is conveniently accomplished by altering the amino acid sequence such that it contains N- or O-linked glycosylation sites. Similarly, removal of glycosylation sites can be accomplished by amino acid alteration within the native glycosylation sites of an antibody.

A subject antibody can be covalently linked to a second moiety (e.g., a lipid, a polypeptide other than a subject antibody, a synthetic polymer, a carbohydrate, and the like) using for example, glutaraldehyde, a homobifunctional cross-linker, or a heterobifunctional cross-linker. Glutaraldehyde cross-links polypeptides via their amino moieties. Homobifunctional cross-linkers (e.g., a homobifunctional imidoester, a homobifunctional N-hydroxysuccinimidyl (NHS) ester, or a homobifunctional sulfhydryl reactive cross-linker) contain two or more identical reactive moieties and can be used in a one-step reaction procedure in which the cross-linker is added to a solution containing a mixture of the polypeptides to be linked. Homobifunctional NHS ester and imido esters cross-link amine containing polypeptides. In a mild alkaline pH, imido esters react only with primary amines to form imidoamides, and overall charge of the cross-linked polypeptides is not affected. Homobifunctional sulfhydryl reactive cross-linkers includes bismaleimidhexane (BMH), 1,5-difluoro-2,4-dinitrobenzene (DFDNB), and 1,4-di-(3′,2′-pyridyldithio) propinoamido butane (DPDPB).

Heterobifunctional cross-linkers have two or more different reactive moieties (e.g., amine reactive moiety and a sulfhydryl-reactive moiety) and are cross-linked with one of the polypeptides via the amine or sulfhydryl reactive moiety, then reacted with the other polypeptide via the non-reacted moiety. Multiple heterobifunctional haloacetyl cross-linkers are available, as are pyridyl disulfide cross-linkers. Carbodiimides are a classic example of heterobifunctional cross-linking reagents for coupling carboxyls to amines, which results in an amide bond.

A subject antibody will in some embodiments comprise a “radiopaque” label, e.g., a label that can be easily visualized using for example x-rays. Radiopaque materials are well known to those of skill in the art. The most common radiopaque materials include iodide, bromide or barium salts. Other radiopaque materials are also known and include, but are not limited to organic bismuth derivatives, radiopaque multiurethanes, organobismuth composites, radiopaque barium multimer complexes, and the like.

In some embodiments, a subject antibody comprises a polyamine modification. A subject antibody can be modified with polyamines that are either naturally occurring or synthetic. Useful naturally occurring polyamines include putrescine, spermidine, spermine, 1,3-deaminopropane, norspermidine, syn-homospermidine, thermine, thermospermine, caldopentamine, homocaldopentamine, and canavalmine. Putrescine, spermidine and spermine are particularly useful. Synthetic polyamines are composed of the empirical formula C_{X}H_{Y}N_{Z}, can be cyclic or acyclic, branched or unbranched, hydrocarbon chains of 3-12 carbon atoms that further include 1-6 NR or N(R)₂ moieties, wherein R is H, (C₁-C₄) alkyl, phenyl, or benzyl. Polyamines can be linked to an antibody using any standard crosslinking method.

Where an anti-MUC1 antibody of the present disclosure comprises a covalently linked heterologous moiety, the heterologous moiety can be linked to the anti-MUC1 heavy and/or light chain directly or via a linker. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Examples of flexible linkers include glycine polymers (G)n, glycine-serine polymers (including, for example, (GS)ₙ, (GSGGS)ₙ (SEQ ID NO:21) and (GGGS)ₙ (SEQ ID NO:22), where n is an integer of at least one), glycine-alanine polymers, alanine-serine polymers, and other flexible linkers known in the art.

### Methods for Modification of Antibodies

An anti-MUC1 antibody conjugate of the present disclosure can include: 1) Ig heavy chain constant region conjugated to a moiety of interest; and an Ig light chain constant region conjugated to a moiety of interest; 2) an Ig heavy chain constant region conjugated to a moiety of interest; and an Ig light chain constant region that is not conjugated to a moiety of interest; or 3) an Ig heavy chain constant region that is not conjugated to a moiety of interest; and an Ig light chain constant region conjugated to a moiety of interest. A subject anti-MUC1 antibody conjugate can also include VH and/or VL domains conjugated to a moiety of interest.

In one example, the antibody can be modified to include a 2-formylglycine residue, which can serve as a chemical handle for attachment of a heterologous moiety. For example, the heavy and/or light chain constant region of an anti-MUC1 of the present disclosure can be modified to include an amino acid sequence of a sulfatase motif which is capable of being converted by action of a 2-formylglycine generating enzyme (FGE) to contain a 2-formylglycine (fGly). Such sulfatase motifs may also be referred to herein as an FGE-modification site. Action of FGE is directed in a sequence-specific manner in that the FGE acts at a sulfatase motif positioned within the immunoglobulin polypeptide. The moiety of interest is provided as a component of a reactive partner for reaction with an aldehyde of the fGly residue of a converted aldehyde tag of the tagged Ig polypeptide. A wide range of commercially available reagents can be used to accomplish attachment of a moiety of interest to an fGly residue of an aldehyde tagged Ig polypeptide. For example, aminooxy, hydrazide, or thiosemicarbazide derivatives of a number of moieties of interest are suitable reactive partners, and are readily available or can be generated using standard chemical methods.

As noted above, the amino acid sequence of an anti-MUC1 antibody can be modified to include a sulfatase motif that contains a serine or cysteine residue that is capable of being converted (oxidized) to a 2-formylglycine (fGly) residue by action of a formylglycine generating enzyme (FGE) either in vivo (e.g., at the time of translation of an aldehyde tag-containing protein in a cell) or in vitro (e.g., by contacting an aldehyde tag-containing protein with an FGE in a cell-free system). Such sulfatase motifs may also be referred to herein as an FGE-modification site.

### Sulfatase Motifs

A minimal sulfatase motif of an aldehyde tag is usually 5 or 6 amino acid residues in length, usually no more than 6 amino acid residues in length. Sulfatase motifs provided in an Ig polypeptide are at least 5 or 6 amino acid residues, and can be, for example, from 5 to 16, 6-16, 5-15, 6-15, 5-14, 6-14, 5-13, 6-13, 5-12, 6-12, 5-11, 6-11, 5-10, 6-10, 5-9, 6-9, 5-8, or 6-8 amino acid residues in length, so as to define a sulfatase motif of less than 16, 15, 14, 13, 12, 11, 10, 9, 8 or 7 amino acid residues in length.

In certain embodiments, polypeptides of interest include those where one or more amino acid residues, such as 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more amino acid residues have been inserted, deleted, substituted (replaced) relative to the native amino acid sequence to provide for a sequence of a sulfatase motif in the polypeptide. In certain embodiments, the polypeptide includes a modification (insertion, addition, deletion, and/or substitution/replacement) of less than 20, 19, 18, 17, 16, 15, 14, 13, 12, 11, 10, 9, 8, 7, 6, 5, 4, 3 or 2 amino acid residues of the amino acid sequence relative to the native amino acid sequence of the polypeptide. Where an amino acid sequence native to the polypeptide (e.g., anti-MUC1 antibody) contains one or more residues of the desired sulfatase motif, the total number of modifications of residues can be reduced, e.g., by site-specification modification (insertion, addition, deletion, substitution/replacement) of amino acid residues flanking the native amino acid residues to provide a sequence of the desired sulfatase motif. In certain embodiments, the extent of modification of the native amino acid sequence of the target anti-MUC1 polypeptide is minimized, so as to minimize the number of amino acid residues that are inserted, deleted, substituted (replaced), or added (e.g., to the N- or C-terminus). Minimizing the extent of amino acid sequence modification of the target anti-MUC1 polypeptide may minimize the impact such modifications may have upon anti-MUC1 function and/or structure.

It should be noted that while aldehyde tags of particular interest are those comprising at least a minimal sulfatase motif (also referred to a “consensus sulfatase motif”), it will be readily appreciated that longer aldehyde tags are both contemplated and encompassed by the present disclosure and can find use in the compositions and methods of the present disclosure. Aldehyde tags can thus comprise a minimal sulfatase motif of 5 or 6 residues, or can be longer and comprise a minimal sulfatase motif which can be flanked at the N- and/or C-terminal sides of the motif by additional amino acid residues. Aldehyde tags of, for example, 5 or 6 amino acid residues are contemplated, as well as longer amino acid sequences of more than 5, 6, 7, 8, 9, 10, 11, 12, 13, 14, 15, 16, 17, 18, 19, 20 or more amino acid residues.

An aldehyde tag can be present at or near the C-terminus of an Ig heavy chain; e.g., an aldehyde tag can be present within 1, 2, 3, 4, 5, 6, 7, 8, 9, or 10 amino acids of the C-terminus of a native, wild-type Ig heavy chain. An aldehyde tag can be present within a CH₁ domain of an Ig heavy chain. An aldehyde tag can be present within a CH₂ domain of an Ig heavy chain. An aldehyde tag can be present within a CH₃ domain of an Ig heavy chain. An aldehyde tag can be present in an Ig light chain constant region, e.g., in a kappa light chain constant region or a lambda light chain constant region.

In certain embodiments, the sulfatase motif used may be described by the formula:

X¹Z¹X²Z2X³Z³ (I′), where
Z¹ is cysteine or serine (which can also be represented by (C/S));
Z² is either a proline or alanine residue (which can also be represented by (P/A));
Z³ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), usually A, G, L, V, or I;
X¹ is present or absent and, when present, can be any amino acid, though usually an aliphatic amino acid, a sulfur-containing amino acid, or a polar, uncharged amino acid, (e.g., other than an aromatic amino acid or a charged amino acid), usually L, M, V, S or T, more usually L, M, S or V, with the proviso that when the sulfatase motif is at the N-terminus of the target polypeptide, X¹ is present; and
X² and X³ independently can be any amino acid, though usually an aliphatic amino acid, a polar, uncharged amino acid, or a sulfur containing amino acid (e.g., other than an aromatic amino acid or a charged amino acid), e.g., S, T, A, V, G or C; e.g., S, T, A, V or G. In one example, the aldehyde tag is of the formula L(C/S)TPSR (SEQ ID NO:5), e.g., LCTPSR (SEQ ID NO:6) or LSTPSR (SEQ ID NO:23). Thus, the present disclosure provides antibodies that include an aldehyde-tagged Ig heavy chain and/or an aldehyde-tagged Ig light chain, where the aldehyde-tagged Ig antibody comprises an Ig constant region amino acid sequence of the heavy and/or light chain contains such a sulfatase motif.

For example, in some embodiments, the amino acid sequence of an anti-MUC1 heavy and/or light chain can be modified to provide a sequence of at least 5 amino acids of the formula X¹Z¹X²Z²X³Z³, where
Z¹ is cysteine or serine;
Z² is a proline or alanine residue;
Z³ is an aliphatic amino acid or a basic amino acid;
X¹ is present or absent and, when present, is any amino acid, with the proviso that when the heterologous sulfatase motif is at an N-terminus of the polypeptide, X¹ is present;
X² and X³ are each independently any amino acid,
where the sequence is within or adjacent a solvent-accessible loop region of the Ig constant region, and wherein the sequence is not at the C-terminus of the Ig heavy chain.

The sulfatase motif is generally selected so as to be capable of conversion by a selected FGE, e.g., an FGE present in a host cell in which the aldehyde tagged polypeptide is expressed or an FGE which is to be contacted with the aldehyde tagged polypeptide in a cell-free in vitro method.

For example, where the FGE is a eukaryotic FGE (e.g., a mammalian FGE, including a human FGE), the sulfatase motif can be of the formula:

X¹CX²PX³Z³ (I″)
where
X¹ may be present or absent and, when present, can be any amino acid, e.g., an aliphatic amino acid, a sulfur-containing amino acid, or a polar, uncharged amino acid, (e.g., other than an aromatic amino acid or a charged amino acid), e.g., L, M, S or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (e.g., other than an aromatic amino acid or a charged amino acid), e.g., S, T, A, V, G, or C, e.g., S, T, A, V or G; and
Z³ is a basic amino acid (e.g., arginine (R), and may be lysine (K) or histidine (H), e.g., lysine), or an aliphatic amino acid (alanine (A), glycine (G), leucine (L), valine (V), isoleucine (I), or proline (P), e.g., A, G, L, V, or I.

Specific examples of sulfatase motifs include LCTPSR (SEQ ID NO:6), MCTPSR (SEQ ID NO:97), VCTPSR (SEQ ID NO:76), LCSPSR (SEQ ID NO:77), LCAPSR (SEQ ID NO:78), LCVPSR (SEQ ID NO:79), LCGPSR (SEQ ID NO:80), ICTPAR (SEQ ID NO:81), LCTPSK (SEQ ID NO:82), MCTPSK (SEQ ID NO:83), VCTPSK (SEQ ID NO:84), LCSPSK (SEQ ID NO:85), LCAPSK (SEQ ID NO:86), LCVPSK (SEQ ID NO:87), LCGPSK (SEQ ID NO:88), LCTPSA (SEQ ID NO:89), ICTPAA (SEQ ID NO:90), MCTPSA (SEQ ID NO:91), VCTPSA (SEQ ID NO:92), LCSPSA (SEQ ID NO:93), LCAPSA (SEQ ID NO:94), LCVPSA (SEQ ID NO:95), and LCGPSA (SEQ ID NO:96).

fGly-Containing Sequences

In general, the FGE used to facilitate conversion of cysteine or serine to fGly in a sulfatase motif of an aldehyde tag of a target polypeptide is selected according to the sulfatase motif present in the aldehyde tag. The FGE can be native to the host cell in which the aldehyde tagged polypeptide is expressed, or the host cell can be genetically modified to express an appropriate FGE. In some embodiments it may be desired to use a sulfatase motif compatible with a human FGE, and express the aldehyde tagged protein in a human cell that expresses the FGE or in a host cell, usually a mammalian cell, genetically modified to express a human FGE. In general, an FGE suitable for use in generating an fGly-modified antibody can be obtained from naturally occurring sources or synthetically produced. For example, an appropriate FGE can be derived from biological sources which naturally produce an FGE or which are genetically modified to express a recombinant gene encoding an FGE. Nucleic acids encoding a number of FGEs are known in the art and readily.

Following action of an FGE on the sulfatase motif, Z¹ is oxidized to generate a 2-formylglycine (fGly) residue. Furthermore, following both FGE-mediated conversion and reaction with a reactive partner comprising a moiety of interest, the fGly position at Z¹ in the formula above is covalently bound to the moiety of interest (e.g., detectable label, water soluble polymer, polypeptide, drug, active agent, etc.). Thus, the present disclosure provides an anti-MUC1 antibody having an amino acid sequence modified to comprise an fGly moiety.

Upon action of FGE on the anti-MUC1 heavy and/or light chain, the serine or the cysteine in the sulfatase motif is modified to fGly. Thus, the fGly-containing sulfatase motif can be of the formula:

X¹(fGly)X²Z²X³Z³ (I′″)
where
   fGly is the formylglycine residue;
   Z² is either a proline or alanine residue (which can also be represented by (P/A));
   Z³ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
   X¹ may be present or absent and, when present, can be any amino acid, e.g., an aliphatic amino acid, a sulfur-containing amino acid, or a polar, uncharged amino acid, (e.g., other than an aromatic amino acid or a charged amino acid), e.g., L, M, V, S or T, e.g., L, M or V, with the proviso that when the sulfatase motif is at the N-terminus of the target polypeptide, X¹ is present; and
   X² and X³ independently can be any amino acid, e.g., an aliphatic amino acid, a sulfur-containing amino acid, or a polar, uncharged amino acid, (e.g., other than an aromatic amino acid or a charged amino acid), e.g., S, T, A, V, G or C, e.g., S, T, A, V or G.

As described above, to produce the conjugate, the polypeptide containing the fGly residue may be conjugated to a drug or active agent by reaction of the fGly with a reactive moiety (e.g., hydrazinyl-indolyl or a hydrazinyl-pyrrolo-pyridinyl coupling moiety, as described above) of a linker attached to the drug or active agent to produce an fGly′-containing sulfatase motif. As used herein, the term fGly′ refers to the amino acid residue of the sulfatase motif that is coupled to the drug or active agent through a linker as described herein. Thus, the present disclosure provides an anti-MUC1 antibody conjugate (also referred to herein as an “anti-MUC1 conjugate”).

In certain embodiments, the anti-MUC1 conjugate comprises an fGly′-containing sulfatase motif of the formula:

X¹(fGly′)X²Z²X³Z³ (II)
where
fGly′ is the amino acid residue coupled to the drug or active agent through a linker as described herein;
Z² is either a proline or alanine residue (which can also be represented by (P/A));
Z³ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (e.g., other than an aromatic amino acid or a charged amino acid), e.g., L, M, V, S or T, e.g., L, M or V, with the proviso that when the sulfatase motif is at the N-terminus of the target polypeptide, X¹ is present; and
X² and X³ independently can be any amino acid, e.g., an aliphatic amino acid, a sulfur-containing amino acid, or a polar, uncharged amino acid, (e.g., other than an aromatic amino acid or a charged amino acid), e.g., S, T, A, V, G or C, e.g., S, T, A, V or G.

In certain embodiments, the sequence of formula (III) is positioned at a C-terminus of a heavy chain constant region of the anti-MUC1 antibody. In some instances, the heavy chain constant region comprises a sequence of the formula (III):

X¹(fGly′)X²Z²X³Z³ (III)
where
fGly′ is the amino acid residue coupled to the drug or active agent through a linker as described herein;
Z² is either a proline or alanine residue (which can also be represented by (P/A));
Z³ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (e.g., other than an aromatic amino acid or a charged amino acid), e.g., L, M, V, S or T, e.g., L, M or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (e.g., other than an aromatic amino acid or a charged amino acid), e.g., S, T, A, V, G or C, e.g., S, T, A, V or G; and
wherein the sequence is C-terminal to the amino acid sequence QKSLSLSPGK (SEQ ID NO:98), and where the sequence may include 1, 2, 3, 4, 5, or from 5 to 10, amino acids that are not present in a native, wild-type heavy Ig chain constant region.

In certain embodiments, the heavy chain constant region comprises the sequence SLSLSPGSL(fGly′)TPSRGS (SEQ ID NO:99) at the C-terminus of the Ig heavy chain, e.g., in place of a native SLSLSPGK (SEQ ID NO:100) sequence.

In certain embodiments, the amino acid residue coupled to the drug or active agent (fGly′) is positioned in a light chain constant region of the anti-MUC1 antibody. In certain embodiments, the light chain constant region comprises a sequence of the formula (III):

X¹(fGly′)X²Z²X³Z³ (III)
where
fGly′ is the amino acid residue coupled to the drug or active agent through a linker as described herein;
Z² is either a proline or alanine residue (which can also be represented by (P/A));
Z³ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (e.g., other than an aromatic amino acid or a charged amino acid), e.g., L, M, V, S or T, e.g., L, M or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (e.g., other than an aromatic amino acid or a charged amino acid), e.g., S, T, A, V, G or C, e.g., S, T, A, V or G; and
wherein the sequence is C-terminal to the amino acid sequence KVDNAL (SEQ ID NO:101) and/or is N-terminal to the amino acid sequence QSGNSQ (SEQ ID NO:102).

In certain embodiments, the light chain constant region comprises the sequence KVDNAL(fGly′)TPSRQSGNSQ (SEQ ID NO: 103).

In certain embodiments, the amino acid residue coupled to the drug or active agent (fGly′) is positioned in a heavy chain CH₁ region of the anti-MUC1 antibody. In certain embodiments, the heavy chain CH₁ region comprises a sequence of the formula (III):

X¹(fGly′)X²Z²X³Z³ (III)

where
fGly′ is the amino acid residue coupled to the drug or active agent through a linker as described herein;
Z² is either a proline or alanine residue (which can also be represented by (P/A));
Z³ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (e.g., other than an aromatic amino acid or a charged amino acid), e.g., L, M, V, S or T, e.g., L, M or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (e.g., other than an aromatic amino acid or a charged amino acid), e.g., S, T, A, V, G or C, e.g., S, T, A, V or G; and
wherein the sequence is C-terminal to the amino acid sequence SWNSGA (SEQ ID NO:104) and/or is N-terminal to the amino acid sequence GVHTFP (SEQ ID NO: 105).

In certain embodiments, the heavy chain CH₁ region comprises the sequence SWNSGAL(fGly′)TPSRGVHTFP (SEQ ID NO: 106).

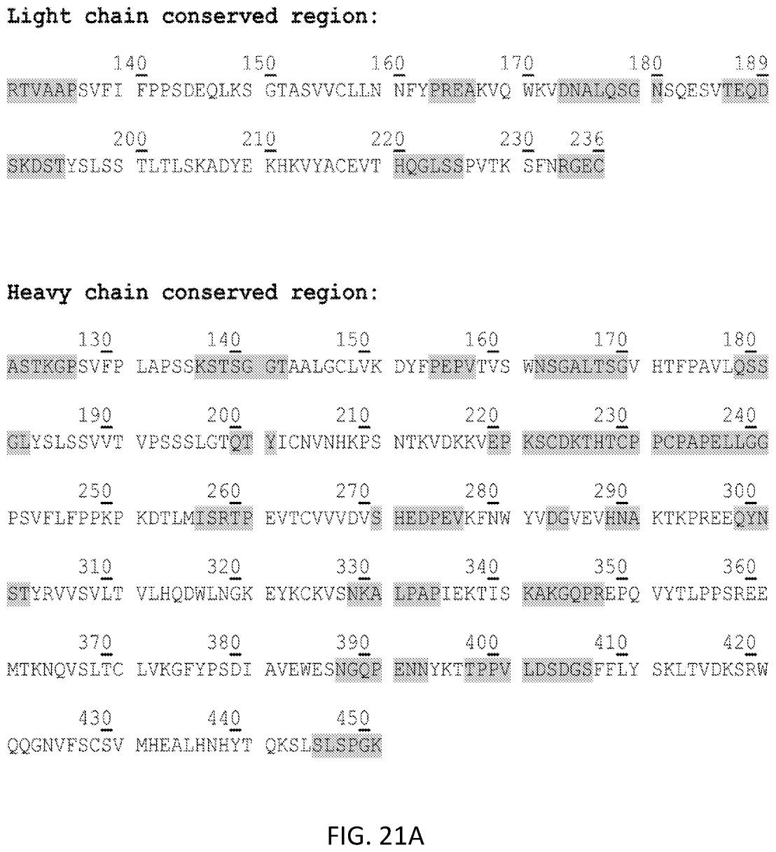

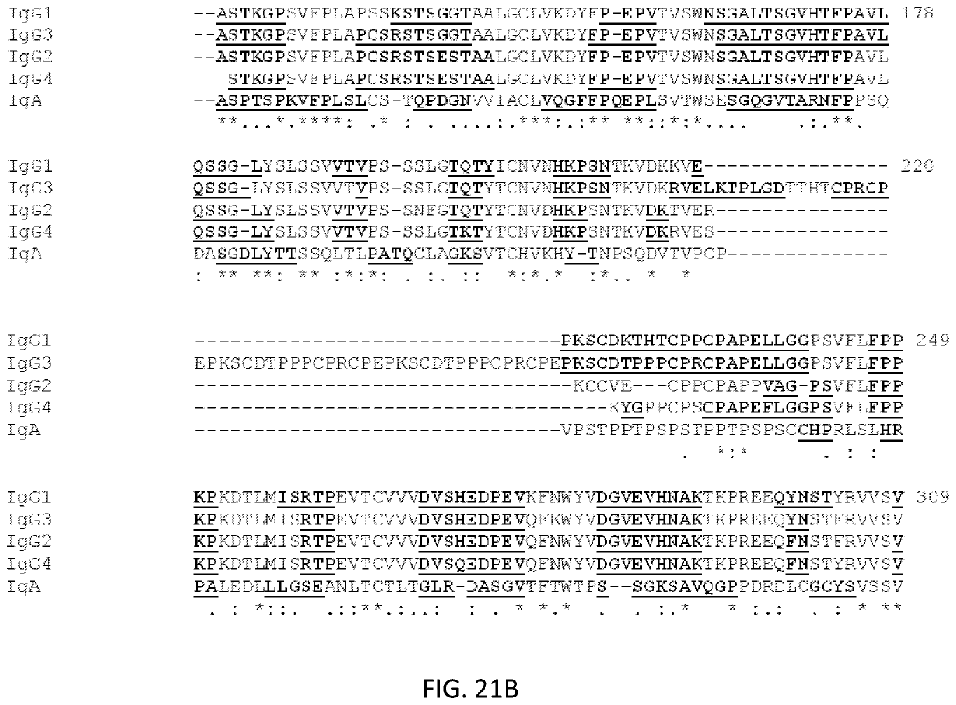

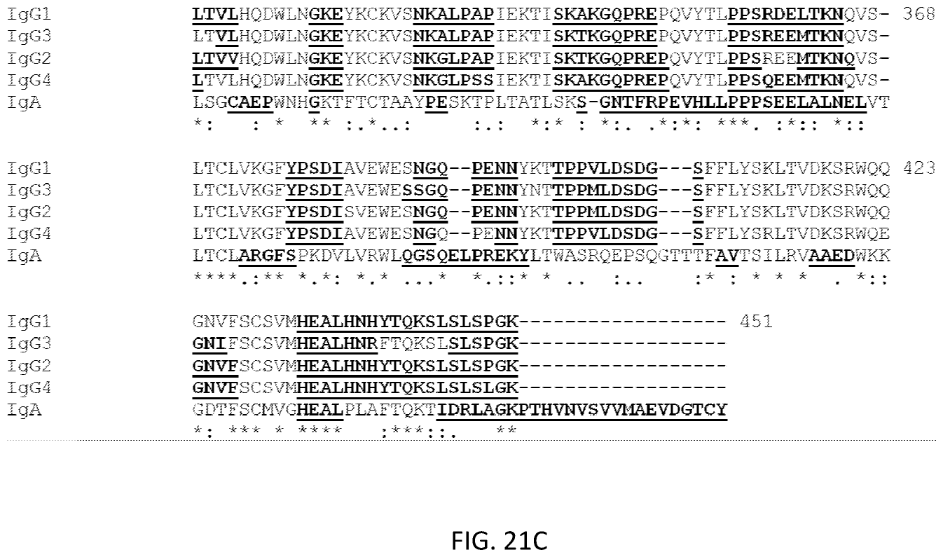

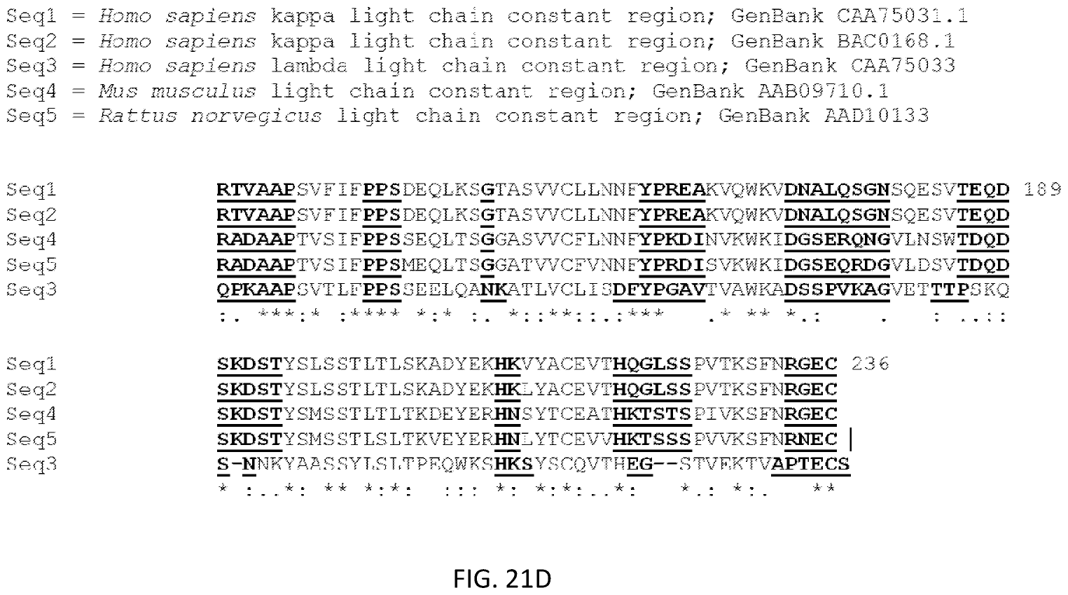

FIG. 21A depicts a site map showing possible modification sites for generation of an aldehyde tagged Ig polypeptide. The upper sequence is the amino acid sequence of the conserved region of an IgG1 light chain polypeptide (SEQ ID NO:74) and shows possible modification sites in an Ig light chain; the lower sequence is the amino acid sequence of the conserved region of an Ig heavy chain polypeptide (SEQ ID NO:75) (GenBank Accession No. AAG00909) and shows possible modification sites in an Ig heavy chain. The heavy and light chain numbering is based on the full-length heavy and light chains.

FIG. 21B depicts an alignment of Homo sapiens immunoglobulin heavy chain constant regions for IgG1 (SEQ ID NO:47; GenBank P01857.1), IgG2 (SEQ ID NO:48; GenBank P01859.2), IgG3 (SEQ ID NO:49; GenBank P01860.2), IgG4 (SEQ ID NO:50; GenBank AAB59394.1), and IgA (SEQ ID NO:51; GenBank AAAT74070), showing modification sites at which aldehyde tags can be provided in an immunoglobulin heavy chain. The heavy and light chain numbering is based on the full heavy and light chains.

FIG. 21C depicts an alignment of immunoglobulin light chain constant regions, showing modification sites at which aldehyde tags can be provided in an immunoglobulin light chain. Seq1=Homo sapiens kappa light chain constant region; GenBank CAA75031.1; SEQ ID NO:52. Seq2=Homo sapiens kappa light chain constant region; GenBank BAC0168.1; SEQ ID NO:53. Seq3=Homo sapiens lambda light chain constant region; GenBank CAA75033; SEQ ID NO:54. Seq4=Mus musculus light chain constant region; GenBank AAB09710.1; SEQ ID NO:55. Seq5=Rattus norvegicus light chain constant region; GenBank AAD10133; SEQ ID NO:56.

In some embodiments the sulfatase motif is at a position other than, or in addition to, the C-terminus of the Ig polypeptide heavy chain. An isolated aldehyde-tagged anti-MUC1 polypeptide can comprise a heavy chain constant region amino acid sequence modified to include a sulfatase motif as described herein, where the sulfatase motif is in or adjacent a surface-accessible loop region of the anti-MUC1 polypeptide heavy chain constant region.

Exemplary surface-accessible loop regions of an IgG1 heavy chain include: 1) ASTKGP (SEQ ID NO:10⁷); 2) KSTSGGT (SEQ ID NO:10⁸); 3) PEPV (SEQ ID NO:10⁹); 4) NSGALTSG (SEQ ID NO:110); 5) NSGALTSGVHTFPAVLQSSGL (SEQ ID NO:111); 6) QSSGL (SEQ ID NO:112); 7) VTV; 8) QTY; 9) TQTY (SEQ ID NO:113); 10) HKPSN (SEQ ID NO:114); 11) EPKSCDKTHTCPPCPAPELLGG (SEQ ID NO:115); 12) FPPKP (SEQ ID NO:116); 13) ISRTP (SEQ ID NO:117); 14) DVSHEDPEV (SEQ ID NO:118); 15) SHEDPEV (SEQ ID NO:119); 16) DG; 17) DGVEVHNAK (SEQ ID NO:120); 18) HNA; 19) QYNST (SEQ ID NO:121); 20) VLTVL (SEQ ID NO:122); 21) GKE; 22) NKALPAP (SEQ ID NO:123); 23) SKAKGQPRE (SEQ ID NO:124); 24) KAKGQPR (SEQ ID NO:125); 25) PPSRKELTKN (SEQ ID NO:126); 26) YPSDI (SEQ ID NO:127); 27) NGQPENN (SEQ ID NO:128); 28) TPPVLDSDGS (SEQ ID NO:129); 29) HEALHNHYTQKSLSLSPGK (SEQ ID NO:130); and 30) SLSPGK (SEQ ID NO:131).

Exemplary surface-accessible loop regions of an IgG2 heavy chain include 1) ASTKGP (SEQ ID NO:10⁷); 2) PCSRSTSESTAA (SEQ ID NO:132); 3) FPEPV (SEQ ID NO:133); 4) SGALTSGVHTFP (SEQ ID NO:134); 5) QSSGLY (SEQ ID NO:135); 6) VTV; 7) TQT; 8) HKP; 9) DK; 10) VAGPS (SEQ ID NO:136); 11) FPPKP (SEQ ID NO:116); 12) RTP; 13) DVSHEDPEV (SEQ ID NO:118); 14) DGVEVHNAK (SEQ ID NO:120); 15) FN; 16) VLTVV (SEQ ID NO:137); 17) GKE; 18) NKGLPAP (SEQ ID NO:138); 19) SKTKGQPRE (SEQ ID NO:139); 20) PPS; 21) MTKNQ (SEQ ID NO:140); 22) YPSDI (SEQ ID NO:127); 23) NGQPENN (SEQ ID NO:128); 24) TPPMLDSDGS (SEQ ID NO:141); 25) GNVF (SEQ ID NO:142); and 26) HEALHNHYTQKSLSLSPGK (SEQ ID NO:130).

Exemplary surface-accessible loop regions of an IgG3 heavy chain include 1) ASTKGP (SEQ ID NO:10⁷); 2) PCSRSTSGGT (SEQ ID NO:143); 3) FPEPV (SEQ ID NO:133); 4) SGALTSGVHTFPAVLQSSG (SEQ ID NO:144); 5) V; 6) TQT; 7) HKPSN (SEQ ID NO:114); 8) RVELKTPLGD (SEQ ID NO:145); 9) CPRCPKP (SEQ ID NO:146); 10) PKSCDTPPPCPRCPAPELLGG (SEQ ID NO:147); 11) FPPKP (SEQ ID NO:116); 12) RTP; 13) DVSHEDPEV (SEQ ID NO:118); 14) DGVEVHNAK (SEQ ID NO:120); 15) YN; 16) VL; 17) GKE; 18) NKALPAP (SEQ ID NO:123); 19) SKTKGQPRE (SEQ ID NO:139); 20) PPSREEMTKN (SEQ ID NO:148); 21) YPSDI (SEQ ID NO:127); 22) SSGQPENN (SEQ ID NO:149); 23) TPPMLDSDGS (SEQ ID NO:141); 24) GNI; 25) HEALHNR (SEQ ID NO:150); and 26) SLSPGK (SEQ ID NO:131).

Exemplary surface-accessible loop regions of an IgG4 heavy chain include 1) STKGP (SEQ ID NO:151); 2) PCSRSTSESTAA (SEQ ID NO:132); 3) FPEPV (SEQ ID NO:133); 4) SGALTSGVHTFP (SEQ ID NO:134); 5) QSSGLY (SEQ ID NO:135); 6) VTV; 7) TKT; 8) HKP; 9) DK; 10) YG; 11) CPAPEFLGGPS (SEQ ID NO:152); 12) FPPKP (SEQ ID NO:116); 13) RTP; 14) DVSQEDPEV (SEQ ID NO:153); 15) DGVEVHNAK (SEQ ID NO:120); 16) FN; 17) VL; 18) GKE; 19) NKGLPSS (SEQ ID NO:154); 20) SKAKGQPREP (SEQ ID NO:155); 21) PPSQEEMTKN (SEQ ID NO:156); 22) YPSDI (SEQ ID NO:127); 23) NG; 24) NN; 25) TPPVLDSDGS (SEQ ID NO:129); 26) GNVF (SEQ ID NO:142); and 27) HEALHNHYTQKSLSLSLGK (SEQ ID NO:157).

Exemplary surface-accessible loop regions of an IgA heavy chain include 1) ASPTSPKVFPLSL (SEQ ID NO:158); 2) QPDGN (SEQ ID NO:159); 3) VQGFFPQEPL (SEQ ID NO:160); 4) SGQGVTARNFP (SEQ ID NO:161); 5) SGDLYTT (SEQ ID NO:162); 6) PATQ (SEQ ID NO:163); 7) GKS; 8) YT; 9) CHP; 10) HRPA (SEQ ID NO:164); 11) LLGSE (SEQ ID NO:165); 12) GLRDASGV (SEQ ID NO:166); 13) SSGKSAVQGP (SEQ ID NO:167); 14) GCYS (SEQ ID NO:168); 15) CAEP (SEQ ID NO:169); 16) PE; 17) SGNTFRPEVHLLPPPSEELALNEL (SEQ ID NO:170); 18) ARGFS (SEQ ID NO:171); 19) QGSQELPREKY (SEQ ID NO:172); 20) AV; 21) AAED (SEQ ID NO:173); 22) HEAL (SEQ ID NO:174); and 23) IDRLAGKPTHVNVSVVMAEVDGTCY (SEQ ID NO:175).

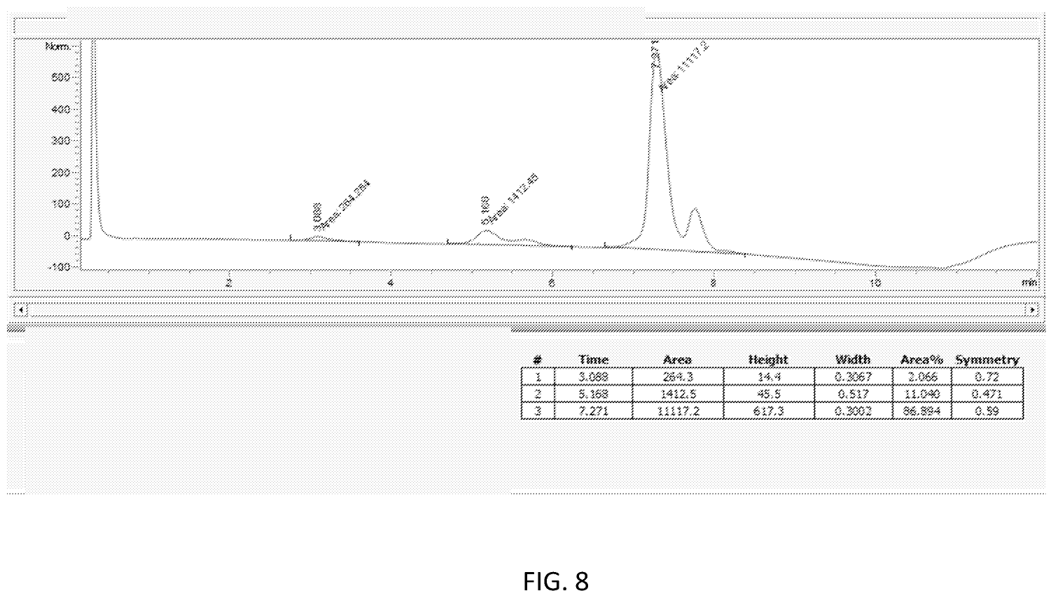

Exemplary surface-accessible loop regions of an Ig light chain (e.g., a human kappa light chain) include: 1) RTVAAP (SEQ ID NO:176); 2) PPS; 3) Gly (see, e.g., Gly at position 150 of the human kappa light chain sequence depicted in FIG. 8C); 4) YPREA (SEQ ID NO:177); 5) PREA (SEQ ID NO:178); 6) DNALQSGN (SEQ ID NO:179); 7) TEQDSKDST (SEQ ID NO:180); 8) HK; 9) HQGLSS (SEQ ID NO:181); and 10) RGEC (SEQ ID NO:182).

Exemplary surface-accessible loop regions of an Ig lambda light chain include QPKAAP (SEQ ID NO:183), PPS, NK, DFYPGAV (SEQ ID NO:184), DSSPVKAG (SEQ ID NO:185), TTP, SN, HKS, EG, and APTECS (SEQ ID NO:186).

The constant region of the HC of an anti-MUC1 antibody as disclosed herein may be selected from one of the following sequences:

CT-Tagged (Aldehyde Tag-in bold)
(SEQ ID NO: 57)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGS
FFLYSKLTVDKSRWQQGNVFSCSVMHEALHNHYTQKSLSLSPGSLCTPSRGS

In SEQ ID NO:57, the italicized residues at the C-terminus of the heavy chain constant region replace a lysine residue at the C-terminus of a standard IgG1 heavy chain. The bolded residues (LCTPSR) among the italicized residues constitute the aldehyde tag, where the C is converted to an fGly residue by FGE upon expression of the heavy chain. The fGly can be converted to fGly′. fGly′ refers to the amino acid residue of the anti-MUC1 antibody that is coupled to the moiety of interest (e.g., a drug). The non-bolded residues among the italicized residues are additional residues that are different from a standard IgG1 heavy chain sequence.

58Q-1 (Aldehyde Tag-in bold and substitution of “EEM” with “DEL”)
(SEQ ID NO: 58)
ASTKGPSVFPLAPSSKSTSGGTAALGCLVKDYFPEPVTVSWNSGALTSGV
HTFPAVLLCTPSRQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK.
61G-1 (Aldehyde Tag-in bold and substitution of “EEM” with “DEL”)
(SEQ ID NO: 59)
ASTKGPSVFPLAPSSKSTSGGTAALGCLVKDYFPEPVTVSWNSGALTSGV
HTFPAVLQSSLCTPSRGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK.
91N-1 (Aldehyde Tag-in bold and substitution of “EEM” with “DEL”)
(SEQ ID NO: 60)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSLCTPSRNTKVDKKVEPKSCD
KTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK.
116E-1 (Aldehyde Tag-in bold and substitution of “EEM” with “DEL”)
(SEQ ID NO: 61)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCP
PCPAPLCTPSRELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK.
58Q-2 (Aldehyde Tag-in bold)
(SEQ ID NO: 62)
ASTKGPSVFPLAPSSKSTSGGTAALGCLVKDYFPEPVTVSWNSGALTSGV
HTFPAVLLCTPSRQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK.
61G-2 (Aldehyde Tag-in bold)
(SEQ ID NO: 63)
ASTKGPSVFPLAPSSKSTSGGTAALGCLVKDYFPEPVTVSWNSGALTSGV
HTFPAVLQSSLCTPSRGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK.
91N-2 (Aldehyde Tag-in bold)
(SEQ ID NO: 64)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSLCTPSRNTKVDKKVEPKSCD
KTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK.
116E-2 (Aldehyde Tag-in bold)
(SEQ ID NO: 65)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCP
PCPAPLCTPSRELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK.
58Q-3 (Aldehyde Tag-in bold and substitution of “KKV” with “KRV” and
substitution of “EEM” with “DEL”)
(SEQ ID NO: 66)
ASTKGPSVFPLAPSSKSTSGGTAALGCLVKDYFPEPVTVSWNSGALTSGV
HTFPAVLLCTPSRQSSGLYSLSSVVTVPSSSLGTQTYICNVNHKPSNTKVDKRVEPKSCD
KTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK.
61G-3 (Aldehyde Tag-in bold and substitution of “KKV” with “KRV” and
substitution of “EEM” with “DEL”)
(SEQ ID NO: 67)
ASTKGPSVFPLAPSSKSTSGGTAALGCLVKDYFPEPVTVSWNSGALTSGV
HTFPAVLQSSLCTPSRGLYSLSSVVTVPSSSLGTQTYICNVNHKPSNTKVDKRVEPKSCD
KTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK.
91N-3 (Aldehyde Tag-in bold and substitution of “KKV” with “KRV” and
substitution of “EEM” with “DEL”)
(SEQ ID NO: 68)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSLCTPSRNTKVDKRVEPKSCD
KTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK.
116E-3 (Aldehyde Tag-in bold and substitution of “KKV” with “KRV” and
substitution of “EEM” with “DEL”)
(SEQ ID NO: 69)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHTCP
PCPAPLCTPSRELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK.
58Q-4 (Aldehyde Tag-in bold and substitution of “KKV” with “KRV”)
(SEQ ID NO: 70)
ASTKGPSVFPLAPSSKSTSGGTAALGCLVKDYFPEPVTVSWNSGALTSGV
HTFPAVLLCTPSRQSSGLYSLSSVVTVPSSSLGTQTYICNVNHKPSNTKVDKRVEPKSCD
KTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK.
61G-4 (Aldehyde Tag-in bold and substitution of “KKV” with “KRV”)
(SEQ ID NO: 71)
ASTKGPSVFPLAPSSKSTSGGTAALGCLVKDYFPEPVTVSWNSGALTSGV
HTFPAVLQSSLCTPSRGLYSLSSVVTVPSSSLGTQTYICNVNHKPSNTKVDKRVEPKSCD
KTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
91N-4 (Aldehyde Tag-in bold and substitution of “KKV” with “KRV”)
(SEQ ID NO: 72)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSLCTPSRNTKVDKRVEPKSCD
KTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK.
116E-4 (Aldehyde Tag-in bold and substitution of “KKV” with “KRV”)
(SEQ ID NO: 73)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHTCP
PCPAPLCTPSRELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIA VEWESNGQPENNYKTTPPV
LDSDGSFFLYSKLTVDKSRWQQGNVFSCSVMHEALHNHYTQKSLSLSPGK.

The bolded residues (LCTPSR) constitute the aldehyde tag, where the C is converted to an fGly residue by FGE upon expression of the heavy chain. The fGly can be converted to fGly′. fGly′ refers to the amino acid residue of the anti-MUC1 antibody that is coupled to the moiety of interest (e.g., a drug).

### Drugs

In some cases, an anti-MUC1 antibody of the present disclosure has a drug (e.g., W¹ in conjugates of formula (I) described herein, or W¹¹ or W¹² in conjugates of formula (II) described herein) covalently linked to the heavy and/or light chain of the antibody. For example, an antibody conjugate of the present disclosure can include as substituent W¹, W¹¹ or W¹² a drug or active agent. Any of a number of drugs are suitable for use, or can be modified to be rendered suitable for use, as a reactive partner to conjugate to an antibody. “Drugs” include small molecule drugs, peptidic drugs, toxins (e.g., cytotoxins), and the like.

“Small molecule drug” as used herein refers to a compound, e.g., an organic compound, which exhibits a pharmaceutical activity of interest and which is generally of a molecular weight of no greater than about 800 Da, or no greater than 2000 Da, but can encompass molecules of up to 5 kDa and can be as large as about 10 kDa. A small inorganic molecule refers to a molecule containing no carbon atoms, while a small organic molecule refers to a compound containing at least one carbon atom.

In certain embodiments, the drug or active agent can be a maytansine. “Maytansine”, “maytansine moiety”, “maytansine active agent moiety” and “maytansinoid” refer to a maytansine and analogs and derivatives thereof, and pharmaceutically active maytansine moieties and/or portions thereof. A maytansine conjugated to the polypeptide can be any of a variety of maytansinoid moieties such as, but not limited to, maytansine and analogs and derivatives thereof as described herein (e.g., deacylmaytansine).

In certain embodiments, the drug or active agent can be an auristatin, or an analog or derivative thereof, or a pharmaceutically active auristatin moiety and/or a portion thereof. An auristatin conjugated to the polypeptide can be any of a variety of auristatin moieties such as, but not limited to, an auristatin and analogs and derivatives thereof as described herein. Examples of drugs that find use in the conjugates and compounds described herein include, but are not limited to an auristatin or an auristatin derivative, such as monomethyl auristatin D (MMAD), monomethyl auristatin E (MMAE), monomethyl auristatin F (MMAF), derivatives thereof, and the like.

In certain embodiments, the drug or active agent can be a duocarmycin, or an analog or derivative thereof, or a pharmaceutically active duocarmycin moiety and/or a portion thereof. A duocarmycin conjugated to the polypeptide can be any of a variety of duocarmycin moieties such as, but not limited to, a duocarmycin and analogs and derivatives thereof as described herein. Examples of drugs that find use in the conjugates and compounds described herein include, but are not limited to a duocarmycin or a duocarmycin derivative, such as duocarmycin A, duocarmycin B1, duocarmycin B2, duocarmycin C1, duocarmycin C2, duocarmycin D, duocarmycin SA, and CC-1065, derivatives thereof, and the like. In some embodiments, the duocarmycin is a duocarmycin analog, such as, but not limited to, adozelesin, bizelesin, or carzelesin.

In certain embodiments, the drug or active agent can be a topoisomerase inhibitor, such as a camptothecine, or an analog or derivative thereof, or a pharmaceutically active camptothecine moiety and/or a portion thereof. A camptothecine conjugated to the subject antibody can be any of a variety of camptothecine moieties such as, but not limited to, a camptothecine and analogs and derivatives thereof as described herein. Examples of drugs that find use in the conjugates described herein include, but are not limited to a camptothecine or a camptothecine derivative, such as SN-38, Belotecan, Exatecan, 9-aminocamptothecin (9-AC), derivatives thereof, and the like.

In certain embodiments, the drug W¹ in formula (I) described herein or W¹¹ or W¹² in formula (II) described herein is the camptothecine, or analog or derivative thereof. For example, in some instances, the camptothecine, or analog or derivative thereof, is a compound of formula (IV):

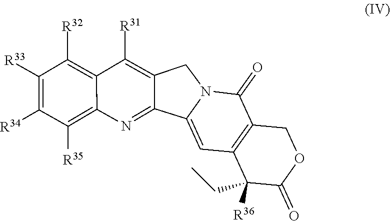

wherein:
   R³¹ and R³² are each independently selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl, or R³¹ and R³² are optionally cyclically linked to form a 5 or 6-membered cycloalkyl or heterocyclyl ring;
   R³³ and R³⁴ are each independently selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl, or R³³ and R³⁴ are optionally cyclically linked to form a 5 or 6-membered cycloalkyl or heterocyclyl ring;
   R³⁵ is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl;
   R³⁶ is selected from OH and OC(O)R³⁷; and
   R³⁷ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl.

In certain embodiments of formula (IV), the linker L in formula (I) or the first linker L^{A} or the second linker L^{B} in formula (II) is attached to a compound of formula (IV) at R³¹, R³², R³³, R³⁴, R³⁵ or R³⁶.

In certain embodiments, R³¹ and R³² are each independently selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl, or R³¹ and R³² are optionally cyclically linked to form a 5 or 6-membered cycloalkyl or heterocyclyl ring.

In certain embodiments, R³¹ is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl. In certain embodiments, R³¹ is hydrogen. In certain embodiments, R³¹ is halogen (e.g., F, Cl, Br, I). In certain embodiments, R³¹ is hydroxy. In certain embodiments, R³¹ is amino or substituted amino. In certain embodiments, R³¹ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³¹ is methyl. In certain embodiments, R³¹ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³¹ is alkynyl or substituted alkynyl. In certain embodiments, R³¹ is alkoxy or substituted alkoxy. In certain embodiments, R³¹ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³¹ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³¹ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³¹ is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³² is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl. In certain embodiments, R³² is hydrogen. In certain embodiments, R³² is halogen (e.g., F, Cl, Br, I). In certain embodiments, R³² is hydroxy. In certain embodiments, R³² is amino or substituted amino. In certain embodiments, R³² is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³² is methyl. In certain embodiments, R³² is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³² is alkynyl or substituted alkynyl. In certain embodiments, R³² is alkoxy or substituted alkoxy. In certain embodiments, R³² is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³² is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³² is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³² is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³¹ and R³² are optionally cyclically linked to form a 5 or 6-membered cycloalkyl or heterocyclyl ring. In certain embodiments, R³¹ and R³² are cyclically linked to form a 5 or 6-membered cycloalkyl. In certain embodiments, R³¹ and R³² are cyclically linked to form a 5 or 6-membered heterocyclyl. In certain embodiments, R³¹ and R³² are cyclically linked to form a 5-membered cycloalkyl. In certain embodiments, R³¹ and R³² are cyclically linked to form a 6-membered cycloalkyl. In certain embodiments, R³¹ and R³² are cyclically linked to form a 5-membered heterocyclyl. In certain embodiments, R³¹ and R³² are cyclically linked to form a 6-membered heterocyclyl.

In certain embodiments, R³³ and R³⁴ are each independently selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl, or R³³ and R³⁴ are optionally cyclically linked to form a 5 or 6-membered cycloalkyl or heterocyclyl ring.

In certain embodiments, R³³ is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl. In certain embodiments, R³³ is hydrogen. In certain embodiments, R³³ is halogen (e.g., F, Cl, Br, I). In certain embodiments, R³³ is hydroxy. In certain embodiments, R³³ is amino or substituted amino. In certain embodiments, R³³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³³ is methyl. In certain embodiments, R³³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³³ is alkynyl or substituted alkynyl. In certain embodiments, R³³ is alkoxy or substituted alkoxy. In certain embodiments, R³³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³³ is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³⁴ is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl. In certain embodiments, R³⁴ is hydrogen. In certain embodiments, R³⁴ is halogen (e.g., F, Cl, Br, I). In certain embodiments, R³⁴ is hydroxy. In certain embodiments, R³⁴ is amino or substituted amino. In certain embodiments, R³⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³⁴ is methyl. In certain embodiments, R³⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³⁴ is alkynyl or substituted alkynyl. In certain embodiments, R³⁴ is alkoxy or substituted alkoxy. In certain embodiments, R³⁴ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³⁴ is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³³ and R³⁴ are optionally cyclically linked to form a 5 or 6-membered cycloalkyl or heterocyclyl ring. In certain embodiments, R³³ and R³⁴ are cyclically linked to form a 5 or 6-membered cycloalkyl. In certain embodiments, R³³ and R³⁴ are cyclically linked to form a 5 or 6-membered heterocyclyl. In certain embodiments, R³³ and R³⁴ are cyclically linked to form a 5-membered cycloalkyl. In certain embodiments, R³³ and R³⁴ are cyclically linked to form a 6-membered cycloalkyl. In certain embodiments, R³³ and R³⁴ are cyclically linked to form a 5-membered heterocyclyl. In certain embodiments, R³³ and R³⁴ are cyclically linked to form a 6-membered heterocyclyl.

In certain embodiments, R³⁵ is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl. In certain embodiments, R³⁵ is hydrogen. In certain embodiments, R³⁵ is halogen (e.g., F, Cl, Br, I). In certain embodiments, R³⁵ is hydroxy. In certain embodiments, R³⁵ is amino or substituted amino. In certain embodiments, R³⁵ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³⁵ is methyl. In certain embodiments, R³⁵ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³⁵ is alkynyl or substituted alkynyl. In certain embodiments, R³⁵ is alkoxy or substituted alkoxy. In certain embodiments, R³⁵ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³⁵ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³⁵ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³⁵ is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³⁶ is selected from OH and OC(O)R³⁷. In certain embodiments, R³⁶ is OH. In certain embodiments, R³⁶ is OC(O)R³⁷.

In certain embodiments, R³⁷ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R³⁷ is hydrogen. In certain embodiments, R³⁷ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³⁷ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³⁷ is alkynyl or substituted alkynyl. In certain embodiments, R³⁷ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³⁷ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³⁷ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³⁷ is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, the compound of formula (IV) has the structure of formula (IVa):

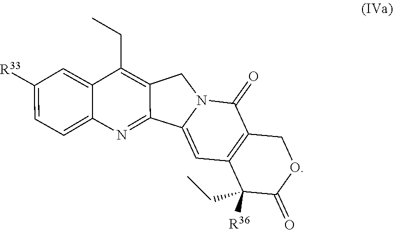

In certain embodiments of the compound of formula (IVa), R³³ is as described above.

In certain embodiments of the compound of formula (IVa), R³⁶ is as described above.

In certain embodiments of the compound of formula (IVa), R³³ is OH and L is attached at R³⁶. In certain embodiments of the compound of formula (IVa), L is attached at R³³ and R³⁶ is OH.

In certain embodiments, the compound of formula (IV) has the structure of formula (IVb):

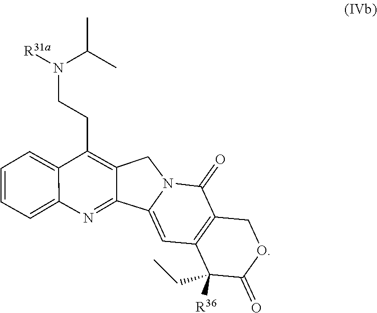

In certain embodiments of the compound of formula (IVb), R^{31a} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl. In certain embodiments, R^{31a} is hydrogen. In certain embodiments, R^{31a} is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R^{31a} is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R^{31a} is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R^{31a} is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R^{31a} is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl. In certain embodiments, R^{31a} is carboxyl. In certain embodiments, R^{31a} is carboxyl ester. In certain embodiments, R^{31a} is acyl. In certain embodiments, R^{31a} is sulfonyl.

In certain embodiments of the compound of formula (IVb), R³⁶ is as described above.

In certain embodiments of the compound of formula (IVb), R^{31a} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl, and L is attached at R³⁶. In certain embodiments of the compound of formula (IVb), L is attached at R^{31a} and R³⁶ is OH.

In certain embodiments, the compound of formula (IV) has the structure of formula (IVc):

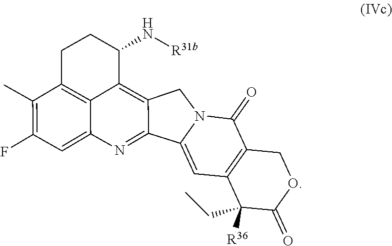

In certain embodiments of the compound of formula (IVc), R^{31b} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl. In certain embodiments, R^{31b} is hydrogen. In certain embodiments, R^{31b} is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R^{31b} is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R^{31b} is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R^{31b} is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R^{31b} is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl. In certain embodiments, R^{31b} is carboxyl. In certain embodiments, R^{31b} is carboxyl ester. In certain embodiments, R^{31b} is acyl. In certain embodiments, R^{31b} is sulfonyl.

In certain embodiments of the compound of formula (IVc), R³⁶ is as described above.

In certain embodiments of the compound of formula (IVc), R^{31b} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl, and L is attached at R³⁶. In certain embodiments of the compound of formula (IVc), L is attached at R^{31b} and R³⁶ is OH.

In certain embodiments, the compound of formula (IV) has the structure of formula (IVd):

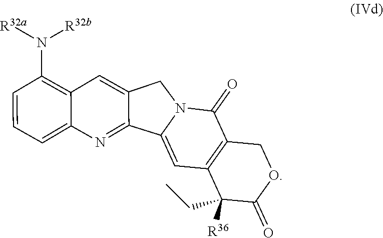

In certain embodiments of the compound of formula (IVd), R^{32a} and R^{32b} are each independently selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl.

In certain embodiments of the compound of formula (IVd), R^{32a} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl. In certain embodiments, R^{32a} is hydrogen. In certain embodiments, R^{32a} is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R^{32a} is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R^{32a} is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R^{32a} is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R^{32a} is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl. In certain embodiments, R^{32a} is carboxyl. In certain embodiments, R^{32a} is carboxyl ester. In certain embodiments, R^{32a} is acyl. In certain embodiments, R^{32a} is sulfonyl.

In certain embodiments of the compound of formula (IVd), R^{32b} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl. In certain embodiments, R^{32b} is hydrogen. In certain embodiments, R^{32b} is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R^{32b} is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R^{32b} is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R^{32b} is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R^{32b} is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl. In certain embodiments, R^{32b} is carboxyl. In certain embodiments, R^{32b} is carboxyl ester. In certain embodiments, R^{32b} is acyl. In certain embodiments, R^{32b} is sulfonyl.

In certain embodiments of the compound of formula (IVd), R³⁶ is as described above.

In certain embodiments of the compound of formula (IVd), R^{32a} and R^{32b} are each independently selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl, and L is attached at R³⁶. In certain embodiments of the compound of formula (IVd), L is attached at R^{32a} or R^{32b} and R³⁶ is OH. In certain embodiments of the compound of formula (IVd), L is attached at R^{32a} and R³⁶ is OH. In certain embodiments of the compound of formula (IVd), L is attached at R^{32b} and R³⁶ is OH.

In certain embodiments, the drug is selected from a cytotoxin, a kinase inhibitor, an immunostimulatory agent, a toll-like receptor (TLR) agonist, an oligonucleotide, an aptamer, a cytokine, a steroid, and a peptide.

For example, a cytotoxin can include any compound that leads to cell death (e.g., necrosis or apoptosis) or a decrease in cell viability.

Kinase inhibitors can include, but are not limited to, Adavosertib, Afatinib, Axitinib, Bosutinib, Cetuximab, Cobimetinib, Crizotinib, Cabozantinib, Dacomitinib, Dasatinib, Entrectinib, Erdafitinib, Erlotinib, Fostamatinib, Gefitinib, Ibrutinib, Imatinib, Lapatinib, Lenvatinib, Mubritinib, Nilotinib, Pazopanib, Pegaptanib, Ruxolitinib, Sorafenib, Sunitinib, Tucatinib, Vandetanib, Vemurafenib, and the like.

Immunostimulatory agents can include, but are not limited to, vaccines (e.g., bacterial or viral vaccines), colony stimulating factors, interferons, interleukins, and the like. TLR agonists include, but are not limited to, imiquimod, resiquimod, and the like.

Oligonucleotide dugs include, but are not limited to, fomivirsen, pegaptanib, mipomersen, eteplirsen, defibrotide, nusinersen, golodirsen, viltolarsen, volanesorsen, inotersen, tofersen, tominersen, and the like.

Aptamer drugs include, but are not limited to, pegaptanib, AS1411, REG1, ARC1779, NU172, ARC1905, E10030, NOX-A12, NOX-E36, and the like.

Cytokines include, but are not limited to, Albinterferon Alfa-2B, Aldesleukin, ALT-801, Anakinra, Ancestim, Avotermin, Balugrastim, Bempegaldesleukin, Binetrakin, Cintredekin Besudotox, CTCE-0214, Darbepoetin alfa, Denileukin diftitox, Dulanermin, Edodekin alfa, Emfilermin, Epoetin delta, Erythropoietin, Human interleukin-2, Interferon alfa, Interferon alfa-2c, Interferon alfa-n1, Interferon alfa-n3, Interferon alfacon-1, Interferon beta-1a, Interferon beta-1b, Interferon gamma-1b, Interferon Kappa, Interleukin-1 alpha, Interleukin-10, Interleukin-7, Lenograstim, Leridistim, Lipegfilgrastim, Lorukafusp alfa, Maxy-G34, Methoxy polyethylene glycol-epoetin beta, Molgramostim, Muplestim, Nagrestipen, Oprelvekin, Pegfilgrastim, Pegilodecakin, Peginterferon alfa-2a, Peginterferon alfa-2b, Peginterferon beta-1a, Peginterferon lambda-1a, Recombinant CD40-ligand, Regramostim, Romiplostim, Sargramostim, Thrombopoietin, Tucotuzumab celmoleukin, Viral Macrophage-Inflammatory Protein, and the like.

Steroid drugs include, but are not limited to, prednisolone, betamethasone, dexamethasone, hydrocortisone, methylprednisolone, deflazacort, and the like.

“Peptide drug” as used herein refers to amino-acid containing polymeric compounds, and is meant to encompass naturally-occurring and non-naturally-occurring peptides, oligopeptides, cyclic peptides, polypeptides, and proteins, as well as peptide mimetics. The peptide drugs may be obtained by chemical synthesis or be produced from a genetically encoded source (e.g., recombinant source). Peptide drugs can range in molecular weight, and can be from 200 Da to 10 kDa or greater in molecular weight. Suitable peptides include, but are not limited to, cytotoxic peptides; angiogenic peptides; anti-angiogenic peptides; peptides that activate B cells; peptides that activate T cells; anti-viral peptides; peptides that inhibit viral fusion; peptides that increase production of one or more lymphocyte populations; anti-microbial peptides; growth factors; growth hormone-releasing factors; vasoactive peptides; anti-inflammatory peptides; peptides that regulate glucose metabolism; an anti-thrombotic peptide; an anti-nociceptive peptide; a vasodilator peptide; a platelet aggregation inhibitor; an analgesic; and the like.

Additional examples of drugs that find use in the conjugates and compounds described herein include, but are not limited to Tubulysin M, Calicheamicin, a STAT3 inhibitor, alpha-Amanitin, an aurora kinase inhibitor, belotecan, and an anthracycline.

In some cases, the drug is a toxin, e.g., a cytotoxin. Ribosome inactivating proteins (RIPs), which are a class of proteins ubiquitous in higher plants, are examples of such cytotoxins. Suitable cytotoxins include, but are not limited to, ricin, abrin, diphtheria toxin, a Pseudomonas exotoxin (e.g., PE35, PE37, PE38, PE40, etc.), saporin, gelonin, a pokeweed anti-viral protein (PAP), botulinum toxin, bryodin, momordin, and bouganin.

In some cases, the drug is a cancer chemotherapeutic agent. Cancer chemotherapeutic agents include non-peptidic (e.g., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (Vinca) alkaloids, and steroid hormones. Peptidic compounds can also be used.

Suitable cancer chemotherapeutic agents include dolastatin and active analogs and derivatives thereof; and auristatin and active analogs and derivatives thereof. Suitable cancer chemotherapeutic agents also include maytansinoids and active analogs and derivatives thereof; and duocarmycins and active analogs and derivatives thereof.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation; therefore, compounds that bind to the estrogen receptor are used to block this activity.

Other suitable chemotherapeutic agents include metal complexes, e.g., cisplatin (cis-DDP), carboplatin, etc.; ureas, e.g., hydroxyurea; and hydrazines, e.g., N-methylhydrazine; epidophyllotoxin; a topoisomerase inhibitor; procarbazine; mitoxantrone; leucovorin; tegafur; etc. Other anti-proliferative agents of interest include immunosuppressants, e.g., mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

Taxanes are suitable for use. “Taxanes” include paclitaxel, as well as any active taxane derivative or pro-drug. “Paclitaxel” (which should be understood herein to include analogues, formulations, and derivatives such as, for example, docetaxel, TAXOL□, TAXOTERE□ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., TAXOTERE□□ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives; piperazino and piperazino derivatives.

Embodiments of the present disclosure include conjugates where an antibody is conjugated to two or more drug moieties, such as 3 drug moieties, 4 drug moieties, 5 drug moieties, 6 drug moieties, 7 drug moieties, 8 drug moieties, 9 drug moieties, 10 drug moieties, 11 drug moieties, 12 drug moieties, 13 drug moieties, 14 drug moieties, 15 drug moieties, 16 drug moieties, 17 drug moieties, 18 drug moieties, 19 drug moieties, or 20 or more drug moieties. The drug moieties may be conjugated to the antibody at one or more sites in the antibody, as described herein. In certain embodiments, the conjugates have an average drug-to-antibody ratio (DAR) (molar ratio) in the range of from 0.1 to 20, or from 0.5 to 20, or from 1 to 20, such as from 1 to 19, or from 1 to 18, or from 1 to 17, or from 1 to 16, or from 1 to 15, or from 1 to 14, or from 1 to 13, or from 1 to 12, or from 1 to 11, or from 1 to 10, or from 1 to 9, or from 1 to 8, or from 1 to 7, or from 1 to 6, or from 1 to 5, or from 1 to 4, or from 1 to 3, or from 1 to 2. In certain embodiments, the conjugates have an average DAR from 1 to 10, such as 1, 2, 3, 4, 5, 6, 7, 8, 9, or 10. In certain embodiments, the conjugates have an average DAR of 1 to 10. In certain embodiments, the conjugates have an average DAR of 1 to 5. In certain embodiments, the conjugates have an average DAR of 5 to 10. By average is meant the arithmetic mean.

Drugs to be conjugated to a polypeptide may be modified to incorporate a reactive partner for reaction with the polypeptide. Where the drug is a peptide drug, the reactive moiety (e.g., aminooxy or hydrazide can be positioned at an N-terminal region, the N-terminus, a C-terminal region, the C-terminus, or at a position internal to the peptide. For example, an example of a method involves synthesizing a peptide drug having an aminooxy group. In this example, the peptide is synthesized from a Boc-protected precursor. An amino group of a peptide can react with a compound comprising a carboxylic acid group and oxy-N-Boc group. As an example, the amino group of the peptide reacts with 3-(2,5-dioxopyrrolidin-1-yloxy)propanoic acid. Other variations on the compound comprising a carboxylic acid group and oxy-N-protecting group can include different number of carbons in the alkylene linker and substituents on the alkylene linker. The reaction between the amino group of the peptide and the compound comprising a carboxylic acid group and oxy-N-protecting group occurs through standard peptide coupling chemistry. Examples of peptide coupling reagents that can be used include, but not limited to, DCC (dicyclohexylcarbodiimide), DIC (diisopropylcarbodiimide), di-p-toluoylcarbodiimide, BDP (1-benzotriazole diethylphosphate-1-cyclohexyl-3-(2-morpholinylethyl)carbodiimide), EDC (1-(3-dimethylaminopropyl-3-ethyl-carbodiimide hydrochloride), cyanuric fluoride, cyanuric chloride, TFFH (tetramethyl fluoroformamidinium hexafluorophosphosphate), DPPA (diphenylphosphorazidate), BOP (benzotriazol-1-yloxytris(dimethylamino)phosphonium hexafluorophosphate), HBTU (O-benzotriazol-1-yl-N,N,N′,N′-tetramethyluronium hexafluorophosphate), TBTU (O-benzotriazol-1-yl-N,N,N′,N′-tetramethyluronium tetrafluoroborate), TSTU (O—(N-succinimidyl)-N,N,N′,N′-tetramethyluronium tetrafluoroborate), HATU (N-[(dimethylamino)-1-H-1,2,3-triazolo[4,5,6]-pyridin-1-ylmethylene]-N-methylmethanaminium hexafluorophosphate N-oxide), BOP—Cl (bis(2-oxo-3-oxazolidinyl)phosphinic chloride), PyBOP ((1-H-1,2,3-benzotriazol-1-yloxy)-tris(pyrrolidino)phosphonium tetrafluorophopsphate), BrOP (bromotris(dimethylamino)phosphonium hexafluorophosphate), DEPBT (3-(diethoxyphosphoryloxy)-1,2,3-benzotriazin-4(3H)-one) PyBrOP (bromotris(pyrrolidino)phosphonium hexafluorophosphate). As a non-limiting example, HOBt and DIC can be used as peptide coupling reagents.

Deprotection to expose the amino-oxy functionality is performed on the peptide comprising an N-protecting group. Deprotection of the N-oxysuccinimide group, for example, occurs according to standard deprotection conditions for a cyclic amide group. Deprotecting conditions can be found in Greene and Wuts, Protective Groups in Organic Chemistry, 3rd Ed., 1999, John Wiley & Sons, NY and Harrison et al. Certain deprotection conditions include a hydrazine reagent, amino reagent, or sodium borohydride. Deprotection of a Boc protecting group can occur with TFA. Other reagents for deprotection include, but are not limited to, hydrazine, methylhydrazine, phenylhydrazine, sodium borohydride, and methylamine. The product and intermediates can be purified by conventional means, such as HPLC purification.

The ordinarily skilled artisan will appreciate that factors such as pH and steric hindrance (e.g., the accessibility of the amino acid residue to reaction with a reactive partner of interest) are of importance. Modifying reaction conditions to provide for optimal conjugation conditions is well within the skill of the ordinary artisan, and is routine in the art. Where conjugation is conducted with a polypeptide present in or on a living cell, the conditions are selected so as to be physiologically compatible. For example, the pH can be dropped temporarily for a time sufficient to allow for the reaction to occur but within a period tolerated by the cell (e.g., from about 30 min to 1 hour). Physiological conditions for conducting modification of polypeptides on a cell surface can be similar to those used in a ketone-azide reaction in modification of cells bearing cell-surface azides (see, e.g., U.S. Pat. No. 6,570,040).

Small molecule compounds containing, or modified to contain, an α-nucleophilic group that serves as a reactive partner with a compound or conjugate disclosed herein are also contemplated for use as drugs in the polypeptide-drug conjugates of the present disclosure. General methods are known in the art for chemical synthetic schemes and conditions useful for synthesizing a compound of interest (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

### Methods of Producing Antibody

A subject antibody can be produced by any known method, e.g., conventional synthetic methods for protein synthesis; recombinant DNA methods, etc.

Where a subject antibody is a single chain polypeptide, it can be synthesized using standard chemical peptide synthesis techniques. Where a polypeptide is chemically synthesized, the synthesis may proceed via liquid-phase or solid-phase. Solid phase polypeptide synthesis (SPPS), in which the C-terminal amino acid of the sequence is attached to an insoluble support followed by sequential addition of the remaining amino acids in the sequence, is an example of a suitable method for the chemical synthesis of a subject antibody. Various forms of SPPS, such as Fmoc and Boc, are available for synthesizing a subject antibody.

Standard recombinant methods can be used for production of a subject antibody. For example, nucleic acids encoding light and heavy chain variable regions, optionally linked to constant regions, are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences. The expression control sequences can be eukaryotic promoter systems in vectors capable of transforming or transfecting eukaryotic host cells (e.g., COS or CHO cells). Once the vector has been incorporated into the appropriate host, the host is maintained under conditions suitable for high level expression of the nucleotide sequences, and the collection and purification of the antibodies.

Because of the degeneracy of the code, a variety of nucleic acid sequences can encode each immunoglobulin amino acid sequence. The desired nucleic acid sequences can be produced by de novo solid-phase DNA synthesis or by polymerase chain reaction (PCR) mutagenesis of an earlier prepared variant of the desired polynucleotide.

Suitable expression vectors are typically replicable in the host organisms either as episomes or as an integral part of the host chromosomal DNA. Commonly, expression vectors contain selection markers (e.g., ampicillin-resistance, hygromycin-resistance, tetracycline resistance, kanamycin resistance or neomycin resistance) to permit detection of those cells transformed with the desired DNA sequences.

Escherichia coli is an example of a prokaryotic host cell that can be used for cloning a subject antibody-encoding polynucleotide. Other microbial hosts suitable for use include bacilli, such as Bacillus subtilis, and other enterobacteriaceae, such as Salmonella, Serratia, and various Pseudomonas species. Other microbes, such as yeast, are also useful for expression. Saccharomyces (e.g., S. cerevisiae) and Pichia are examples of suitable yeast host cells.

In addition to microorganisms, mammalian cells (e.g., mammalian cells grown in in vitro cell culture) can also be used to express and produce the polypeptides of the present invention (e.g., polynucleotides encoding immunoglobulins or fragments thereof). Suitable mammalian host cells include CHO cell lines, various Cos cell lines, HeLa cells, myeloma cell lines, and transformed B-cells or hybridomas. Expression vectors for these cells can include expression control sequences, such as an origin of replication, a promoter, and an enhancer, and necessary processing information sites, such as ribosome binding sites, RNA splice sites, polyadenylation sites, and transcriptional terminator sequences. Examples of suitable expression control sequences are promoters derived from immunoglobulin genes, SV40, adenovirus, bovine papilloma virus, cytomegalovirus and the like.

Once synthesized (either chemically or recombinantly), the whole antibodies, their dimers, individual light and heavy chains, or other forms of a subject antibody (e.g., scFv, etc.) can be purified according to standard procedures of the art, including ammonium sulfate precipitation, affinity columns, column chromatography, high performance liquid chromatography (HPLC) purification, gel electrophoresis, and the like (see generally Scopes, Protein Purification (Springer-Verlag, N.Y., (1982)). A subject antibody can be substantially pure, e.g., at least about 80% to 85% pure, at least about 85% to 90% pure, at least about 90% to 95% pure, or 98% to 99%, or more, pure, e.g., free from contaminants such as cell debris, macromolecules other than a subject antibody, etc.

### Compositions

The conjugates of the present disclosure can be formulated in a variety of different ways. In general, where the conjugate is a polypeptide-drug conjugate, the conjugate is formulated in a manner compatible with the drug conjugated to the polypeptide, the condition to be treated, and the route of administration to be used.

In some embodiments, provided is a pharmaceutical composition that includes any of the conjugates of the present disclosure and a pharmaceutically-acceptable excipient.

The conjugate (e.g., polypeptide-drug conjugate) can be provided in any suitable form, e.g., in the form of a pharmaceutically acceptable salt, and can be formulated for any suitable route of administration, e.g., oral, topical or parenteral administration. Where the conjugate is provided as a liquid injectable (such as in those embodiments where they are administered intravenously or directly into a tissue), the conjugate can be provided as a ready-to-use dosage form, or as a reconstitutable storage-stable powder or liquid composed of pharmaceutically acceptable carriers and excipients.

Methods for formulating conjugates can be adapted from those readily available. For example, conjugates can be provided in a pharmaceutical composition comprising a therapeutically effective amount of a conjugate and a pharmaceutically acceptable carrier (e.g., saline). The pharmaceutical composition may optionally include other additives (e.g., buffers, stabilizers, preservatives, and the like). In some embodiments, the formulations are suitable for administration to a mammal, such as those that are suitable for administration to a human.

For example, the present disclosure provides a composition comprising a subject antibody conjugate. A subject antibody conjugate composition can comprise, in addition to a subject antibody conjugate, one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

In certain embodiments, the present disclosure provides compositions, including pharmaceutical compositions, comprising a subject antibody conjugate. In general, a formulation comprises an effective amount of a subject antibody conjugate. An “effective amount” means a dosage sufficient to produce a desired result, e.g., reduction in the number of cancerous cells. In some cases, the desired result is at least a reduction in a symptom of a malignancy, as compared to a control.

### Formulations

In the subject methods, a subject antibody conjugate can be administered to the host using any convenient means capable of resulting in the desired therapeutic effect or diagnostic effect. Thus, the antibody conjugate can be incorporated into a variety of formulations for therapeutic administration. More particularly, a subject antibody conjugate can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, a subject antibody conjugate can be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, a subject antibody conjugate can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

A subject antibody conjugate can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions comprising a subject antibody conjugate are prepared by mixing the antibody conjugate having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however, solutions comprising antibacterial agents may be used for the production of pharmaceutical compositions for parenteral administration.

Exemplary antibody conjugate concentrations in a subject pharmaceutical composition may range from about 1 mg/mL to about 200 mg/ml or from about 50 mg/mL to about 200 mg/mL, or from about 150 mg/mL to about 200 mg/mL.

An aqueous formulation of the antibody conjugate may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A lyoprotectant may also be added in order to protect the labile active ingredient (e.g., a protein) against destabilizing conditions during the lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 nM to 500 nM.

In some embodiments, a subject formulation includes a subject antibody conjugate, and one or more agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

For example, a subject formulation can be a liquid or lyophilized formulation suitable for parenteral administration, and can comprise: about 1 mg/mL to about 200 mg/mL of a subject antibody conjugate; about 0.001% to about 1% of at least one surfactant; about 1 mM to about 100 mM of a buffer; optionally about 10 mM to about 500 mM of a stabilizer; and about 5 mM to about 305 mM of a tonicity agent; and has a pH of about 4.0 to about 7.0.

As another example, a subject parenteral formulation is a liquid or lyophilized formulation comprising about 1 mg/mL to about 200 mg/mL of a subject antibody conjugate; 0.04% Tween 20 w/v; 20 mM L-histidine; and 250 mM Sucrose; and has a pH of 5.5.

The term “unit dosage form,” as used herein, refers to physically discrete units suitable as unitary dosages for human and animal subjects, each unit containing a predetermined quantity of an antibody conjugate of the present disclosure calculated in an amount sufficient to produce the desired effect in association with a pharmaceutically acceptable diluent, carrier or vehicle. The specifications for a subject antibody conjugate may depend on the particular antibody conjugate employed and the effect to be achieved, and the pharmacodynamics associated with each antibody conjugate in the host.

A subject antibody conjugate can be administered as an injectable formulation. Typically, injectable compositions are prepared as liquid solutions or suspensions; solid forms suitable for solution in, or suspension in, liquid vehicles prior to injection may also be prepared. The preparation may also be emulsified or the antibody conjugate encapsulated in liposome vehicles.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, a subject antibody conjugate is formulated in a controlled release formulation. Sustained-release preparations may be prepared using methods well known in the art. Suitable examples of sustained-release preparations include semipermeable matrices of solid hydrophobic polymers containing the antibody conjugate in which the matrices are in the form of shaped articles, e.g., films or microcapsules. Examples of sustained-release matrices include polyesters, copolymers of L-glutamic acid and ethyl-L-glutamate, non-degradable ethylene-vinyl acetate, hydrogels, polylactides, degradable lactic acid-glycolic acid copolymers and poly-D-(−)-3-hydroxybutyric acid. Possible loss of biological activity and possible changes in immunogenicity of antibodies comprised in sustained-release preparations may be prevented by using appropriate additives, by controlling moisture content and by developing specific polymer matrix compositions.

Physical systems include, but are not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., nonerodible, erodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous).

### Dosages

A suitable dosage can be determined by an attending physician or other qualified medical personnel, based on various clinical factors. As is well known in the medical arts, dosages for any one patient depend upon many factors, including the patient's size, body surface area, age, the particular compound to be administered, sex of the patient, time, and route of administration, general health, and other drugs being administered concurrently. A subject antibody conjugate may be administered in amounts between 1 ng/kg body weight and 20 mg/kg body weight per dose, e.g., between 0.1 mg/kg body weight to 10 mg/kg body weight, e.g., between 0.5 mg/kg body weight to 5 mg/kg body weight; however, doses below or above this exemplary range are envisioned, especially considering the aforementioned factors. If the regimen is a continuous infusion, it can also be in the range of 1 g to 10 mg per kilogram of body weight per minute.

Those of skill will readily appreciate that dose levels can vary as a function of the specific antibody conjugate, the severity of the symptoms and the susceptibility of the subject to side effects. Preferred dosages for a given compound are readily determinable by those of skill in the art by a variety of means.

### Routes of Administration

A subject antibody conjugate is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, subcutaneous, intradermal, topical application, intravenous, intraarterial, rectal, nasal, oral, and other enteral and parenteral routes of administration. Routes of administration may be combined, if desired, or adjusted depending upon the antibody conjugate and/or the desired effect. A subject antibody conjugate composition can be administered in a single dose or in multiple doses. In some embodiments, a subject antibody conjugate composition is administered orally. In some embodiments, a subject antibody conjugate composition is administered via an inhalational route. In some embodiments, a subject antibody conjugate composition is administered intranasally. In some embodiments, a subject antibody conjugate composition is administered locally. In some embodiments, a subject antibody conjugate composition is administered intracranially. In some embodiments, a subject antibody conjugate composition is administered intravenously.

The antibody conjugate can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, intrahepatic, and intravenous routes, e.g., any route of administration other than through the alimentary canal. Parenteral administration can be carried to effect systemic or local delivery of a subject antibody. Where systemic delivery is desired, administration typically involves invasive or systemically absorbed topical or mucosal administration of pharmaceutical preparations.

A subject antibody conjugate can also be delivered to the subject by enteral administration. Enteral routes of administration include, but are not necessarily limited to, oral and rectal (e.g., using a suppository) delivery.

By treatment is meant at least an amelioration of the symptoms associated with the pathological condition afflicting the host, where amelioration is used in a broad sense to refer to at least a reduction in the magnitude of a parameter, e.g., symptom, associated with the pathological condition being treated, such as a breast cancer, pancreatic cancer, or lung cancer. As such, treatment also includes situations where the pathological condition, or at least symptoms associated therewith, are completely inhibited, e.g., prevented from happening, or stopped, e.g., terminated, such that the host no longer suffers from the pathological condition, or at least the symptoms that characterize the pathological condition.

In some embodiments, a subject antibody conjugate is administered by injection, e.g., for systemic delivery (e.g., intravenous infusion) or to a local site.

A variety of hosts (wherein the term “host” is used interchangeably herein with the terms “subject,” “individual,” and “patient”) are treatable according to the subject methods. Generally, such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the hosts will be humans.

### Treatment Methods

The present disclosure provides methods of treating a disease or disorder associated with or caused by a MUC1-positive cell, e.g., a cancerous MUC1-positive cell or an autoreactive MUC1-positive cell.

### Treating Malignancies

The present disclosure provides methods of treating a malignancy, including a solid tumor or a hematologic malignancy, the methods generally involving administering to an individual in need thereof (e.g., an individual having a malignancy) an effective amount of a subject antibody conjugate, alone (e.g., in monotherapy) or in combination (e.g., in combination therapy) with one or more additional therapeutic agents.

Malignancies include, e.g., HCC, non-Hodgkin's lymphoma, Burkitt's lymphoma, multiple myeloma, chronic lymphocytic leukemia, hairy cell leukemia, prolymphocytic leukemia, anal cancer, appendix cancer, bile duct cancer (e.g., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, vulvar cancer, and the like.

In some embodiments, an effective amount of a subject antibody conjugate is an amount that, when administered alone (e.g., in monotherapy) or in combination (e.g., in combination therapy) with one or more additional therapeutic agents, in one or more doses, is effective to reduce the number of cancerous cells in an individual by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the number of cancerous cells in the individual in the absence of treatment with the antibody conjugate.

In some instances, a breast cancer is triple-negative for estrogen, progesterone, and HER2. In some instances, a triple-negative breast cancer is metastatic triple negative breast cancer. In some instances, a triple-negative breast cancer is a relapsed or refractory triple negative breast cancer. In some instances, a triple-negative breast cancer is a relapsed or refractory metastatic triple negative breast cancer.

Aspects of the present disclosure include a method of delivering a drug to a target site in a subject. The method includes administering to the subject a pharmaceutical composition comprising a conjugate according to the present disclosure, where the administering is effective to release a therapeutically effective amount of the drug from the conjugate at the target site in the subject.

In some embodiments, multiple doses of an antibody-drug conjugate are administered. The frequency of administration of an antibody-drug conjugate can vary depending on any of a variety of factors, e.g., severity of the symptoms, condition of the subject, etc. For example, in some embodiments, an antibody-drug conjugate is administered once per month, twice per month, three times per month, every other week, once per week (qwk), twice per week, three times per week, four times per week, five times per week, six times per week, every other day, daily (qd/od), twice a day (bds/bid), or three times a day (tds/tid), etc.

### Combination Therapy

In some embodiments, a subject method of treating a malignancy involves administering a subject antibody conjugate and one or more additional therapeutic agents. Suitable additional therapeutic agents include, but are not limited to, a cancer chemotherapeutic agent (as described above).

In some embodiments, the treatment method may include administering to the subject a therapeutically effective amount of an immunomodulatory therapeutic agent. The immunomodulatory therapeutic agent may be an immune checkpoint inhibitor or interleukin. The immune checkpoint inhibitor may inhibit A2AR, B7-H3, B7- H4, BTLA, CTLA-4, CD277, IDO, KIR, PD-1, LAG-3, TIM-3, TIGIT or VISTA. The immune checkpoint inhibitor that inhibits PD-1 signaling may be an anti-PD-1 antibody. The anti-PD-1 antibody may be nivolumab, pembrolizumab, atezolizumab, durvalumab, or avelumab. The immune checkpoint inhibitor that inhibits CTLA-4 may be an anti-CTLA-4 antibody. The anti-CTLA-4 antibody may be ipilimumab.

### Subjects Suitable for Treatment

A variety of subjects are suitable for treatment with a subject method. Suitable subjects include any individual, e.g., a human, who has a malignancy; who has been diagnosed with a malignancy; who has had a malignancy and is at risk for recurrence of the malignancy; who has been treated for a malignancy with an agent other than a subject anti-MUC1 antibody conjugate (e.g., who has been treated with a cancer chemotherapeutic agent) and who has not responded to the agent; or who has been treated for a malignancy with an agent other than a subject anti-MUC1 antibody conjugate (e.g., who has been treated with a cancer chemotherapeutic agent) and who initially responded to the agent but subsequently ceased to respond (e.g., relapsed).

### Embodiments

Certain embodiments of the present disclosure are described in the clauses listed below. These embodiments are illustrative only and not intended to be limiting in scope.

1. A conjugate of formula (I):

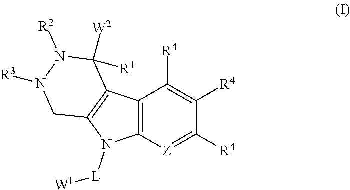

wherein
   Z is CR⁴ or N;
   R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
   R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
   each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
   L is a linker;
   W¹ is a drug; and
   W² is an anti-MUC1 antibody.
   2. The conjugate of clause 1, wherein L comprises:

-(T¹-V¹)ₐ-(T²-V²)_{b}-(T³-V³)_{c}-(T⁴-V⁴)_{d}-(T⁵-V⁵)ₑ-(T⁶-V⁶)_{f}-,

wherein
a, b, c, d, e and f are each independently 0 or 1, wherein the sum of a, b, c, d, e and f is 1 to 6;
T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each m is an integer from 1 to 12;
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl;
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

3. The conjugate of clause 2, wherein:
T¹ is selected from a (C₁-C₁₂)alkyl and a substituted (C₁-C₁₂)alkyl;
T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, 4-amino-piperidine (4AP), MABO, MABC, PABO, PABC, PAB, PABA, PAP, PHP, an acetal group, a hydrazine, and an ester; and
V¹, V², V³, V⁴, V⁵ and V⁶ are each independently selected from the group consisting of a covalent bond, —CO—, —NR¹⁵—, —NR⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, and —P(O)OH—;
wherein:
(PEG)ₙ is

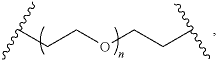

n where n is an integer from 1 to 30;
EDA is an ethylene diamine moiety having the following structure:

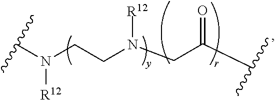

where y is an integer from 1 to 6 and r is 0 or 1;
4-amino-piperidine (4AP) is

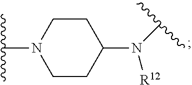

and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring.

4. The conjugate of any of clauses 2-3, wherein MABO, MABC, PABO, PABC, PAB, PABA, PAP and PHP are each optionally substituted with a glycoside.

5. The conjugate of any of clause 4, wherein the glycoside is selected from a glucuronide, a galactoside, a glucoside, a mannoside, a fucoside, O-GlcNAc, and O-GalNAc.

6. The conjugate of any of clauses 2-5,

wherein:
T¹ is (C₁-C₁₂)alkyl and V¹ is —CO—;
T² is an amino acid analog and V² is —NH—;
T³ is (PEG)ₙ and V³ is —CO—;
T⁴ is AA and V⁴ is absent;
T⁵ is PABC and V⁵ is absent; and
f is 0.

7. The conjugate of any of clauses 1-6, wherein the drug is monomethyl auristatin E (MMAE).

8. The conjugate of any one of clauses 1-7, wherein the conjugate has the structure:

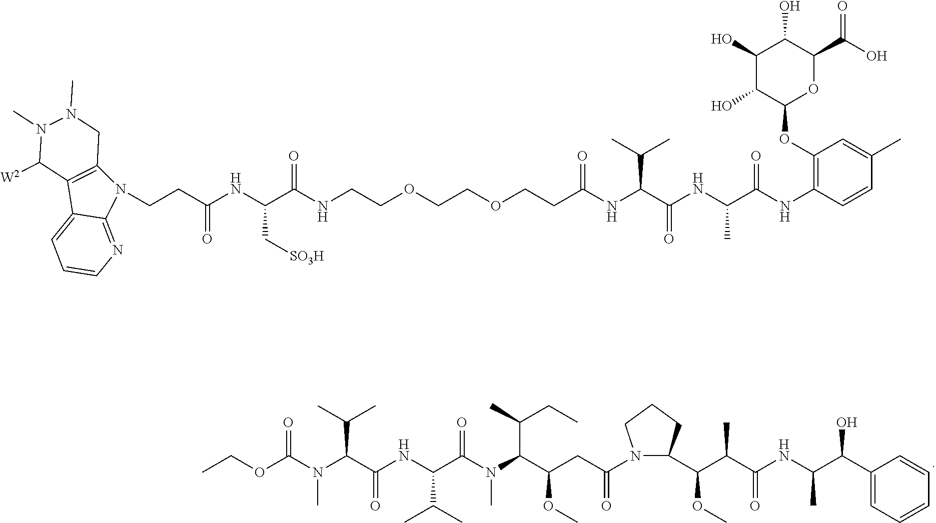

9. A conjugate of formula (II)

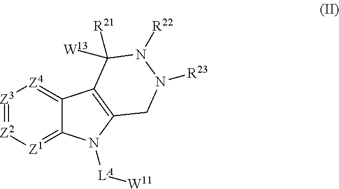

wherein:
Z¹, Z², Z³ and Z⁴ are each independently selected from CR²⁴, N and C-L^{B}-W¹², wherein at least one Z¹, Z², Z³ and Z⁴ is C-L^{B}-W¹²;
R²¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R²² and R²³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R²² and R²³ are optionally cyclically linked to form a 5 or 6-membered heterocyclyl;
each R²⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L^{A} is a first linker;
L^{B} is a second linker;
W¹¹ is a first drug;
W¹² is a second drug; and
W¹³ is an anti-MUC1 antibody.

10. The conjugate of clause 9, wherein Z¹ is CR²⁴.

11. The conjugate of clause 9, wherein Z¹ is N.

12. The conjugate of clause 9, wherein Z³ is C-L^{B}-W¹².

13. The conjugate of any of Claims 9-12, wherein L^{A} comprises:

-(T¹-V¹)ₐ-(T²-V²)ₑ-(T³-V³)_{c}-(T⁴-V⁴)_{d}-(T⁵-V⁵)ₑ-(T⁶-V⁶)_{f}-,

wherein
a, b, c, d, e and f are each independently 0 or 1;
T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

14. The conjugate of clause 13, wherein MABO, MABC, PABO, PABC, PAB, PABA, PAP and PHP are each optionally substituted with a glycoside.

15. The conjugate of clause 14, wherein the glycoside is selected from a glucuronide, a galactoside, a glucoside, a mannoside, a fucoside, O-GlcNAc, and O-GalNAc.

16. The conjugate of any of clauses 9-15,

wherein:
T¹ is (C₁-C₁₂)alkyl and V¹ is —CONH—;
T² is substituted (C₁-C₁₂)alkyl and V² is —CO—;
T³ is AA and V³ is absent;
T⁴ is PABC and V⁴ is absent; and
e and f are each 0.

17. The conjugate of any of clauses 9-16, wherein L^{B} comprises:

-(T⁷-V⁷)_{g}-(T⁸-V⁸)ₕ-(T⁹-V⁹)ᵢ-(T¹⁰-V¹⁰)ⱼ-(V¹¹-V¹¹)ₖ-(T¹²-V¹²)ₗ-(T¹³-V¹³)ₘ—,

wherein
g, h, i, j, k, l and m are each independently 0 or 1;
T⁷, T⁸, T⁹, T¹⁰, T¹¹, T¹² and T¹³ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V⁷, V⁸, V⁹, V¹⁰, V¹¹, V¹² and V¹³ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

18. The conjugate of clause 17, wherein MABO, MABC, PABO, PABC, PAB, PABA, PAP and PHP are each optionally substituted with a glycoside.

19. The conjugate of any of clauses 17-18, wherein the glycoside is selected from a glucuronide, a galactoside, a glucoside, a mannoside, a fucoside, 0-GlcNAc, and O-GalNAc.

20. The conjugate of any of clauses 17-19,

wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is AA and V¹⁰ is absent;
T¹¹ is PABC and V¹¹ is absent; and
1 and m are each 0.

21. The conjugate of clause 9, wherein the conjugate has the structure:

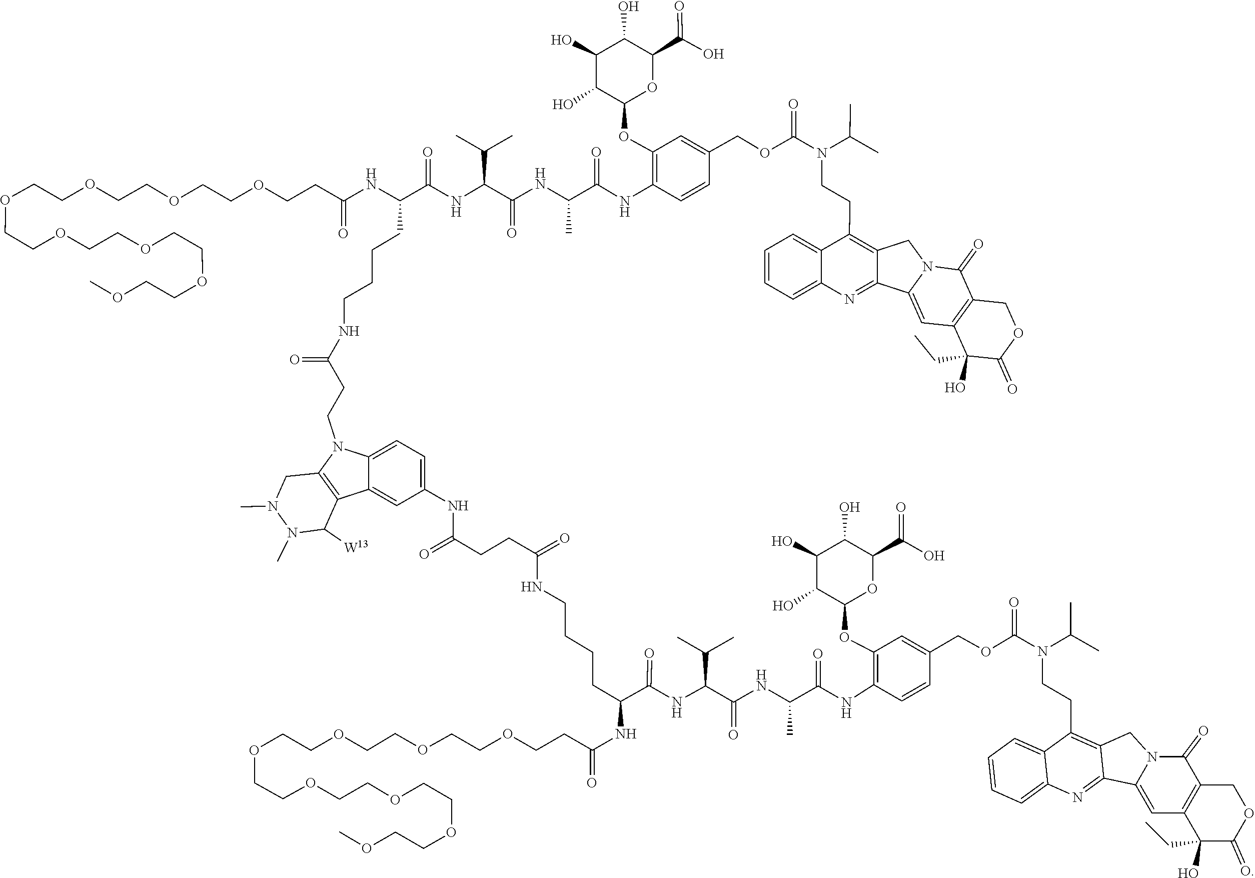

22. The conjugate of any one of clauses 1 to 21, wherein the anti-MUC1 antibody is an IgG1 antibody.

23. The conjugate of clause 22, wherein the anti-MUC1 antibody is an IgG1 kappa antibody.

24. The conjugate of any one of clauses 1 to 23, wherein the anti-MUC1 antibody comprises a sequence of the formula (III):

X¹(fGly′)X²Z²X³Z³ (III),

wherein
X¹ is present or absent and, when present, can be any amino acid, with the proviso that when the sequence is at the N-terminus of the conjugate, X¹ is present;
fGly′ is the amino acid residue coupled to the drug through the linker;
X² and X³ are each independently any amino acid;
Z² is either a proline or alanine residue; and
Z³ is a basic amino acid or an aliphatic amino acid.

25. The conjugate of clause 24, wherein the sequence is L(fGly′)TPSR (SEQ ID NO:24).

26. The conjugate of clause 24, wherein
Z³ is selected from R, K, H, A, G, L, V, I, and P;
X¹ is selected from L, M, S, and V; and
X² and X³ are each independently selected from S, T, A, V, G, and C.

27. The conjugate of any one of clauses 1 to 26, wherein the sequence is positioned at a C-terminus of a heavy chain constant region of the anti-MUC2 antibody.

28. The conjugate of clause 27, wherein the heavy chain constant region comprises a sequence of the formula (III):

X¹(fGly′)X²Z²X³Z³ (III),

wherein
X¹ is present or absent and, when present, can be any amino acid, with the proviso that when the sequence is at the N-terminus of the conjugate, X¹ is present;
fGly′ is the amino acid residue coupled to the drug through the linker;
X² and X³ are each independently any amino acid;
Z² is either a proline or alanine residue;
Z³ is a basic amino acid or an aliphatic amino acid, and
wherein the sequence is C-terminal to the amino acid sequence SLSLSPG (SEQ ID NO:187).

29. The conjugate of clause 28, wherein the heavy chain constant region comprises the sequence SPGSL(fGly′)TPSRGS (SEQ ID NO:188).

30. The conjugate of clause 28, wherein
Z³ is selected from R, K, H, A, G, L, V, I, and P;
X¹ is selected from L, M, S, and V; and
X² and X³ are each independently selected from S, T, A, V, G, and C.

31. The conjugate of any one of clauses 27 to 30, wherein the heavy chain constant region of the anti-MUC1 antibody comprises an amino acid sequence at least 85%, at least 90%, at least 95%, at least 96%, at least 97%, at least 98%, or at least 99% identical to the amino acid sequence set forth in SEQ ID NO:57 and comprises the fGly′ residue at amino acid position 332 instead of C.

32. The conjugate of any one of clauses 1 to 26, wherein the heavy chain constant region of the anti-MUC1 antibody comprises an amino acid sequence at least 85%, at least 90%, at least 95%, at least 96%, at least 97%, at least 98%, or at least 99% identical to the amino acid sequence set forth in any one of SEQ ID NOs:58, 62, 66, and 70 and comprises the fGly′ residue at amino acid position 59 instead of C.

33. The conjugate of any one of clauses 1 to 26, wherein the heavy chain constant region of the anti-MUC1 antibody comprises an amino acid sequence at least 85%, at least 90%, at least 95%, at least 96%, at least 97%, at least 98%, or at least 99% identical to the amino acid sequence set forth in any one of SEQ ID NOs:59, 63, 67, and 71 and comprises the fGly′ residue at amino acid position 62 instead of C.

34. The conjugate of any one of clauses 1 to 26, wherein the heavy chain constant region of the anti-MUC1 antibody comprises an amino acid sequence at least 85%, at least 90%, at least 95%, at least 96%, at least 97%, at least 98%, or at least 99% identical to the amino acid sequence set forth in any one of SEQ ID NOs:60, 64, 68, and 72 and comprises the fGly′ residue at amino acid position 92 instead of C.

35. The conjugate of any one of clauses 1 to 26, wherein the heavy chain constant region of the anti-MUC1 antibody comprises an amino acid sequence at least 85%, at least 90%, at least 95%, at least 96%, at least 97%, at least 98%, or at least 99% identical to the amino acid sequence set forth in any one of SEQ ID NOs:61, 65, 69, and 73 and comprises the fGly′ residue at amino acid position 117 instead of C.

36. The conjugate of any one of clauses 1 to 26, wherein the fGly′ residue is positioned in a light chain constant region of the anti-MUC1 antibody.

37. The conjugate of clause 36, wherein the light chain constant region comprises a sequence of the formula (III):

X¹(fGly′)X²Z²X³Z³ (III),
wherein
X¹ is present or absent and, when present, can be any amino acid, with the proviso that when the sequence is at the N-terminus of the conjugate, X¹ is present;
fGly′ is the amino acid residue coupled to the drug through the linker;
X² and X³ are each independently any amino acid;
Z² is either a proline or alanine residue;
Z³ is a basic amino acid or an aliphatic amino acid, and
wherein the sequence is C-terminal to the amino acid sequence KVDNAL (SEQ ID NO:101), and/or is N-terminal to the sequence QSGNSQ (SEQ ID NO:102).

38. The conjugate of clause 37, wherein the light chain constant region comprises the sequence KVDNAL(fGly′)TPSRQSGNSQ (SEQ ID NO:103).

39. The conjugate of clause 36, wherein
Z³ is selected from R, K, H, A, G, L, V, I, and P;
X¹ is selected from L, M, S, and V; and
X² and X³ are each independently selected from S, T, A, V, G, and C.

40. The conjugate of any one of clauses 1 to 26, wherein the fGly′ residue is positioned in a heavy chain CH₁ region of the anti-MUC1 antibody.

41. The conjugate of clause 40, wherein the light chain constant region comprises a sequence of the formula (III):

X¹(fGly′)X²Z²X³Z³ (III),
wherein
X¹ is present or absent and, when present, can be any amino acid, with the proviso that when the sequence is at the N-terminus of the conjugate, X¹ is present;
fGly′ is the amino acid residue coupled to the drug through the linker;
X² and X³ are each independently any amino acid;
Z² is either a proline or alanine residue;
Z³ is a basic amino acid or an aliphatic amino acid, and
wherein the sequence is C-terminal to the amino acid sequence SWNSGA (SEQ ID NO:104) and/or is N-terminal to the amino acid sequence GVHTFP (SEQ ID NO:105).

42. The conjugate of clause 41, wherein the heavy chain CH₁ region comprises the sequence SWNSGAL(fGly′)TPSRGVHTFP (SEQ ID NO:106).

43. The conjugate of clause 42, wherein
Z³ is selected from R, K, H, A, G, L, V, I, and P;
X¹ is selected from L, M, S, and V; and
X² and X³ are each independently selected from S, T, A, V, G, and C.

44. The conjugate of any one of clauses 1 to 26, wherein the fGly′ residue is positioned in a heavy chain CH₂ region of the anti-MUC1 antibody.

45. The conjugate of any one of clauses 1 to 26, wherein the fGly′ residue is positioned in a heavy chain CH₃ region of the anti-MUC1 antibody.

46. The conjugate of any one of clauses 1 to 45, wherein the anti-MUC1 antibody competes for binding to MUC1 with an anti-MUC1 antibody comprising:
a variable heavy chain (VH) chain comprising heavy chain CDRs1-3 (HCDRs1-3) of a VH chain having the sequence:
   EVQLVQSGAEVKKPGATVKISCKVSGYTFTDHTMHWIKQRPGKGLEWM GYFYPRDDSTNYNEKFKGRVTLTADKSTDTAYMELSSLRSEDTAVYYCARGLR YALDYWGQGTLVTVSS (SEQ ID NO:1); and
a variable light chain (VL) chain comprising light chain CDRs1-3 (LCDRs1-3) of a VL chain having the sequence:

(SEQ ID NO: 2)
EIVLTQSPATLSLSPGERATLSCRASSSVSSSYLYWYQQKPGQAPRLWI
YGTSNLASGVPARFSGSGSGTDYTLTISSLEPEDAAVYYCHQYAWSPPT
FGQGTKLEIK;
(SEQ ID NO: 3)
EIVLTQSPATLSLSPGERATLSCRASSSVGSSNLYWYQQKPGQAPRLWI
YRSTKLASGVPARFSGSGSGTDYTLTISSLEPEDAAVYYCHQYRWSPPT
FGQGTKLEIK;
or
(SEQ ID NO: 4)
EIVLTQSPATLSLSPGERATLSCRASSSVSSSYLYWYQQKPGQAPRLWI
IGTSNLASGVPARFSGSGSGTDYTLTISSLEPEDAAVYYCHQYSWSPPT
FGQGTKLEIK.

47. The conjugate of any one of clauses 1 to 45, wherein the anti-MUC1 antibody comprises:
a variable heavy chain (VH) chain comprising heavy chain CDRs1-3 (HCDRs1-3) of a VH chain having the sequence:
   EVQLVQSGAEVKKPGATVKISCKVSGYTFTDHTMHWIKQRPGKGLEWM GYFYPRDDSTNYNEKFKGRVTLTADKSTDTAYMELSSLRSEDTAVYYCARGLR YALDYWGQGTLVTVSS (SEQ ID NO:1); and
a variable light chain (VL) chain comprising light chain CDRs1-3 (LCDRs1-3) of a VL chain having the sequence:

(SEQ ID NO: 2)
EIVLTQSPATLSLSPGERATLSCRASSSVSSSYLYWYQQKPGQAPRLWI
YGTSNLASGVPARFSGSGSGTDYTLTISSLEPEDAAVYYCHQYAWSPPT
FGQGTKLEIK;
(SEQ ID NO: 3)
EIVLTQSPATLSLSPGERATLSCRASSSVGSSNLYWYQQKPGQAPRLWI
YRSTKLASGVPARFSGSGSGTDYTLTISSLEPEDAAVYYCHQYRWSPPT
FGQGTKLEIK;
or
(SEQ ID NO: 4)
EIVLTQSPATLSLSPGERATLSCRASSSVSSSYLYWYQQKPGQAPRLWI
IGTSNLASGVPARFSGSGSGTDYTLTISSLEPEDAAVYYCHQYSWSPPT
FGQGTKLEIK.

48. The conjugate of clauses 46 or 47, wherein the VH polypeptide comprises an amino acid sequence having at least 80% identity to the amino acid sequence set forth in SEQ ID NO:1.

49. The conjugate of any one of clauses 46 to 48, wherein the VL polypeptide comprises an amino acid sequence having at least 80% identity to the amino acid sequence set forth in SEQ ID NO:2, 3, or 4.

50. The conjugate of any one of clauses 46 to 49, wherein:
the HCDR1 comprises the amino acid sequence DHTMH (SEQ ID NO:17);
the HCDR2 comprises the amino acid sequence YFYPRDDSTNYNEKFKG (SEQ ID NO:18);
the HCDR3 comprises the amino acid sequence GLRYALDY (SEQ ID NO:9);
the LCDR1 comprises the amino acid sequence RASSSVSSSYLY (SEQ ID NO:10);
the LCDR2 comprises the amino acid sequence GTSNLAS (SEQ ID NO:11); and the LCDR3 comprises the amino acid sequence HQYAWSPPT (SEQ ID NO:12), as per Kabat definition.

51. The conjugate of any one of clauses 46 to 49, wherein:
the HCDR1 comprises the amino acid sequence DHTMH (SEQ ID NO:17);
the HCDR2 comprises the amino acid sequence YFYPRDDSTNYNEKFKG (SEQ ID NO:18);
the HCDR3 comprises the amino acid sequence GLRYALDY (SEQ ID NO:9);
the LCDR1 comprises the amino acid sequence RASSSVGSSNLY (SEQ ID NO:13);
the LCDR2 comprises the amino acid sequence RSTKLAS (SEQ ID NO:14); and the LCDR3 comprises the amino acid sequence HQYRWSPPT (SEQ ID NO:15), as per Kabat definition.

52. The conjugate of any one of clauses 46 to 49, wherein:
the HCDR1 comprises the amino acid sequence DHTMH (SEQ ID NO:17);
the HCDR2 comprises the amino acid sequence YFYPRDDSTNYNEKFKG (SEQ ID NO:18);
the HCDR3 comprises the amino acid sequence GLRYALDY (SEQ ID NO:9);
the LCDR1 comprises the amino acid sequence RASSSVSSSYLY (SEQ ID NO:10);
the LCDR2 comprises the amino acid sequence GTSNLAS (SEQ ID NO:11); and the LCDR3 comprises the amino acid sequence HQYSWSPPT (SEQ ID NO:16), as per Kabat definition.

53. A pharmaceutical composition comprising:
a conjugate of any one of clauses Error!Reference source not found. to 52; and a pharmaceutically-acceptable excipient.

54. A method comprising:
administering to a subject an effective amount of the conjugate of any one of clauses 1 to 52.

55. A method of treating cancer in a subject, the method comprising:
administering to the subject a therapeutically effective amount of a pharmaceutical composition of clause 53, wherein the administering is effective to treat cancer in the subject.

56. The method according to clause 55, wherein the cancer is a breast cancer, an ovarian, a lung cancer, or a gastric cancer.

57. The method according to clause 56, wherein the cancer is characterized by cancer cells expressing glycosylated MUC.

58. The method according to clause 56, wherein the conjugate binds to the glycosylated MUC1.

59. The method according to any one of clauses 55 to 58, wherein the breast cancer is triple-negative for estrogen, progesterone, and HER2.

60. The method according to clause 59, wherein the triple-negative breast cancer is metastatic triple negative breast cancer.

61. The method according to clause 59 or 60, wherein the triple-negative breast cancer is a relapsed or refractory triple negative breast cancer.

62. The method of any of clauses 55 to 61, further comprising administering to the subject a therapeutically effective amount of an immunomodulatory therapeutic agent.

63. The method of clause 62, wherein the immunomodulatory therapeutic agent is an immune checkpoint inhibitor or interleukin.

64. The method of clause 63, wherein the immune checkpoint inhibitor inhibits A2AR, B7-H3, B7- H4, BTLA, CTLA-4, CD277, IDO, KIR, PD-1, LAG-3, TIM-3, TIGIT and VISTA

65. The method of clause 64, wherein the immune checkpoint inhibitor that inhibits PD-1 signaling is an anti-PD-1 antibody.

66. The method of clause 65, wherein the anti-PD-1 antibody is nivolumab, pembrolizumab, atezolizumab, durvalumab, or avelumab.

67. The method of clause 64, wherein the immune checkpoint inhibitor that inhibits CTLA-4 is an anti-CTLA-4 antibody.

68. The method of clause 67, wherein the anti-CTLA-4 antibody is ipilimumab.

69. A method of delivering a drug to a target site in a subject, the method comprising: administering to the subject a pharmaceutical composition of clause 53, wherein the administering is effective to release a therapeutically effective amount of the drug from the conjugate at the target site in the subject.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like. Commercially available reagents referred to in the Examples were used according to manufacturer's instructions unless otherwise indicated. The source of cells identified in the Examples and throughout the specification by ECACC accession numbers is the European Collection of Cell Cultures (ECACC), Salisbury, England. Unless otherwise defined, all technical and scientific terms used herein have the same meaning as commonly understood by one of ordinary skill in the art to which this invention belongs. Exemplary methods and materials are described below although methods and materials similar or equivalent to those described herein can also be used in the practice or testing of the present invention. The materials, methods, and examples are illustrative only and not intended to be limiting in scope.

### Example 1: Anti-MUC1 Monoclonal Antibodies

### Materials and Methods

SEC HPLC: To determine aggregation, samples were analyzed using analytical size exclusion chromatography (SEC; Tosoh #08541) with a mobile phase of 300 mM NaCl, 25 mM sodium phosphate pH 6.8.

MUC1 ELISA: Antigens were coated directly on a Streptavidin (Pierce, 15500) or Maxisorp (VWR, 62409-024) 96-well plate at 100 ng/well in PBS. Coated plates were incubated at 4° C. overnight. The plates were blocked with casein blocking buffer (Thermo Fisher, 37528) and washed with PBS-Tween-20. Antibodies were serially diluted in PBS, added to the coated wells and incubated for 1 h at room temperature with shaking. To test for stickiness, antibodies were also added to uncoated, blocked wells. Peroxidase (HRP)-conjugated anti-Fc secondary (Jackson Immunoresearch, #109-035-098) was used for detection, followed by a TMP substrate (Thermo Fisher, 34028) and H2SO₄ quench. Absorbance was read at 450 nM on a Molecular Devices plate reader.

Flow cytometric analysis: Cell lines were harvested with Versene, transferred to PBS with 2% FBS (PBS/FBS) and chilled. Cells were incubated for 20-30 minutes on ice with specified Abs (1 μg/test). Following a 1× wash with PBS/FBS, AlexaFluor488 conjugated anti-human IgG-Fc antibody & the dye 7-AAD (used to exclude dead cells) was added and cells were incubated on ice for 20 mins. Samples were washed 2× with PBS/FBS followed by flow cytometric analysis on a FACS Canto™ instrument running FACSDiva™ software. Analysis was performed by excluding doublets and dead cells and gating on the FSC/SSC cell population. The Geometric Mean Fluorescence Intensity (gMFI) of the AlexaFluor488 channel was determined for each antibody. All samples were run in triplicate. Controls included a MUC1-negative cell line (HCT-116), cells labeled with secondary antibody alone, and unstained cells.

Differential Scanning Fluorimetry. Antibody (10 μL at 1 mg/mL) was used for protein melting temperature measurement using the Protein Thermal Shift Kit (Applied Biosystems). The antibody was mixed with 5 μL of buffer and 2.5 μL of 8× fluorescent dye for a 20 μL reaction. A QuantStudio3 (Applied Biosystems) real-time PCR machine was used to generate a melting curve. The setting was: 25° C. hold for 2 min, followed by 0.05° C./sec. temperature increase to 99° C., followed by a 2 min hold at 99° C. The raw data were analyzed by Protein Thermal Shift software (Applied Biosystems).

### Results

Three anti-MUC1 monoclonal antibodies, MUC1 gB06, MUC1 G12, and MUC1 H02 were produced. The three antibodies share the same heavy chain sequence and have different light chain sequences.

The variable heavy chain region sequence with framework regions (underlined) and HCDRs (bold) demarcated based on Chothia definition, Kabat definition, and IMGT definition are shown:

(SEQ ID NO: 1)
EVQLVQSGAEVKKPGATVKISCKVSGYTFTDHTMHWIKQRPGKGLEWM
GYFYPRDDSTNYNEKFKGRVTLTADKSTDTAYMELSSLRSEDTAVYYCA
RGLRYALDYWGQGTLVTVSS (Chothia definition)
(SEQ ID NO: 1)
EVQLVQSGAEVKKPGATVKISCKVSGYTFTDHTMHWIKQRPGKGLEWM
GYFYPRDDSTNYNEKFKGRVTLTADKSTDTAYMELSSLRSEDTAVYYCA
RGLRYALDYWGQGTLVTVSS (Kabat definition)
(SEQ ID NO: 1)
EVQLVQSGAEVKKPGATVKISCKVSGYTFTDHTMHWIKQRPGKGLEWM
GYFYPRDDSTNYNEKFKGRVTLTADKSTDTAYMELSSLRSEDTAVYYCA
RGLRYALDYWGQGTLVTVSS (IMGT definition)

The variable light chain region sequence with framework regions (underlined) and LCDRs (bold) demarcated based on Chothia definition, Kabat definition, and IMGT definition are shown:

gB06, VL:
(SEQ ID NO: 2)
EIVLTQSPATLSLSPGERATLSCRASSSVSSSYLYWYQQKPGQAPRLWI
YGTSNLASGVPARFSGSGSGTDYTLTISSLEPEDAAVYYCHQYAWSPPT
FGQGTKLEIK (Chothia and Kabat definition)
G12, VL:
(SEQ ID NO: 3)
EIVLTQSPATLSLSPGERATLSCRASSSVGSSNLYWYQQKPGQAPRLWI
YRSTKLASGVPARFSGSGSGTDYTLTISSLEPEDAAVYYCHQYRWSPPT
FGQGTKLEIK (Chothia and Kabat definition)
H02, VL:
(SEQ ID NO: 4)
EIVLTQSPATLSLSPGERATLSCRASSSVSSSYLYWYQQKPGQAPRLWI
IGTSNLASGVPARFSGSGSGTDYTLTISSLEPEDAAVYYCHQYSWSPPT
FGQGTKLEIK (Chothia and Kabat definition)
gB06, VL:
(SEQ ID NO: 2)
EIVLTQSPATLSLSPGERATLSCRASSSVSSSYLYWYQQKPGQAPRLWI
YGTSNLASGVPARFSGSGSGTDYTLTISSLEPEDAAVYYCHQYAWSPPT
FGQGTKLEIK (IMGT definition)
G12, VL:
(SEQ ID NO: 3)
EIVLTQSPATLSLSPGERATLSCRASSSVGSSNLYWYQQKPGQAPRLWI
YRSTKLASGVPARFSGSGSGTDYTLTISSLEPEDAAVYYCHQYRWSPPT
FGQGTKLEIK (IMGT definition)
H02, VL:
(SEQ ID NO: 4)
EIVLTQSPATLSLSPGERATLSCRASSSVSSSYLYWYQQKPGQAPRLWI
IGTSNLASGVPARFSGSGSGTDYTLTISSLEPEDAAVYYCHQYSWSPPT
FGQGTKLEIK (IMGT definition)

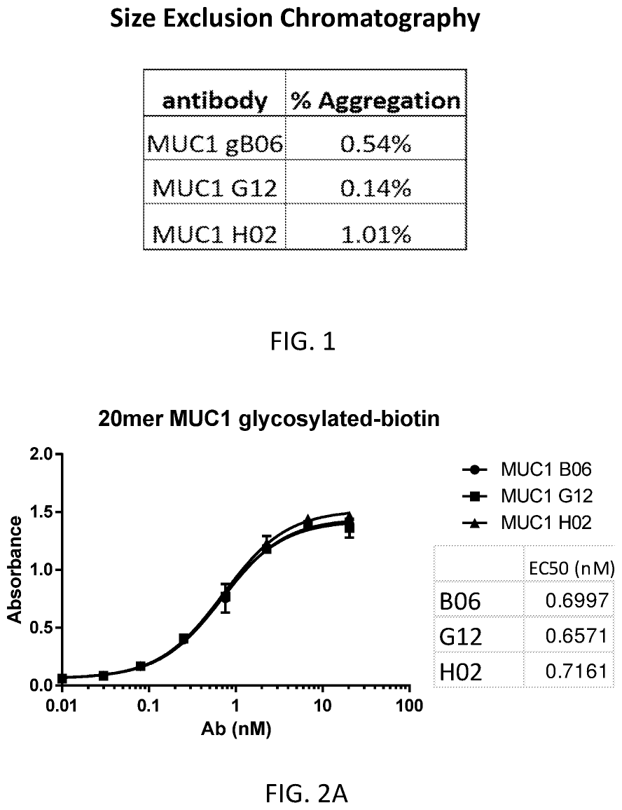

FIG. 1 shows that anti-MUC1 monoclonal antibodies, MUC1 gB06, MUC1 G12, and MUC1 H02 are more than 99%, more than 99%, and more than 98% monomeric, respectively, as determined by size exclusion chromatography (SEC).

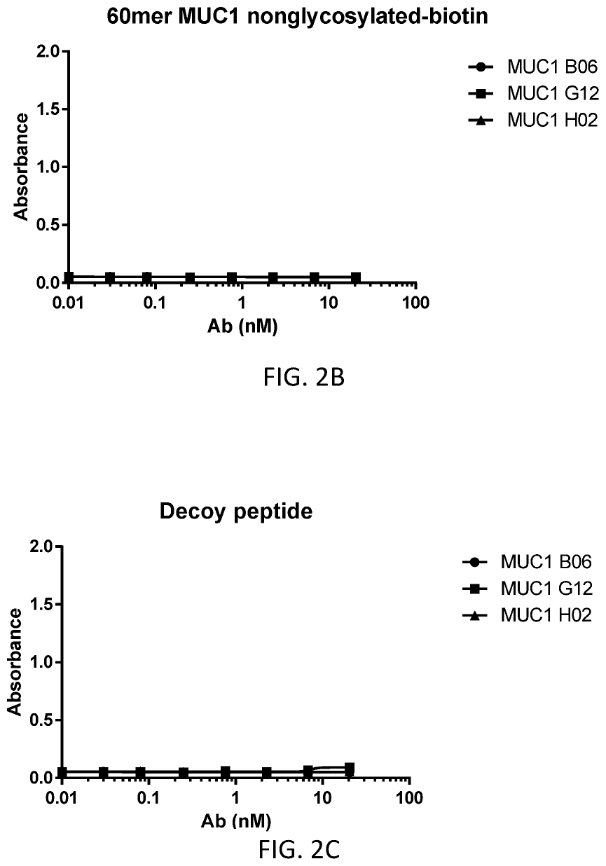

FIGS. 2A-2C show that anti-MUC1 monoclonal antibodies, MUC1 gB06, MUC1 G12, and MUC1 H02 bind to recombinant 20mer MUC1 glycosylated-biotin but not to recombinant 60mer MUC1 non-glycosylated-biotin or to a decoy peptide as assessed by ELISA. 20mer MUC1 glycosylated-biotin refers to a peptide comprising the sequence VTSAPDTRPAPGSTAPPAHG (SEQ ID NO:26) with Tn (GalNac) antigen or sialyl Tn (Neu5Acα2-6GalNAc) antigen modifications on some of the S/T residues, where biotin in conjugated to the N-terminus. 60mer MUC1 non-glycosylated-biotin refers to a peptide comprising the sequence VTSAPDTRPAPGSTAPPAHGVTSAPDTRPAPGSTAPPAHGVTSAPDTRPAPGSTAPPAHG (SEQ ID NO:27), where biotin in conjugated to the N-terminus. Decoy peptide refers to a peptide comprising the sequence PLPVTSTSSASTGHATPLAV (SEQ ID NO:28), with Tn (GalNac) antigen or sialyl Tn (Neu5Acα2-6GalNAc) antigen modifications on some of the S/T residues.

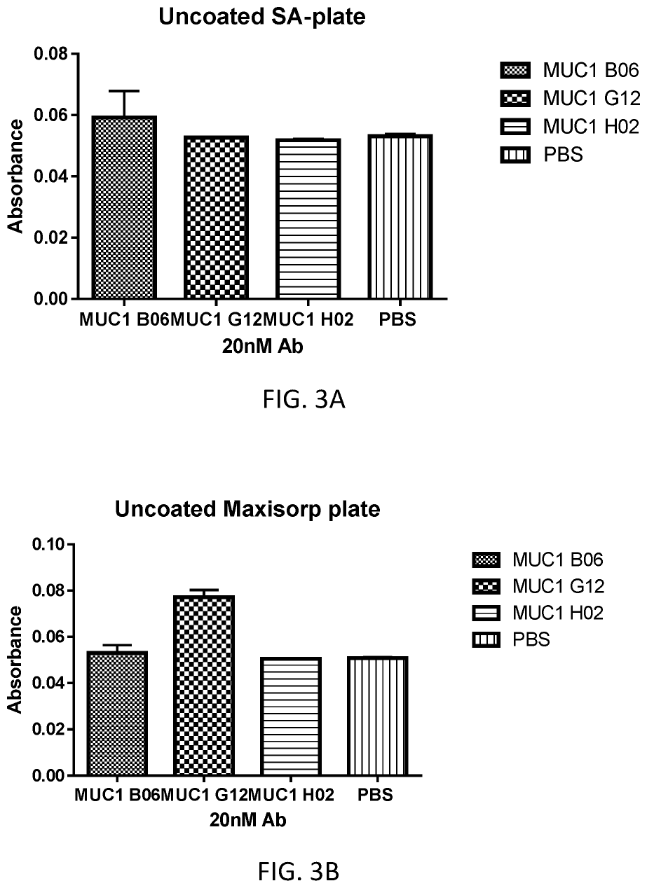

FIGS. 3A-3B show level of binding by the anti-MUC1 antibodies, MUC1 gB06, MUC1 G12, and MUC1 H02 to uncoated streptavidin or Maxisorp plate.

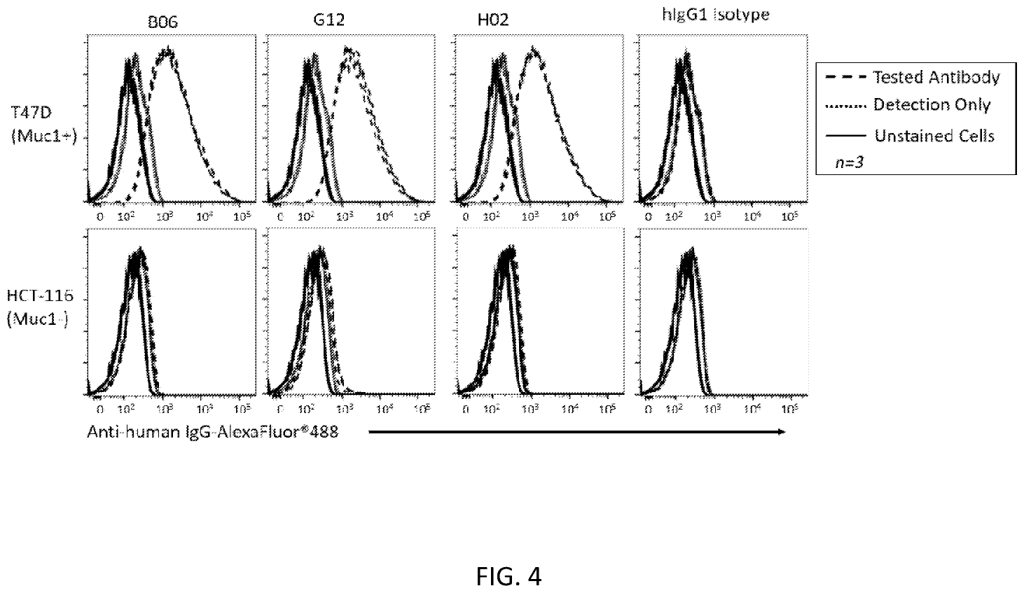

FIG. 4 shows superimposed histograms for indicated antibodies tested in triplicates.

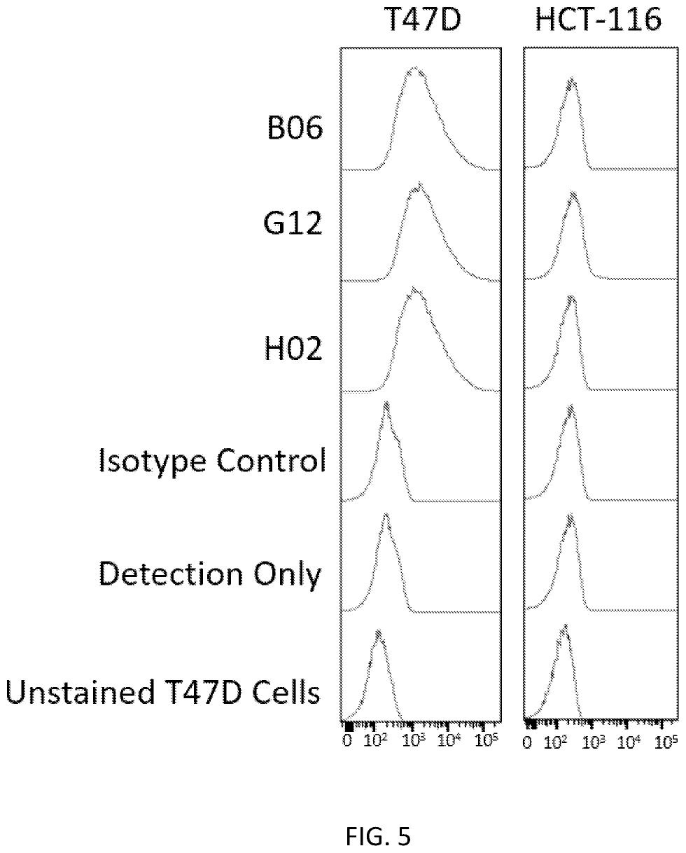

FIG. 5 shows staggered histograms for indicated antibodies.

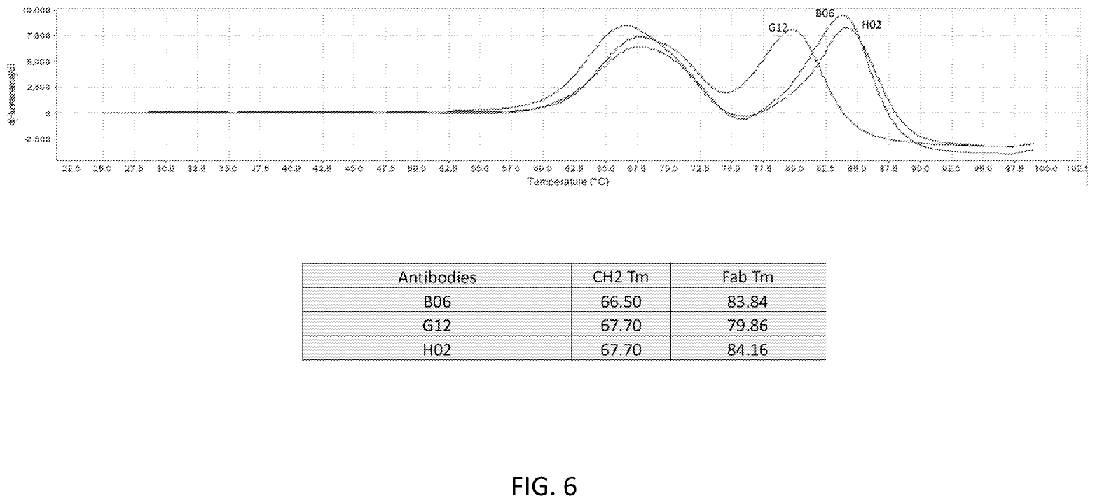

FIG. 6 shows the melting temperature of CH2 and Fab regions of the B06, G12, and H02 anti-MUC1 antibodies as determined by differential scanning fluorimetry.

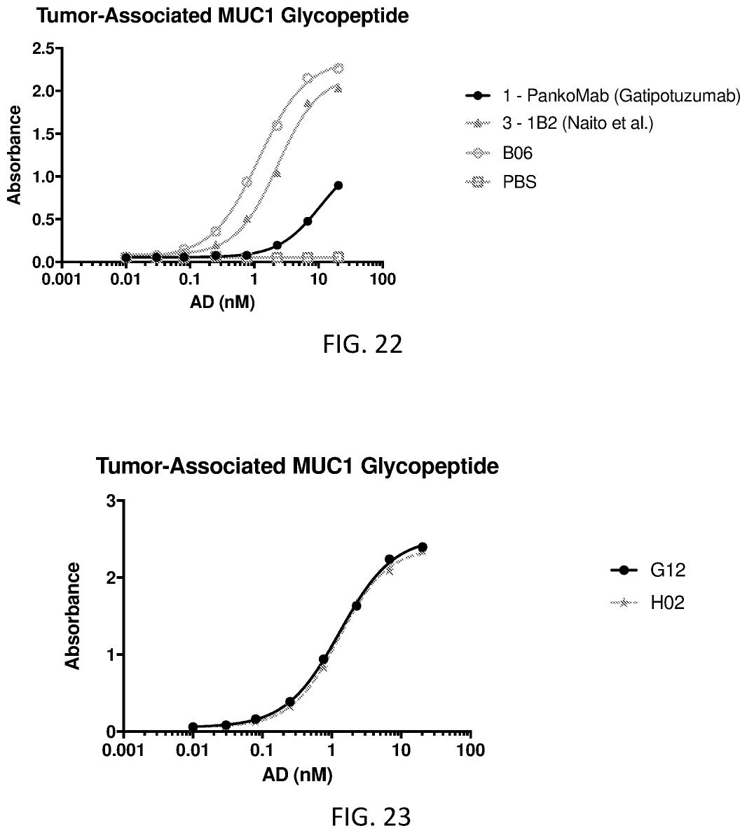

FIG. 22. Binding of anti-MUC1 affinity-matured variant B06 and comparator antibodies, PankoMab and 1B2, to 20mer MUC1 glycosylated-biotin peptide as assessed by ELISA.

FIG. 23. Binding of anti-MUC1 affinity-matured variants G12 and H02 to 20mer MUC1 glycosylated-biotin peptide as assessed by ELISA.

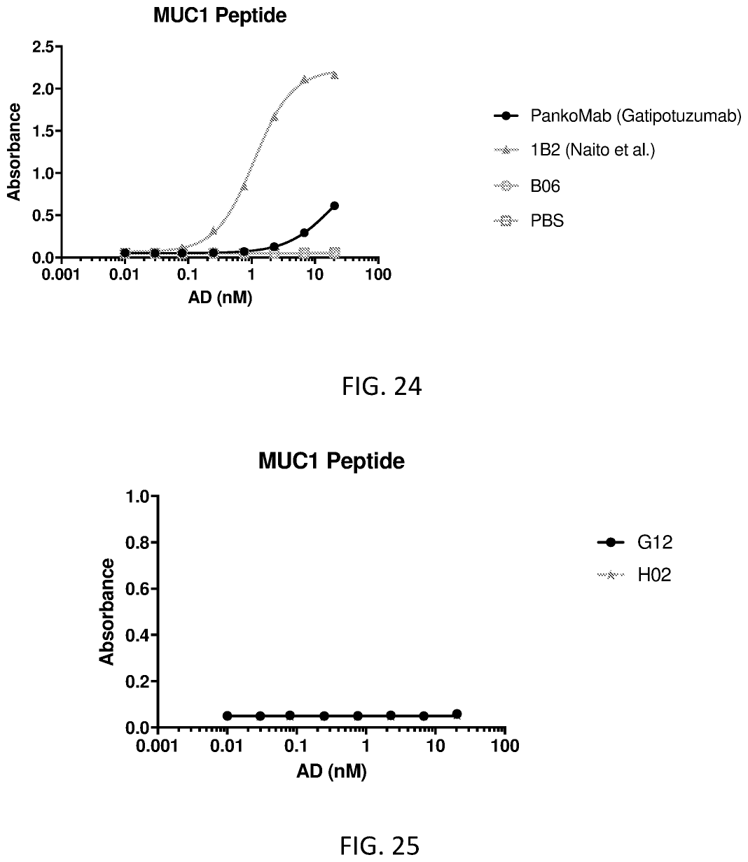

FIG. 24. Binding of anti-MUC1 affinity-matured variant B06 and comparator antibodies, PankoMab and 1B2, to 60mer MUC1 non-glycosylated-biotin peptide as assessed by ELISA.

FIG. 25. Binding of anti-MUC1 affinity-matured variants G12 and H02 to 60mer MUC1 non-glycosylated-biotin peptide as assessed by ELISA.

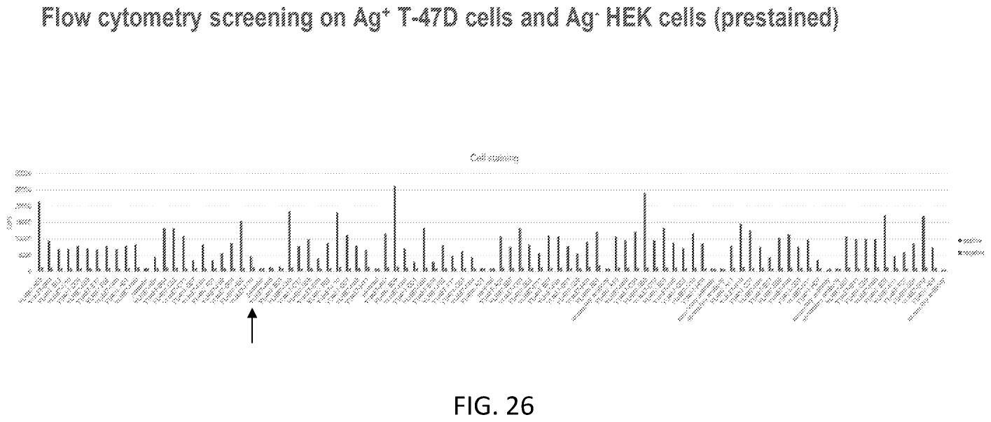

FIG. 26 shows binding of anti-MUC1 affinity-matured variants and parental antibody to antigen-positive T47D cells or antigen-negative HEK cells as assessed by flow cytometry.

### Example 2

### General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any purification protocol known in the art, including chromatography, such as HPLC, preparative thin layer chromatography, flash column chromatography and ion exchange chromatography. Any suitable stationary phase can be used, including normal and reversed phases as well as ionic resins. In certain embodiments, the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4^{th} edition, Vol. 15/1, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

The subject compounds can be synthesized via a variety of different synthetic routes using commercially available starting materials and/or starting materials prepared by conventional synthetic methods. A variety of examples of synthetic routes that can be used to synthesize the compounds disclosed herein are described in the schemes below.

### Synthesis of MMAE Construct 8

Compounds 1 and 4 were obtained commercially from Shanghai Medicilon and used as received. Monomethylauristatin A 5 (MMAE) was purchased from BroadPharm. All other reagents were obtained from commercial sources and used without purification.

### Preparation of (R)-2-(3-(2-((2-(((9H-fluoren-9-yl)methoxy)carbonyl)-1,2-dimethylhydrazineyl)methyl)-1H-pyrrolo[2,3-b]pyridin-1-yl)propanamido)-3-oxo-3-((2-(2-(3-oxo-3-(perfluorophenoxy)propoxy)ethoxy)ethyl)amino)propane-1-sulfonic acid (3)

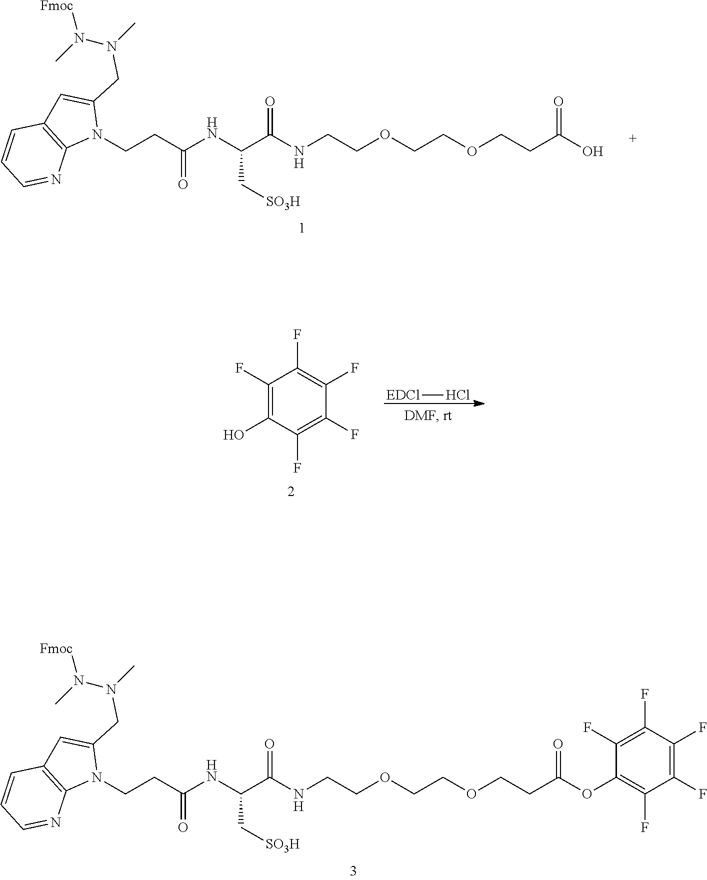

Carboxylic acid 1 (1.33 g, 1.67 mmol) was combined with pentafluorophenol 2 (1.23 g, 6.68 mmol) in 6.5 mL of anhydrous DMF. This mixture was treated with EDCI-HCl (0.64 g, 3.34 mmol) in one portion at room temperature and stirred for 20 h until 1 was fully consumed as judged by HPLC analysis. Reaction mixture was directly purified by reversed-phase chromatography (C18 column, 0-80% acetonitrile-water with 0.05% TFA). Pure fractions were combined, concentrated under vacuum until murky, and lyophilized to give PFP-ester product 3 (1.40 g, 1.46 mmol, 87% yield) as a tan powder. LRMS (ESI): m/z 961.2 [M+H]⁺, Calcd for C₄₄H₄₅F₅N₆O₁₁S m/z 961.3.

### Preparation of (2S,3R,4S,5S,6S)-2-(2-((S)-2-((S)-2-((((9H-fluoren-9-yl)methoxy)carbonyl)amino)-3-methylbutanamido)propanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (6)

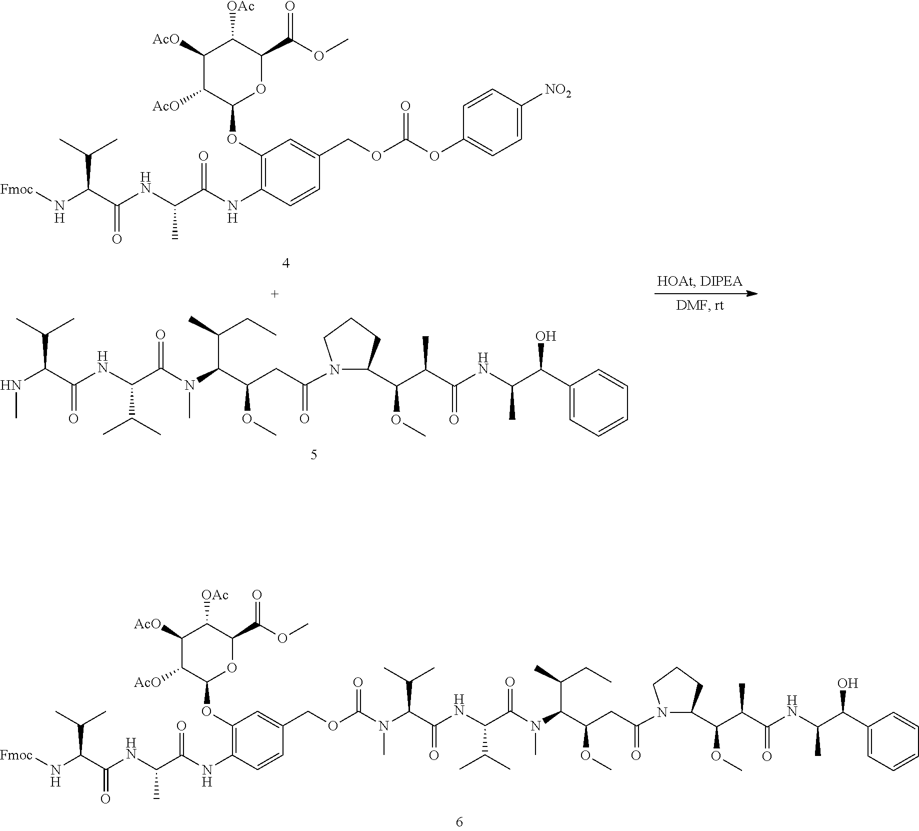

In a 20 mL glass vial were combined monomethyl auristatin A 5 (720 mg, 1.0 mmol), 5 mL of anhydrous DMF, and 0.35 mL of DIPEA (2.0 mmol) at room temperature. The resulting mixture was stirred and treated with PNP carbonate 4 (1014 mg, 1.0 mmol) as a solid in a few small portions, followed by the addition of HOAt (136 mg, 1.0 mmol) in one portion at room temperature. Reaction mixture was stirred for 6 h until reaction was judged complete (HPLC). Reaction mixture was poured into 30 mL of water, and the resulting precipitate was separated by spinning and collected, washed with 5 mL of water, and dried briefly under high vacuum to give 1.87 g of crude product 6 as a yellowish solid, which was taken to the next step without purification.

### Preparation of (2S,3S,4S,5R,6S)-6-(2-((S)-2-((S)-2-amino-3-methylbutanamido)propanamido)-5-((5S,8S,11S,12R)-11-((S)-sec-butyl)-12-(2-((S)-2-((R,2R)-3-((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3, 6,9-trioxo-2,13-dioxa-4,7,10-triazatetradecyl)phenoxy)-3,4,5-trihydroxytetrahydro-2H-pyran-2-carboxylic acid (7)

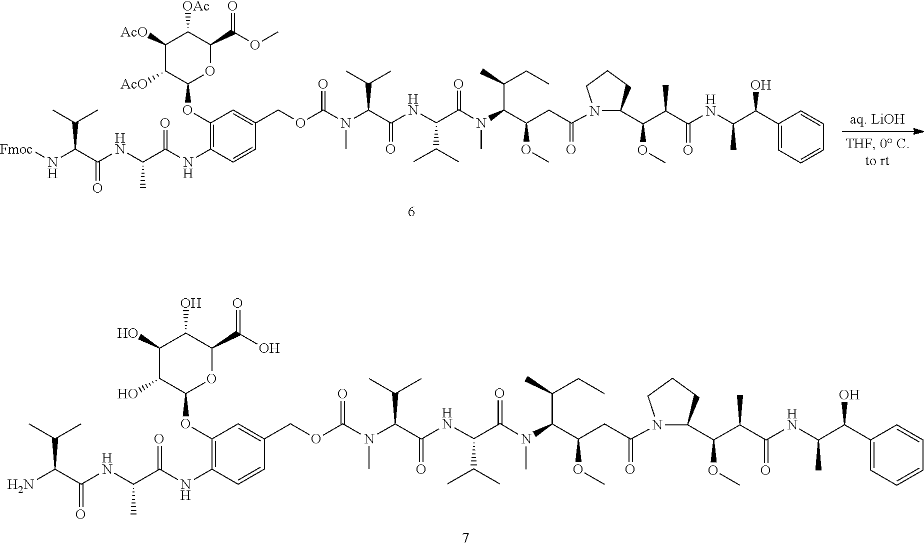

A solution of crude compound 6 (1.87 g) in 15 mL of THF was cooled down to 0° C. in an ice bath and treated slowly with 1 M aqueous lithium hydroxide solution (3 mL). Reaction mixture was stirred at 0° C. for 3 hours, then warmed up to ambient temperature, treated with 3 mL of 1 M aqueous lithium hydroxide and diluted with 3 mL of methanol. The resulting mixture was stirred at room temperature for 3 hours until hydrolysis was complete (HPLC), then quenched by adding 1 M aqueous HCl solution to pH 7. Reaction mixture was then concentrated under reduced pressure and washed with 10 mL of MTBE. Aqueous layer was purified by reversed-phase chromatography (C18 column, 0-40% acetonitrile-water with 0.05% TFA). Pure product fractions were combined, concentrated under reduced pressure, and lyophilized to give compound 7 as a white powder (735 mg, 0.60 mmol, 60% yield over 2 steps). LRMS (ESI): m/z 1229.7 [M+H]⁺, Calcd for C₆₁H₉₆N₈O₁₈ m/z 1229.7.

### Preparation of (2S,3S,4S,5R,6S)-6-(5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3, 6,9-trioxo-2,13-dioxa-4,7,10-triazatetradecyl)-2-((2S,5S,18R)-22-(2-((1,2-dimethylhydrazineyl)methyl)-1H-pyrrolo[2,3-b]pyridin-1-yl)-5-isopropyl-2-methyl-4,7,17,20-tetraoxo-18-(sulfomethyl)-10,13-dioxa-3,6,16,19-tetraazadocosanamido)phenoxy)-3,4,5-trihydroxytetrahydro-2H-pyran-2-carboxylic acid (8)

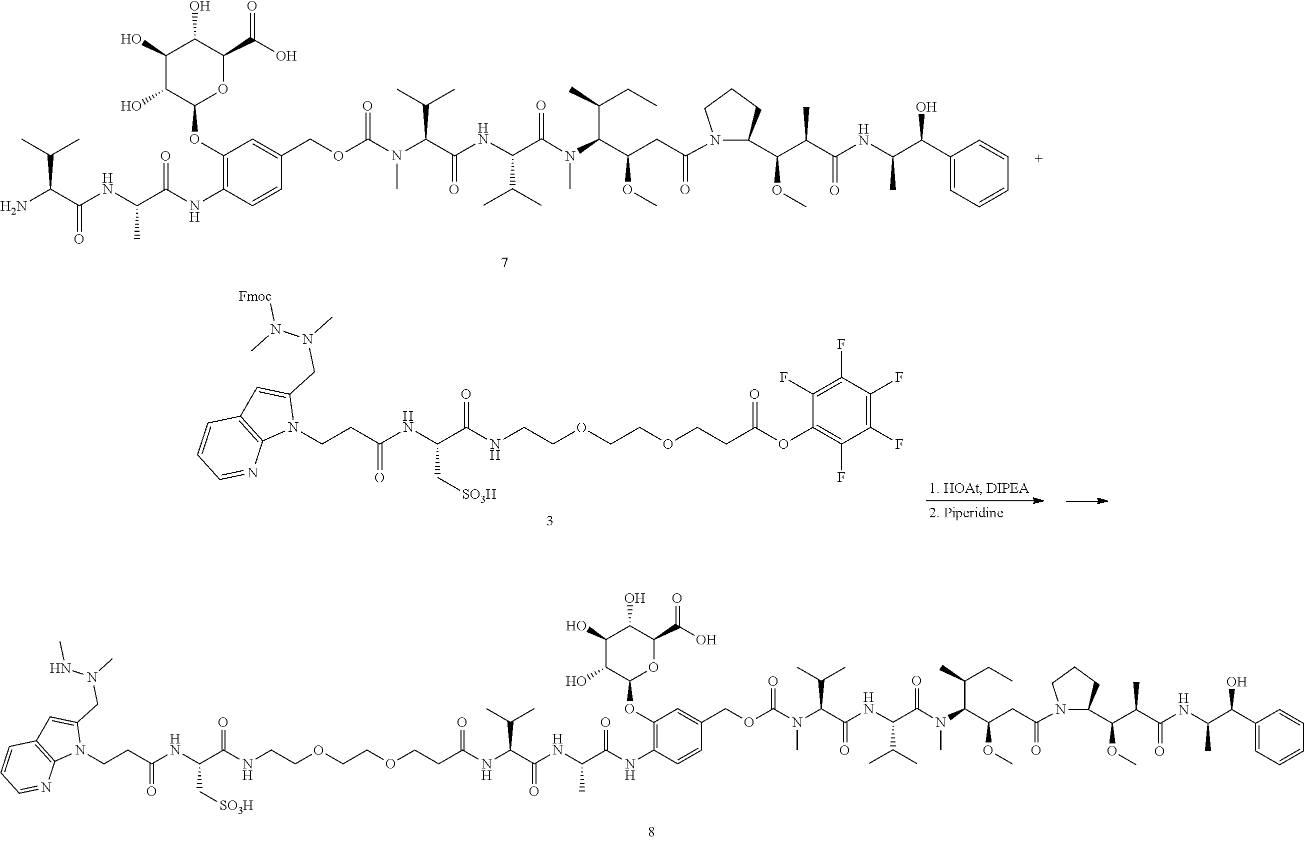

To a stirred solution of compound 7 (735 mg, 0.60 mmol) in 3 mL of anhydrous DMA were added DIPEA (0.21 mL, 1.2 mmol) and a solution PFP-ester 3 (575 mg, 0.60 mmol) in 2 mL of DMA at room temperature, followed by the addition of HOAt (84 mg, 0.60 mmol). The resulting mixture was stirred for 30 minutes until coupling was judged complete (HPLC analysis), then treated directly with 1.2 mL of piperidine at room temperature. After 15 minutes, reaction mixture was purified by reversed-phase chromatography (C18 column, 0-40% gradient of acetonitrile-water). Pure fractions were combined, concentrated under reduced pressure and room temperature, and lyophilized to give compound 8 (808 mg, 0.45 mmol, 75% yield) as a white fluffy powder. LRMS (ESI): m/z 1783.9 [M+H]⁺, Calcd for C₈₄H₁₃₀N₁₄O₂₆S m/z 1783.9.

### Synthesis of Belotecan Construct 21

Synthetic intermediates 4 and 9 were obtained commercially from Shanghai Medicilon and used as received. Belotecan 15 was purchased from AstaTech. All other reagents were obtained from commercial sources and used without purification.

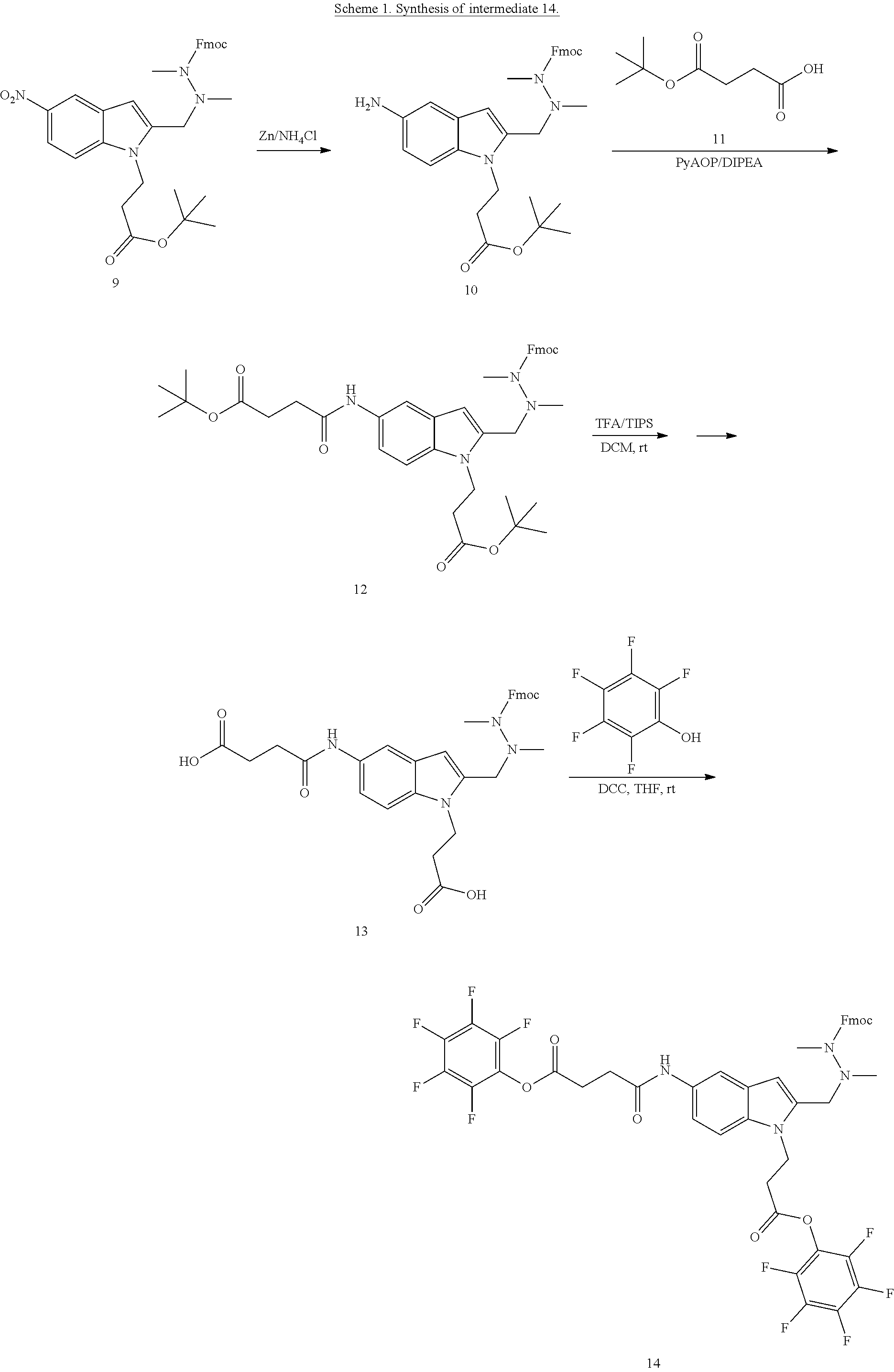

### Preparation of (9H-fluoren-9-yl)methyl 2-((5-amino-1-(3-(tert-butoxy)-3-oxopropyl)-1H-indol-2-yl)methyl)-1,2-dimethylhydrazine-1-carboxylate (10)

Nitro compound 9 (116 mg, 0.20 mmol) was dissolved in 1 mL of THF and combined with a solution of ammonium chloride (85 mg, 1.6 mmol) in 0.5 mL of water and 1 mL of methanol. The resulting mixture was vigorously stirred at room temperature and treated with zinc powder (104 mg, 1.6 mmol) in small portions over 5 minutes. Reaction mixture was stirred for 2 hours, solids were filtered off, filtrate was diluted with 20 mL of saturated aqueous ammonium chloride solution and extracted with ethyl acetate (2×25 mL). Organic extracts were dried over sodium sulfate, solvents removed under vacuum to give crude product 10 which was taken to the next step without purification. LRMS (ESI): m/z 555.3 [M+H]⁺, Calcd for C₃₃H₃₈N₄O₄ m/z 555.3.

### Preparation of (9H-fluoren-9-yl)methyl 2-((1-(3-(tert-butoxy)-3-oxopropyl)-5-(4-(tert-butoxy)-4-oxobutanamido)-1H-indol-2-yl)methyl)-1,2-dimethylhydrazine-1-carboxylate (12)

Crude compound 10 (˜0.20 mmol) was combined with 4-(tert-butoxy)-4-oxobutanoic acid 11 (40 mg, 0.23 mmol) in 2 mL of DMF. To this mixture were added DIPEA (0.12 mL, 0.6 mmol), followed by PyAOP (110 mg, 0.21 mmol) in one portion at room temperature. After 30 minutes, reaction was quenched by pouring into saturated aqueous ammonium chloride, extracted with ethyl acetate, washed with brine, dried over sodium sulfate. Solvent was removed under vacuum to give 120 mg (0.17 mmol, 85% yield over 2 steps) of product 12 as a dark oil which was used further without additional purification. LRMS (ESI): m/z 733.4 [M+Na]⁺, Calcd for C₄₁H₅₀N₄O₇ m/z 733.4.

### Preparation of 4-((2-((2-(((9H-fluoren-9-yl)methoxy)carbonyl)-1,2-dimethylhydrazinyl)methyl)-1-(2-carboxyethyl)-1H-indol-5-yl)amino)-4-oxobutanoic acid (13)

Bis-tert-butyl ester compound 12 (120 mg, 0.17 mmol) was dissolved in a mixture of 2 mL of anhydrous DCM, 2 mL of TFA, and 0.5 mL of trisopropylsilane. The resulting mixture was allowed to stand at room temperature for 4 hours. Solvents were removed under vacuum, and the residue was purified by reversed phase chromatography (C18 column, 0-70% v/v gradient of CH₃CN/H₂O with 0.05% TFA) to obtain 53 mg (0.09 mmol, 53% yield) of diacid product 13. LRMS (ESI): m/z 599.3 [M+H]⁺, Calcd for C₃₃H₃₄N₄O₇ m/z 599.2.

### Preparation of (9H-fluoren-9-yl)methyl 1,2-dimethyl-2-((1-(3-oxo-3-(perfluorophenoxy)propyl)-5-(4-oxo-4-(perfluorophenoxy)butanamido)-1H-indol-2-yl)methyl)hydrazine-1-carboxylate (14)

To a mixture of diacid 13 (50 mg, 0.084 mmol) and pentafluorophenol (46 mg, 0.25 mmol) in 2 mL of anhydrous THF were added DCC (51 mg, 0.25 mmol) in one portion at room temperature. The resulting mixture was stirred for 16 hours, solids were filtered off, filtrate concentrated, and purified by reversed phased chromatography (C18 column, 0-100% v/v gradient of CH₃CN/H₂O with 0.05% TFA). Fractions containing product were concentrated to about 20 mL, poured into 50 mL of 10% aqueous citric acid, and extracted with ethyl acetate (2×20 mL), dried over sodium sulfate. Solvents were removed under vacuum to give 67 mg (0.072 mmol, 86% yield) of bis-PFP ester product 14 as a dark viscous oil. LRMS (ESI): m/z 953.1 [M+Na]⁺, Calcd for C₄₅H₃₂F₁₀N₄O₇ m/z 953.2.

### Preparation of (2S,3S,4S,5R,6S)-6-(2-((S)-2-((S)-2-amino-3-methylbutanamido)propanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (16)

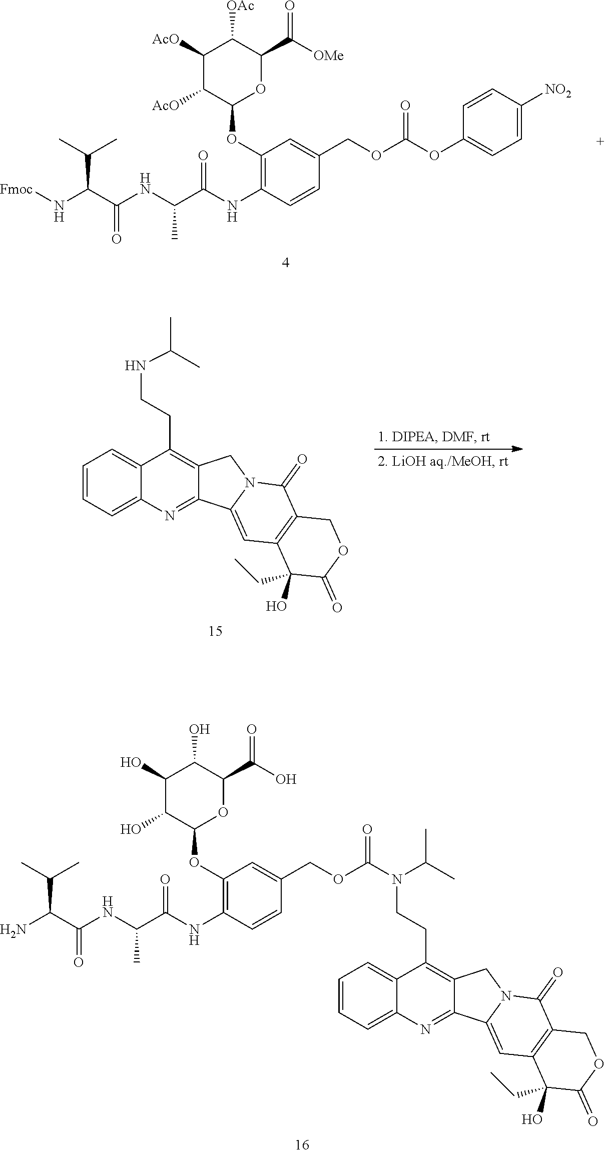

To a solution of belotecan 15 (HCl salt, 20 mg, 43 μmol) in 2 mL DMF were added 15 μL of DIPEA (86 μmol) and 6 mg of HOAt (43 μmol). The resulting mixture was treated with PNP carbonate 4 (43 mg, 43 μmol) at room temperature and stirred for one hour, then DMF was removed under vacuum. The residue was dissolved in 1 mL of MeOH and treated with 1 mL of 1M aqueous LiOH. After 10 minutes, 1 mL of 1M aqueous HCl was added to the mixture, followed by 1 mL of 0.5 M pH 4.7 acetate buffer. The resulting mixture was stirred for 30 minutes at room temperature and directly purified by reversed phase HPLC (C18 column, 0-50% v/v gradient of CH₃CN/H₂O with 0.05% TFA). Solvent was removed under vacuum to give 17 mg (18 μmol, 43% yield) of compound 16 as a glassy yellow solid. LRMS (ESI): m/z 945.4 [M+H]⁺, Calcd for C₄₇H₅₆N₆O₁₅ m/z 945.4.

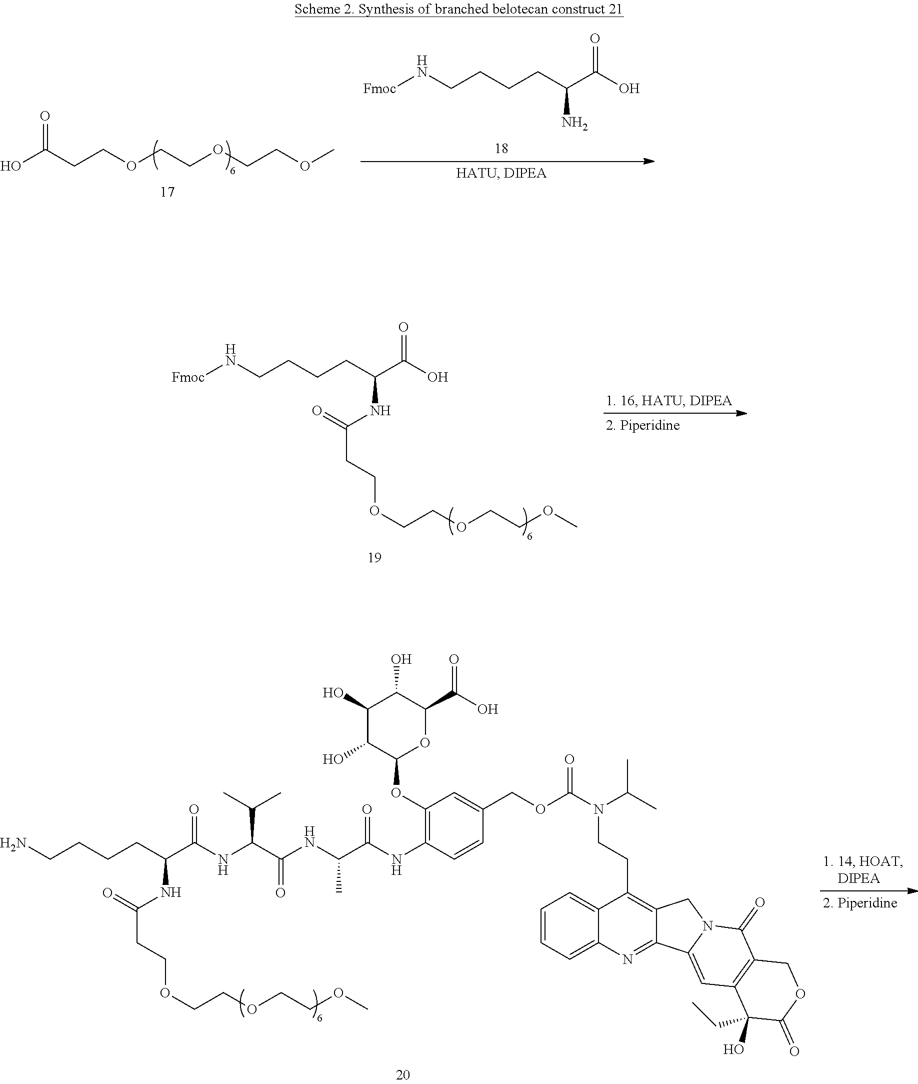

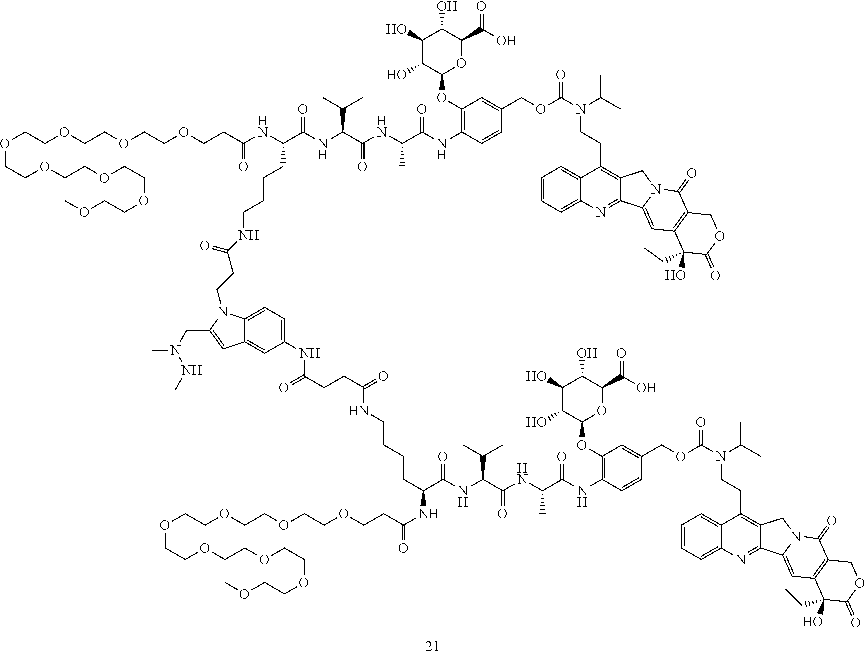 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCC(=O)Nc2ccc3c(c2)cc(CN(C)NC)n3CCC(=O)NCCCC[C@H](NC(=O)CCOCCOCCOCCOCCOCCOCCOCCOC)C(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)NC(=O)CCOCCOCCOCCOCCOCCOCCOCCOC)C(C)C)c(O[C@@H]2O[C@H](C(=O)O)[C@@H](O)[C@H](O)[C@H]2O)c1)C(C)C]

### Preparation of N⁶-(((9H-fluoren-9-yl)methoxy)carbonyl)-N²-(3-(2-(2-methoxyethoxy)ethoxy)propanoyl)-L-lysine 19)

To a solution of mPEG8-acid 17 (100 mg, 0.24 mmol) in 2 mL of anhydrous DMF were added DIPEA (0.13 mL, 0.72 mmol) and HATU (93 mg, 0.24 mmol) at room temperature. The resulting mixture was stirred for one hour, then Lys(Fmoc)-OH 18 (89 mg, 0.24 mmol) was added to the mixture, and stirring continued for one hour. Reaction mixture was directly purified by reversed-phase chromatography HPLC (C18, 0-70% v/v MeCN—H₂O with 0.05% TFA) to give 120 mg of compound 19 (0.16 mmol, 67% yield) as a colorless oil. LRMS (ESI): m/z 763.4 [M+H]⁺, Calcd for C₃₉H₅₈N₂O₁₃ m/z 763.4.

### Preparation of (2S,3S,4S,5R,6S)-6-(2-((28S,31 S, 34S)-28-(4-aminobutyl)-31-isopropyl-34-methyl-26,29,32-trioxo-2,5,8,11,14,17,20,23-octaoxa-27,30,33-triazapentatriacontan-35-amido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (20)

To a solution of carboxylic acid 23 (45 mg, 59 μmol) in 3 mL of anhydrous DMF were added DIPEA (21 μL, 120 μmol) and HATU (22 mg, 59 μmol) at room temperature. The resulting mixture was stirred for 20 minutes and combined with amine 16 (55 mg, 58 μmol) in 1 mL of DMF. Reaction mixture was stirred for 30 minutes, then piperidine (115 μL, 1.2 mmol) was added to the mixture at room temperature. After 20 minutes, reaction mixture was directly purified by reversed-phase prep HPLC (C18, 0-50% v/v MeCN—H₂O with 0.05% TFA). Lyophilization of pure fractions afforded 34 mg (23 μmol, 40% yield) of compound 20 as a yellow powder. LRMS (ESI): m/z 1467.7 [M+H]⁺, Calcd for C₇₁H₁₀₂N₈O₂₅ m/z 1467.7.

### Preparation of (2S,3S,4S,5R,6S)-6-(2-((28S,31S,34S)-28-(4-(3-(5-((S)-28-(((S)-1-(((S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1-oxopropan-2-yl)amino)-3-methyl-1-oxobutan-2-yl)carbamoyl)-26,34-dioxo-2,5,8,11,14,17,20,23-octaoxa-27,33-diazaheptatriacontan-37-amido)-2-((1,2-dimethylhydrazineyl)methyl)-1H-indol-1-yl)propanamido)butyl)-31-isopropyl-34-methyl-26,29,32-trioxo-2,5,8,11,14,17,20,23-octaoxa-27,30,33-triazapentatriacontan-35-amido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (21)

To a mixture of compound 20 (34 mg, 23 μmol) and DIPEA (8 μL, 46 μmol) in 2 mL of DMA were added bis-PFP ester 14 (9.4 mg, 10.5 μmol), followed by HOAt (3 mg, 23 μmol) at room temperature. The resulting mixture was allowed to stand for 30 minutes at room temperature, then piperidine (21 μL, 0.21 mmol) was added to the mixture at room temperature. After 20 minutes, reaction mixture was directly purified by reversed phase prep HPLC (C_{18, 0}-50% v/v MeCN—H₂O with 0.05% TFA). Pure fractions were combined and lyophilized to afford compound 21 as a yellow solid (23 mg, 7 μmol, 67% yield). LRMS (ESI): m/z 1638.3 [M+H]²⁺, Calcd for C₁₆₀H₂₂₄N₂₀O₅₃ m/z 1638.8.

### Example 3: Anti-MUC1 Monoclonal Antibody-Drug Conjugate

### Bioconjugation, Purification, and HPLC Analytics

Methods. Antibodies (15 mg/mL) bearing one aldehyde tag were conjugated to linker-payloads at 1.1 mM, respectively. Reactions proceeded for 72 h at 37° C. in 20 mM sodium citrate, 50 mM NaCl pH 5.5 (20/50 buffer) containing 0.85-2.5% DMA. After conjugation, free drug was removed using a 30 kD MWCO 0.5 mL Amicon spin concentrator. Samples were added to the spin concentrator, centrifuged at 15,000×g for 7 min, then diluted with 450 μL 20 mM sodium citrate, 50 mM NaCl pH 5.5 and centrifuged again. The process was repeated 10 times. To determine the DAR of the final product, ADCs were examined by analytical chromatography using hydrophobic interaction (HIC) (Tosoh #14947) or PLRP-RP (Agilent PL1912-1802 1000A, 8 μm, 50×2.1 mm) columns. HIC analysis used mobile phase A: 1.5 M ammonium sulfate, 25 mM sodium phosphate pH 7.0, and mobile phase B: 25% isopropanol, 18.75 mM sodium phosphate pH 7.0. PLRP analysis used mobile phase A: 0.1% trifluoroacetic acid in water, and mobile phase B: 0.1% trifluoroacetic acid in acetonitrile. Prior to PLRP analysis, sample was denatured with the addition of 50 mM DTT, 4 M guanidine HCl (final concentrations) and heating at 37° C. for 30 min. To determine aggregation, samples were analyzed using analytical size exclusion chromatography (SEC; Tosoh #08541) with a mobile phase of 300 mM NaCl, 25 mM sodium phosphate pH 6.8 with 5% isopropanol.

In vitro cytotoxicity assays. Cell lines were plated in 96-well plates (Costar 3610) at a density of 5×10⁴ cells/well in 100 μL of growth media. The next day cells were treated with L of test articles serially-diluted in media. After incubation at 37° C. with 5% CO₂ for 5 days, viability was measured using the Promega CellTiter Glo® reagent according to the manufacturer's recommendations. GI50 curves were calculated in GraphPad Prism normalized to the payload concentration.

### Xenograft Studies

Methods: Female NCG mice (10/group) were implanted with estrogen pellets (0.36 mg/90 days, 17□-estradiol), and then were inoculated subcutaneously with 20 million T47D cells in PBS with Matrigel (1:1 vol/vol). On the day before treatment began (Day 0), all animals received an intravenous dose of 10 mg/kg human IgG.

Treatment began when the tumors reached an average of 223 mm³ (Day 1). For treatment, animals were dosed intravenously with vehicle alone, B06- or H02-derived ADCs conjugated with MMAE, or with unconjugated B06 antibody. ADCs were dosed at 10 mg/kg, and the B06 ADC was also dosed at 3 mg/kg. Treatment dosing occurred weekly for 4 total doses. The animals were monitored twice weekly for body weight and tumor size. Animals were euthanized when tumors reached 2000 mm³.

Results: Tumors in the vehicle control group grew slowly but consistently throughout the study. Animals dosed with 10 mg/kg of either the B06 or the H02 ADC saw continued tumor volumes steadily decrease from baseline throughout the duration of the study. Treatment with 3 mg/kg B06 ADC slightly reduced tumor volume. A dose-response effect was observed between the 3 and 10 mg/kg B06 ADC groups.

### Immunohistochemistry

Human tumor microarrays (frozen and FFPE) and tissue section panels of normal human and cynomolgus monkey (frozen) were purchased from Biochain. Tissue sections were reacted with the B06 ADC or with an isotype control ADC. Primary antibodies were detected using the Human-on-Human HRP polymer system from Biocare Medical. Binding was visualized by DAB, which deposits a brown stain on the tissues.

Table 6. Summary of B06 ADC reactivity against primary human tumor samples in ovarian, lung, and breast tumor microarrays.

| | Tissue | Pathological | % of Tumors binding |
|---|---|---|---|
| Tumor type | state | diagnosis | B06 ADC |
| Ovary | Frozen | Adenocarcinoma | 61% |
| Ovary | Frozen | Other | 71% |
| Lung | Frozen | Adenocarcinoma | 80% |
| Lung | Frozen | Squamous cell | 55% |
| | | carcinoma | |
| Breast | Frozen | Ductal carcinoma | 73% |
| Ovary | FFPE | Adenocarcinoma | 85% |

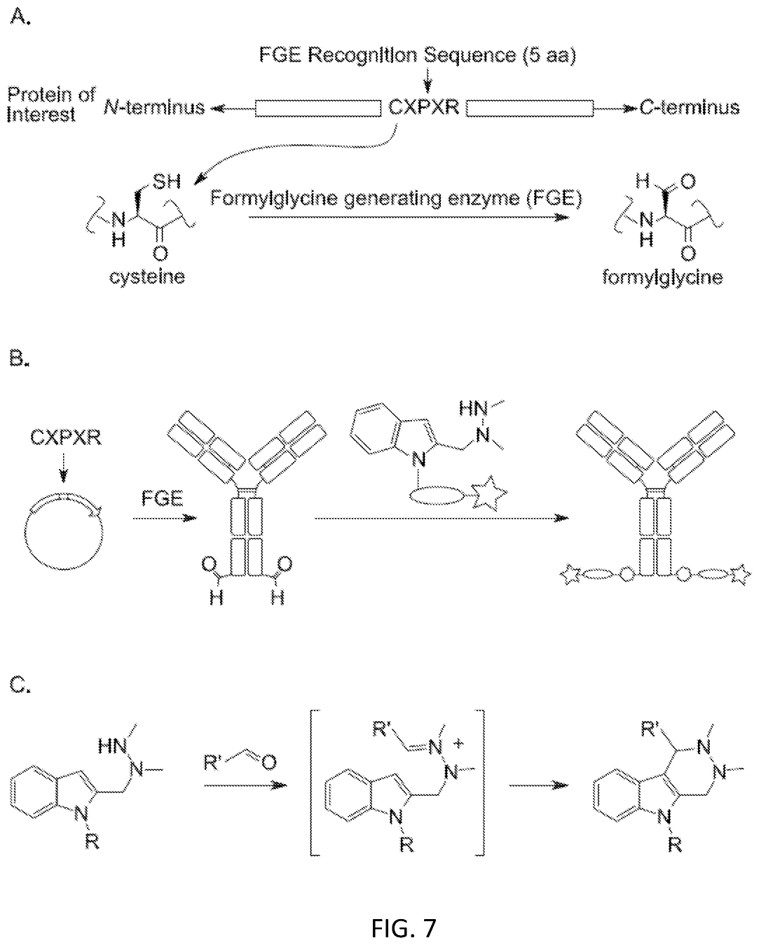

FIG. 7. Aldehyde-tagged antibody production and ADC generation using HIPS-mediated conjugation. (A) The formylglycine recognition sequence (CXPXR) is genetically encoded into the antibody. (B) Co-translationally formylglycine-generating enzyme converts the cysteine within the recognition sequence to a formylglycine residue containing an aldehyde functional group that can be specifically conjugated with (C) the Hydrazino-iso-Pictet-Spengler (HIPS) conjugation element.

FIG. 8. CT-tagged B06 antibody conjugated to RED-601 yields a DAR (drug antibody ratio) of 1.85 as determined by HIC.

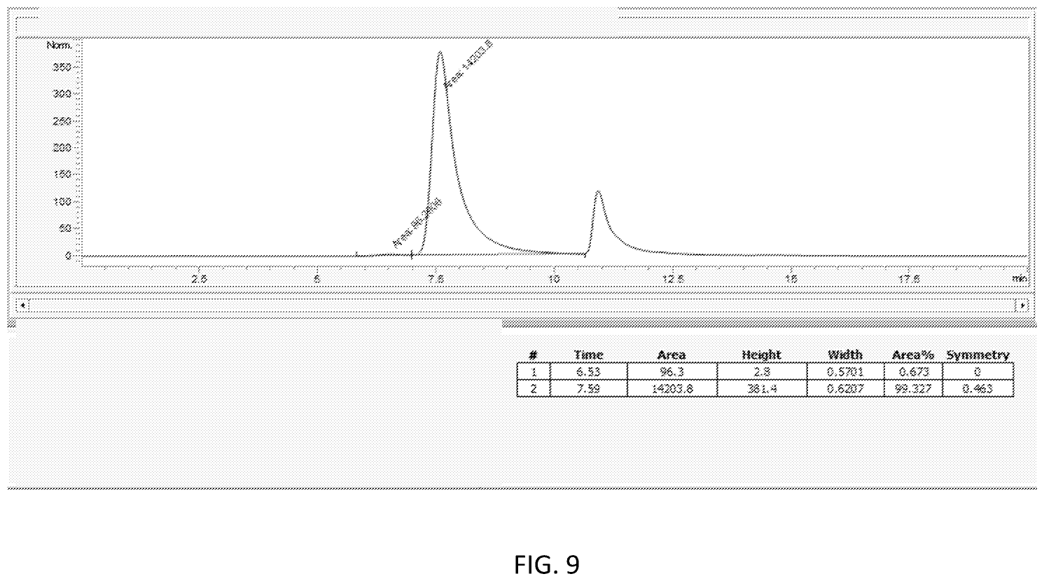

FIG. 9. CT-tagged B06 antibody conjugated to RED-601 is 99.3% monomeric as determined by SEC.

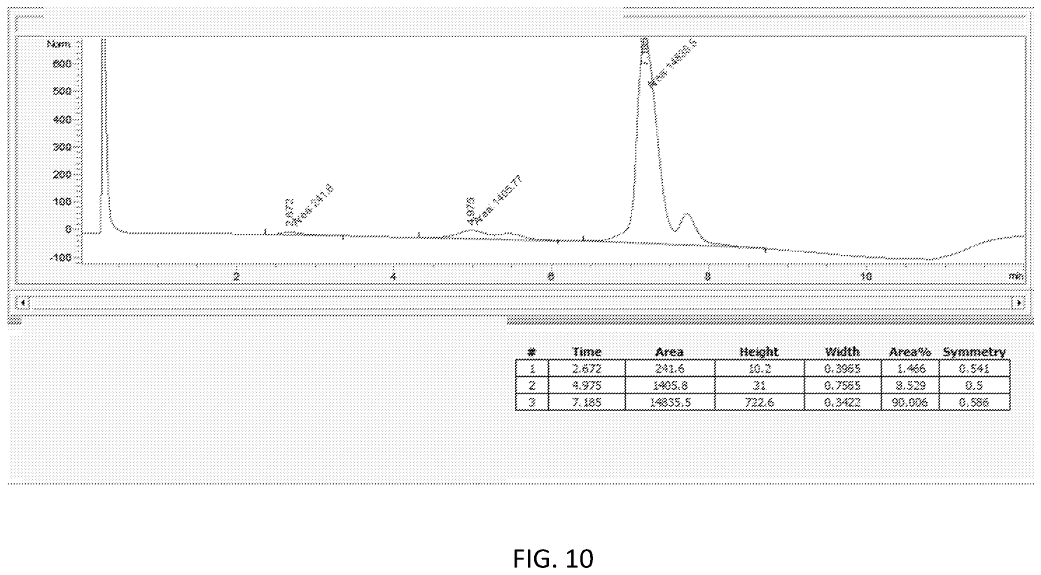

FIG. 10. CT-tagged G12 antibody conjugated to RED-601 yields a DAR of 1.89 as determined by HIC.

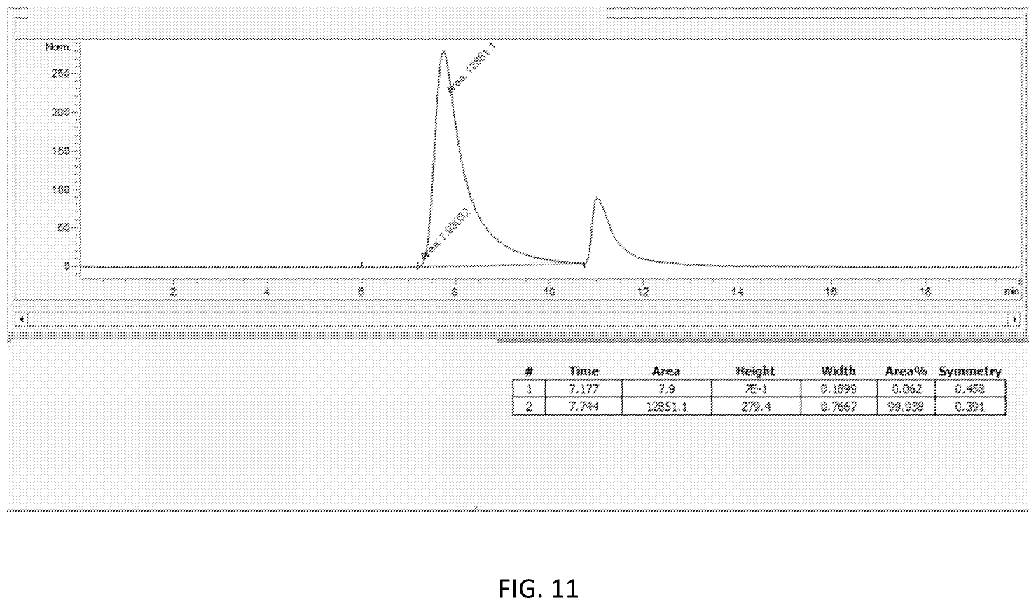

FIG. 11. CT-tagged G12 antibody conjugated to RED-601 is 99.9% monomeric as determined by SEC.

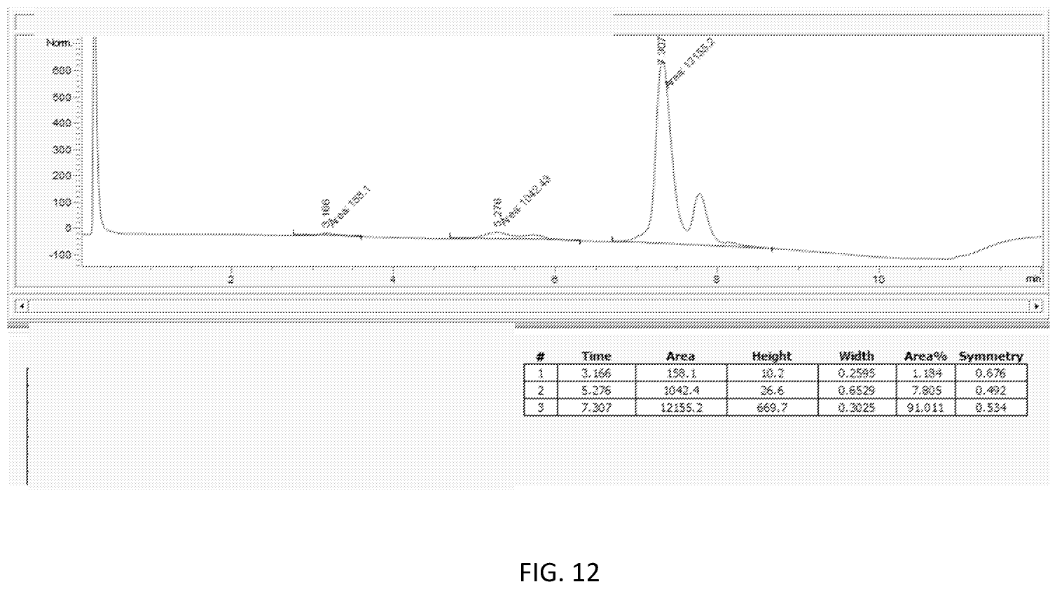

FIG. 12. CT-tagged H02 antibody conjugated to RED-601 yields a DAR of 1.90 as determined by HIC.

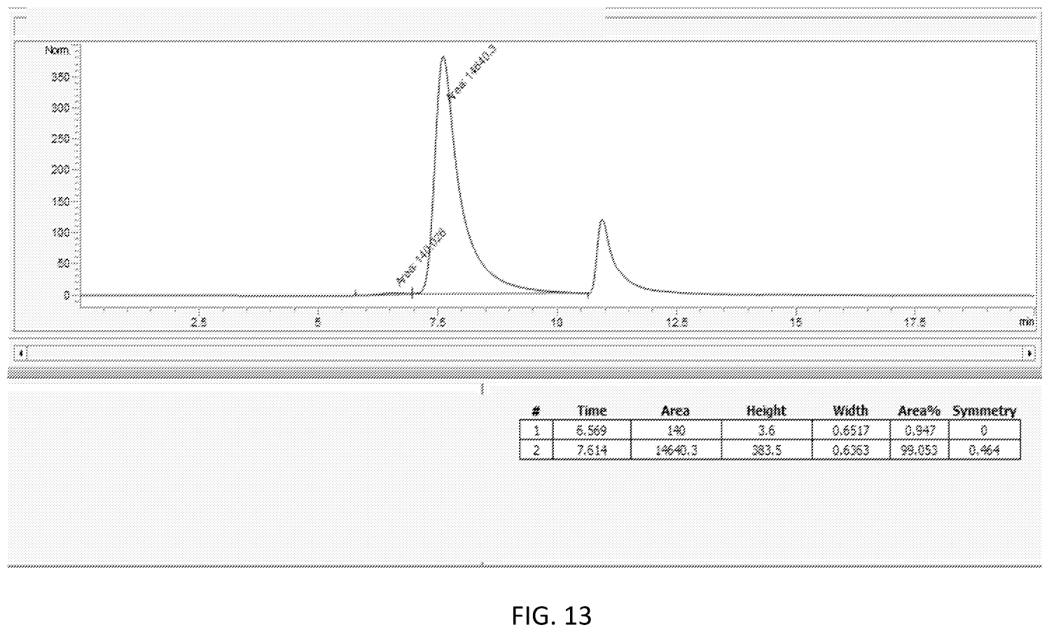

FIG. 13. CT-tagged H02 antibody conjugated to RED-601 is 99.1% monomeric as determined by SEC.

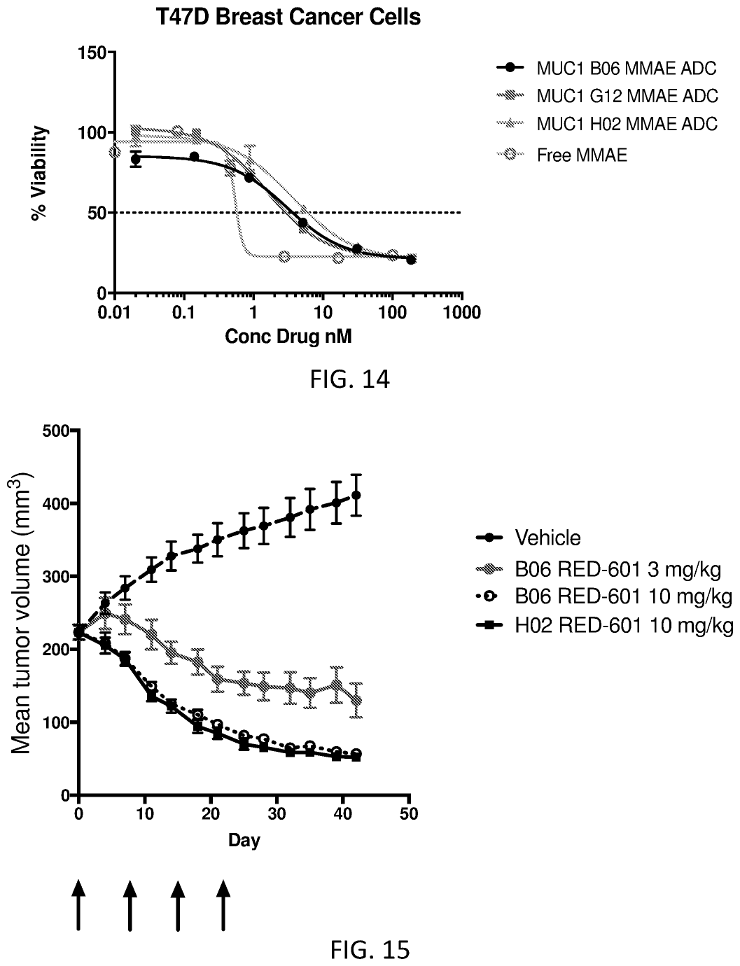

FIG. 14. In vitro potency against T47D cells of MMAE-conjugated anti-MUC1 ADCs made from the B06, G12, or H02 variant antibodies. Free MMAE is included as a benchmark for potency of the payload.

FIG. 15. In vivo efficacy against a T47D xenograft of MMAE-conjugated anti-MUC1ADCs-B06 RED-601 and H02 RED-601-carrying an MMAE payload. n=10 mice/group; dosing is indicated by arrows.

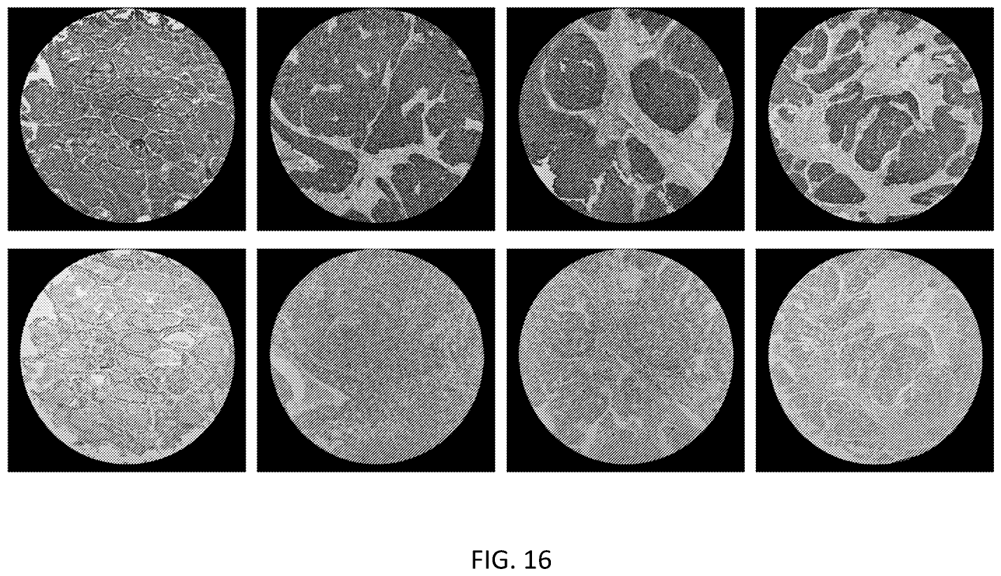

FIG. 16. Representative data showing B06 ADC binding to primary human ovarian adenocarcinomas. Four ovarian adenocarcinoma specimens were reacted with B06 ADC (top row) or isotype control ADC (bottom row). Brown color indicates ADC binding.

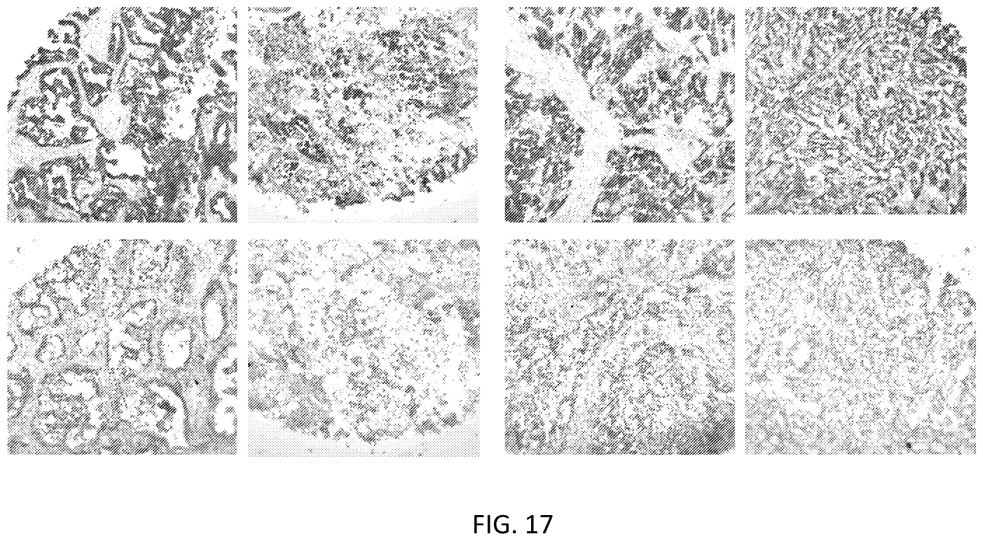

FIG. 17. Representative data showing B06 ADC binding to primary human lung tumors. Four lung cancer specimens were reacted with B06 ADC (top row) or isotype control ADC (bottom row). Adenocarcinoma two left columns; squamous cell carcinoma two right columns. Brown color indicates ADC binding.

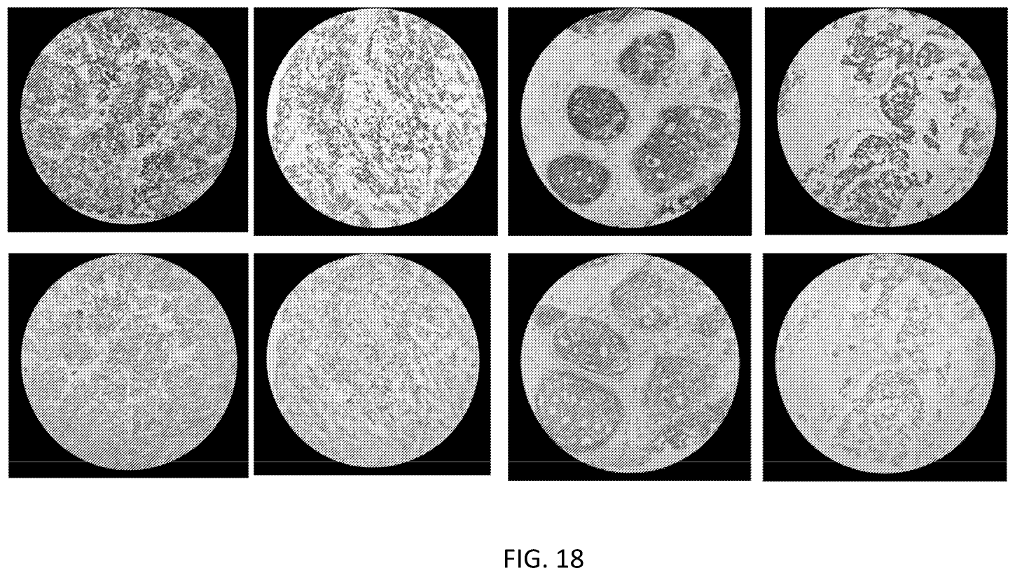

FIG. 18. Representative data showing B06 ADC binding to primary human breast tumors. Four breast ductal carcinoma specimens were reacted with B06 ADC (top row) or isotype control ADC (bottom row). Brown color indicates ADC binding.

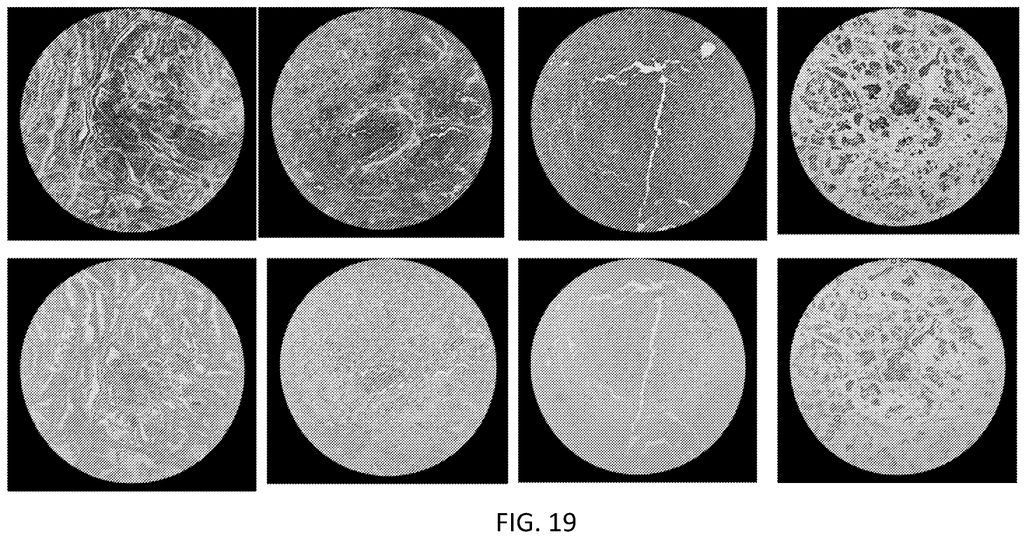

FIG. 19. B06 ADC binds strongly to patient-derived xenograft (PDX) tumor models. Four Charles River Laboratories PDX specimens were reacted with B06 ADC (top row) or isotype control ADC (bottom row). Brown color indicates ADC binding. Tumor origin from left to right: gastric, breast, lung, gastric.

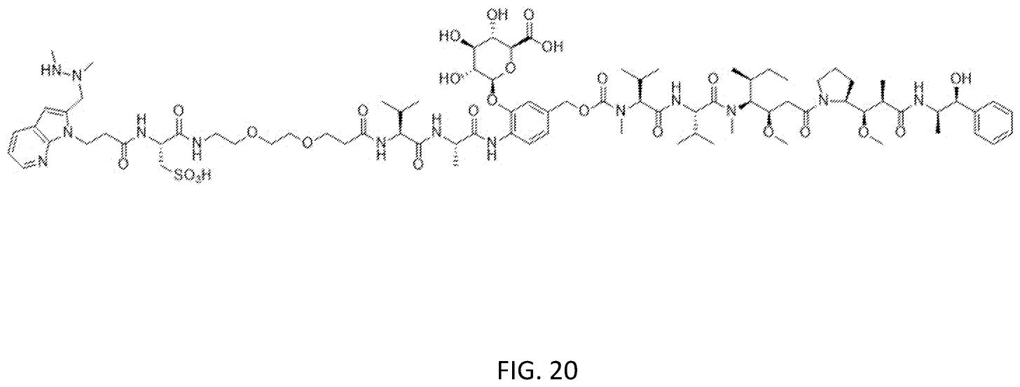

FIG. 20. Structure for RED-601, a linker-payload conjugated to the anti-MUC1 antibodies (see Compound 8 in Example 2).

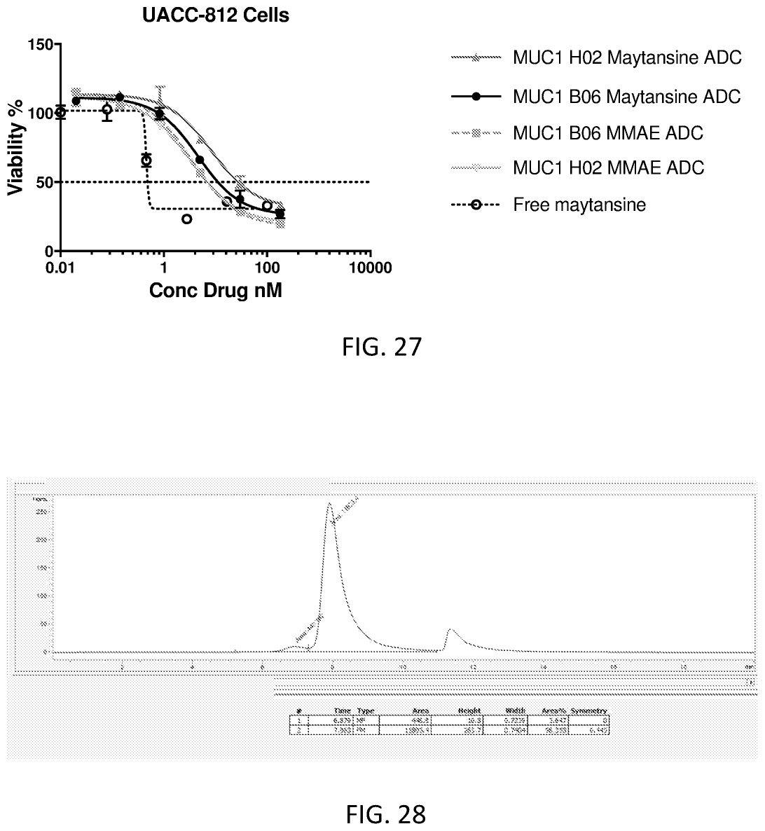

FIG. 27. In vitro potency against UACC-812 cells of maytansine or monomethyl auristatin E (MMAE)-conjugated anti-MUC1 ADCs made from the B06 or H02 variant antibodies. Free maytansine was included as a benchmark for potency of the payload.

FIG. 28. Single-tagged B06 antibody conjugated at 91N to a branched MMAE linker-payload was 96.4% monomeric as determined by SEC.

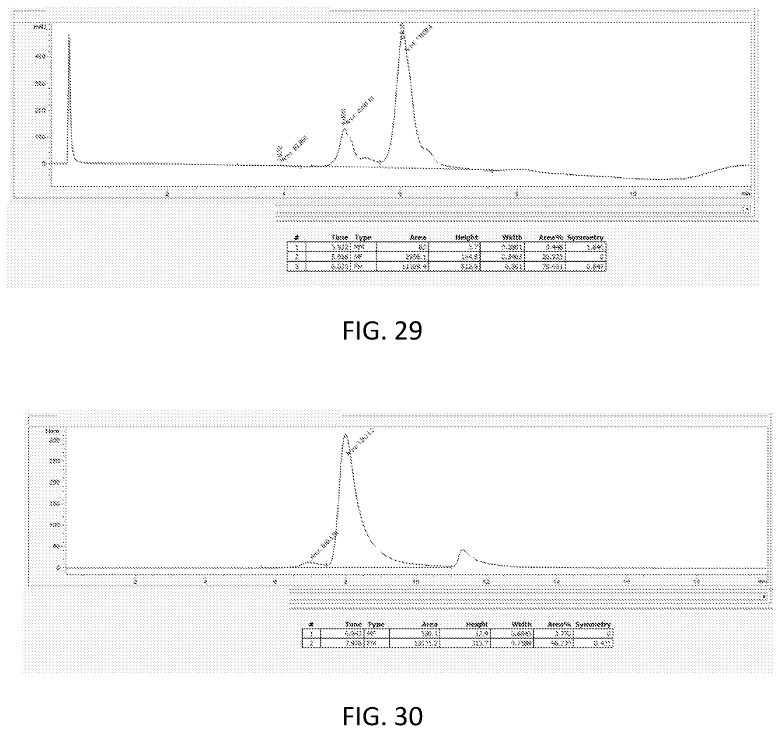

FIG. 29. Single-tagged B06 antibody conjugated at 91N to Compound 8 yields a DAR of 1.78 as determined by HIC.

FIG. 30. Single-tagged B06 antibody conjugated at 91N to Compound 8 is 96.2% monomeric as determined by SEC.

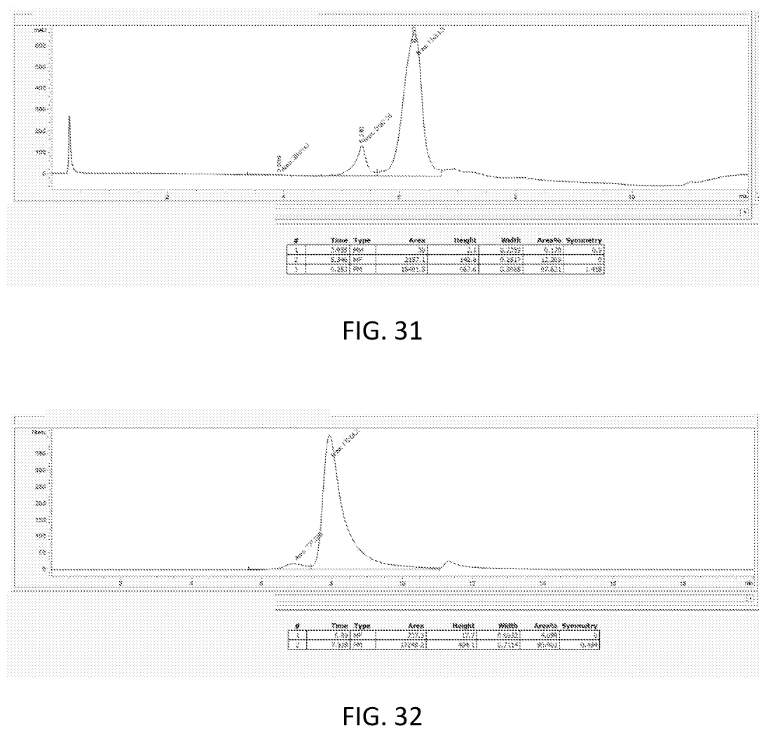

FIG. 31. Single-tagged B06 antibody conjugated at 91N to Compound 21 yields a DAR of 3.74 as determined by PLRP.

FIG. 32. Single-tagged B06 antibody conjugated at 91N to Compound 21 is 95.9% monomeric as determined by SEC.

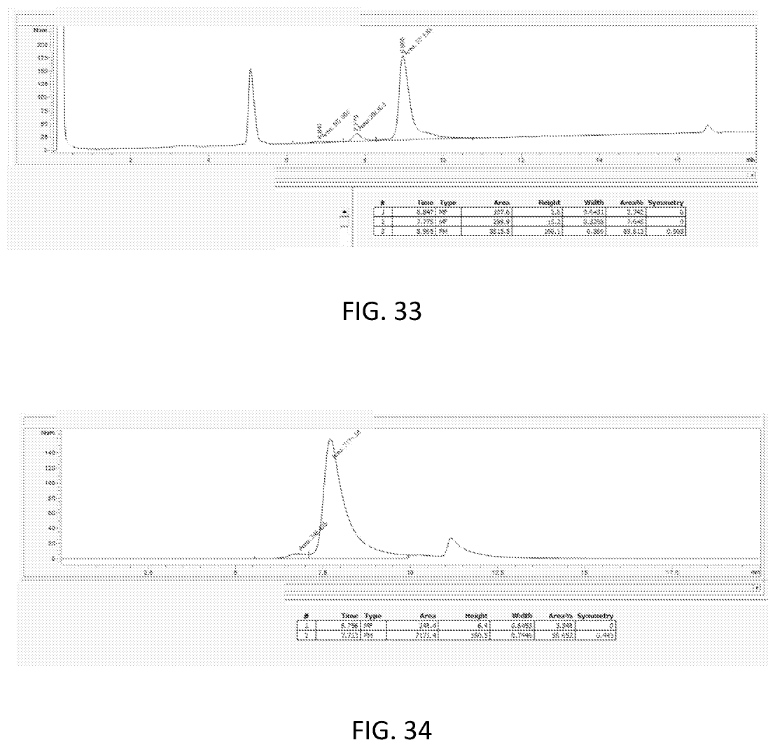

FIG. 33. Double-tagged B06 antibody conjugated to Compound 21 yields a DAR of 7.47 as determined by PLRP.

FIG. 34. Double-tagged B06 antibody conjugated to Compound 21 is 96.7% monomeric as determined by SEC.

TABLE 7
Sequence of CT-tagged Heavy Chain (HC) of the B06, G12, or H02
antibodies. CT-tag is underlined. VH region is italicized. FRs1-4 are
underlined and CDRs1-3 as per Chothia definition are in bold.

| | |
|---|---|
| CT-tagged | EVQLVQSGAEVKKPGATVKISCKVSGYTFTDHTMHWIKQRPGKGLEWMGYFYP |
| HC | RDDSTNYNEKFKGRVTLTADKSTDTAYMELSSLRSEDTAVYYCARGLRYALDYW |
| | GQGTLVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVD |
| | KKVEPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQDWL |
| | NGKEYKCKVSNKALPAPIEKTISKAKGQPREPQVYTLPPSREEMTKNQVSLT |
| | CLVKGFYPSDIAVEWESNGQPENNYKTTPPVLDSDGSFFLYSKLTVDKSRW |
| | QQGNVFSCSVMHEALHNHYTQKSLSLSPGSLCTPSRGS (SEQ ID NO: 57) |

### Example 4: Cell Staining

### Antibody Production

Sequence unique clones were selected and used for production of new Fab culture supernatants. For that purpose, the respective clones were first cultivated in 2YT medium, supplemented with Ampicillin, Tetracyclin and Glucose, at 37° C., 300 rpm for 16 h. After an overnight culture was established, 15 μl was used for the inoculation of production medium (2YT, supplemented with Ampicillin, Tetracycline and IPTG). Production of antibody fragments was performed at 30° C., 300 rpm for 16 h.

The antibody containing culture supernatants were mixed in a 1:1 ratio with FACS buffer, separated from cells by centrifugation (4000 g, 10 min) and used for cell staining.

### Cell Staining

Ag+ T-47D cells and Ag− HEK cells were cultivated according to the supplier. After harvesting of the cells, the HEK cell population was labelled with CFSE dye and mixed in a 1:1 ratio with the T47-D cells. The cell mix was transferred into a 96-well plate (100,000 cell mix/well). After centrifugation at 300 g for 5 min, cells were stained with the antibody culture supernatants for 15 min at 4° C. After centrifugation and washing of the cells, a secondary Alexa Fluor 647 conjugated antibody was added (Invitrogen, A-21445) for detection of the Fab fragments and incubated for 15 min at 4° C. After another washing step, the cells were resuspended in FACS buffer, supplemented with PI, to enable live/dead staining. Cell measurement was performed at the iQue Screener.

### Data Analysis

Based on the CFSE signal, gating was done one the Ag+ and Ag− population. From each population, the MFI values of the antibody binding signal (Alexa Fluor 647 signal) were calculated. MFI values were plotted in a graph and antibody binding signals to the Ag+ and Ag− population were compared to each other.

Binding of anti-MUC1 affinity-matured variants and parental antibody to antigen-positive (Ag+) T47D cells or antigen-negative (Ag−) HEK cells were assessed by flow cytometry. Results are shown in FIG. 26.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A conjugate of formula (I): 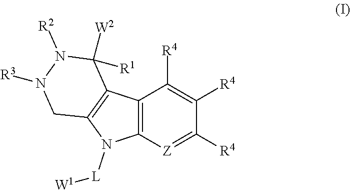
wherein
Z is CR⁴ or N;
R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L is a linker;
W¹ is a drug; and
W² is an anti-MUC1 antibody.

2. The conjugate of claim 1, wherein L comprises:
-(T¹-V¹)ₐ-(T²-V²)_{b}-(T³-V³)_{c}-(T⁴-V⁴)_{d}-(T⁵V⁵)ₑ-(T⁶-V⁶)_{f}-,
wherein
a, b, c, d, e and f are each independently 0 or 1, wherein the sum of a, b, c, d, e and f is 1 to 6;
T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each m is an integer from 1 to 12;
V¹, V², V³, V⁴, V⁵ and V⁶ are each independently selected from the group consisting of a covalent bond, —CO—, —NR¹⁵, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl;
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

3. The conjugate of claim 2, wherein:
T¹ is selected from a (C₁-C₁₂)alkyl and a substituted (C₁-C₁₂)alkyl;
T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, 4-amino-piperidine (4AP), MABO, MABC, PABO, PABC, PAB, PABA, PAP, PHP, an acetal group, a hydrazine, and an ester; and
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, and —P(O)OH—;
wherein:
(PEG)ₙ is 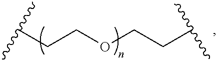
where n is an integer from 1 to 30;
EDA is an ethylene diamine moiety having the following structure: 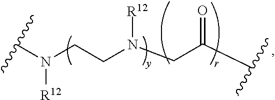
where y is an integer from 1 to 6 and r is 0 or 1;
4-amino-piperidine (4AP) is 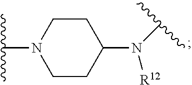
and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring.

4. The conjugate of claim 2, wherein MABO, MABC, PABO, PABC, PAB, PABA, PAP and PHP are each optionally substituted with a glycoside.

5. The conjugate of any of claim 4, wherein the glycoside is selected from a glucuronide, a galactoside, a glucoside, a mannoside, a fucoside, O-GlcNAc, and O-GalNAc.

6. The conjugate of claim 2,
wherein:
T¹ is (C₁-C₁₂)alkyl and V¹ is —CO—;
T² is an amino acid analog and V² is —NH—;
T³ is (PEG)ₙ and V³ is —CO—;
T⁴ is AA and V⁴ is absent;
T⁵ is PABC and V⁵ is absent; and
f is0.

7. The conjugate of claim 1, wherein the drug is monomethyl auristatin E (MMAE).

8. The conjugate of claim 1, wherein the conjugate has the structure:

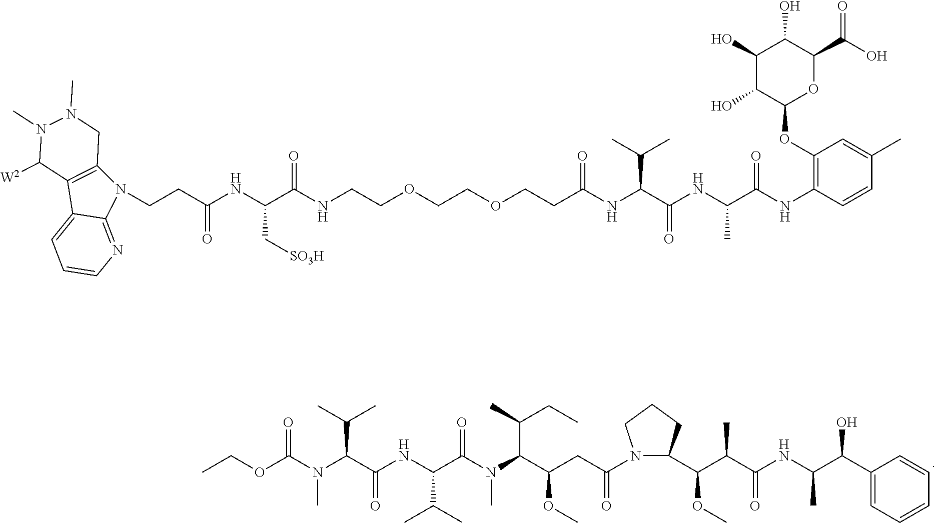

9. A conjugate of formula (II): 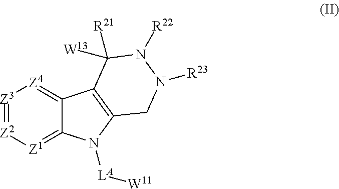
wherein:
Z¹, Z², Z³ and Z⁴ are each independently selected from CR²⁴, N and C-L^{B}-W¹², wherein at least one Z¹, Z², Z³ and Z⁴ is C-L^{B}-W¹²;
R²¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R²² and R²³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R²² and R²³ are optionally cyclically linked to form a 5 or 6-membered heterocyclyl;
each R²⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L^{A} is a first linker;
L^{B} is a second linker;
W¹¹ is a first drug;
W¹² is a second drug; and
W¹³ is an anti-MUC1 antibody.

10.-21. (canceled)

22. The conjugate of claim 1, wherein the anti-MUC1 antibody is an IgG1 antibody.

23. The conjugate of claim 22, wherein the anti-MUC1 antibody is an IgG1 kappa antibody.

24. The conjugate of claim 1, wherein the anti-MUC1 antibody comprises a sequence of the formula (III):
X¹(fGly′)X²Z²X³Z³(III),
wherein
X¹ is present or absent and, when present, can be any amino acid, with the proviso that when the sequence is at the N-terminus of the conjugate, X¹ is present;
fGly′ is the amino acid residue coupled to the drug through the linker;
X² and X³ are each independently any amino acid;
Z² is either a proline or alanine residue; and
Z³ is a basic amino acid or an aliphatic amino acid.

25. The conjugate of claim 24, wherein the sequence is L(fGly′)TPSR (SEQ ID NO:24).

26. The conjugate of claim 24, wherein
Z³ is selected from R, K, H, A, G, L, V, I, and P;
X¹ is selected from L, M, S, and V; and
X² and X³ are each independently selected from S, T, A, V, G, and C.

27. The conjugate of claim 1, wherein the sequence is positioned at a C-terminus of a heavy chain constant region of the anti-MUC2 antibody.

28. The conjugate of claim 27, wherein the heavy chain constant region comprises a sequence of the formula (III):
X¹(fGly′)X²Z²X³Z³(III),
wherein
X¹ is present or absent and, when present, can be any amino acid, with the proviso that when the sequence is at the N-terminus of the conjugate, X¹ is present;
fGly′ is the amino acid residue coupled to the drug through the linker;
X² and X³ are each independently any amino acid;
Z² is either a proline or alanine residue;
Z³ is a basic amino acid or an aliphatic amino acid, and
wherein the sequence is C-terminal to the amino acid sequence SLSLSPG (SEQ ID NO:187).

29. The conjugate of claim 28, wherein the heavy chain constant region comprises the sequence SPGSL(fGly′)TPSRGS (SEQ ID NO:188).

30. The conjugate of claim 28, wherein
Z³ is selected from R, K, H, A, G, L, V, I, and P;
X¹ is selected from L, M, S, and V; and
X² and X³ are each independently selected from S, T, A, V, G, and C.

31. The conjugate of claim 27, wherein the heavy chain constant region of the anti-MUC1 antibody comprises an amino acid sequence at least 85%, at least 90%, at least 95%, at least 96%, at least 97%, at least 98%, or at least 99% identical to the amino acid sequence set forth in SEQ ID NO:57 and comprises the fGly′ residue at amino acid position 332 instead of C.

32. The conjugate of claim 1, wherein the heavy chain constant region of the anti-MUC1 antibody comprises an amino acid sequence at least 85%, at least 90%, at least 95%, at least 96%, at least 97%, at least 98%, or at least 99% identical to the amino acid sequence set forth in any one of SEQ ID NOs:58, 62, 66, and 70 and comprises the fGly′ residue at amino acid position 59 instead of C.

33. The conjugate of claim 1, wherein the heavy chain constant region of the anti-MUC1 antibody comprises an amino acid sequence at least 85%, at least 90%, at least 95%, at least 96%, at least 97%, at least 98%, or at least 99% identical to the amino acid sequence set forth in any one of SEQ ID NOs:59, 63, 67, and 71 and comprises the fGly′ residue at amino acid position 62 instead of C.

34. The conjugate of claim 1, wherein the heavy chain constant region of the anti-MUC1 antibody comprises an amino acid sequence at least 85%, at least 90%, at least 95%, at least 96%, at least 97%, at least 98%, or at least 99% identical to the amino acid sequence set forth in any one of SEQ ID NOs:60, 64, 68, and 72 and comprises the fGly′ residue at amino acid position 92 instead of C.

35. The conjugate of claim 1, wherein the heavy chain constant region of the anti-MUC1 antibody comprises an amino acid sequence at least 85%, at least 90%, at least 95%, at least 96%, at least 97%, at least 98%, or at least 99% identical to the amino acid sequence set forth in any one of SEQ ID NOs:61, 65, 69, and 73 and comprises the fGly′ residue at amino acid position 117 instead of C.

36. The conjugate of claim 1, wherein the fGly′ residue is positioned in a light chain constant region of the anti-MUC1 antibody.

37. The conjugate of claim 36, wherein the light chain constant region comprises a sequence of the formula (III):
X¹(fGly′)X²Z²X³Z³(III),
wherein
X¹ is present or absent and, when present, can be any amino acid, with the proviso that when the sequence is at the N-terminus of the conjugate, X¹ is present;
fGly′ is the amino acid residue coupled to the drug through the linker;
X² and X³ are each independently any amino acid;
Z² is either a proline or alanine residue;
Z³ is a basic amino acid or an aliphatic amino acid, and
wherein the sequence is C-terminal to the amino acid sequence KVDNAL (SEQ ID NO:101), and/or is N-terminal to the sequence QSGNSQ (SEQ ID NO:102).

38. The conjugate of claim 37, wherein the light chain constant region comprises the sequence KVDNAL(fGly′)TPSRQSGNSQ (SEQ ID NO:103).

39. The conjugate of claim 36, wherein
Z³ is selected from R, K, H, A, G, L, V, I, and P;
X¹ is selected from L, M, S, and V; and
X² and X³ are each independently selected from S, T, A, V, G, and C.

40. The conjugate of claim 1, wherein the fGly′ residue is positioned in a heavy chain CH₁ region of the anti-MUC1 antibody.

41. The conjugate of claim 40, wherein the light chain constant region comprises a sequence of the formula (III):
X¹(fGly′)X²Z²X³Z³(III),
wherein
X¹ is present or absent and, when present, can be any amino acid, with the proviso that when the sequence is at the N-terminus of the conjugate, X¹ is present;
fGly′ is the amino acid residue coupled to the drug through the linker;
X² and X³ are each independently any amino acid;
Z² is either a proline or alanine residue;
Z³ is a basic amino acid or an aliphatic amino acid, and
wherein the sequence is C-terminal to the amino acid sequence SWNSGA (SEQ ID NO:104) and/or is N-terminal to the amino acid sequence GVHTFP (SEQ ID NO:105).

42. The conjugate of claim 41, wherein the heavy chain CH₁ region comprises the sequence SWNSGAL(fGly′)TPSRGVHTFP (SEQ ID NO:106).

43. The conjugate of claim 42, wherein
Z³ is selected from R, K, H, A, G, L, V, I, and P;
X¹ is selected from L, M, S, and V; and
X² and X³ are each independently selected from S, T, A, V, G, and C.

44. The conjugate of claim 1, wherein the fGly′ residue is positioned in a heavy chain CH₂ region of the anti-MUC1 antibody.

45. The conjugate of claim 1, wherein the fGly′ residue is positioned in a heavy chain CH₃ region of the anti-MUC1 antibody.

46. The conjugate of claim 1, wherein the anti-MUC1 antibody competes for binding to MUC1 with an anti-MUC1 antibody comprising:
a variable heavy chain (VH) chain comprising heavy chain CDRs1-3 (HCDRs1-3) of a VH chain having the sequence:
EVQLVQSGAEVKKPGATVKISCKVSGYTFTDHTMHWIKQRPGKGLEWMGYFYPRDDSTN YNEKFKGRVTLTADKSTDTAYMELSSLRSEDTAVYYCARGLRYALDYWGQGTLVTVSS (SEQ ID NO:1); and
a variable light chain (VL) chain comprising light chain CDRs1-3 (LCDRs1-3) of a VL chain having the sequence:
(SEQ ID NO: 2)
EIVLTQSPATLSLSPGERATLSCRASSSVSSSYLYWYQQKPGQAPRLWI
YGTSNLASGVPARFSGSGSGTDYTLTISSLEPEDAAVYYCHQYAWSPPT
FGQGTKLEIK;
(SEQ ID NO: 3)
EIVLTQSPATLSLSPGERATLSCRASSSVGSSNLYWYQQKPGQAPRLWI
YRSTKLASGVPARFSGSGSGTDYTLTISSLEPEDAAVYYCHQYRWSPPT
FGQGTKLEIK;
or
(SEQ ID NO: 4)
EIVLTQSPATLSLSPGERATLSCRASSSVSSSYLYWYQQKPGQAPRLWI
IGTSNLASGVPARFSGSGSGTDYTLTISSLEPEDAAVYYCHQYSWSPPT
FGQGTKLEIK.

47. The conjugate of claim 1, wherein the anti-MUC1 antibody comprises:
a variable heavy chain (VH) chain comprising heavy chain CDRs1-3 (HCDRs1-3) of a VH chain having the sequence:
EVQLVQSGAEVKKPGATVKISCKVSGYTFTDHTMHWIKQRPGKGLEWMGYFYPRDDSTN YNEKFKGRVTLTADKSTDTAYMELSSLRSEDTAVYYCARGLRYALDYWGQGTLVTVSS (SEQ ID NO:1); and
a variable light chain (VL) chain comprising light chain CDRs1-3 (LCDRs1-3) of a VL chain having the sequence:
(SEQ ID NO: 2)
EIVLTQSPATLSLSPGERATLSCRASSSVSSSYLYWYQQKPGQAPRLWI
YGTSNLASGVPARFSGSGSGTDYTLTISSLEPEDAAVYYCHQYAWSPPT
FGQGTKLEIK;
(SEQ ID NO: 3)
EIVLTQSPATLSLSPGERATLSCRASSSVGSSNLYWYQQKPGQAPRLWI
YRSTKLASGVPARFSGSGSGTDYTLTISSLEPEDAAVYYCHQYRWSPPT
FGQGTKLEIK;
or
(SEQ ID NO: 4)
EIVLTQSPATLSLSPGERATLSCRASSSVSSSYLYWYQQKPGQAPRLWI
IGTSNLASGVPARFSGSGSGTDYTLTISSLEPEDAAVYYCHQYSWSPPT
FGQGTKLEIK.

48. The conjugate of claim 1, wherein the VH polypeptide comprises an amino acid sequence having at least 80% identity to the amino acid sequence set forth in SEQ ID NO:1.

49. The conjugate of claim 46, wherein the VL polypeptide comprises an amino acid sequence having at least 80% identity to the amino acid sequence set forth in SEQ ID NO:2, 3, or 4.

50. The conjugate of claim 46, wherein:
the HCDR1 comprises the amino acid sequence DHTMH (SEQ ID NO:17);
the HCDR2 comprises the amino acid sequence YFYPRDDSTNYNEKFKG (SEQ ID NO:18);
the HCDR3 comprises the amino acid sequence GLRYALDY (SEQ ID NO:9);
the LCDR1 comprises the amino acid sequence RASSSVSSSYLY (SEQ ID NO:10);
the LCDR2 comprises the amino acid sequence GTSNLAS (SEQ ID NO:11); and
the LCDR3 comprises the amino acid sequence HQYAWSPPT (SEQ ID NO:12), as per Kabat definition.

51. The conjugate of claim 46, wherein:
the HCDR1 comprises the amino acid sequence DHTMH (SEQ ID NO:17);
the HCDR2 comprises the amino acid sequence YFYPRDDSTNYNEKFKG (SEQ ID NO:18);
the HCDR3 comprises the amino acid sequence GLRYALDY (SEQ ID NO:9);
the LCDR1 comprises the amino acid sequence RASSSVGSSNLY (SEQ ID NO:13);
the LCDR2 comprises the amino acid sequence RSTKLAS (SEQ ID NO:14); and
the LCDR3 comprises the amino acid sequence HQYRWSPPT (SEQ ID NO:15), as per Kabat definition.

52. The conjugate of claim 46, wherein:
the HCDR1 comprises the amino acid sequence DHTMH (SEQ ID NO:17);
the HCDR2 comprises the amino acid sequence YFYPRDDSTNYNEKFKG (SEQ ID NO:18);
the HCDR3 comprises the amino acid sequence GLRYALDY (SEQ ID NO:9);
the LCDR1 comprises the amino acid sequence RASSSVSSSYLY (SEQ ID NO:10);
the LCDR2 comprises the amino acid sequence GTSNLAS (SEQ ID NO:11); and
the LCDR3 comprises the amino acid sequence HQYSWSPPT (SEQ ID NO:16), as per Kabat definition.

53. A pharmaceutical composition comprising:
a conjugate of claim 1; and
a pharmaceutically-acceptable excipient.

54. A method comprising:
administering to a subject an amount of the conjugate of claim 1.

55. A method of treating cancer in a subject, the method comprising:
administering to the subject a therapeutically effective amount of a pharmaceutical composition of claim 53, wherein the administering is effective to treat cancer in the subject.

56. The method according to claim 55, wherein the cancer is a breast cancer, an ovarian, a lung cancer, or a gastric cancer.

57. The method according to claim 56, wherein the cancer is characterized by cancer cells expressing glycosylated MUC1.

58. The method according to claim 56, wherein the conjugate binds to the glycosylated MUC1.

59. The method according to claim 55, wherein the breast cancer is triple-negative for estrogen, progesterone, and HER2.

60. The method according to claim 59, wherein the triple-negative breast cancer is metastatic triple negative breast cancer.

61. The method according to claim 59, wherein the triple-negative breast cancer is a relapsed or refractory triple negative breast cancer.

62. A method of delivering a drug to a target site in a subject, the method comprising:
administering to the subject a pharmaceutical composition of claim 53, wherein the administering is effective to release a therapeutically effective amount of the drug from the conjugate at the target site in the subject.


SEQUENCE LISTING (182 sequences)

SEQ ID NO: 1 - protein, 22 aa, Saccharomyces cerevisiae
MFSLKALLPLALLLVSANQVAA

SEQ ID NO: 2 - protein, 54 aa, Saccharomyces cerevisiae
KVHKAKIYKHELSDEMKEVTFEQHLAHLGQKYLTQFEKANPEVVFSREHPFFTE

SEQ ID NO: 3 - protein, 18 aa, Homo sapiens
MVRMVPVLLSLLLLLGPA

SEQ ID NO: 4 - protein, 19 aa, Kluyveromyces marxianus
MLFSNTLLIAAASALLAEA

SEQ ID NO: 5 - protein, 19 aa, Saccharomycopsis fibuligera
MKFGVLFSVFAAIVSALPA

SEQ ID NO: 6 - protein, 6 aa, Saccharomyces cerevisiae
GRRAKL

SEQ ID NO: 7-8 - skipped (2 entries)

SEQ ID NO: 9 - protein, 9 aa, Saccharomyces cerevisiae
RRMLSSKQL

SEQ ID NO: 10 - protein, 709 aa, synthetic construct
  REGION 1..709: note = Arthrobacter
MTEVVDRASSPASPGSTTAAADGAKVAVEPRVDVAALGEQLLGRWADIRLHARDLAGREVVQKVEGLTHTEHRSRVFGQLKYLVDNNAVHRAFPSRLGGSDDHGGNIAGFEELVTADPSLQIKAGVQWGLFGSAVMHLGTREHHDKWLPGIMSLEIPGCFAMTETGHGSDVASIATTATYDEETQEFVIDTPFRAAWKDYIGNAANDGLAAVVFAQLITRKVNHGVHAFYVDLRDPATGDFLPGIGGEDDGIKGGLNGIDNGRLHFTNVRIPRTNLLNRYGDVAVDGTYSSTIESPGRRFFTMLGTLVQGRVSLDGAAVAASKVALQSAIHYAAERRQFNATSPTEEEVLLDYQRHQRRLFTRLATTYAASFAHEQLLQKFDDVFSGAHDTDADRQDLETLAAALKPLSTWHALDTLQECREACGGAGFLIENRFASLRADLDVYVTFEGDNTVLLQLVAKRLLADYAKEFRGANFGVLARYVVDQAAGVALHRTGLRQVAQFVADSGSVQKSALALRDEEGQRTLLTDRVQSMVAEVGAALKGAGKLPQHQAAALFNQHQNELIEAAQAHAELLQWEAFTEALAKVDDAGTKEVLTRLRDLFGLSLIEKHLSWYLMNGRLSMQRGRTVGTYINRLLVKIRPHALDLVDAFGYGAEHLRAAIATGAEATRQDEARTYFRQQRASGSAPADEKTLLAIKAGKSRGRRAKL

SEQ ID NO: 11 - protein, 700 aa, Yarrowia lipolytica
MNPNNTGTIEINGKEYNTFTEPPVAMAQERAKTSFPVREMTYFLDGGEKNTLKNEQIMEEIERDPLFNNDNYYDLNKEQIRELTMERVAKLSLFVRDQPEDDIKKRFALIGIADMGTYTRLGVHYGLFFGAVRGTGTAEQFGHWISKGAGDLRKFYGCFSMTELGHGSNLAGLETTAIYDEETDEFIINTPHIAATKWWIGGAAHTATHTVVFARLIVKGKDYGVKTFVVQLRNINDHSLKVGISIGDIGKKMGRDGIDNGWIQFTNVRIPRQNLLMKYTKVDREGNVTQPPLAQLTYGSLITGRVSMASDSHQVGKRFITIALRYACIRRQFSTTPGQPETKIIDYPYHQRRLLPLLAYVYALKMTADEVGALFSRTMLKMDDLKPDDKAGLNEVVSDVKELFSVSAGLKAFSTWACADVIDKTRQACGGHGYSGYNGFGQAYADWVVQCTWEGDNNILTLSAGRALIQSAVALRKGEPVGNAVSYLKRYKDLANAKLNGRSLTDPKVLVEAWEVAAGNIINRATDQYEKLIGEGLNADQAFEVLSQQRFQAAKVHTRRHLIAAFFSRIDTEAGEAIKQPLLNLALLFALWSIEEDSGLFLREGFLEPKDIDTVTELVNKYCTTVREEVIGYTDAFNLSDYFINAPIGCYDGDAYRHYFQKVNEQNPARDPRPPYYASTLKPFLFREEEDDDICELDEE

SEQ ID NO: 12 - protein, 661 aa, Rattus norvegicus
MNPDLRKERASATFNPELITHILDGSPENTRRRREIENLILNDPDFQHEDYNFLTRSQRYEVAVKKSATMVKKMREYGISDPEEIMWFKKLYLANFVEPVGLNYSMFIPTLLNQGTTAQQEKWMRPSQELQIIGTYAQTEMGHGTHLRGLETTATYDPKTQEFILNSPTVTSIKWWPGGLGKTSNHAIVLAQLITQGECYGLHAFVVPIREIGTHKPLPGITVGDIGPKFGYEEMDNGYLKMDNYRIPRENMLMKYAQVKPDGTYVKPLSNKLTYGTMVFVRSFLVGNAAQSLSKACTIAIRYSAVRRQSEIKQSEPEPQILDFQTQQYKLFPLLATAYAFHFVGRYMKETYLRINESIGQGDLSELPELHALTAGLKAFTTWTANAGIEECRMACGGHGYSHSSGIPNIYVTFTPACTFEGENTVMMLQTARFLMKIYDQVRSGKLVGGMVSYLNDLPSQRIQPQQVAVWPTMVDINSLEGLTEAYKLRAARLVEIAAKNLQTHVSHRKSKEVAWNLTSVDLVRASEAHCHYVVVKVFSDKLPKIQDKAVQAVLRNLCLLYSLYGISQKGGDFLEGSIITGAQLSQVNARILELLTLIRPNAVALVDAFDFKDMTLGSVLGRYDGNVYENLFEWAKKSPLNKTEVHESYHKHLKPLQSKL

SEQ ID NO: 13 - protein, 661 aa, Rattus norvegicus
MNPDLRKERASATFNPELITHILDGSPENTRRRREIENLILNDPDFQHEDYNFLTRSQRYEVAVKKSATMVKKMREYGISDPEEIMWFKNSVHRGHPEPLDLHLGMFLPTLLHQATAEQQERFFMPAWNLEITGTYAQTEMGHGTHLRGLETTATYDPKTQEFILNSPTVTSIKWWPGGLGKTSNHAIVLAQLITQGECYGLHAFVVPIREIGTHKPLPGITVGDIGPKFGYEEMDNGYLKMDNYRIPRENMLMKYAQVKPDGTYVKPLSNKLTYGTMVFVRSFLVGNAAQSLSKACTIAIRYSAVRRQSEIKQSEPEPQILDFQTQQYKLFPLLATAYAFHFVGRYMKETYLRINESIGQGDLSELPELHALTAGLKAFTTWTANAGIEECRMACGGHGYSHSSGIPNIYVTFTPACTFEGENTVMMLQTARFLMKIYDQVRSGKLVGGMVSYLNDLPSQRIQPQQVAVWPTMVDINSLEGLTEAYKLRAARLVEIAAKNLQTHVSHRKSKEVAWNLTSVDLVRASEAHCHYVVVKVFSDKLPKIQDKAVQAVLRNLCLLYSLYGISQKGGDFLEGSIITGAQLSQVNARILELLTLIRPNAVALVDAFDFKDMTLGSVLGRYDGNVYENLFEWAKKSPLNKTEVHESYHKHLKPLQSKL

SEQ ID NO: 14 - protein, 664 aa, Glycine max
MEDGVDHLAFERNKAQFDVEDMKIIWAGSRQDFELSDRISRLVASDPAFRKDDRTRLIGRLFKNTLRKAAYAWKRINELRLNEQEAYKLRSFVDQPAFTDLHWGMFVPAIQGQGTDEQQQKWLPLAYGMQIIGCYAQTELGHGSNVQGLETTATFDPKTDEFVIHSPTLTSSKWWPGGLGKISTHAVAYARLIIGGEDHGVHGFIVQLRSLDDHLPLPGITIGDIGMKFGNAAYNTMDNGVLRFDHVRIPRNQMLMRVSQVTREGRYVSSNVPRQLVYGTMVNVRQKIVADASVALSRAVCIATRYSAVRRQFGSHNGGLETQVIDYKTQQARLFPLLASAYAFRFVGGWLKWLYMDVTERLQANDFSTLPEAHACTAGLKSLTTTATADGIEECRKLCGGHGYLCSSGLPELFAVYVPACTYEGDNVVLLLQVARHLMKTVSQLGSGNKPVGTTAYMARVEQLMQYHSDVEKAEDWLKPNVVLEAFEARASRMSVACAQNLSKFANPEEGFQELAADLVDAAVAHCQLIVVSKFIEKLQQDIPGKGVKKQLEVLCSIYALFLLHKHLGDFLSTGCINPKQGSLASEQLRNLYSQVRPNAIALVDAFNYTDHYLGSILGRYDGNVYPKMNEEAWKDPLNDSVVPDGFKEYIQPMLKQQLRNARL

SEQ ID NO: 15 - protein, 665 aa, Glycine max
MEGMVDHLAFERNNSQFDVDEMKIVWAGSRHAFEVSDKMARLVASDPAFRKDDRVVLDRKALFKNTLRKAAYAWKRIIELRLSEEEAAMLRSFVDQPAFTDLHWGMFVPAIKGQGTEEQQKKWLPLAHKMQIIGCYAQTELGHGSNVQGLETTATFDPRTDEFVIHSPTLTSSKWWPGGLGKVSTHAVVYARLITDGQDHGVHGFIVQLRSLDDHLPLPGITVGDIGMKFGNGAYNSMDNGMLRFDHVRIPRNQMLMRVSQVTREGKYVQSSVPRQLVYGTMVYVRQTIVSDASVALSRAVCIATRYSAVRRQFGSKEGGLETQVIDYKTQQARLFPLLASAYAFRFVGEWLKWLYMDVMKRLQASDFSTLPEAHACTAGLKSLTTSATADGIEECRKLCGGHGYLCSSGLPELFAVYIPTCTYEGDNTVLLLQVARHLIKTISQLGSRNKPVGTTSYIGRVEQLMQYRSDVQKVEDWLKPNAVLGAFEARAAKKVVACAQNLSKFTNPEEGFQELSVDLVEAAVAHCQLIVVSKFIEKLQQDIPGKGVKQQLELLCSIYALFLLHKHLGDFLATGCITPKQGSLANELLRSLYSQVRPNAIALVDAFNYTDHYLGSVLGRYDGDVYPKLYEEAWKDPLNDSVVPDGFQEYIRPMLKQQLRNARL

SEQ ID NO: 16 - protein, 684 aa, Beuvaria bassiana
MSFPDNLKPKEPSGSSLLEKERRQSPVDVDALGKHIFAGTSFLERQARVLRAIEQEPLFDKSRQQQLSRVERVKLGLARGKLMRRLQDRHGWDMDDYHMAAYLVGEQSPYRLHVGMFRTTVEEQSSDAQRAYWMPRVNGWEVSGAYSQTELGHGSNVRGVELEARWDPAAREFVVHSPTLTAAKWWNGSLGRTANHAILMAQLMVPDPKREGQYISHGPQAFIAQIRDLKTNLPLEGVVIGDIGVKIGFTSMDNGYMLFNQFRIPHSALLSRYVQLDPETGVFSKSPNPALAYGTMTSIRTMLVEEAGTHLARAVTIAIRYTAIRQQFRDKDSQDPSSAELQVLDYPTVQVRLFPLLAAAFALQYTGKVMRQDYAKTRGEVEKGNLEGLAVMHSNSSGLKSLSTEITNAGIETCRRAMGGHGYGSGSGLVEMQKDYQAKPILEGDNWMITQQTSSFLIKRMTAAAKTRNEPPKDQIDAQLKTFLHQKDKGRTFDILNSDSDIEESFKWRAASMTYDAYEARVIKKKRHNDLLIQFHKLSHAHSQSIMVSSFLTTLTSSNDLAHETKEIVFDLYRLFAYTTIQAESYEFLRCGAASSKDLDALPERIQALLTRIRPHAVKLVDAWKIPDYLLDSALGRYDGNVYEDLFNRAHRLNPLNDIVFNPDYKDDEIVKGSGERKPLSPKL

SEQ ID NO: 17 - protein, 696 aa, Aspergillus nidulans
MPNPPPAWVQALKPASPQGTELLTQERAQSNIDVDTLGDLLHTKEALKKQDEILSVLKSEKVFDKSRNHVLGRTEKIQLALARGKRLQQLKKAHNWSDEDVHVANDLVSEPTPYGLHASMFLVTLREQGTPEQHKLFYERARNYEIIGCYAQTELGHGSNVRGLETTATWDPSDQTFIIHSPTLTASKWWIGSLGRTANHAVVMAQLYIGGKNYGPHPFVVQIRDMETHQPLENVYVGDIGPKFGYNTMDNGFLLFNKLKIPHVNMLARFAQVDKATNKYIRPASPSLMYGTMTWVRSNIVLQAGGVLARGVTIAVRYCAVRRQFQDRDAKANAEENQVLNYKMVQIRLLPLLAAMYALHFTGRGMMRLYEENQERMKAAAQADQEKRGAGPEQLRAGSDLLADLHATSCGLKALASTTAGEGLEVCRRACGGHGYSNYSGIGPWYADYLPTLTWEGDNYMLTQQVARYLLKSARAVLAGKGTANDTSRILQAYLARRDKGASFDILGNDADIVAAFAWRTAHLTFETLKYRDVEKRSWNSLLINFWRLSTALSQYLVVKNFYEAVNSPEIRSSLDKDTASTLRSLFRLHALHTLDREASEFFSSAAVTVRQIGLTQTSEVPKLLDEIRPHAVRLVDSWKIPDWQLDSALGRSDGDVYPDLFKRASMQNPVNDLVFDPYPWNENVLKNAGEIKSKL

SEQ ID NO: 18 - protein, 646 aa, Arabidopsis thaliana
MESRREKNPMTEEESDGLIAARRIQRLSLHLSPSLTPSPSLPLVQTETCSARSKKLDVNGEALSLYMRGKHIDIQEKIFDFFNSRPDLQTPIEISKDDHRELCMNQLIGLVREAGVRPFRYVADDPEKYFAIMEAVGSVDMSLGIKMGVQYSLWGGSVINLGTKKHRDKYFDGIDNLDYTGCFAMTELHHGSNVQGLQTTATFDPLKDEFVIDTPNDGAIKWWIGNAAVHGKFATVFARLILPTHDSKGVSDMGVHAFIVPIRDMKTHQTLPGVEIQDCGHKVGLNGVDNGALRFRSVRIPRDNLLNRFGDVSRDGTYTSSLPTINKRFGATLGELVGGRVGLAYASVGVLKISATIAIRYSLLRQQFGPPKQPEVSILDYQSQQHKLMPMLASTYAYHFATVYLVEKYSEMKKTHDEQLVADVHALSAGLKSYVTSYTAKALSVCREACGGHGYAAVNRFGSLRNDHDIFQTFEGDNTVLLQQVAADLLKRYKEKFQGGTLTVTWSYLRESMNTYLSQPNPVTARWEGEDHLRDPKFQLDAFRYRTSRLLQNVAARLQKHSKTLGGFGAWNRCLNHLLTLAESHIETVILAKFIEAVKNCPDPSAKAALKLACDLYALDRIWKDIGTYRNVDYVAPNKAKVCFLV

SEQ ID NO: 19 - protein, 692 aa, Arabidopsis thaliana
MESRREKNPMTEEESDGLIAARRIQRLSLHLSPSLTPSPSLPLVQTETCSARSKKLDVNGEALSLYMRGKHIDIQEKIFDFFNSRPDLQTPIEISKDDHRELCMNQLIGLVREAGVRPFRYVADDPEKYFAIMEAVGSVDMSLGIKMGVQYSLWGGSVINLGTKKHRDKYFDGIDNLDYTGCFAMTELHHGSNVQGLQTTATFDPLKDEFVIDTPNDGAIKWWIGNAAVHGKFATVFARLILPTHDSKGVSDMGVHAFIVPIRDMKTHQTLPGVEIQDCGHKVGLNGVDNGALRFRSVRIPRDNLLNRFGDVSRDGTYTSSLPTINKRFGATLGELVGGRVGLAYASVGVLKISATIAIRYSLLRQQFGPPKQPEVSILDYQSQQHKLMPMLASTYAYHFATVYLVEKYSEMKKTHDEQLVADVHALSAGLKSYVTSYTAKALSVCREACGGHGYAAVNRFGSLRNDHDIFQTFEGDNTVLLQQVAADLLKRYKEKFQGGTLTVTWSYLRESMNTYLSQPNPVTARWEGEDHLRDPKFQLDAFRYRTSRLLQNVAARLQKHSKTLGGFGAWNRCLNHLLTLAESHIETVILAKFIEAVKNCPDPSAKAALKLACDLYALDRIWKDIGTYRNVDYVAPNKAKAIHKLTEYLSFQVRNVAKELVDAFELPDHVTRAPIAMQSDAYSQYTQVVGF

SEQ ID NO: 20 - protein, 711 aa, Candida viswanathii
  VARIANT 711: note = Xaa = Any Amino Acid
  VARIANT 711: note = Xaa = Any Amino Acid
MTFTKKNVSVSQGPDPRTSIQTERANSKFDPVTMNYFLEGSKERSELMKSLAQQIERDPILFTDGSYYDLTKDQQRELTVLKINRLSRYREGDSVDTFNKRLSIMGVVDPQVATRIGVNLGLFLSCISGNGTAEQFKYWAIDKGTHNIQGLYGCFGMTELGHGSNVAGVETTATFDKETDEFVINTPHIGATKWWIGGAAHSATHCSVYARLVVDGKDYGVKTFVVPLRDSNHDLMPGVTVGDIGAKMGRDGIDNGWIQFSNVRIPRFFMLQKFCKVSAEGEVVLPPLEQLSYSALLGGRVMMVLDSYRMLARVSTIALRYAIGRRQFKGDNVDQNDPNALETQLIDYPLHQKRLFPYLAAAYVVSTGALKVEHTIQSTLATLDAAVENNDTTAIFKSIDDMKSLFIDSGSLKATTTWLAAEAIDQCRQACGGHGYSSYNGFAKAFNDWVVQCTWEGDNNVLSLSVGKPIIKQIIGIEDNGKTVRGSTAFLNQVKDFTGSNASKVVLNNTSDLNDINKVIKSIEVAIIRLAHEAAISVRKESLDFAGAELVQISKLKAHHYLLTEFVKRVGEFEHKELVPFLNTIGRLYSATVVLDKFAGVFLTFNVASPQAITDLASTQIPKLCAEVRPNVVAYTDSFQQSDMVINSAIGKYDGDVYENYFDLVKQLNPPKNTKAPYTAALEGMLNRPSLEARERYEKSDETAAILSKPX

SEQ ID NO: 21 - protein, 662 aa, Candida viswanathii
MPTELQKERELTKFNPKELNYFLEGSQERSEIISNMVEQMQKDPILKVDASYYNLTKDQQREVTAKKIARLSRYFEHEYPDQQAQRLSILGVFDPQVFTRIGVNLGLFVSCVRGNGTNSQFFYWTINKGIDKLRGIYGCFGMTELAHGSNVQGIETTATFDEDTDEFVINTPHIGATKWWIGGAAHSATHCSVYARLKVKGKDYGVKTFVVPLRDSNHDLEPGVTVGDIGAKMGRDGIDNGWIQFSNVRIPRFFMLQKYCKVSRSGEVTMPPSEQLSYSALIGGRVTMMMDSYRMTSRFITIALRYAIHRRQFKKKDTDTIETKLIDYPLHQKRLFPFLAAAYLFSQGALYLEQTMNATNDKLDEAVSAGEKEAIDAAIVESKKLFVASGCLKSTCTWLTAEAIDEARQACGGHGYSSYNGFGKAYSDWVVQCTWEGDNNILAMNVAKPMVRDLLKEPEQKGLVLSSVADLDDPAKLVKAFDHALSGLARDIGAVAEDKGFDITGPSLVLVSKLNAHRFLIDGFFKRITPEWSEVLRPLGFLYADWILTNFGATFLQYGIITPDVSRKISSEHFPALCAKVRPNVVGLTDGFNLTDMMTNAAIGRYDGNVYEHYFETVKALNPPENTKAPYSKALEDMLNRPDLEVRERGEKSEEAAEILSS

SEQ ID NO: 22 - protein, 689 aa, Yarrowia lipolytica
MTTNTFTDPPVEMAKERGKTQFTVRDVTNFLNGGEEETQIVEKIMSSIERDPVLSVTADYDCNLQQARKQTMERVAALSPYLVTDTEKLSLWRAQLHGMVDMSTRTRLSIHNNLFIGSIRGSGTPEQFKYWVKKGAVAVKQFYGCFAMTELGHGSNLKGLETTATYDQDSDQFIINTPHIGATKWWIGGAAHTSTHCVCFAKLIVHGKDYGTRNFVVPLRNVHDHSLKVGVSIGDIGKKMGRDGVDNGWIQFTNVRIPRQNMLMRYAKVSDTGVVTKPALDQLTYGALIRGRVSMIADSFHVSKRFLTIALRYACVRRQFGTSGDTKETKIIDYPYHQRRLLPLLAYCYAMKMGADEAQKTWIETTDRILALNPNDPAQKNDLEKAVTDTKELFAASAGMKAFTTWGCAKIIDECRQACGGHGYSGYNGFGQGYADWVVQCTWEGDNNVLCLSMGRGLVQSALQILAGKHVGASIQYVGDKSKISQNGQGTPREQLLSPEFLVEAFRTASRNNILRTTDKYQELVKTLNPDQAFEELSQQRFQCARIHTRQHLISSFYARIATAKDDIKPHLLKLANLFALWSIEEDTGIFLRENILTPGDIDLINSLVDELCVAVRDQVIGLTDAFGLSDFFINAPIGSYDGNVYEKYFAKVNQQNPATNPRPPYYESTLKPFLFREEEDDEICDLDE

SEQ ID NO: 23 - protein, 700 aa, Yarrowia lipolytica; identical to SEQ ID NO: 11

SEQ ID NO: 24 - protein, 700 aa, Yarrowia lipolytica
MISPNLTANVEIDGKQYNTFTEPPKALAGERAKVKFPIKDMTEFLHGGEENVTMIERLMTELERDPVLNVSGDYDMPKEQLRETAVARIAALSGHWKKDTEKEALLRSQLHGIVDMGTRIRLGVHTGLFMGAIRGSGTKEQYDYWVRKGAADVKGFYGCFAMTELGHGSNVAGLETTATYIQDTDEFIINTPNTGATKWWIGGAAHSATHTACFARLLVDGKDYGVKIFVVQLRDVSSHSLMPGIALGDIGKKMGRDAIDNGWIQFTNVRIPRQNMLMKYAKVSSTGKVSQPPLAQLTYGALIGGRVTMIADSFFVSQRFITIALRYACVRRQFGTTPGQPETKIIDYPYHQRRLLPLLAFTYAMKMAADQSQIQYDQTTDLLQTIDPKDKGALGKAIVDLKELFASSAGLKAFTTWTCANIIDQCRQACGGHGYSGYNGFGQAYADWVVQCTWEGDNNVLCLSMGRGLIQSCLGHRKGKPLGSSVGYLANKGLEQATLSGRDLKDPKVLIEAWEKVANGAIQRATDKFVELTKGGLSPDQAFEELSQQRFQCAKIHTRKHLVTAFYERINASAKADVKPYLINLANLFTLWSIEEDSGLFLREGFLQPKDIDQVTELVNHYCKEVRDQVAGYTDAFGLSDWFINAPIGNYDGDVYKHYFAKVNQQNPAQNPRPPYYESTLRPFLFREDEDDDICELDEE

SEQ ID NO: 25 - protein, 701 aa, Yarrowia lipolytica
MITPNPANDIVHDGKLYDTFTEPPKLMAQERAQLDFDPRDITYFLDGSKEETELLESLMLMYERDPLFNNQNEYDESFETLRERSVKRIFQLSKSIAMDPEPMSFRKIGFLGILDMGTYARLGVHYALFCNSIRGQGTPDQLMYWLDQGAMVIKGFYGCFAMTEMGHGSNLSRLETIATFDKETDEFIINTPHVGATKWWIGGAAHTATHTLAFARLQVDGKDYGVKSFVVPLRNLDDHSLRPGIATGDIGKKMGRDAVDNGWIQFTNVRVPRNYMLMKHTKVLRDGTVKQPPLAQLTYGSLITGRVQMTTDSHNVSKKFLTIALRYATIRRQFSSTPGEPETRLIDYLYHQRRLLPLMAYSYAMKLAGDHVRELFFASQEKAESLKEDDKAGVESYVQDIKELFSVSAGLKAATTWACADIIDKARQACGGHGYSAYNGFGQAFQDWVVQCTWEGDNTVLTLSAGRALIQSALVYRKEGKLGNATKYLSRSKELANAKRNGRSLEDPKLLVEAWEAVSAGAINAATDAYEELSKQGVSVDECFEQVSQERFQAARIHTRRALIEAFYSRIATADEKVKPHLIPLANLFALWSIEEDSALFLAEGYFEPEDIIEVTSLVNKYCGIVRKNVIGYTDAFNLSDYFINAAIGRYDGDVYKNYFEKVKQQYPPEGGKPHYYEDVMKPFLHRERIPDVPMEPEDIQ

SEQ ID NO: 26 - protein, 699 aa, Yarrowia lipolytica
MNNNPTNVILGGKEYDTFTEPPAQMELERAKTQFKVRDVTNFLTGSEQETLLTERIMREIERDPVLNVAGDYDADLPTKRRQAVERIGALARYLPKDSEKEAILRGQLHGIVDMGTRTRIAVHYGLFMGAIRGSGTKEQYDYWVAKGAATLHKFYGCFAMTELGHGSNVAGLETTATLDKDTDEFIINTPNSGATKWWIGGAAHSATHTACLARLIVDGKDYGVKIFIVQLRDLNSHSLLNGIAIGDIGKKMGRDAIDNGWIQFTDVRIPRQNMLMRYDRVSRDGEVTTSELAQLTYGALLSGRVTMIAESHLLSARFLTIALRYACIRRQFGAVPDKPETKLIDYPYHQRRLLPLLAYTYAMKMGADEAQQQYNSSFGALLKLNPVKDAEKFAVATADLKALFASSAGMKAFTTWAAAKIIDECRQACGGHGYSGYNGFGQAYADWVVQCTWEGDNNVLCLSMGRSLIQSCIAMRKKKGHVGKSVEYLQRRDELQNARVDNKPLTDPAVLITAWEKVACEAINRATDSFIKLTQEGLSPDQAFEELSQQRFECARIHTRKHLITSFYARISKAKARVKPHLTVLANLFAVWSIEEDSGLFLREGCFEPAEMDEITALVDELCCEAREQVIGFTDAFNLSDFFINAPIGRFDGDAYKHYMDEVKAANNPRNTHAPYYETKLRPFLFRPDEDEEICDLDE

SEQ ID NO: 27 - protein, 689 aa, Yarrowia lipolytica
MLSQQSLNTFTEPPVEMARERNQTSFNPRLLTYFLDGGEKNTLLMDRLMQEYERDPVFRNEGDYDITDVAQSRELAFKRIAKLIEYVHTDDEETYLYRCMLLGQIDMGAFARYAIHHGVWGGAIRGAGTPEQYEFWVKKGSLSVKKFYGSFSMTELGHGSNLVGLETTATLDKNADEFVINTPNVAATKWWIGGAADTATHTAVFARLIVDGEDHGVKTFVVQLRDVETHNLMPGIAIGDCGKKMGRQGTDNGWIQFTHVRIPRQNMLMRYCHVDSDGNVTEPMMAQMAYGALLAGRVGMAMDSYFTSRKFLTIALRYATIRRAFAAGGGQETKLIDYPYHQRRLLPLMAQTYAIKCTADKVRDQFVKVTDMLLNLDVSDQEAVPKAIAEAKELFSVSAGVKATTTWACAHTIDQCRQACGGHGYSAYNGFGRAYSDWVIQCTWEGDNNILCLSAGRALVQSNRAVRAGKPIGGPTAYLAAPAGSPKLAGRNLYDPKVMIGAWETVSRALINRTTDEFEVLAKKGLSTAQAYEELSQQRFLCTRIHTRLYMVKNFYERIAEEGTEFTKEPLTRLANLYAFWSVEEEAGIFLREGYITPQELKYISAEIRKQLLEVRKDVIGYTDAFNVPDFFLNSAIGRADGDVYKNYFKVVNTQNPPQDPRPPYYESVIRPFLFRKDEDEEICSLEDE

SEQ ID NO: 28 - protein, 906 aa, Candida viswanathii
MSPVDFKDKVVIITGAGGGLGKYYSLEFAKLGAKVVVNDLGGALNGQGGNSKAADVVVDEIVKNGGVAVADYNNVLDGDKIVETAVKNFGTVHVIINNAGILRDASMKKMTEKDYKLVIDVHLNGAFAVTKAAWPYFQKQKYGRIVNTSSPAGLYGNFGQANYASAKSALLGFAETLAKEGAKYNIKANAIAPLARSRMTESILPPPMLEKLGPEKVAPLVLYLSSAENELTGQFFEVAAGFYAQIRWERSGGVLFKPDQSFTAEVVAKRFSEILDYDDSRKPEYLKNQYPFMLNDYATLTNEARKLPANDASGAPTVSLKDKVVLITGAGAGLGKEYAKWFAKYGAKVVVNDFKDATKTVDEIKAAGGEAWPDQHDVAKDSEAIIKNVIDKYGTIDILVNNAGILRDRSFAKMSKQEWDSVQQVHLIGTFNLSRLAWPYFVEKQFGRIINITSTSGIYGNFGQANYSSSKAGILGLSKTMAIEGAKNNIKVNIVAPHAETAMTLTIFREQDKNLYHADQVAPLLVYLGTDDVPVTGETFEIGGGWIGNTRWQRAKGAVSHDEHTTVEFIKEHLNEITDFTTDTENPKSTTESSMAILSAVGGDDDDDDEDEEEDEGDEEEDEEDEEEDDPVWRFDDRDVILYNIALGATTKQLKYVYENDSDFQVIPTFGHLITFNSGKSQNSFAKLLRNFNPMLLLHGEHYLKVHSWPPPTEGEIKTTFEPIATTPKGTNVVIVHGSKSVDNKSGELIYSNEATYFIRNCQADNKVYADRPAFATNQFLAPKRAPDYQVDVPVSEDLAALYRLSGDRNPLHIDPNFAKGAKFPKPILHGMCTYGLSAKALIDKFGMFNEIKARFTGIVFPGETLRVLAWKESDDTIVFQTHVVDRGTIAINNAAIKLVGDKAKI

SEQ ID NO: 29 - protein, 472 aa, Candida viswanathii
MMWKFLIAIGLIFSYCCNAQLLDSLSFDNNWVNTHYIRTIDLSKGFVKETDLIQIKNINDKPQDEYYFVVNDGFDSIDELSIFSAFVGDQALEVEVDEVVPDKVFKLKLPVPIAPNSDLELRINFVYIDSLVSVPSKIAMDATQQLLYKTNKFPFSPYVTQEYTLALSGMSKGQEMDLHIDVEDTPGLPDLKPRVESQVLKYGPIAEDIPAFALKPMGLMYDHNRPLTKAVSLNRSIWLPASDINKVSIEEYYELTNTGAELDKGFSRVDWMKGRFESTRNHWALSHLEIPLLERGFDDYYYTDKVGVVSTHKIFKNHLLLQPRYPVFGGWKYNFTLGWSEELSKFLHKLHDNQDEYIIKFPILNSLRDVTYQDVYLEFYLPENAEFQNISSPIAFESISIENELSYLDVSKGHTKITVHYTNLFDDLHKLDVFVKYQYTQVAFIYKIAKISGFVFLGLVSYYLLGLLDLSI

SEQ ID NO: 30 - protein, 519 aa, Saccharomyces fibuligera
MKFGVLFSVFAAIVSALPLQEGPLNKRAYPSFEAYSNYKVDRTDLETFLDKQKEVSLYYLLQNIAYPEGQFNNGVPGTVIASPSTSNPDYYYQWTRDSAITFLTVLSELEDNNFNTTLAKAVEYYINTSYNLQRTSNPSGSFDDENHKGLGEPKFNTDGSAYTGAWGRPQNDGPALRAYAISRYLNDVNSLNEGKLVLTDSGDINFSSTEDIYKNIIKPDLEYVIGYWDSTGFDLWEENQGRHFFTSLVQQKALAYAVDIAKSFDDGDFANTLSSTASTLESYLSGSDGGFVNTDVNHIVENPDLLQQNSRQGLDSATYIGPLLTHDIGESSSTPFDVDNEYVLQSYYLLLEDNKDRYSVNSAYSAGAAIGRYPEDVYNGDGSSEGNPWFLATAYAAQVPYKLAYDAKSASNDITINKINYDFFNKYIVDLSTINSAYQSSDSVTIKSGSDEFNTVADNLVTFGDSFLQVILDHINDDGSLNEQLNRYTGYSTGAYSLTWSSGALLEAIRLRNKVKALA

SEQ ID NO: 31 - protein, 403 aa, Rattus norvegicus
MIHTNLKKKFSLFILVFLLFAVICVWKKGSDYEALTLQAKEFQMPKSQEKVAMGSASQVVFSNSKQDPKEDIPILSYHRVTAKVKPQPSFQVWDKDSTYSKLNPRLLKIWRNYLNMNKYKVSYKGPGPGVKFSVEALRCHLRDHVNVSMIEATDFPFNTTEWEGYLPKENFRTKVGPWQRCAVVSSAGSLKNSQLGREIDNHDAVLRFNGAPTDNFQQDVGSKTTIRLMNSQLVTTEKRFLKDSLYTEGILIVWDPSVYHADIPKWYQKPDYNFFETYKSYRRLNPSQPFYILKPQMPWELWDIIQEISADLIQPNPPSSGMLGIIIMMTLCDQVDIYEFLPSKRKTDVCYYHQKFFDSACTMGAYDPLLFEKNMVKHLNEGTDEDIYLFGKATLSGFRNIRC

SEQ ID NO: 32 - protein, 219 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQISF

SEQ ID NO: 33 - protein, 815 aa, Candida viswanathii
MTLLLKPTSELDATSRKIIERIQSNSPTFQHLFDLLLNLLPFFDKTVSLLGSIGYCDYEVAYVTYQTCIQVVGLMKPKTNSLNQDIFKGVQLQTRKRASTFKAILSYFAEPETQEEDPLLNRFKSLSGGGSKTKSSQDEVFHEWITSSELQRELSSKKVLLIDFRPRKDYLNNHIKYKDLVHIEPTQLETLLDSASDQDLETLVKKSAPYDQYHIFLERHKYDLIVVYNYNYGSESTDRLLGIIDVVSKPNPFTKLITILMNNKYISSRLKVKPLFLSGGVLNWYKTFGIEYLERTLVQNGVAHTSDNQYLKSFNDYVSTSKETPKTQVKTQNGDYIRPSQRKVNQFDPVPVKSGPTVFASAKVDLPPTPGSPAVSTPSPPRAPAPPTKTTSLTHVPEKEAKSPSPVTKEVTVSSKKSQFLELYTTGLVNLGNSCYMNCVVQCLAAAPQLTSFFFPTITESFSDHSYRQHINSNNKLGTKGELTTSFVELILNMLNNNGKAFSPTKFKRTMGSLSPSQQFLTYDQQDCIEFLNFLLDALHEDLNNVTITDPSERKLITDLSPEQEKSRETLPVRLASTIEWERYLKLNFSVIVDYFQGQHLSQLKCLECGFTSTTYNAFSILSLPIPQKLNNLGKVLLKDCLEEFVTTELLDDNNKWYCPQCKRFTRLTKKIAITRLPQVLIVNFNRFKMTNTGGFNKLETFVTYPVNEELDMTPYWPDVGSRINENSTMSIEMEQDLLQSFPIRNQTPPFKYKLFGVANHFGNLTTGHYTSYVYKHSDSKKTRNWCYFDDSKITYNVSPSQVVNKNAYCLFFQR

SEQ ID NO: 34 - protein, 421 aa, Candida viswanathii
MQLSLSVLSTVATALLSLTTAVDAKSHNIKLSKLSNEETLDASTFQEYTSSLANKYMNLFNAAHGNPTSFGLQHVLSNQEAEVPFVTPQKGGKYDAPLTNYLNAQYFTEIEIGTPGQPFKVILDTGSSNLWVPSQDCTSLACFLHSKYDHDASSTYKANGSEFSIQYGSGSMEGYISQDILTIGDLVIPKQDFAEATSEPGLAFAFGKFDGILGLAYDSISVNHIVPPVYNAINQGLLDKPQVSFYLGNTEKDENDGGLATFGGYDASLFQGKITWLPVRRKAYWEVSFEGIGLGDEYAELQKTGAAIDTGTSLITLPSSLAEIINAKIGATKSWSGQYQIDCAKRDELPDLTLTFAGHNFTLTAHDYILEVSGSCISVFTPMDFPKPIGDLAIIGDAFLRKYYSIYDLDKNAVGLAPSKA

SEQ ID NO: 35 - protein, 551 aa, Candida viswanathii
MGITNETQALLGGDSLSCLNKKKSNTKRNLSYLLNIITVSIIAYLCFFATHNHHNDSGIPKVDPHKKKNIIMMVTDGMGPASLSAARSFRQFRDKLAINDILTLDQYLIGSSRTRSSSSLVTDSAAGATAFSCALKSYNGAIGVSPDKSPCGTILEALKLQGYYTGLVVTTRITDATPAAFSAHVDYRFQEDLIAEHQLGEYPFGRAVDLILGGGRCHFLPTAQGGCRADDRNLIKESSDTWQYVGDRQQFDQLKGGKNVSLPLLGLLANTDIPYAIDRDEKEYPSLAEQVKVALTALSDATKDSDQGFFLLIEGSRIDHAGHHNDPTAQVREVLAYDEAFGEVIKFIDSTDVETVAISTSDHETGGLVVSRQVTPEYPDYIWYPEVLLNSTHSGDYLAHKIADYKNKDDTAKLTKFIKHEILETDLGVTDYTDKDVQAILDKVNDPANLLYVLNDIVSFRAQIGWTTHGHSAVDVNIYAHTNSPAIRAKLASAKAYHGLSGNHENIEIGAFMEEITGSNLSRVTELIKKTAHSPSLSKKEFSVDEFHGNV

SEQ ID NO: 36 - protein, 140 aa, Saccharomyces cerevisiae
MCCYCVCCTVSDFILYIVAFFFPPAAVLLRSGPCSSDFLLNVLLTLLGFLPGMLHAFYYITITSPLRNAEYVYYYQQGWVDSERNVPSNRPQNSQTPQNRPQQGSSARNVYPSVETPLLQGAAPHDNKQSLVESPPPYVP

SEQ ID NO: 37 - protein, 377 aa, Candida viswanathii
MRLKDIKLILIGILTISVTYFLISSFSGPRAYTTSDPNSSKMQFLRALESHPNWKETGLNFQPTKKLEVDDSSTPVRQQLAARFPYDPTQPFPKNIWQTWKVGIEDETFPKRYLKFQLSWDTKNPEYKHHVIPDDQCDELVAQLFEDVPDVARAYKVMPKSILKADFFRYLILFARGGVYTDIDTVGLKPIDTWMSNMELLWGEPNRAGLVVGIEADPDRPDWADWYARRIQFCQWTIQLKKGHPMLRELITKITDITLTREKRNELKKVLGKDEGGDIMNWTGPGIFTDTVFSYMNAILQAPEVITGKYKWDNIVDWKVFTGMQMPIAIDDVLVLPITSFSPDVSQMGSKSSTDPMAYAKHMFLGSWKDDGMPEME

SEQ ID NO: 38 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 39 - protein, 385 aa, Cannabis sativa
MNHLRAEGPASVLAIGTANPENILIQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSDSDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLDLKKEKFVDSRHVLSEHGNMSSSTVLFVMDELRKRSLEEGKSTTGDGFEWGVLFGFGPGLTVERVVVRSVPIKY

SEQ ID NO: 40 - protein, 391 aa, synthetic construct
  REGION 1..391: note = Cannabis sativa
MNHLRAEGPASVLAIGTANPENILIQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSDSDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLDLKKEKFVDSRHVLSEHGNMSSSTVLFVMDELRKRSLEEGKSTTGDGFEWGVLFGFGPGLTVERVVVRSVPIKYGRRAKL

SEQ ID NO: 41 - protein, 105 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRKLKPK

SEQ ID NO: 42 - protein, 111 aa, synthetic construct
  REGION 1..111: note = Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRKLKPKGRRAKL

SEQ ID NO: 43 - protein, 393 aa, Cannabis sativa
MGLSSVCTFSFQTNYHTLLNPHNNNPKTSLLCYRHPKTPIKYSYNNFPSKHTKSFHLQNKCSESLSIAKNSIRAATTNQTEPPESDNHSVATKILNFGKACWKLQRPYTIIAFTSCACGLFGKELLHNTNLISWSLMFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 44 - protein, 321 aa, synthetic construct
  REGION 1..321: note = Cannabis sativa
MAATTNQTEPPESDNHSVATKILNFGKACWKLQRPYTIIAFTSCACGLFGKELLHNTNLISWSLMFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 45 - protein, 398 aa, Cannabis sativa
MGLSLVCTFSFQTNYHTLLNPHNKNPKNSLLSYQHPKTPIIKSSYDNFPSKYCLTKNFHLLGLNSHNRISSQSRSIRAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 46 - protein, 394 aa, Cannabis sativa
MGLSSVCTFSFQTNYHTLLNPHNNNPKTSLLYRHPKTPIKYSYNNFPSKHCSTKSFHLQNKCSESLSIAKNSIRAATTNQTEPPESDNHSVATKILNFGKACWKLQRPYTIIAFTSCACGLFGKELLHNTNLISWSLMFKAFFFLVAVLCIASFTTTINQIYDLHIDRINKPDLPLASGEISVNTAWIMSIIVALFGLIITIKMKGGPLYIFGYCFGIFGGTVYSVPPFRWKQNPSTAFLLNFLAHIITNFTFYHASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 47 - protein, 397 aa, Cannabis sativa
MELSSICNFSFQTNYHTLLNPHNKNPKSSLLSHQHPKTPIITSSYNNFPSNYCSNKNFHLQNRCSKSLLIAKNSIRTDTANQTEPPESNTKYSVVTKILSFGHTCWKLQRPYTFIGVISCACGLFGRELFHNTNLLSWSLMLKAFSSLMVILSVNLCTNIINQITDLDIDRINKPDLPLASGEMSIETAWIMSIIVALTGLILTIKLNCGPLFISLYCVSILVGALYSVPPFRWKQNPNTAFSSYFMGLVIVNFTCYYASRAAFGLPFEMSPPFTFILAFVKSMGSALFLCKDVSDIEGDSKHGISTLATRYGAKNITFLCSGIVLLTYVSAILAAIIWPQAFKSNVMLLSHATLAFWLIFQTREFALTNYNPEAGRKFYEFMWKLHYAEYLVYVFI

SEQ ID NO: 48 - protein, 400 aa, Cannabis sativa
MVFSSVCSFPSSLGTNFKLVPRSNFKASSSHYHEINNFINNKPIKFSYFSSRLYCSAKPIVHRENKFTKSFSLSHLQRKSSIKAHGEIEADGSNGTSEFNVMKSGNAIWRFVRPYAAKGVLFNSAAMFAKELVGNLNLFSWPLMFKILSFTLVILCIFVSTSGINQIYDLDIDRLNKPNLPVASGEISVELAWLLTIVCTISGLTLTIITNSGPFFPFLYSASIFFGFLYSAPPFRWKKNPFTACFCNVMLYVGTSVGVYYACKASLGLPANWSPAFCLLFWFISLLSIPISIAKDLSDIEGDRKFGIITFSTKFGAKPIAYICHGLMLLNYVSVMAAAIIWPQFFNSSVILLSHAFMAIWVLYQAWILEKSNYATETCQKYYIFLWIIFSLEHAFYLFM

SEQ ID NO: 49 - protein, 384 aa, Cannabis sativa
MELSLSLGGPTIFPRYRASYTSTKLTTHFSNFPSKFSTKNFHQTLSFYGPTRGSKSLLNTHQWRNSIRACAEAGAAGSNPVLNKVSDFRDACWRFLRPHTIRGTTLGSIALVARALIENPNLIKWSLLLKAFSGLLALICGNGYIVGINQIYDIGIDKVNKPYLPIAAGDLSVQSAWYLVILFAVAGLLTVGFNFGPFITSLYCLGLVLGTIYSVPPFRMKRFPVAAFLIIATVRGFLLNFGVYYATRAALGLTFEWSSAVAFITTFVTLFALVIAITKDLPDVEGDRKFQISTFATKLGVRNIAYLGSGLLLLNYIGAIAAAIYMPQAFKRNLMLPIHTILALSLVFQAWVLEQANYTKEAIAGFYRFIWNLFYVEYIIFPFI

SEQ ID NO: 50 - protein, 387 aa, Cannabis sativa
MAIALWLPRISRSTTRRFLKPSSSLTLFSVSHSHNYIVTSNRSPIPRLFTVPNQSHGREWVSVSEVRLGYVSHISTAGKSDENRSRDAQVADVSWIDLYLPRQIHPYVRLARLDKPIGTWLLAWPCMWSISLAANPGHLPDIKMMTLFGCGALLLRGAGCTINDLLDRDIDTMVERTKLRPVASGIITPFQGICFLGFQLLLGLGILLQLNNYSRILGASSLLLVFSYPLMKRLTFWPQAYLGLTFNWGALLGWAAVKGNIDPAIVLPLYASGVFWTLVYDTIYAHQDKEDDVRVGIKSTALRFGDLTKQWNMGFGAACISSLALSGYNAEIGWPFYASLVAASGQLAWQISTVDLSSRDDCNKKFVSNKWFGAIIFSGIVLARISS

SEQ ID NO: 51 - protein, 308 aa, Cannabis sativa
MSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLGAYYHITDVQRGLLKAFDSLEDG

SEQ ID NO: 52 - protein, 852 aa, Streptomyces
MSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLGAYYHITDVQRGLLKAFDSLEDGMKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 53 - protein, 573 aa, synthetic construct
  REGION 1..573: note = Cannabis sativa
MFLKHIFVALAFALLADATPAQKRSPGFVALDFDIVKVQKNVTANDDAAAIVAKRQTNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 54 - protein, 543 aa, synthetic construct
  REGION 1..543: note = Cannabis sativa
MQLSLSVLSTVATALLSLTTAVDAKSHNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 55 - protein, 608 aa, synthetic construct
  REGION 1..608: note = Cannabis sativa
MQLSLSVLSTVATALLSLTTAVDAKSHNIKLSKLSNEETLDASTFQEYTSSLANKYMNLFNAAHGNPTSFGLQHVLSNQEAEVPFVTPQKGGNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 56 - protein, 523 aa, synthetic construct
  REGION 1..523: note = Cannabis sativa
MNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRHGRRAKL

SEQ ID NO: 57 - protein, 545 aa, Cannabis sativa
MNCSAFSFWFVCKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGMSYISQVPFVVVDLRNMHSIKIDVHSQTAWVEAGATLGEVYYWINEKNENFSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGVGMYVLYPYGGIMEEISESAIPFPHRAGIMYELWYTASWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPHHH

SEQ ID NO: 58 - protein, 574 aa, synthetic construct
  REGION 1..574: note = Cannabis sativa
MFLKHIFVALAFALLADATPAQKRSPGFVALDFDIVKVQKNVTANDDAAAIVAKRQTNPQENFLKCFSEYIPNNPANPKFIYTQHDQLYMSVLNSTIQNLRFTSDTTPKPLVIVTPSNVSHIQASILCSKKVGLQIRTRSGGHDAEGMSYISQVPFVVVDLRNMHSIKIDVHSQTAWVEAGATLGEVYYWINEKNENFSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGVGMYVLYPYGGIMEEISESAIPFPHRAGIMYELWYTASWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPHHH

SEQ ID NO: 59 - protein, 544 aa, synthetic construct
  REGION 1..544: note = Cannabis sativa
MQLSLSVLSTVATALLSLTTAVDAKSHNPQENFLKCFSEYIPNNPANPKFIYTQHDQLYMSVLNSTIQNLRFTSDTTPKPLVIVTPSNVSHIQASILCSKKVGLQIRTRSGGHDAEGMSYISQVPFVVVDLRNMHSIKIDVHSQTAWVEAGATLGEVYYWINEKNENFSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGVGMYVLYPYGGIMEEISESAIPFPHRAGIMYELWYTASWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPHHH

SEQ ID NO: 60 - protein, 609 aa, synthetic construct
  REGION 1..609: note = Cannabis sativa
MQLSLSVLSTVATALLSLTTAVDAKSHNIKLSKLSNEETLDASTFQEYTSSLANKYMNLFNAAHGNPTSFGLQHVLSNQEAEVPFVTPQKGGNPQENFLKCFSEYIPNNPANPKFIYTQHDQLYMSVLNSTIQNLRFTSDTTPKPLVIVTPSNVSHIQASILCSKKVGLQIRTRSGGHDAEGMSYISQVPFVVVDLRNMHSIKIDVHSQTAWVEAGATLGEVYYWINEKNENFSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGVGMYVLYPYGGIMEEISESAIPFPHRAGIMYELWYTASWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPHHH

SEQ ID NO: 61 - protein, 525 aa, synthetic construct
  REGION 1..525: note = Cannabis sativa
MSNPQENFLKCFSEYIPNNPANPKFIYTQHDQLYMSVLNSTIQNLRFTSDTTPKPLVIVTPSNVSHIQASILCSKKVGLQIRTRSGGHDAEGMSYISQVPFVVVDLRNMHSIKIDVHSQTAWVEAGATLGEVYYWINEKNENFSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGVGMYVLYPYGGIMEEISESAIPFPHRAGIMYELWYTASWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPHHHGRRAKL

SEQ ID NO: 62 - protein, 545 aa, Cannabis sativa
MNCSTFSFWFVCKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAACKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 63 - DNA, 567 bp, Candida viswanathii
  source 1..567: mol_type = other DNA
gaggatgaagaagacgaagacgaattggatgaagatgaagcgtatgagtattatgagtactgtcggacgttggaaggtggcagagttaagcccgagaaagcaaggaaggagtgggagatgatgagtgatgcggccaagaggatgtgaaggctgcgtatctgtttttgatagctggtggtagccgaatagaggaaggcaagcttgttcatattggatgatgatggtagatggtggctgccaaagtggttgtaaatagaaaaaagtgggtttgggtctgttgatagttagtggtggcggctgtctgtgattacgtcagcaagtagcacctcggcagttaaaacagcagcaacagaaaaaaaatgtgtgaaagtttgattcccccacagtctaccacacccagagttccatttatccataatatcacaagcaatagaaaaataaaaaattatcaacaaatcacaacgaaaagattctgcaaaattattttcacttcttcttttgacttcctcttcttcttgttaggttctttccatattttccccttaaacccatacacaacgcagccat

SEQ ID NO: 64 - DNA, 529 bp, synthetic construct
  misc_feature 1..529: note = PoX4_Promoter_CV
  source 1..529: mol_type = other DNA
gagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataa

SEQ ID NO: 65 - DNA, 861 bp, Candida viswanathii
  source 1..861: mol_type = other DNA
gtgccgacggagtaacaaaaatgttacagtgcgcactacttatcccgctaaggtgataaactgcaaaaacacaccgtcaaggagaagtcgacgttttgccgccacttgtgaagggaagaagagtcgttgagttgatgtaattaagctggcacgtagataccagaaggttctagagtagagcttgggtggtgtttggccctgtttggaccacggatagagatggagaatcccttggttagagcggagaggaaaaaattgaaactttgcatatcccacttcattatccttgatgtaaccgttttatggggtaattaaagtgtggaaaaataatcagggagacatattcccgatcaattgggtggtggtcgctcaatttctgtgagtagtaggctcagtggtgtgtattgggattggtagtagtctgtataagcagtgttatataacccattgcttgttgattcctattttgctggcaaaagtgacaactgtagttgtgagataatcctcggttattacgcctgggggggcagacagccaaagttgtgcccgtgcgacaatggcatcagaagaaacagaaaaaaaaaacacaggcatttttatccacatgcacactacccccactattcctgtctgcagtgtgcttgtgtgtggccccccgcagaatcaacagggcaaactctggagcctgaatctttatataaacttcaggcattggccccccttttcacaattcttcacatccaccattttttttcttctttcctaccatattagtttttttttattcttttcctacctatctgattattatcaaacatctggtcatcctcaaaagaaagaaagaaactataacaatcaatc

SEQ ID NO: 66 - DNA, 599 bp, Candida viswanathii
  source 1..599: mol_type = unassigned DNA
tggattatgtaatcacaggctgtttctccattcctgcatgtaggctggcccgcggtatcaaccatcgtcccgcttcttctggttttttttttttttccgctatgatatttttgatctcttgggggatttggtgggtctgccccccccgctactacaagctcaaacacccgaaaccttacaacacacacacacacatcccgcttagttgcgggttgaagaacgtgtattcccgtagggttaatggtgtgtccccccctagtcacccgcttctgccattctgggtttgccttcaaagctggcataaatgacgaaaaaaaagcacagcatcctgcacacaaccctgctcagtgtgacaggtggtggtgtaatagaaaacctcggcttaaaacctctggtcagagcatcaactgcaatcttgtctttttctgctgccctcacattctccccacacattcccaccctcaagatttcacaggcaaaaactgcaaatatatataaatctacaaccaattctttccccagatggaaaatctaatttttgttcaccctttttctttctcctgctatcactgctactgcacatattcaacaccacaacc

SEQ ID NO: 67 - DNA, 502 bp, synthetic construct
  misc_feature 1..502: note = TEF1_promoter_CV
  source 1..502: mol_type = other DNA
gactgggagaacagaccaaggatgcccatacaccattaataacaagcacaccttgataaatctctagtgttgacaaattggtgatttgaacatgactgtagagagagagacaagtaaccactgatggattggtggtggcaaataaccactttaaataaagaccaactcacacacaaagacagcaggtcgtgttcctatttccaattttcacaaggagaagaataaaaatttttcaatgagattaactaaagaaacagacaggcagcccacaagaagaagaagaagaagaggaagaggaagaagaagaagaagaagaagaagaagagaaaaaaaatttttccctctgcgttgcgttgggcttgggttgccagcacccaccatatataactctcatcaaatacccagtagagaaaaatttttcctccctctttttctttctttcttctccttcttttgctactctttctgtttttcatcaaaaagatatatataatcaatcatg

SEQ ID NO: 68 - DNA, 502 bp, Candida viswanathii
  source 1..502: mol_type = other DNA
accaaacgttactttttttttgcaatcggatggtatgggtctggggttcacctgttttgtaaagctacagaaggtggcatatttctctgatcaggtgttttttttttcggctgctgctgctcgtggtggtgtagtggtagtggtgtgtgtgtgtgtgtgtgcgtgcgtgtggaaggacgctttttgctctctgactcctcccaatcagaagttgctatagtggtgaaacaacaatggatgataatgccccgggcggtgcgtgtccgacacaaaccactacattttttagctgggagcctactgccactacgacccacccacccatggtcaacaaaaaaattctgacaaattataaaataacccttgaattcccccttggaaaaatttttggtatttctctctctcttttcctttccctcttctttttctctccatcaatcaattgacgttcagtaactcaattaattacatcacatccctcaattaaagaatttaaacaatg

SEQ ID NO: 69 - DNA, 1006 bp, synthetic construct
  misc_feature 1..1006: note = POX2_Promoter_Yl
  source 1..1006: mol_type = other DNA
acgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgcttgagtatcgaagtcttgtacctccctgtcaatcatggcaccactggtcttgacttgtctattcatactggacaagcgccagagttaagcttgtagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggaggcctaattttgacgtgcaaggggcgaattggggcgagaaacacgtcgtggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacgacgccatga

SEQ ID NO: 70 - DNA, 1004 bp, Yarrowia lipolytica
  source 1..1004: mol_type = other DNA
aaacttctccgagtctgtgccttcaggtgggcatagttgatgggtgttttgaagttaatagtggggaagaactatggcaaacaagcagatgcaggcaccttgtaactgcagaccggttcttgtctaccgactccgctgcacctgtgccgcggtacatgtcgtcacaggctgcggggttcggaggcccccttgcaacctcctttgatagttgctatggccccaaagagttatacgagatagacccacagatctacttgactgttgtcacagaacctgctaggtttgcttattgtacccgctttgtagctactgtacaacgacaacgtcaaaaattgagacgcgaacaaactccagatgcagaacccaaacctctctctcagagtttcgagtgcttctacctcacagtaaagtggaggtggacctgcaagggaattcagtcacaaggccccgaatgtctccgaaactccaatcggaccgtttaaacagactaatatcacgtcattgattgatattagcatccggcaagagccgcaaggttatctcctcaccaatgagcctgttgtacggctcattccgcatctgcggctgattcagtttcgagtggggatggtagacttcattgcagcattcctaaccttctacttggtccgtggagatgtcatggacatcgattttgggctgagaagccttttgacgatgttgatatcactgaccgctaatttactctggcagtttctccggctctcgaggcatcgtcgatcaccaaacactatctgctagtctaaatgtccgacacgacagcttttgatcgccgtgaacggcgcagacctcatgcaccatgcaccagggccaaatcaattacgggtcgcttagcgttgcagtcggggcattatggtggaagttccgatacggcacagacacattccatagtggggggattggattataaaagggccatagaaagccctcaattgatacccaagtaccagctctcctcactatga

SEQ ID NO: 71 - DNA, 1367 bp, synthetic construct
  misc_feature 1..1367: note = ICL1_Promoter_Yk
  source 1..1367: mol_type = other DNA
gaccccctccttttgccagtatatccaccgcaacacccaccatgagcgacatctgataccgtgccgcgaccactaccccaaataagctccaactaatatgccgaggcaggtgggaaactatgcactccagacgacgctgtagaagcacatggaaggtgcggaggcggtggcaacgaggggcatgagccatcaacgagtaaccacagacaaggcaaggggggaaacgcgaccggaatctctcgcggtcacgtgacccgcccgggttccactcgtccatgttgtgtctctggtgtcttcggccgactcgcattggttaaacttccaccaccgcaatcacgtcccactggccaaactttttctgctttctctgactttttctggccaaaaggcaacgtcggaaagggtcgggaggattcggaaccgacgaaaatcggccggctccagcgggggtagttcggcagtcctggtgggagctctaggggagctgtggtctgtgtagggcgcgggtccgggtttgttgggtgtcaaatcacgtgtttttgcccccccgctgagccggactccgacaaccgtgtctccaacggcctgactaagctgctcccagcactctgccgtagcgttggtctgtcctgtcgcactctgttcaaagacagaagaaagaaaaagctaacctccacgtcagagacaatggtagaaggcttgttccttgcaaccgaggagagtgagtgttctcggcacgagcatcatgggcgatctggagggtatttttgaggggaaaaaacgggatcaggacaaacagaggccacagaccgggaatctgggccccaaaacggccttttcccgtcgcaaaaccggtctacatacaccccttcggcccgccacaggccggtgtgaaaaaccctaaagcttgcttcaaaccagacggacgcacagcaagacacatcatgaagagtcacctgcagtatatatagatctggggattcccagtagactgaccaagcatacaaaagtgagtatccaacagcgacacgtgagatggcagagacacagagacgtgtctacatggttggacaagtctccacattcgccagagacgtatccacatacaaacacaatctcacagctgatctgctcctgtgacagcacagtacatgttagtggatgaggtgttgtgtagtgggttaaatgggtggactgattcagtggcatcggtggcgacaccctctactcttcatgtcgtcacctaccgttcggaatcccaattatctgatgaactaaacgatttctggccaaaacacaattttgccaaagaagtcgttctcaccaatgcaagtgtcacatcaaacatctgtcccgtactaacccag

SEQ ID NO: 72 - DNA, 1004 bp, synthetic construct
  misc_feature 1..1004: note = POT1_Promoter+Yl
  source 1..1004: mol_type = other DNA
ccacaataccccacagtgtgcatatcaaacctaccggttgttgctctctccagccttactaagaaggaggcgacgtggcagtggctcgcgggaggatcggcgggaaactccgggatatccgtcgagagtttacacgtgaatgggcagcgcaatccgttgacgacgatacgactggcaaagtagcgacgatacctgccagacaggtgacatgtgcaggccgcactaacaaggaaacgggcgctggggggggcgggcttctagactttgcccttgaacaggaatctagtgggggcttgtctttccgccaatgggggagcgcctgttgagcgaccgtgcatgctggaacgccaagtgtatgtacagctggtgttctcgcagcggtatgtgacgggacttacatctctcgttttttcatgaccacgttttcacaggctcggaggtacgttaaagttttgaaggctgcatctgaaccgaggtatgggggagtttgaggagcaacagtgttggggctgagggggccaagatcggggcaagcagaggtcttagatcaattgtgggaatcccaaagggctcgttatcacctttttccacccaattcgggtcccaattgatccactactggcttgcccaagttaccccagaaatgccgccccggatttctccaaaaacctaataagcttcatggaacttggtggaagtgactttctacagagtggagagaaccgtggacacgtggcaatggcgctgaccgtgtccccgagccgaatcgacgtgaggggagaacggagtatctgcggtcatgtgaccttccagagcggcgtcgccagtgtgcacgcggtgacccccagtttggttctctgtcacacgcatactacctcggctctccacatgctgaactttatctttcgtggggatcataccgaaagttgcaactaccaggtgtatataaagcctggtagactccccccactttggacctcatccaaccaagacacacaaaaatg

SEQ ID NO: 73 - DNA, 174 bp, Candida viswanathii
  source 1..174: mol_type = other DNA
gagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgac

SEQ ID NO: 74 - DNA, 200 bp, Candida viswanathii
  source 1..200: mol_type = other DNA
agctccaggcttgttatgactctagagagaagtgtgtgtgtttgcgtttgttttactatacattcaacatgttctttttcttttttgatatttattccaactataattatacacagattcgtatatactttactttaccctctttcgtagttttttaatttgatgatttttgagtttcatatccaaggtcaaaacccgac

SEQ ID NO: 75 - DNA, 251 bp, Yarrowia lipolytica
  source 1..251: mol_type = other DNA
taggcaattaacagatagtttgccggtgataattctcttaacctcccacactcctttgacataacgatttatgtaacgaaactgaaatttgaccagatattgttgtaaatagaaaatctggcttgtaggtggcaaaatgcggcgtctttgttcatcaattccctctgtgactactcgtcatccctttatgttcgactgtcgtatttcttattttccatacatatgcaagtgagatgcccgtgtccgaattc

SEQ ID NO: 76 - DNA, 308 bp, synthetic construct
  misc_feature 1..308: note = TDH3_terminator_YL
  source 1..308: mol_type = other DNA
cttccgagtagctatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatatta

SEQ ID NO: 77 - DNA, 2133 bp, synthetic construct
  misc_feature 1..2133: note = LEU2_Marker_Yl
  source 1..2133: mol_type = other DNA
aacgtaccactgtcctccactacaaacacacccaatctgcttcttctagtcaaggttgctacaccggtaaattataaatcatcatttcattagcagggctgggccctttttatagagtcttatacactagcggaccctgccggtagaccaacccgcaggcgcgtcagtttgctccttccatcaatgcgtcgtagaaacgacttactccttcttgagcagctccttgaccttgttggcaacaaagtctccgacctcggaggtggaggaggagcctccgatatcggcggtagtgataccagcctcgacggactccttgacggcagcctcaacagcgtcaccggcgggcttcatgttaagagagaacttgagcatcatggcggcagacagaatggtggcaatggggttgaccttctgcttgccgagatcgggggcagatccgtgacagggctcgtacagaccgaacgcctcgttggtgtcgggcagagaagccagagaggcggagggcagcagacccagagaaccggggatgacggaggcctcgtcggagatgatatcgccaaacatgttggtggtgatgatgataccattcatcttggagggctgcttgatgaggatcatggcggccgagtcgatcagctggtggttgagctccagctgggggaattcgtccttgaggactcgggtgacggtctttcgccaaagtcgagaggaggccagcacgttggccttgtcaagggaccacacgggaagaggggggttgtgctgaagggccaggaaggcggccattcgggcaattcgctcaacctcaggaacggagtaagtctcagtgtcggaagcgacgccagatccgtcatcctcctttcgctctccaaagtagatacctccgacgagctctcggacaatgatgaagtcggtgccctcaacgtttcggatgggggagagatcggcgagcttgggcgacagcagctggcagggtcgcaggttggcgtacaggttcaggtcctttcgcagcttgagaagaccctgctcgggtcgcacgtcggttcgtccgtcgggagtggtccatacggtgttggcagcgcctccgacagcaccgagcataatagagtcagcctttcggcagatgtcgagagtagcgtcggtgatgggctcgccctccttctcaatggcagctcctccaatgagtcggtcctcaaacacaaactcggtgccggaggcctcagcaacagacttgagcaccttgacggcctcggcaatcacctcggggccacagaagtcgccgccgagaagaacaatcttcttggagtcagtcttggtcttcttagtttcgggttccattgtggatgtgtgtggttgtatgtgtgatgtggtgtgtggagtgaaaatctgtggctggcaaacgctcttgtatatatacgcacttttgcccgtgctatgtggaagactaaacctccgaagattgtgactcaggtagtgcggtatcggctagggacccaaaccttgtcgatgccgatagcgctatcgaacgtaccccagccggccgggagtatgtcggaggggacatacgagatcgtcaagggtttgtggccaactggtaaataaatgatgactcaggcgacgacggaattcgacagcaactactcctttcaccaaccatgtgcattttagctcgaataacattcacaggcttggtgatctacatccatggtgtctggccgattaccgtggtgttttggcagtaacgagaatattgagtgaactcttcccatcaccaataaagactcatactacaatcacgagcgcttcagctgccactatagtgttggtgacacaatacccctcgatgctgggcattactgtagcaagagatattatttcatggcgcattttccagtctacctgactttttagtgtgatttcttctccacattttatgctcagtgtgaaaagttggagtgcacacttaattatcgccggttttcggaaagtactatgtgctcaaggttgcaccccacgttacgtatgcagcacattgagcagcctttggaccgtggagataacggtgtggagatagcaacgggtagtcttcgtattaattcaatgcattgttagttttatatgatatggtgtcga

SEQ ID NO: 78 - DNA, 1142 bp, synthetic construct
  misc_feature 1..1142: note = URA3_Marker_Yl
  source 1..1142: mol_type = other DNA
tttctaatttggaccgatagccgtatagtccagtctatctataagttcaactaactcgtaactattaccataacatatacttcactgccccagataaggttccgataaaaagttgtgcagactaaatttatttcagtctcctcttcaccaccaaaatgccctcctacgaagcgcgagctaacgtccacaagtccgcctttgccgcccgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggaactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggatgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagatgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtcatttaactgtgtatatagagagcgtgcaagtatggagcgcttgttcagcttgtatgatggtcagacgacctgtctgatcgagtatgtatgatactgcacaacctg

SEQ ID NO: 79 - protein, 9 aa, synthetic construct
  REGION 1..9: note = FLAG
DYKDDDDKG

SEQ ID NO: 80 - protein, 14 aa, paramyxovirus simian parainfluenza virus 5
GKPIPNPLLGLDST

SEQ ID NO: 81 - protein, 10 aa, Homo sapiens
EQKLISEEDL

SEQ ID NO: 82 - protein, 11 aa, Herpes simplex virus
QPELAPEDPED

SEQ ID NO: 83 - protein, 9 aa, Vesicular stomatitis virus
YPYDVPDYA

SEQ ID NO: 84 - protein, 261 aa, Influenza
MTKFIGCIDLHNGEVKQIVGGTLTSKKEDVPKTNFVSQHPSSYYAKLYKDRDVQGCHVIKLGPNNDDAAREALQESPQFLQVGGGINDTNCLEWLKWASKVIVTSWLFTKEGHFQLKRLERLTELCGKDRIVVDLSCRKTQDGRWIVAMNKWQTLTDLELNADTFRELRKYTNEFLIHAADVEGLCGGIDELLVSKLFEWTKDYDDLKIVYAGGAKSVDDLKLVDELSHGKVDLTFGSSLDIFGGNLVKFEDCCRWNEKQG

SEQ ID NO: 85 - protein, 5 aa, synthetic construct
  VARIANT 3: note = Xaa = Any Amino Acid
  REGION 1..5: note = Cysteine-rich tag
CCXCC

SEQ ID NO: 86 - protein, 6 aa, synthetic construct
  REGION 1..6: note = VSV-G
CCPGCC

SEQ ID NO: 87 - protein, 6 aa, synthetic construct
  REGION 1..6: note = polyhistidine element
CCPGCC

SEQ ID NO: 88 - protein, 261 aa, Saccharomyces cerevisiae; identical to SEQ ID NO: 84

SEQ ID NO: 89 - protein, 6 aa, Cricetulus griseus
LVPRGS

SEQ ID NO: 90 - protein, 5 aa, synthetic construct
  REGION 1..5: note = enterokinase
DDDDK

SEQ ID NO: 91 - protein, 7 aa, synthetic construct
  REGION 1..7: note = TEV protease
ENLYFQG

SEQ ID NO: 92 - protein, 8 aa, synthetic construct
  REGION 1..8: note = PreScission protease
LEVLFQGP

SEQ ID NO: 93 - DNA, 2130 bp, synthetic construct
  misc_feature 1..2130: note = CvACO1P Gene
  source 1..2130: mol_type = other DNA
atgacagaagtagtagatagagcaagttccccagcaagtccaggatctacgaccgccgccgcagacggtgctaaggtggcggtggagccacgcgtagatgtagcggcccttggcgagcagttgctagggcgatgggctgacatcagattgcacgcacgagacttagcaggccgcgaagtggtccaaaaggttgaaggacttacgcacactgagcatcggagtagagtctttggacagttgaagtacttggtagacaacaatgctgttcacagagcttttccctccaggctaggtggatcagatgaccatggcggtaatatagctggattcgaggaattagttactgctgatccatcattgcaaataaaggccggcgttcagtggggtttgtttggttctgcagtgatgcacttgggaacccgtgaacatcatgacaagtggttgccaggtattatgtcgttagaaataccggggtgtttcgcgatgacagaaaccgggcacggtagcgacgtggcctctattgctacaacagcaacttatgatgaggaaacccaagagtttgttattgataccccgttcagagccgcttggaaagattatatcggtaatgcagcgaacgatggtttggcggcagttgttttcgcacaattaatcacgaggaaagtgaaccatggtgtacacgccttttacgtggatctcagagatcctgcgactggagacttcctacccggaataggaggagaggacgatggaatcaaggggggattgaatggcattgacaacggtagactacattttacgaacgtacgcattcctagaactaatcttcttaacagatatggcgatgtggcggtcgacggcacatacctgtcgaccatcgaatcaccagggcgccggttctttacgatgcttggtactctagtccagggtagagttagtctcgatggagcagctgtcgctgcactgaaggtcgcattgcaaagtgcaattcactacgctgcggagaggagacaatttaatgcgacttcacctactgaagaagaggtccttcttgattatcagaggcatcaaaggagactctttacacgacttgcaacgacgtacgccgcatctttcgcccacgagcagctattgcaaaagttcgatgatgtcttttcaggggcacatgataccgacgccgaccggcaggacttggaaaccctagccgccgctttgaagccattgagcacatggcatgcacttgacacgttacaagaatgcagagaggcctgtgggggggccggatttttgatagaaaaccgtttcgcgagcttgcgtgctgacttggacgtttacgtcacattcgagggtgataacacagttttattgcaattggttgctaaacggctcttggcagactacgcaaaagagttcagaggggccaacttcggcgttcttgccaggtatgtggttgaccaagccgcgggagtggcgctccaccgaacaggactaaggcaagtcgctcaatttgttgcagacagcgggtccgttcagaagtcggctcttgcgcttcgcgatgaagagggtcaacgaacattgttaacggacagagtacagagcatggttgccgaagtgggggctgccttgaaaggcgcaggcaaattaccccaacatcaagcagctgcattgttcaaccaacaccagaacgaacttattgaggctgcccaggcccatgcagaactcctccaatgggaggcatttacagaagctctcgctaaagtcgacgatgctggtacaaaggaagtgcttactcgattgcgagatctctttggtttgtccttgattgaaaaacacttgctgtggtatcttatgaatggacgtttgtccatgcaaagaggcaggacagttggaacttacattaatcgtttacttgtcaagatccgtccacacgcactagacttggttgatgccttcggttacggcgcggagcatttgcgtgctgctatcgccaccggagcggaagcaacccgacaggatgaagcccgaacgtattttagacaacaacgggcatcgggactggccccggccgatgaaaagaccttactcgctatcaaagctggtaaatcaagagggcgaagggcaaagctatga

SEQ ID NO: 94 - DNA, 2163 bp, synthetic construct
  misc_feature 1..2163: note = CvHXS1 Gene
  source 1..2163: mol_type = other DNA
atgggaaaaaattataaatctttggactcagttgtggctagtgacttcattgcacttgggatcacatcagaagttgctgagacattgcacggacgcttggcagagatagtttgcaactacggcgccgcaacacctcagacctggattaacatcgcaaaccatattctaagtccagatcttccatttagtctccatcagatgttgttctacggttgttataaggactttggtccagcacccccagcttggataccagaccccgaaaaagtaaagtccacgaacttaggtgccttgttagaaaagcggggaaaggagtttctaggcgttaagtataaggacccaataagtctgttttctcacttccaggagtttagcgttcgaaatccggaagtctactggcggacggtacttatggatgaaatgaagatactgttcagcaaagatcccgaatgtatcctcagacgcgacgacattaacaacccagggggctctgagtggctaccaggtggatatctcaacctggccaagaactgtttgaatgtaaatagtaacaaaaaacttaacgacactatgatagtgtggagagatgaaggaaatgacgatctcccattgaataaattgactcttgatcaattacgaaaacgagtctggttggttggatacgccctagaagagatgggccttgagaagggatgtgcgattgcaattgacatgcccatgcacgtagatgcggttgtgatctatttagctatcgtcttggcaggctacgtcgttgtctccattgcagattcattctcagcaccggaaatttccacaagattgcgtctatcaaaggctaaggctatttttacacaagatcatatcatccgagggaaaaagcgtatacctttgtacctgcgtgtcgtcgaggccaagtctccgatggcaatagttatcccgtgttcgggttcaaatattggtgcggaattgcgggatggtgatattctgtgggattacttcttagaacgcgcaaaggaatttaagaactgcgaatttacagcccgtgaacagccagtggacgcgtacacaaatattttgttctcatcgggaaccaccggagagccaaaggcgataccatggactcaagctacgcctctcaaggcggctgctgatggttggtcacacttggacattagaaagggtgacgtaattgtatggcctaccaatttggggtggatgatggggccttggttggtctatgcttcactccttaacggggcaagcatcgcattgtataacggatctccactagtgtccggctttgccaaattcgttcaagatgcgaaagttactatgctaggagttgtcccctccatcgtacgaagctggaaaagcactaattgcgttagtgggtacgattggtctacaatcagatgcttctcctcatcgggtgaggcatcgaatgtcgatgaatacttatggctaatgggaagggctaactacaaaccggtcatcgaaatgtgcggtggcacagagatcgggggtgccttcagcgccggttcgtttttacaagcccaatctttgagtagcttctcatcccaatgtatgggatgcaccttgtacattctcgacaagaatggctacccgatgccaaagaacaagccgggtataggtgaattggccttgggacccgtgatgttcggtgcttccaagactttacttaacggaaaccatcatgacgtttatttcaaaggcatgcccaccttgaacggagaagtcttgaggagacacggagatatcttcgaactcacttcgaacggctattatcacgctcatggtagagcagatgacacgatgaatatcggggggattaaaatttcctcaatcgagattgaaagggtgtgtaatgaagttgacgatagagtgtttgagactacggccattggagtgcctccattgggcggaggtccagagcagctcgttatcttttttgttcttaaggacagcaatgatacgaccatcgacctaaaccaattgcgacttagttttaatcttgggttacaaaagaaattgaacccactttttaaggtgacgagggttgtgccactttcgctgttgcctaggacagccaccaacaaaataatgagaagagtgcttagacagcaatttagtcatttcgagtga

SEQ ID NO: 95 - DNA, 1158 bp, synthetic construct
  misc_feature 1..1158: note = CvTKS1 Gene
  source 1..1158: mol_type = other DNA
atgaatcatttaagagcagaaggacccgcatcagtgttagcgataggtacagctaacccagagaatatcttaatccaagatgaatttcctgactactatttccgtgttactaaatcggaacatatgactcaacttaaagagaagttccggaaaatctgcgataaatccatgatccgaaagagaaactgtttccttaacgaagaacatctcaagcaaaacccgaggttggtagagcacgaaatgcagaccttggatgctaggcaggacatgttggtggtcgaagtgccaaaactcggcaaggacgcgtgcgctaaggcaatcaaggagtggggtcaaccgaagtctaaaatcacgcatctaatatttacatctgcactgacaaccgacatgccgggtgccgattatcactgcgccaagctacttggattgagtccactggttaagagagttatgatgtatcaattggggtgttacggagggggcacagtcctcagaattgctaaggatattgcggaaaataacaagggcgcgagggtccttgctgtatgttgtgatattatggcctgtttgtttcgcgggccctcggattcagatttggaattgcttgtcggacaggcaatttttggtgacggggccgcagcagtcatagtgggagccgaaccagacgaaagcgtgggtgaaagaccaatctttgagttggttctgaccggacaaacgatcttacctaactcggaaggtacgattggaggacatattagagaagccggcctaattttcgatcttcacaaagacgttccaatgttaatctccaataacatagaaaagtgcttgatagaagcatttactcccattggtattagtgactggaacagcattttctggatcacccaccctggaggaaaagctatactcgataaggttgaagagaaactcgacttgaaaaaggagaaattcgttgactcacgacatgtgttatcagagcacgggaatatgagttcatccacagtcttgttcgtaatggatgaattgcgaaaacgctctcttgaggagggaaagagcacaaccggtgacgggtttgagtggggcgtgctattcggttttggcccaggtttgactgtcgagcgggttgttgttcgtagtgtaccaattaagtactga

SEQ ID NO: 96 - DNA, 1176 bp, synthetic construct
  misc_feature 1..1176: note = CvTKS1P Gene
  source 1..1176: mol_type = other DNA
atgaatcatttaagagcagaaggacccgcatcagtgttagcgataggtacagctaacccagagaatatcttaatccaagatgaatttcctgactactatttccgtgttactaaatcggaacatatgactcaacttaaagagaagttccggaaaatctgcgataaatccatgatccgaaagagaaactgtttccttaacgaagaacatctcaagcaaaacccgaggttggtagagcacgaaatgcagaccttggatgctaggcaggacatgttggtggtcgaagtgccaaaactcggcaaggacgcgtgcgctaaggcaatcaaggagtggggtcaaccgaagtctaaaatcacgcatctaatatttacatctgcactgacaaccgacatgccgggtgccgattatcactgcgccaagctacttggattgagtccactggttaagagagttatgatgtatcaattggggtgttacggagggggcacagtcctcagaattgctaaggatattgcggaaaataacaagggcgcgagggtccttgctgtatgttgtgatattatggcctgtttgtttcgcgggccctcggattcagatttggaattgcttgtcggacaggcaatttttggtgacggggccgcagcagtcatagtgggagccgaaccagacgaaagcgtgggtgaaagaccaatctttgagttggttctgaccggacaaacgatcttacctaactcggaaggtacgattggaggacatattagagaagccggcctaattttcgatcttcacaaagacgttccaatgttaatctccaataacatagaaaagtgcttgatagaagcatttactcccattggtattagtgactggaacagcattttctggatcacccaccctggaggaaaagctatactcgataaggttgaagagaaactcgacttgaaaaaggagaaattcgttgactcacgacatgtgttatcagagcacgggaatatgagttcatccacagtcttgttcgtaatggatgaattgcgaaaacgctctcttgaggagggaaagagcacaaccggtgacgggtttgagtggggcgtgctattcggttttggcccaggtttgactgtcgagcgggttgttgttcgtagtgtaccaattaagtacggaagaagggcaaagttgtga

SEQ ID NO: 97 - DNA, 318 bp, synthetic construct
  misc_feature 1..318: note = CvOAC1 Gene
  source 1..318: mol_type = other DNA
atggcagtcaaacacctaatagttctcaaatttaaagacgagattactgaagctcagaaggaagagttctttaagacatatgttaacttagtcaacatcatccccgcgatgaaggacgtctactggggcaaggatgtgacgcaaaaaaataaggaagaaggatacacacatatcgttgaggtgacctttgagagtgtggaaactattcaagattatattattcacccagcccatgtagggttcggtgacgtttatcgatcattctgggaaaagttgcttatatttgattacaccccaagaaaattgaagcctaagtga

SEQ ID NO: 98 - DNA, 336 bp, synthetic construct
  misc_feature 1..336: note = CvOACP1 Gene
  source 1..336: mol_type = other DNA
atggcagtcaaacacctaatagttctcaaatttaaagacgagattactgaagctcagaaggaagagttctttaagacatatgttaacttagtcaacatcatccccgcgatgaaggacgtctactggggcaaggatgtgacgcaaaaaaataaggaagaaggatacacacatatcgttgaggtgacctttgagagtgtggaaactattcaagattatattattcacccagcccatgtagggttcggtgacgtttatcgatcattctgggaaaagttgcttatatttgattacaccccaagaaaattgaagcctaagggaagacgagctaagttgtga

SEQ ID NO: 99 - DNA, 1188 bp, synthetic construct
  misc_feature 1..1188: note = CvPTS1 Gene
  source 1..1188: mol_type = other DNA
atgggtttatcgtcagtgtgcactttttcttttcaaacaaactaccacaccctcctaaaccctcacaataataacccaaaaacctccttgctatgttacagacatccaaagacaccgatcaagtattcatacaacaattttcccagtaaacattgctcaacgaagtccttccacttgcaaaacaaatgcagcgaatcattgtcgatagctaaaaactcgatacgtgcggcaaccactaaccaaactgagccaccagagagcgataatcattcagtcgccaccaagattttgaactttggaaaagcctgttggaaacttcaaaggccttacaccattatcgcatttaccagttgcgcatgtggtttgttcgggaaggaattattacacaacacaaatttgatcagctggagcctaatgtttaaggcatttttcttcttagttgcaattttgtgtatagcttcgtttacaacgaccattaatcagatttacgaccttcacatcgatcggatcaataaaccagacttgccccttgcctctggggaaatctctgtaaatactgcatggatcatgctgataatcgtggctttgtttggattgattattacaattaagatgaaggggggtccattatatatattcgggtactgcttcggcattttcggtggtatcgtttactccgtcccaccctttagatggaaacagaaccccagtacggcctttctactcaatttcttggctcatatcatcacaaacttcacattctattatgcaagccgagcggcgcttggtttgccgttcgaactcagaccgagttttacatttctccttgccttcatgaaactgatgggactggcccttgcattgatcaaggatgcgtcagatgtcgaaggcgacactaagttcggcattctgacgcttgcttccaagtatggaagtagaaatctaacgcttttttgttcaggaatagtgctacttagttatgttgctgctatactcgctggcattatttggcctcaggccttcaactctaacgtaatgttgttatcccatgctattttggcgttctggttgatcttgcaaacgcgagattttgcactcactaactacgacccagaggcaggaaggcgcttttacgagtttatgtggaagttgtattatgccgaatacttggtttatgttttcatttga

SEQ ID NO: 100 - DNA, 966 bp, synthetic construct
  misc_feature 1..966: note = CVPTS1dN Gene
  source 1..966: mol_type = other DNA
atggcggcaaccactaaccaaactgagccaccagagagcgataatcattcagtcgccaccaagattttgaactttggaaaagcctgttggaaacttcaaaggccttacaccattatcgcatttaccagttgcgcatgtggtttgttcgggaaggaattattacacaacacaaatttgatcagctggagcctaatgtttaaggcatttttcttcttagttgcaattttgtgtatagcttcgtttacaacgaccattaatcagatttacgaccttcacatcgatcggatcaataaaccagacttgccccttgcctctggggaaatctctgtaaatactgcatggatcatgctgataatcgtggctttgtttggattgattattacaattaagatgaaggggggtccattatatatattcgggtactgcttcggcattttcggtggtatcgtttactccgtcccaccctttagatggaaacagaaccccagtacggcctttctactcaatttcttggctcatatcatcacaaacttcacattctattatgcaagccgagcggcgcttggtttgccgttcgaactcagaccgagttttacatttctccttgccttcatgaaactgatgggactggcccttgcattgatcaaggatgcgtcagatgtcgaaggcgacactaagttcggcattctgacgcttgcttccaagtatggaagtagaaatctaacgcttttttgttcaggaatagtgctacttagttatgttgctgctatactcgctggcattatttggcctcaggccttcaactctaacgtaatgttgttatcccatgctattttggcgttctggttgatcttgcaaacgcgagattttgcactcactaactacgacccagaggcaggaaggcgcttttacgagtttatgtggaagttgtattatgccgaatacttggtttatgttttcatttga

SEQ ID NO: 101 - DNA, 1197 bp, synthetic construct
  misc_feature 1..1197: note = CvPTS2 Gene
  source 1..1197: mol_type = other DNA
atggggttgtccttagtttgtacgttcagtttccaaactaactaccacacactactaaatccgcacaacaaaaacccgaaaaattcattgctctcctatcagcacccaaaaacacccattatcaagtctagttacgacaactttccatcaaaatactgtctaacgaaaaactttcatttgttgggcttaaattctcataatcgtatttccagtcagtcccgatcgatcagggccgggagtgaccaaattgaaggttctccacatcatgaaagtgacaattcaattgctacgaagattttaaactttgggcatacatgctggaagctacagcgaccgtatgtagttaaggggatgatcagcattgcctgcggcctattcggaagggaactcttcaataatagacatcttttttcttggggtttaatgtggaaagcttttttcgctttggttcctatccttagttttaacttcttcgccgctattatgaatcaaatttacgatgttgacatcgaccgtattaacaaacccgatctcccccttgtttcaggcgagatgtccattgaaacggcatggattttgtccatcattgttgcgcttactggcttgattgttaccattaagcttaaaagcgctcccttgttcgtttttatatacattttcggcatttttgccggattcgcatacagtgtcccgcctatacgttggaaacaatatccattcacgaacttcttgatcacgatctcatcacatgttggattggcctttacgtcctacagtgctaccacatctgcccttggattgcctttcgtttggaggcctgccttctcgtttatcattgcatttatgacagtgatgggaatgactatcgcatttgctaaagatatcagcgacatagagggcgatgcaaaatatggggtgagtactgttgcgacgaagttgggcgcccgaaatatgaccttcgttgtttccggcgttcttttacttaactatttagtatcgattagcatcgggatcatctggccacaggtgtttaaatcaaatattatgatcttgtcgcatgccatcctagctttctgtcttatatttcaaacaagagaattagccctagcgaactacgcctcagcaccaagtcgtcagttcttcgaatttatatggctactctactacgccgaatacttcgtctatgtcttcatttag

SEQ ID NO: 102 - DNA, 1635 bp, synthetic construct
  misc_feature 1..1635: note = CvCBD1 Gene
  source 1..1635: mol_type = other DNA
atgaagtgttctacgtttagtttttggtttgtttgtaaaattatattcttctttttttccttcaacattcagacatcaatcgccaacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacactga

SEQ ID NO: 103 - DNA, 1722 bp, synthetic construct
  misc_feature 1..1722: note = CBD1dNS1
  source 1..1722: mol_type = other DNA
atgttcttgaaacacatttttgttgctctcgcttttgccttgttagctgacgctaccccagcccagaagagatctcccggcttcgttgctttagactttgacatcgtcaaggttcaaaagaacgtgactgccaacgacgacgccgctgccattgttgccaagagacagaccaacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacactga

SEQ ID NO: 104 - DNA, 1632 bp, synthetic construct
  misc_feature 1..1632: note = CBD1dNS2
  source 1..1632: mol_type = other DNA
atgcaattgtcattgtcagttttgtcaacagttgcaacagcattgttgtcattgacaacagcagttgatgcaaagtcacataacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacactga

SEQ ID NO: 105 - DNA, 1827 bp, synthetic construct
  misc_feature 1..1827: note = CBD1dNV1
  source 1..1827: mol_type = other DNA
atgcaattgtccttgtcggttttatcaaccgttgccacggccttgttgtccctaaccaccgccgtcgatgctaagtcccacaacatcaagttgtccaagttgtccaacgaagaaacattggacgcctccacattccaagaatacacgagctccttggccaacaagtacatgaacttgttcaacgccgctcacggtaacccaaccagctttggcttgcaacacgtcttgtccaaccaagaagctgaagtcccattcgttaccccacaaaagggtggcaacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacactga

SEQ ID NO: 106 - DNA, 1572 bp, synthetic construct
  misc_feature 1..1572: note = CvCBD1dNP1
  source 1..1572: mol_type = other DNA
atgaacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacacggaagaagggcaaagttgtaa

SEQ ID NO: 107 - DNA, 1638 bp, synthetic construct
  misc_feature 1..1638: note = CvTHC1 Gene
  source 1..1638: mol_type = other DNA
atgaattgttcagcatttagtttttggtttgtttgtaagattattttcttctttttgtcatttaacattcaaatttcaattgcaaacccacaagaaaactttttgaagtgtttttcagaatacattccaaacaatccagctaacccaaagtttatttacacacaacatgatcaattgtacatgtcagttttgaactcaacaattcaaaacttgagatttacatcagataccacaccaaagccattggttattgttacaccatcaaacgtttcccatattcaagcatcaatcttgtgttcaaagaaggttggattgcaaattagaaccagatcaggaggacacgatgcagaaggaatgtcatacatttcacaagttccattcgttgttgttgatttgagaaacatgcactcaattaagattgatgttcattcacaaacagcatgggttgaagcaggagcaacattgggtgaagtttactactggattaacgaaaagaacgaaaacttcagttttccaggaggttactgtccaacagttggagttggaggacatttttcaggtggaggatacggagcattgatgagaaactacggattggcagcagataacattattgatgcacacttggttaacgttgatggaaaggttttggatagaaagtcaatgggagaagatttgttttgggcaattagaggaggtggtggagagaactttggaattattgcagcatggaagatcaagttggttgcagttccatcaaagtcaacaatcttttcagttaagaagaacatggaaattcatggtttggttaagttgtttaacaagtggcaaaacattgcatacaagtacgataaggatttggttttgatgacacattttattacaaagaacattacagataaccatggaaagaacaagacaacagttcacggatacttttcatcaatttttcacggaggagttgattcattggttgacttgatgaacaagtcatttccagaattgggaatcaagaagacagattgtaaggaattttcatggattgatacaacaattttctactcaggagttgttaactttaacacagcaaactttaagaaggaaattttgttggacagatcagcaggaaagaagaccgcattttccattaagttggattacgttaagaaaccaattccagaaacagcaatggttaagattttggaaaagttgtacgaagaagatgttggtgttggaatgtacgttttgtacccatacggaggaattatggaagaaatctcagaatcagcaattccatttccacatagagcaggtattatgtacgaattgtggtacacagcatcatgggaaaagcaagaagataatgaaaagcatattaactgggttagatcagtttacaactttacaacaccatacgtttcacaaaacccaagattggcatacttgaactacagagatttggatttgggaaagacaaacccagaatcaccaaacaactatacacaagctagaatttggggagaaaagtactttggtaagaacttcaacagattggttaaagttaagacaaaggcagatccaaataacttctttagaaacgaacaatcaattccaccattgccaccacatcatcattaa

SEQ ID NO: 108 - DNA, 1728 bp, synthetic construct
  misc_feature 1..1728: note = THC1dNS1 Gene
  source 1..1728: mol_type = other DNA
atgttcttgaaacacatttttgttgctctcgcttttgccttgttagctgacgctaccccagcccagaagagatctcccggcttcgttgctttagactttgacatcgtcaaggttcaaaagaacgtgactgccaacgacgacgccgctgccattgttgccaagagacagaccaacccacaagaaaactttttgaagtgtttttcagaatacattccaaacaatccagctaacccaaagtttatttacacacaacatgatcaattgtacatgtcagttttgaactcaacaattcaaaacttgagatttacatcagataccacaccaaagccattggttattgttacaccatcaaacgtttcccatattcaagcatcaatcttgtgttcaaagaaggttggattgcaaattagaaccagatcaggaggacacgatgcagaaggaatgtcatacatttcacaagttccattcgttgttgttgatttgagaaacatgcactcaattaagattgatgttcattcacaaacagcatgggttgaagcaggagcaacattgggtgaagtttactactggattaacgaaaagaacgaaaacttcagttttccaggaggttactgtccaacagttggagttggaggacatttttcaggtggaggatacggagcattgatgagaaactacggattggcagcagataacattattgatgcacacttggttaacgttgatggaaaggttttggatagaaagtcaatgggagaagatttgttttgggcaattagaggaggtggtggagagaactttggaattattgcagcatggaagatcaagttggttgcagttccatcaaagtcaacaatcttttcagttaagaagaacatggaaattcatggtttggttaagttgtttaacaagtggcaaaacattgcatacaagtacgataaggatttggttttgatgacacattttattacaaagaacattacagataaccatggaaagaacaagacaacagttcacggatacttttcatcaatttttcacggaggagttgattcattggttgacttgatgaacaagtcatttccagaattgggaatcaagaagacagattgtaaggaattttcatggattgatacaacaattttctactcaggagttgttaactttaacacagcaaactttaagaaggaaattttgttggacagatcagcaggaaagaagaccgcattttccattaagttggattacgttaagaaaccaattccagaaacagcaatggttaagattttggaaaagttgtacgaagaagatgttggtgttggaatgtacgttttgtacccatacggaggaattatggaagaaatctcagaatcagcaattccatttccacatagagcaggtattatgtacgaattgtggtacacagcatcatgggaaaagcaagaagataatgaaaagcatattaactgggttagatcagtttacaactttacaacaccatacgtttcacaaaacccaagattggcatacttgaactacagagatttggatttgggaaagacaaacccagaatcaccaaacaactatacacaagctagaatttggggagaaaagtactttggtaagaacttcaacagattggttaaagttaagacaaaggcagatccaaataacttctttagaaacgaacaatcaattccaccattgccaccacatcatcattaataa

SEQ ID NO: 109 - DNA, 1638 bp, synthetic construct
  misc_feature 1..1638: note = THC1dNS2
  source 1..1638: mol_type = other DNA
atgcaattgtcattgtcagttttgtcaacagttgcaacagcattgttgtcattgacaacagcagttgatgcaaagtcacataacccacaagaaaactttttgaagtgtttttcagaatacattccaaacaatccagctaacccaaagtttatttacacacaacatgatcaattgtacatgtcagttttgaactcaacaattcaaaacttgagatttacatcagataccacaccaaagccattggttattgttacaccatcaaacgtttcccatattcaagcatcaatcttgtgttcaaagaaggttggattgcaaattagaaccagatcaggaggacacgatgcagaaggaatgtcatacatttcacaagttccattcgttgttgttgatttgagaaacatgcactcaattaagattgatgttcattcacaaacagcatgggttgaagcaggagcaacattgggtgaagtttactactggattaacgaaaagaacgaaaacttcagttttccaggaggttactgtccaacagttggagttggaggacatttttcaggtggaggatacggagcattgatgagaaactacggattggcagcagataacattattgatgcacacttggttaacgttgatggaaaggttttggatagaaagtcaatgggagaagatttgttttgggcaattagaggaggtggtggagagaactttggaattattgcagcatggaagatcaagttggttgcagttccatcaaagtcaacaatcttttcagttaagaagaacatggaaattcatggtttggttaagttgtttaacaagtggcaaaacattgcatacaagtacgataaggatttggttttgatgacacattttattacaaagaacattacagataaccatggaaagaacaagacaacagttcacggatacttttcatcaatttttcacggaggagttgattcattggttgacttgatgaacaagtcatttccagaattgggaatcaagaagacagattgtaaggaattttcatggattgatacaacaattttctactcaggagttgttaactttaacacagcaaactttaagaaggaaattttgttggacagatcagcaggaaagaagaccgcattttccattaagttggattacgttaagaaaccaattccagaaacagcaatggttaagattttggaaaagttgtacgaagaagatgttggtgttggaatgtacgttttgtacccatacggaggaattatggaagaaatctcagaatcagcaattccatttccacatagagcaggtattatgtacgaattgtggtacacagcatcatgggaaaagcaagaagataatgaaaagcatattaactgggttagatcagtttacaactttacaacaccatacgtttcacaaaacccaagattggcatacttgaactacagagatttggatttgggaaagacaaacccagaatcaccaaacaactatacacaagctagaatttggggagaaaagtactttggtaagaacttcaacagattggttaaagttaagacaaaggcagatccaaataacttctttagaaacgaacaatcaattccaccattgccaccacatcatcattaataa

SEQ ID NO: 110 - DNA, 1833 bp, synthetic construct
  misc_feature 1..1833: note = THC1dNV1
  source 1..1833: mol_type = other DNA
atgcaattgtccttgtcggttttatcaaccgttgccacggccttgttgtccctaaccaccgccgtcgatgctaagtcccacaacatcaagttgtccaagttgtccaacgaagaaacattggacgcctccacattccaagaatacacgagctccttggccaacaagtacatgaacttgttcaacgccgctcacggtaacccaaccagctttggcttgcaacacgtcttgtccaaccaagaagctgaagtcccattcgttaccccacaaaagggtggcaacccacaagaaaactttttgaagtgtttttcagaatacattccaaacaatccagctaacccaaagtttatttacacacaacatgatcaattgtacatgtcagttttgaactcaacaattcaaaacttgagatttacatcagataccacaccaaagccattggttattgttacaccatcaaacgtttcccatattcaagcatcaatcttgtgttcaaagaaggttggattgcaaattagaaccagatcaggaggacacgatgcagaaggaatgtcatacatttcacaagttccattcgttgttgttgatttgagaaacatgcactcaattaagattgatgttcattcacaaacagcatgggttgaagcaggagcaacattgggtgaagtttactactggattaacgaaaagaacgaaaacttcagttttccaggaggttactgtccaacagttggagttggaggacatttttcaggtggaggatacggagcattgatgagaaactacggattggcagcagataacattattgatgcacacttggttaacgttgatggaaaggttttggatagaaagtcaatgggagaagatttgttttgggcaattagaggaggtggtggagagaactttggaattattgcagcatggaagatcaagttggttgcagttccatcaaagtcaacaatcttttcagttaagaagaacatggaaattcatggtttggttaagttgtttaacaagtggcaaaacattgcatacaagtacgataaggatttggttttgatgacacattttattacaaagaacattacagataaccatggaaagaacaagacaacagttcacggatacttttcatcaatttttcacggaggagttgattcattggttgacttgatgaacaagtcatttccagaattgggaatcaagaagacagattgtaaggaattttcatggattgatacaacaattttctactcaggagttgttaactttaacacagcaaactttaagaaggaaattttgttggacagatcagcaggaaagaagaccgcattttccattaagttggattacgttaagaaaccaattccagaaacagcaatggttaagattttggaaaagttgtacgaagaagatgttggtgttggaatgtacgttttgtacccatacggaggaattatggaagaaatctcagaatcagcaattccatttccacatagagcaggtattatgtacgaattgtggtacacagcatcatgggaaaagcaagaagataatgaaaagcatattaactgggttagatcagtttacaactttacaacaccatacgtttcacaaaacccaagattggcatacttgaactacagagatttggatttgggaaagacaaacccagaatcaccaaacaactatacacaagctagaatttggggagaaaagtactttggtaagaacttcaacagattggttaaagttaagacaaaggcagatccaaataacttctttagaaacgaacaatcaattccaccattgccaccacatcatcattaataa

SEQ ID NO: 111 - DNA, 1638 bp, synthetic construct
  misc_feature 1..1638: note = CvCBC1 Gene
  source 1..1638: mol_type = other DNA
atgaattgtagcactttctcattctggtttgtttgtaagattattttctttttcttgtcatttaacattcaaatttcaattgcaaacccacaagagaactttttgaagtgtttctcagaatacattccaaacaacccagctaacccaaagtttatttacacccaacacgatcaattgtacatgtcagttttgaactcaacaattcaaaacttgagatttacatcagatacaacaccaaagccattggttattgttacaccatcaaacgttagtcatattcaagcatcaatcttgtgttcaaagaaggttggattgcaaattagaactagatcaggaggacatgatgcagaaggattgtcatacatttcacaagttccatttgcaattgttgatttgagaaacatgcacacagttaaggttgatattcattcacaaacagcatgggttgaagcaggagcaacattgggtgaagtttactactggattaacgaaatgaacgaaaacttctcatttccaggaggatactgtccaacagttggtgttggaggacacttttcaggtggtggatacggagcattgatgagaaactacggattggcagcagataacattattgatgcacatttggttaacgttgatggaaaggttttggatagaaagtcaatgggagaagatttgttttgggcaattagaggaggtggaggagaaaactttggaatcattgcagcatgtaagatcaagttggttgttgttccatcaaaggcaacaatcttttcagttaagaagaacatggaaatccatggattggttaagttgtttaacaagtggcaaaacattgcatacaagtacgataaggatttgatgttgacaacacattttagaacaagaaacattacagataaccacggaaagaataagacaacagttcatggatacttttcatcaattttcttgggaggagttgattcattggttgacttgatgaacaagagttttccagaattgggaatcaagaagacagattgtaaggaattgtcatggatcgatacaaccattttctactcaggagttgttaactacaacacagctaactttaagaaggaaattttgttggacagatcagcaggtaaaaagacagcattttcaattaagttggattacgttaagaaattgattccagaaacagcaatggttaagattttggaaaagttgtacgaagaagaagttggagttggaatgtacgttttgtacccatacggaggaattatggatgaaatttcagaatcagcaattccatttccacatagagcaggtattatgtacgaattgtggtacacagcaacatgggaaaagcaagaagataacgaaaagcatattaactgggttagatcagtttacaactttacaaccccatacgtttcacaaaacccaagattggcatacttgaactacagagatttggatttgggaaagacaaacccagaatcaccaaataactacacacaagctagaatttggggagaaaagtactttggtaagaactttaacagattggtgaaggttaagacaaaggcagacccaaacaatttctttagaaacgaacaatcaattccaccattgccaccaagacatcattaa

SEQ ID NO: 112 - DNA, 2130 bp, synthetic construct
  misc_feature 1..2130: note = YlACO1P Gene
  source 1..2130: mol_type = other DNA
atgaccgaagtagttgacagagcctcatcccccgcatcccctggctcaactacggccgccgcagacggtgctaaggtggccgtcgagccccgagtagatgtggctgcgctgggagagcagctgctgggccgatgggctgatatccgtctccacgcccgggaccttgcgggacgagaggtagttcagaaggtggagggtctgactcatacagagcaccgctctcgcgtctttggccagctcaagtacttggtcgataacaacgcagttcaccgagcctttccttctcgactgggtggtagtgacgaccacggcggaaacatcgctggttttgaggagcttgtcacggcggacccctccctccagatcaaggccggcgtccagtggggactgttcggctccgctgttatgcacttgggaactagggagcaccacgacaagtggctcccaggcatcatgtctctggaaatccctggttgctttgccatgactgagactggccatggctccgatgtcgcttccattgctacaacggccacctatgatgaggaaacccaggagttcgttattgacaccccgttccgagccgcctggaaggactacattggaaacgccgctaacgacggtttggccgctgtcgtgtttgcccaactgattactcgaaaggttaaccatggagtgcacgccttctacgtcgatctgagagatcccgccaccggagactttctccctggtattggtggagaggacgacggtattaaggggggactaaacggaattgataacggacgtctccatttcaccaatgttcgcattccccgaaccaacctgcttaaccgttacggcgatgttgccgtcgatggcacctacagctcaaccatcgaatctccggggcgaagattctttacaatgctaggtacgctggtccagggccgagtcagcctggacggtgctgcagtggctgcatcgaaggttgctctgcaatccgccatccactacgccgctgagcgaagacagttcaacgccacttcgcccacagaggaggaggtgctcctggattaccagcgacaccagcggcgcctctttacccgactcgccaccacctacgccgcatcgttcgcccatgagcaactgctgcagaaattcgacgacgtgttctcgggtgctcatgatactgacgccgaccgtcaggaccttgagacactggctgctgctctgaagcccctttctacctggcatgctctcgataccctacaagagtgccgagaagcgtgtgggggtgcaggttttctgattgagaaccgattcgcttctctccgggccgatctcgacgtctacgtgaccttcgaaggagacaacaccgtgcttcttcagttggtggccaagaggctgctcgctgactatgctaaggagttccgaggtgccaacttcggcgtgctcgcgcggtacgtcgtggaccaggctgccggagtcgcgctacaccgaaccggactgcgacaggtcgctcagttcgtggccgacagtggatctgtccagaaatctgctcttgccctccgagacgaagaaggtcagcgaactctgctgaccgacagagtccagtccatggttgcagaggttggcgctgctctcaaaggcgcgggcaagctcccccagcaccaggcggcagcactgttcaatcagcatcaaaacgaactgatcgaggctgcccaggcccacgctgagcttttacagtgggaggcctttactgaggctttggccaaggtggacgacgctggcactaaggaagtgttgacccgattgcgtgacctttttggtctgtcccttatcgagaagcacctcagctggtatctgatgaacggtaggctctcgatgcagagaggccgaacggtcggcacttacattaatcgtcttctcgttaagatccgaccacacgcacttgatctggttgatgccttcggctacggagccgagcaccttcgggccgctatcgccaccggcgctgaggccacccgacaggacgaggcccgaacctacttcagacagcaacgagcctccggtagcgcccctgctgacgagaagacactcctcgctatcaaggccggcaagtctcggggacgacgagccaaactgtaa

SEQ ID NO: 113 - DNA, 2163 bp, synthetic construct
  misc_feature 1..2163: note = YlHXS1 Gene
  source 1..2163: mol_type = other DNA
atgggaaagaattacaaaagtctagattctgtcgttgccagtgacttcatcgcgttaggcattacatccgaggtcgctgagactctgcacggacggcttgccgagattgtgtgcaactacggagccgctacccctcagacttggattaacatcgccaaccacattctgtcgccggacctccccttctctttgcaccagatgttattctacggatgctacaaggattttggccctgcacctcctgcctggattccggaccccgaaaaagtcaagtccaccaacctaggtgccctgctggaaaagcgaggaaaggagttccttggtgtcaagtacaaggaccccatttcttctttttctcatttccaggaattttcggtgcgtaatcctgaggtgtattggcgaactgtgctcatggacgagatgaaaatctccttcagcaaggacccagagtgtatcctgcgacgagacgacattaacaacccaggaggctcggagtggcttcccggcggatacctaaactcagctaagaattgtctcaacgtgaactctaacaagaagttgaacgacaccatgatcgtgtggcgtgacgaaggcaacgacgacctgcccctgaacaagttgactctggaccagctgcgaaagagggtctggttggttggctacgccctcgaagagatgggcttagagaagggttgcgctattgctattgatatgcccatgcacgtcgatgctgtagtgatctaccttgccattgtgttagccggttacgtggtcgtatcgattgccgattcgttctccgctccggagatttccacccgactcagacttagcaaggccaaggcaatctttactcaagatcacatcatccgaggtaagaagagaatccctctctattctcgcgtggttgaggccaagtccccaatggctatcgtcataccttgcagcggatcaaacatcggggctgagctacgggacggtgatatctcctgggattacttcctggagcgagccaaagagttcaagaactgcgagtttacagcgcgtgagcagcccgtcgatgcctacacgaacattctattctcatcgggcacaacgggagagcccaaggccatcccctggacccaagctacccccttgaaagctgccgctgatggttggtcccatctcgacatcagaaaaggcgatgtgatcgtttggcccactaacctgggctggatgatgggtccttggctggtatatgccagcctactgaacggcgcttcaatcgcactgtacaacggatctccactcgtcagcggctttgccaagtttgttcaagacgccaaagtcaccatgctgggtgttgttccttcaatcgtgcgaagttggaagagtaccaactgtgtctctggatacgactggagcaccattcgatgcttcagttcctccggcgaggcttccaacgttgatgagtacctctggcttatgggtcgtgcgaattacaagcctgtgatcgagatgtgtggtggaacagaaattggtggtgctttttcggccgggtcctttcttcaggctcagtctctctcctctttctcttcccagtgtatgggatgcaccctgtatattctcgacaagaacggttaccccatgccgaagaataaacccggtattggggagcttgctcttggccccgtcatgtttggtgcatcgaagaccctcctgaacggaaaccatcacgacgtctacttcaagggcatgcccacacttaacggcgaagttcttcggcgacatggagacattttcgaacttacatcgaacggatactaccacgcccacgggcgagcagatgatacgatgaacatcgggggcatcaagatatcttctatcgagattgaaagagtgtgtaacgaggtagacgaccgcgtcttcgagactactgcgatcggcgtcccccccctgggcggtggcccggagcagctagtcattttttttgtgctcaaggactctaacgacacgaccatcgacctcaatcagctgcgactctccttcaaccttggattgcagaagaagctgaaccctctcttcaaggtcactcgggttgttcccttgtcctctcttcctcgaaccgccaccaacaagattatgcgacgagtgctccgacagcagttctcccacttcgagtaa

SEQ ID NO: 114 - DNA, 1158 bp, synthetic construct
  misc_feature 1..1158: note = YlTKS1 Gene
  source 1..1158: mol_type = other DNA
atgaatcatctaagagccgaaggaccagcaagtgtactcgctattggtactgctaaccccgagaacattcttattcaggatgagttcccggactactatttcagggttaccaagagcgagcatatgacccagcttaaagagaagttccgcaagatatgcgacaagtccatgatccgaaagcggaactgctttctcaatgaggagcacttgaagcaaaacccccgactggttgagcacgagatgcagaccttggacgcgcgacaagacatgctcgtcgtcgaggtgcccaaactcggtaaggatgcttgcgctaaggccattaaggagtggggtcagcccaagtcgaagattacccacctaatcttcacctccgcaagcactacagacatgcccggtgcagactaccactgtgccaagctgctcggactgtcaccgtcggtcaagcgagtgatgatgtaccagctcggctgttacgggggtggaaccgttctccgtatcgccaaggatatcgctgagaacaacaaaggagctcgtgtcctggctgtgtgttgcgacatcatggcctgcttgttcagaggccctagtgattccgatctggaattacttgtcggtcaggccatctttggagatggcgccgccgctgtcatcgtgggtgccgaacccgacgagtctgttggagaaagacccatctttgagcttgtctccacgggccagaccatcctccctaacagcgagggcacaattggaggccatattcgagaggccggtctgatttttgacctgcataaggacgtgcctatgctgatttcgaacaacatcgagaagtgtctcatcgaggccttcactcccatcggcatttcggactggaactcaatcttctggatcacccacccaggaggcaaggcgattctggataaagttgaggaaaagctcgaccttaagaaggagaagtttgtggattctcgacacgtcctgtctgaacacggtaacatgtcttcctctactgtcctgttcgtaatggacgagcttcgaaagcgatctctggaggaaggaaagtccacgaccggcgacggttttgagtggggcgtgctgttcgggttcggtcctggcctcactgtggagcgagttgttgtccggtccgtgcctattaagtactaa

SEQ ID NO: 115 - DNA, 1176 bp, synthetic construct
  misc_feature 1..1176: note = YlTKS1P Gene
  source 1..1176: mol_type = other DNA
atgaatcatctaagagccgaaggaccagcaagtgtactcgctattggtactgctaaccccgagaacattcttattcaggatgagttcccggactactatttcagggttaccaagagcgagcatatgacccagcttaaagagaagttccgcaagatatgcgacaagtccatgatccgaaagcggaactgctttctcaatgaggagcacttgaagcaaaacccccgactggttgagcacgagatgcagaccttggacgcgcgacaagacatgctcgtcgtcgaggtgcccaaactcggtaaggatgcttgcgctaaggccattaaggagtggggtcagcccaagtcgaagattacccacctaatcttcacctccgcaagcactacagacatgcccggtgcagactaccactgtgccaagctgctcggactgtcaccgtcggtcaagcgagtgatgatgtaccagctcggctgttacgggggtggaaccgttctccgtatcgccaaggatatcgctgagaacaacaaaggagctcgtgtcctggctgtgtgttgcgacatcatggcctgcttgttcagaggccctagtgattccgatctggaattacttgtcggtcaggccatctttggagatggcgccgccgctgtcatcgtgggtgccgaacccgacgagtctgttggagaaagacccatctttgagcttgtctccacgggccagaccatcctccctaacagcgagggcacaattggaggccatattcgagaggccggtctgatttttgacctgcataaggacgtgcctatgctgatttcgaacaacatcgagaagtgtctcatcgaggccttcactcccatcggcatttcggactggaactcaatcttctggatcacccacccaggaggcaaggcgattctggataaagttgaggaaaagctcgaccttaagaaggagaagtttgtggattctcgacacgtcctgtctgaacacggtaacatgtcttcctctactgtcctgttcgtaatggacgagcttcgaaagcgatctctggaggaaggaaagtccacgaccggcgacggttttgagtggggcgtgctgttcgggttcggtcctggcctcactgtggagcgagttgttgtccggtccgtgcctattaagtacggaagaagggcaaagttgtaa

SEQ ID NO: 116 - DNA, 318 bp, synthetic construct
  misc_feature 1..318: note = YlOAC1 Gene
  source 1..318: mol_type = other DNA
atggccgtcaaacaccttattgtcctcaagttcaaagatgagatcactgaagcccagaaggaggagtttttcaagacctacgtcaatttggtcaacatcattccagcaatgaaggatgtgtactggggcaaggacgtgacccagaagaacaaggaagagggttatacccatatcgttgaggttacgttcgagtctgtggagacaatccaagactacatcattcaccccgctcacgtgggctttggagacgtttacagatccttctgggagaagctcctgatttttgactacactcctcgaaagctgaagcccaagtaa

SEQ ID NO: 117 - DNA, 336 bp, synthetic construct
  misc_feature 1..336: note = YlOAC1P Gene
  source 1..336: mol_type = other DNA
atggccgtcaaacaccttattgtcctcaagttcaaagatgagatcactgaagcccagaaggaggagtttttcaagacctacgtcaatttggtcaacatcattccagcaatgaaggatgtgtactggggcaaggacgtgacccagaagaacaaggaagagggttatacccatatcgttgaggttacgttcgagtctgtggagacaatccaagactacatcattcaccccgctcacgtgggctttggagacgtttacagatccttctgggagaagctcctgatttttgactacactcctcgaaagctgaagcccaagggaagaagggcaaagttgtaa

SEQ ID NO: 118 - DNA, 1197 bp, synthetic construct
  misc_feature 1..1197: note = YlPTS2 Gene
  source 1..1197: mol_type = other DNA
atgggcctctctctagtatgtaccttctctttccagaccaactatcacactctactgaacccccataacaagaaccctaaaaattctcttctcagttaccagcaccccaagacgcctatcattaagtcctcctacgacaactttccctctaagtactgcctgaccaaaaacttccatctcctgggactgaactctcataacagaattagtagccagtcccgatctatccgagctggctctgaccagattgagggctcccctcaccatgaatccgacaacagcatcgctaccaagattttgaattttggtcacacatgctggaagctccagcgaccgtacgtcgtgaagggtatgatctcgattgcctgtggactgttcggacgtgagctttttaataatcgacacttgttttcatggggcctcatgtggaaggcttttttcgccctcgtgcccattctgtctttcaacttctttgccgctattatgaaccaaatctacgacgttgatattgataggatcaacaagcctgacctgccgctcgtctcgggggagatgtctatcgagacagcgtggattctttcgattatcgtcgcgctgactggccttatcgttaccataaagttgaagtctgcacccctcttcgtgtttatctacattttcggtatttttgctggattcgcgtactccgttccccctatcagatggaagcagtacccctttactaactttctgattactatcagcagccacgtcggtttagcctttacctcatattcggccaccaccagtgcactgggcctccccttcgtctggcgacctgcattttcattcatcatcgccttcatgactgtgatgggtatgaccatcgctttcgctaaggacatctccgacatcgagggtgatgctaaatatggagtgtccaccgtggccactaagctgggagcccggaacatgacgttcgtcgtctctggtgttctgctccttaactacttggtttcgatctccattggcattatctggccacaagtcttcaagtccaacattatgattctgtcccacgccattcttgccttttgcctgatcttccagacacgcgaactcgctctcgctaactacgcctccgccccatcgcgacagttcttcgagttcatctggctgctttactacgccgagtacttcgtttacgtgttcatctaa

SEQ ID NO: 119 - DNA, 1635 bp, synthetic construct
  misc_feature 1..1635: note = YlCBD1 Gene
  source 1..1635: mol_type = other DNA
atgaagtgttcgacgttttctttttggtttgtttgtaaaatcattttctttttcttttctttcaacatccaaacgtcgatcgcaaaccctagagagaactttcttaagtgcttctcgcagtacatccctaataacgctaccaaccttaagctggtgtacacccagaacaaccctctttacatgtctgttctaaacagcaccatccacaatcttagattcacatcagacaccactcccaagccgctcgtcatcgtgaccccgagtcatgtgtcccatatccaaggcactatcctgtgctctaaaaaggtcggtctgcagattcggactcgctccggtggacatgattcggagggcatgtcctacattagccaggtcccctttgtgatcgtggacctgaggaacatgcggtctattaagattgatgtgcactcacagaccgcttgggtcgaggctggtgcgacattgggtgaggtgtactactgggtgaacgagaagaacgagaacctgagcctcgccgctggctactgtcccaccgtttgtgccggtggacacttcggcggaggcggatacggtccacttatgcgaaactacgggctcgcagctgataatatcatcgacgcacaccttgttaacgttcacggcaaggtgctggaccgaaaaagcatgggtgaggacctattttgggccttgcgaggcggtggtgccgaatccttcggaattatcgtggcctggaagatccgactggtcgctgtgccaaagtccactatgttctccgtcaagaaaattatggagatccacgaactcgtaaagctcgtcaataagtggcagaacatcgcctacaagtatgacaaggatctgctgctcatgactcacttcatcacgcgaaacattacagacaaccagggaaagaacaagaccgctatccatacctacttctcctctgtcttccttgggggtgtcgattccctcgttgatctcatgaacaaatcttttccagagctcggaatcaagaagaccgactgccgacagctctcttggatcgacaccattattttctactcaggagtcgtaaactacgatactgacaactttaacaaggagattctgttagatcgatcggccggccagaacggtgccttcaagatcaagctcgactatgtcaaaaagcccattcctgaatccgtcttcgttcaaattcttgaaaagttgtacgaggaggatatcggcgccggaatgtacgcgctgtacccctacggtggcattatggacgagatttctgaaagtgctattcccttcccccaccgtgctggcattctgtatgagctgtggtacatttgctcctgggaaaagcaggaggacaacgagaagcacttgaactggatacgaaacatttacaatttcatgaccccctatgtttcgaagaaccctcgactggcctacctgaattaccgcgacctcgacatcggaattaacgaccctaagaaccccaataactatactcaggccagaatctggggcgagaagtacttcggcaagaactttgaccgtctggttaaggtcaagaccctcgtggaccctaacaacttcttccgaaacgagcagtctatcccccctctgccccgacaccggcattaa

SEQ ID NO: 120 - DNA, 1638 bp, synthetic construct
  misc_feature 1..1638: note = YlTHC1 Gene
  source 1..1638: mol_type = other DNA
atgaattgttcagctttctctttttggtttgtctgtaagatcattttcttctttctatcctttcacatccaaatttccatagccaaccctcgtgagaacttccttaagtgcttttccaagcacattccaaataacgtcgccaatcccaagctggtgtacacgcagcatgaccagctctacatgtcgatcctcaattccaccattcaaaaccttagattcattagtgacaccactcccaagcccctagtcattgtcaccccttcgaacaactcgcatattcaggcaactattctctgctccaagaaggttggtttacagatccgaacccggtcaggtggtcacgacgctgagggcatgtcttacatttcccaggtcccctttgtggtggtcgatctgcgcaacatgcactccattaaaatcgacgtccactcgcagactgcctgggtcgaggctggagccacccttggcgaggtctactactggattaacgagaagaacgaaaacctgtcgttccctggcggctactgtccgactgttggagtcggcggacacttttctggtggcggatatggtgctctcatgcgaaactacggactggcggcagacaacatcatcgatgcccaccttgtgaacgttgacggtaaggtactggaccgaaagtctatgggcgaggacttgttttgggccatccgaggtggaggtggtgagaacttcgggatcatcgccgcctggaagatcaagctggtggatgtgcccagtaagtctaccatttttagcgtgaagaagaacatggagatccacgggctggtgaagctgttcaacaagtggcagaatattgcgtacaaatacgacaaggacctggtgcttatgacccatttcatcaccaagaacatcacggataaccacggtaaaaacaagactactgttcacggttacttctcttcaattttccatggtggtgtggattccctcgttgatttgatgaacaagtccttcccagagctgggcattaagaagacagactgcaaggaatttagctggattgataccaccatcttctactctggagttgtcaacttcaacaccgcaaacttcaagaaggaaatcctcttggaccgatctgccggcaagaagacagctttttcgattaaactggattacgtgaagaagcccatccctgagacagctatggtcaagatccttgaaaaactttatgaggaggacgtcggagccggaatgtacgttctctatccttacggcggcatcatggaggaaatttctgagtctgctatccccttcccccatcgagccggaatcatgtacgagctgtggtacaccgctagttgggagaagcaggaggataacgagaaacatatcaattgggtccgtagcgtatacaatttcacgacaccctacgtgtcccagaaccctcgactcgcttacctgaactatagggacctggacctcggcaagactaaccacgctagcccgaacaactacacccaggccagaatttggggcgaaaagtacttcggaaagaacttcaaccgactcgttaaggttaagaccaaagttgaccccaacaactttttccggaacgagcagtccatccctccactccctccccaccatcactga

SEQ ID NO: 121 - DNA, 26 bp, synthetic construct
  misc_feature 1..26: note = CvACT1
  source 1..26: mol_type = other DNA
cccaattcctgtggtgggttgattcg

SEQ ID NO: 122 - DNA, 29 bp, synthetic construct
  misc_feature 1..29: note = CvACT1
  source 1..29: mol_type = other DNA
ctctcaattcgttgtagaaggtgtggtgc

SEQ ID NO: 123 - DNA, 29 bp, synthetic construct
  misc_feature 1..29: note = CvHXS1
  source 1..29: mol_type = other DNA
ctttggactcagttgtggctagtgacttc

SEQ ID NO: 124 - DNA, 34 bp, synthetic construct
  misc_feature 1..34: note = CvHXS1
  source 1..34: mol_type = other DNA
gatcaagagtcaatttattcaatgggagatcgtc

SEQ ID NO: 125 - DNA, 23 bp, synthetic construct
  misc_feature 1..23: note = CvTKS1
  source 1..23: mol_type = other DNA
gagcagaaggacccgcatcagtg

SEQ ID NO: 126 - DNA, 28 bp, synthetic construct
  misc_feature 1..28: note = CvTKS1
  source 1..28: mol_type = other DNA
gtcaccaaaaattgcctgtccgacaagc

SEQ ID NO: 127 - DNA, 31 bp, synthetic construct
  misc_feature 1..31: note = CvOAC1
  source 1..31: mol_type = other DNA
cacgacataatggcagtcaaacacctaatag

SEQ ID NO: 128 - DNA, 31 bp, synthetic construct
  misc_feature 1..31: note = CvOAC1
  source 1..31: mol_type = other DNA
ctagttttgtgttcggagtatgcatacaacg

SEQ ID NO: 129 - DNA, 25 bp, synthetic construct
  misc_feature 1..25: note = CvPTS1
  source 1..25: mol_type = other DNA
ctaccacaccctcctaaaccctcac

SEQ ID NO: 130 - DNA, 31 bp, synthetic construct
  misc_feature 1..31: note = CvPTS1
  source 1..31: mol_type = other DNA
cgaagcagtacccgaatatatataatggacc

SEQ ID NO: 131 - DNA, 29 bp, synthetic construct
  misc_feature 1..29: note = CvCBD1
  source 1..29: mol_type = other DNA
ccttcaacattcagacatcaatcgccaac

SEQ ID NO: 132 - DNA, 22 bp, synthetic construct
  misc_feature 1..22: note = CvCBD1
  source 1..22: mol_type = other DNA
cacccaaggttgcccctgcttc

SEQ ID NO: 133 - DNA, 24 bp, synthetic construct
  misc_feature 1..24: note = CvACO1
  source 1..24: mol_type = other DNA
gagcaagttccccagcaagtccag

SEQ ID NO: 134 - DNA, 26 bp, synthetic construct
  misc_feature 1..26: note = CvACO1
  source 1..26: mol_type = other DNA
catgatgttcacgggttcccaagtgc

SEQ ID NO: 135 - DNA, 25 bp, synthetic construct
  misc_feature 1..25: note = CvPTS2
  source 1..25: mol_type = other DNA
catacatgctggaagctacagcgac

SEQ ID NO: 136 - DNA, 27 bp, synthetic construct
  misc_feature 1..27: note = CvPTS2
  source 1..27: mol_type = other DNA
cagtactcaccccatattttgcatcgc

SEQ ID NO: 137 - DNA, 36 bp, synthetic construct
  misc_feature 1..36: note = CvTHC1
  source 1..36: mol_type = other DNA
gttaagttgtttaacaagtggcaaaacattgcatac

SEQ ID NO: 138 - DNA, 34 bp, synthetic construct
  misc_feature 1..34: note = CvTHC1
  source 1..34: mol_type = other DNA
ccaaaatcttaaccattgctgtttctggaattgg

SEQ ID NO: 139 - DNA, 29 bp, synthetic construct
  misc_feature 1..29: note = Candida viswanathii
  source 1..29: mol_type = other DNA
gatttgttttgggcaattagaggaggtgg

SEQ ID NO: 140 - DNA, 32 bp, synthetic construct
  misc_feature 1..32: note = Candida viswanathii
  source 1..32: mol_type = other DNA
caactcctgagtagaaaatggttgtatcgatc

SEQ ID NO: 141 - DNA, 29 bp, synthetic construct
  misc_feature 1..29: note = Yarrowia lipolytica
  source 1..29: mol_type = other DNA
ttgttaccaactgggatgacatggagaag

SEQ ID NO: 142 - DNA, 28 bp, synthetic construct
  misc_feature 1..28: note = Yarrowia lipolytica
  source 1..28: mol_type = other DNA
ctgatggacgtgtttttcgacatgaacc

SEQ ID NO: 143 - DNA, 34 bp, synthetic construct
  misc_feature 1..34: note = Yarrowia lipolytica
  source 1..34: mol_type = other DNA
gaaaggaacatagtcatttccaaacttgaaagtc

SEQ ID NO: 144 - DNA, 24 bp, synthetic construct
  misc_feature 1..24: note = Yarrowia lipolytica
  source 1..24: mol_type = other DNA
cagacggagtactttcgctcgagg

SEQ ID NO: 145 - DNA, 29 bp, synthetic construct
  misc_feature 1..29: note = Yarrowia lipolytica
  source 1..29: mol_type = other DNA
cccaaatttagctgcatcattcatcaacc

SEQ ID NO: 146 - DNA, 27 bp, synthetic construct
  misc_feature 1..27: note = Yarrowia lipolytica
  source 1..27: mol_type = other DNA
ccgtgcttaagagcaagttccttgagg

SEQ ID NO: 147 - DNA, 23 bp, synthetic construct
  misc_feature 1..23: note = Yarrowia lipolytica
  source 1..23: mol_type = other DNA
ctacgacatgcccaaggagcagc

SEQ ID NO: 148 - DNA, 26 bp, synthetic construct
  misc_feature 1..26: note = Yarrowia lipolytica
  source 1..26: mol_type = other DNA
ggattcgcacattggtgaactggatc

SEQ ID NO: 149 - DNA, 24 bp, synthetic construct
  misc_feature 1..24: note = Yarrowia lipolytica
  source 1..24: mol_type = other DNA
ccaagcgacgacaagctgttgagc

SEQ ID NO: 150 - DNA, 21 bp, synthetic construct
  misc_feature 1..21: note = Yarrowia lipolytica
  source 1..21: mol_type = other DNA
cgtgtgggtagcagagtgggc

SEQ ID NO: 151 - DNA, 26 bp, synthetic construct
  misc_feature 1..26: note = Yarrowia lipolytica
  source 1..26: mol_type = other DNA
ctttgccatgactgagactggccatg

SEQ ID NO: 152 - DNA, 18 bp, synthetic construct
  misc_feature 1..18: note = Yarrowia lipolytica
  source 1..18: mol_type = other DNA
gaggcgccgctggtgtcg

SEQ ID NO: 153 - DNA, 27 bp, synthetic construct
  misc_feature 1..27: note = Yarrowia lipolytica
  source 1..27: mol_type = other DNA
tccgaaagcggaactgctttctcaatg

SEQ ID NO: 154 - DNA, 23 bp, synthetic construct
  misc_feature 1..23: note = Yarrowia lipolytica
  source 1..23: mol_type = other DNA
cagaccggcctctcgaatatggc

SEQ ID NO: 155 - DNA, 26 bp, synthetic construct
  misc_feature 1..26: note = Yarrowia lipolytica
  source 1..26: mol_type = other DNA
ctagactacacgggcaaccttaaccc

SEQ ID NO: 156 - DNA, 25 bp, synthetic construct
  misc_feature 1..25: note = Yarrowia lipolytica
  source 1..25: mol_type = other DNA
cttgggcttcagctttcgaggagtg

SEQ ID NO: 157 - DNA, 4435 bp, synthetic construct
  misc_feature 1..4435: note = pLD1
  source 1..4435: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataagaagagcccgggtctagatgtgtgctcttccgagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 158 - DNA, 5564 bp, synthetic construct
  misc_feature 1..5564: note = pLD10
  source 1..5564: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgaatcatttaagagcagaaggacccgcatcagtgttagcgataggtacagctaacccagagaatatcttaatccaagatgaatttcctgactactatttccgtgttactaaatcggaacatatgactcaacttaaagagaagttccggaaaatctgcgataaatccatgatccgaaagagaaactgtttccttaacgaagaacatctcaagcaaaacccgaggttggtagagcacgaaatgcagaccttggatgctaggcaggacatgttggtggtcgaagtgccaaaactcggcaaggacgcgtgcgctaaggcaatcaaggagtggggtcaaccgaagtctaaaatcacgcatctaatatttacatctgcactgacaaccgacatgccgggtgccgattatcactgcgccaagctacttggattgagtccactggttaagagagttatgatgtatcaattggggtgttacggagggggcacagtcctcagaattgctaaggatattgcggaaaataacaagggcgcgagggtccttgctgtatgttgtgatattatggcctgtttgtttcgcgggccctcggattcagatttggaattgcttgtcggacaggcaatttttggtgacggggccgcagcagtcatagtgggagccgaaccagacgaaagcgtgggtgaaagaccaatctttgagttggttctgaccggacaaacgatcttacctaactcggaaggtacgattggaggacatattagagaagccggcctaattttcgatcttcacaaagacgttccaatgttaatctccaataacatagaaaagtgcttgatagaagcatttactcccattggtattagtgactggaacagcattttctggatcacccaccctggaggaaaagctatactcgataaggttgaagagaaactcgacttgaaaaaggagaaattcgttgactcacgacatgtgttatcagagcacgggaatatgagttcatccacagtcttgttcgtaatggatgaattgcgaaaacgctctcttgaggagggaaagagcacaaccggtgacgggtttgagtggggcgtgctattcggttttggcccaggtttgactgtcgagcgggttgttgttcgtagtgtaccaattaagtactgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 159 - DNA, 4724 bp, synthetic construct
  misc_feature 1..4724: note = pLD12
  source 1..4724: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatggcagtcaaacacctaatagttctcaaatttaaagacgagattactgaagctcagaaggaagagttctttaagacatatgttaacttagtcaacatcatccccgcgatgaaggacgtctactggggcaaggatgtgacgcaaaaaaataaggaagaaggatacacacatatcgttgaggtgacctttgagagtgtggaaactattcaagattatattattcacccagcccatgtagggttcggtgacgtttatcgatcattctgggaaaagttgcttatatttgattacaccccaagaaaattgaagcctaagtgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 160 - DNA, 4742 bp, synthetic construct
  misc_feature 1..4742: note = pLD14
  source 1..4742: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatggcagtcaaacacctaatagttctcaaatttaaagacgagattactgaagctcagaaggaagagttctttaagacatatgttaacttagtcaacatcatccccgcgatgaaggacgtctactggggcaaggatgtgacgcaaaaaaataaggaagaaggatacacacatatcgttgaggtgacctttgagagtgtggaaactattcaagattatattattcacccagcccatgtagggttcggtgacgtttatcgatcattctgggaaaagttgcttatatttgattacaccccaagaaaattgaagcctaagggaagacgagctaagttgtgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 161 - DNA, 6569 bp, synthetic construct
  misc_feature 1..6569: note = pLD16
  source 1..6569: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgggaaaaaattataaatctttggactcagttgtggctagtgacttcattgcacttgggatcacatcagaagttgctgagacattgcacggacgcttggcagagatagtttgcaactacggcgccgcaacacctcagacctggattaacatcgcaaaccatattctaagtccagatcttccatttagtctccatcagatgttgttctacggttgttataaggactttggtccagcacccccagcttggataccagaccccgaaaaagtaaagtccacgaacttaggtgccttgttagaaaagcggggaaaggagtttctaggcgttaagtataaggacccaataagtctgttttctcacttccaggagtttagcgttcgaaatccggaagtctactggcggacggtacttatggatgaaatgaagatactgttcagcaaagatcccgaatgtatcctcagacgcgacgacattaacaacccagggggctctgagtggctaccaggtggatatctcaacctggccaagaactgtttgaatgtaaatagtaacaaaaaacttaacgacactatgatagtgtggagagatgaaggaaatgacgatctcccattgaataaattgactcttgatcaattacgaaaacgagtctggttggttggatacgccctagaagagatgggccttgagaagggatgtgcgattgcaattgacatgcccatgcacgtagatgcggttgtgatctatttagctatcgtcttggcaggctacgtcgttgtctccattgcagattcattctcagcaccggaaatttccacaagattgcgtctatcaaaggctaaggctatttttacacaagatcatatcatccgagggaaaaagcgtatacctttgtacctgcgtgtcgtcgaggccaagtctccgatggcaatagttatcccgtgttcgggttcaaatattggtgcggaattgcgggatggtgatattctgtgggattacttcttagaacgcgcaaaggaatttaagaactgcgaatttacagcccgtgaacagccagtggacgcgtacacaaatattttgttctcatcgggaaccaccggagagccaaaggcgataccatggactcaagctacgcctctcaaggcggctgctgatggttggtcacacttggacattagaaagggtgacgtaattgtatggcctaccaatttggggtggatgatggggccttggttggtctatgcttcactccttaacggggcaagcatcgcattgtataacggatctccactagtgtccggctttgccaaattcgttcaagatgcgaaagttactatgctaggagttgtcccctccatcgtacgaagctggaaaagcactaattgcgttagtgggtacgattggtctacaatcagatgcttctcctcatcgggtgaggcatcgaatgtcgatgaatacttatggctaatgggaagggctaactacaaaccggtcatcgaaatgtgcggtggcacagagatcgggggtgccttcagcgccggttcgtttttacaagcccaatctttgagtagcttctcatcccaatgtatgggatgcaccttgtacattctcgacaagaatggctacccgatgccaaagaacaagccgggtataggtgaattggccttgggacccgtgatgttcggtgcttccaagactttacttaacggaaaccatcatgacgtttatttcaaaggcatgcccaccttgaacggagaagtcttgaggagacacggagatatcttcgaactcacttcgaacggctattatcacgctcatggtagagcagatgacacgatgaatatcggggggattaaaatttcctcaatcgagattgaaagggtgtgtaatgaagttgacgatagagtgtttgagactacggccattggagtgcctccattgggcggaggtccagagcagctcgttatcttttttgttcttaaggacagcaatgatacgaccatcgacctaaaccaattgcgacttagttttaatcttgggttacaaaagaaattgaacccactttttaaggtgacgagggttgtgccactttcgctgttgcctaggacagccaccaacaaaataatgagaagagtgcttagacagcaatttagtcatttcgagtgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 162 - DNA, 6041 bp, synthetic construct
  misc_feature 1..6041: note = pLD20
  source 1..6041: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgaagtgttctacgtttagtttttggtttgtttgtaaaattatattcttctttttttccttcaacattcagacatcaatcgccaacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacactgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 163 - DNA, 6536 bp, synthetic construct
  misc_feature 1..6536: note = pLD22
  source 1..6536: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgacagaagtagtagatagagcaagttccccagcaagtccaggatctacgaccgccgccgcagacggtgctaaggtggcggtggagccacgcgtagatgtagcggcccttggcgagcagttgctagggcgatgggctgacatcagattgcacgcacgagacttagcaggccgcgaagtggtccaaaaggttgaaggacttacgcacactgagcatcggagtagagtctttggacagttgaagtacttggtagacaacaatgctgttcacagagcttttccctccaggctaggtggatcagatgaccatggcggtaatatagctggattcgaggaattagttactgctgatccatcattgcaaataaaggccggcgttcagtggggtttgtttggttctgcagtgatgcacttgggaacccgtgaacatcatgacaagtggttgccaggtattatgtcgttagaaataccggggtgtttcgcgatgacagaaaccgggcacggtagcgacgtggcctctattgctacaacagcaacttatgatgaggaaacccaagagtttgttattgataccccgttcagagccgcttggaaagattatatcggtaatgcagcgaacgatggtttggcggcagttgttttcgcacaattaatcacgaggaaagtgaaccatggtgtacacgccttttacgtggatctcagagatcctgcgactggagacttcctacccggaataggaggagaggacgatggaatcaaggggggattgaatggcattgacaacggtagactacattttacgaacgtacgcattcctagaactaatcttcttaacagatatggcgatgtggcggtcgacggcacatacctgtcgaccatcgaatcaccagggcgccggttctttacgatgcttggtactctagtccagggtagagttagtctcgatggagcagctgtcgctgcactgaaggtcgcattgcaaagtgcaattcactacgctgcggagaggagacaatttaatgcgacttcacctactgaagaagaggtccttcttgattatcagaggcatcaaaggagactctttacacgacttgcaacgacgtacgccgcatctttcgcccacgagcagctattgcaaaagttcgatgatgtcttttcaggggcacatgataccgacgccgaccggcaggacttggaaaccctagccgccgctttgaagccattgagcacatggcatgcacttgacacgttacaagaatgcagagaggcctgtgggggggccggatttttgatagaaaaccgtttcgcgagcttgcgtgctgacttggacgtttacgtcacattcgagggtgataacacagttttattgcaattggttgctaaacggctcttggcagactacgcaaaagagttcagaggggccaacttcggcgttcttgccaggtatgtggttgaccaagccgcgggagtggcgctccaccgaacaggactaaggcaagtcgctcaatttgttgcagacagcgggtccgttcagaagtcggctcttgcgcttcgcgatgaagagggtcaacgaacattgttaacggacagagtacagagcatggttgccgaagtgggggctgccttgaaaggcgcaggcaaattaccccaacatcaagcagctgcattgttcaaccaacaccagaacgaacttattgaggctgcccaggcccatgcagaactcctccaatgggaggcatttacagaagctctcgctaaagtcgacgatgctggtacaaaggaagtgcttactcgattgcgagatctctttggtttgtccttgattgaaaaacacttgctgtggtatcttatgaatggacgtttgtccatgcaaagaggcaggacagttggaacttacattaatcgtttacttgtcaagatccgtccacacgcactagacttggttgatgccttcggttacggcgcggagcatttgcgtgctgctatcgccaccggagcggaagcaacccgacaggatgaagcccgaacgtattttagacaacaacgggcatcgggactggccccggccgatgaaaagaccttactcgctatcaaagctggtaaatcaagagggcgaagggcaaagctatgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 164 - DNA, 5582 bp, synthetic construct
  misc_feature 1..5582: note = pLD24
  source 1..5582: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgaatcatttaagagcagaaggacccgcatcagtgttagcgataggtacagctaacccagagaatatcttaatccaagatgaatttcctgactactatttccgtgttactaaatcggaacatatgactcaacttaaagagaagttccggaaaatctgcgataaatccatgatccgaaagagaaactgtttccttaacgaagaacatctcaagcaaaacccgaggttggtagagcacgaaatgcagaccttggatgctaggcaggacatgttggtggtcgaagtgccaaaactcggcaaggacgcgtgcgctaaggcaatcaaggagtggggtcaaccgaagtctaaaatcacgcatctaatatttacatctgcactgacaaccgacatgccgggtgccgattatcactgcgccaagctacttggattgagtccactggttaagagagttatgatgtatcaattggggtgttacggagggggcacagtcctcagaattgctaaggatattgcggaaaataacaagggcgcgagggtccttgctgtatgttgtgatattatggcctgtttgtttcgcgggccctcggattcagatttggaattgcttgtcggacaggcaatttttggtgacggggccgcagcagtcatagtgggagccgaaccagacgaaagcgtgggtgaaagaccaatctttgagttggttctgaccggacaaacgatcttacctaactcggaaggtacgattggaggacatattagagaagccggcctaattttcgatcttcacaaagacgttccaatgttaatctccaataacatagaaaagtgcttgatagaagcatttactcccattggtattagtgactggaacagcattttctggatcacccaccctggaggaaaagctatactcgataaggttgaagagaaactcgacttgaaaaaggagaaattcgttgactcacgacatgtgttatcagagcacgggaatatgagttcatccacagtcttgttcgtaatggatgaattgcgaaaacgctctcttgaggagggaaagagcacaaccggtgacgggtttgagtggggcgtgctattcggttttggcccaggtttgactgtcgagcgggttgttgttcgtagtgtaccaattaagtacggaagaagggcaaagttgtgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 165 - DNA, 5606 bp, synthetic construct
  misc_feature 1..5606: note = pLD56
  source 1..5606: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatggggttgtccttagtttgtacgttcagtttccaaactaactaccacacactactaaatccgcacaacaaaaacccgaaaaattcattgctctcctatcagcacccaaaaacacccattatcaagtctagttacgacaactttccatcaaaatactgtctaacgaaaaactttcatttgttgggcttaaattctcataatcgtatttccagtcagtcccgatcgatcagggccgggagtgaccaaattgaaggttctccacatcatgaaagtgacaattcaattgctacgaagattttaaactttgggcatacatgctggaagctacagcgaccgtatgtagttaaggggatgatcagcattgcctgcggcctattcggaagggaactcttcaataatagacatcttttttcttggggtttaatgtggaaagcttttttcgctttggttcctatccttagttttaacttcttcgccgctattatgaatcaaatttacgatgttgacatcgaccgtattaacaaacccgatctcccccttgtttcaggcgagatgtccattgaaacggcatggattttgtccatcattgttgcgcttactggcttgattgttaccattaagcttaaaagcgctcccttgttcgtttttatatacattttcggcatttttgccggattcgcatacagtgtcccgcctatacgttggaaacaatatccattcacgaacttcttgatcacgatctcatcacatgttggattggcctttacgtcctacagtgctaccacatctgcccttggattgcctttcgtttggaggcctgccttctcgtttatcattgcatttatgacagtgatgggaatgactatcgcatttgctaaagatatcagcgacatagagggcgatgcaaaatatggggtgagtactgttgcgacgaagttgggcgcccgaaatatgaccttcgttgtttccggcgttcttttacttaactatttagtatcgattagcatcgggatcatctggccacaggtgtttaaatcaaatattatgatcttgtcgcatgccatcctagctttctgtcttatatttcaaacaagagaattagccctagcgaactacgcctcagcaccaagtcgtcagttcttcgaatttatatggctactctactacgccgaatacttcgtctatgtcttcatttagtaataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 166 - DNA, 5071 bp, synthetic construct
  misc_feature 1..5071: note = pLD87
  source 1..5071: mol_type = other DNA
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctttaattaagtcgacttgatgtttagagtgtccagatccgcaagatcggctcgcacttgtgttgtgttgtttcaaatcagcctgtcgttttgtgtcgtttgagatcattctgtctcactcttaggctcgcttagaaccgacaacggagaatccgggctcggtttttcggtcggccttgatctgggccttggacttgtactggtcggccatctccacgttgaccagctccttgaccttgtagagctgaccggcgataccaggagacaccttgtagtacttctgggagccgaccttgcccagaccgagggtcttgagcacgtcacgtgttctccacggcattcgcaggatagatcggacctgtgtgactttgtagaacatggcgtttcaggtggttgcgtgagtgtgtaaaatcgtgtctttcagaagttacaaatttcaccgcatttagagtttatgcagatgggcggtgtgtggttgggagttcgatttccgtgcgtgcatttgatcttgatgaattggatttgtacatggggaatagcacgtcaagaaccgcctactgcaaactcgtgaatattgagattattgaggaaattcaaggaaaattcagatcagatttgagagcaaagtccaacaatactacacaatccctttcctgtattcttccaccatcgtcatcgtcgtctgtcttctcttcagctttttaatttcactccccacaaacccaaatttagctgcatcattcatcaacctccaattataactatacatcgcgacacgaacacgaaacacgaaccacgaaccgccgctttgtcgacggagtgagacgggagagagaccatggtctctcgctcgtctcacgcttagcggccgcgtcgacataacttcgtatagcatacattatacgaagttattttctaatttggaccgatagccgtatagtccagtctatctataagttcaactaactcgtaactattaccataacatatacttcactgccccagataaggttccgataaaaagttgtgcagactaaatttatttcagtctcctcttcaccaccaaaatgccctcctacgaagcgcgagctaacgtccacaagtccgcctttgccgcccgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggaactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggatgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagatgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtcatttaactgtgtatatagagagcgtgcaagtatggagcgcttgttcagcttgtatgatggtcagacgacctgtctgatcgagtatgtatgatactgcacaacctgataacttcgtatagcatacattatacgaagttatctcgagggatccctagggaggcacatctaaacgaataacgaatattaatgataccatcatatctcagaacatgtatgactgctgcttccaaacgatatgaggatgagtcctctttcagattaagatagagtacaaatatattatctatatactggtgtctgtgcgatgtcgtatgagcggtgaatcatgtgactgtcacgtggtttggcccaagttacaccgtagctacgcctttcttgaccgactccatggtcttctgggcgggttgacagtttccactggatgagcgtccgcctcctgttcctgtcgttgtccctgcagctcagcctcaatcttctgaccgagctcggagtccagggaaatgccaacaggttgtccaagcaacatcatggtttggtgggcagccgtgatctcatcgtcgttggataccattcggtacttggcctcaatctgcacaaagtagcggtaccactggtttcgagcaaaccgctccaattgagcctctccgtcgagagagagagtaggtgattgctccaacttgcggccaaaatgaagttctcgactcacctttttgaagcggttcttcttgcccatcttggtggcgaaagtagtggctagtggtggatgactttgtataatgtaccgatgaagagggttgtatttgctcagtaagaagtagcgagtgaaatcagatcacttaacgagagcaaagggcaatggaatacctgctgcctgattaacaacagcttctgtgtcgtttctctcttgtgaatgagtgtgttgctagaggtaggttggcactccaatgttacttaattaa

SEQ ID NO: 167 - DNA, 5096 bp, synthetic construct
  misc_feature 1..5096: note = pLD101
  source 1..5096: mol_type = other DNA
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctttaattaacgatccgcgtaaattcagagcaatgccaagatccttatttccgataacagcgagatacacatcagaagaggtggagactctggccccgcacgaccaccacgtcgtcactcaaagcaaagtcacgatgtagaaatcacaatcgtccccataagcacgtggattcccctggttttgtttcggcctcggcagtggcaattctggggtatataaaccagcaaggttatgacctgattgacctgcttgtggcctgataaaccgcctgacttttgtgataaggttgggagcatgcggctcgggccattgtgagcattttcgtcaaggactggccaagtccaactggaagagacatgggcaaaatgtcgactttacagatgtacccgaaattgcttcaattgctctcagctgccggaaccggatctatcggtgcaagtatcgtacagtagacatgtgctattggtaaccctcggtattggctaggtttcgtatcagggatacagttcaacgctgatcgcatatggcatgattccggctcgacacagcgaccaagaaccaagcgtgtatgtcgtagacttgcaaatcatgtggggcttatccccggatttccccaagtcacgttttcacaaaggctgtctcccgaatgcatgagccgaggcaggctaaactggtttgttcatgtaccccacacaacgtaaagatgcaccccatgtgcagtgaaataccacaagtatatatataccgacctacccgagatagcaaattgattctacacttacactaccaattcttacatcaaaccaaaccgcttgagaccatccggtctctgatcatcctcgagataacttcgtataatgtatgctatacgaagttatcaggttgtgcagtatcatacatactcgatcagacaggtcgtctgaccatcatacaagctgaacaagcgctccatacttgcacgctctctatatacacagttaaatgacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcatctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacatcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagttccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgggcggcaaaggcggacttgtggacgttagctcgcgcttcgtaggagggcattttggtggtgaagaggagactgaaataaatttagtctgcacaactttttatcggaaccttatctggggcagtgaagtatatgttatggtaatagttacgagttagttgaacttatagatagactggactatacggctatcggtccaaattagaaaataacttcgtataatgtatgctatacgaagttatgtcgacgcggccgcacaagcactacatggacgaggtcaaggctgccaacaaccctcgtaacacccatgctccttactacgagacaaagctgcgacccttcctgttccgacccgatgaggacgaggagatttgcgacctggacgagtaggttgttgtaatactatgatttattgtgtttatatgttattgatactattgaaagagttattgtgtaattttagatgctgtatgttaactagaagctcagattctacaaagagatcctcagatctgaggaatgatctacgttctgcaatagaagggacaactgcagctctgaatgaccacaaaaagaatacaccacaagcagttgtaactgagctattagccttgctttcgcaccattcgcttttctggatggtagccctttactacaagtagctaatatggaatgtacattaccgtctcattacaatatgtatatgcaagttcatggcacttcatgcacaccagccccttcgttaagtaccttccaaaaagtgatcaatgataagtgatatatctaatttagagatctggacacacgaacaagtcgggaacacaaatcccgagatgattgcctgctcagaggagtccaattagtctttacaccattcgtgctaatgaagggcacagaatattccactttgaaagtttaagattaagctcggctcgcaattatgcatgaaaaatatgtagggagagaacgatcccacgagttctgtttggttgcgagagtgttcgggttttctcctaaaaagaataggggagggaaaaattatcgcctaagtcaccattaattaa

SEQ ID NO: 168 - DNA, 5096 bp, synthetic construct
  misc_feature 1..5096: note = pLD102
  source 1..5096: mol_type = other DNA
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctttaattaattggcaaattttactgtggccttcagaacggtaaaaatagaccaatcagaattctgaaaagcacatcttgatctcctcattgcggggagtccaacggtggtcttattcccccgaatttcccgctcaatctcgttccagaccgacccggacacagtgcttaacgccgttccgaaactctaccgcagatatgctccaacggactgggctgcatagatgtgatcctcggcttggagaaatggataaaagccggccaaaaaaaaagcggaaaaaagcggaaaaaaagagaaaaaaaatcgcaaaatttgaaaaatagggggaaaagacgcaaaaacgcaaggaggggggagtatatgacactgataagcaagctcacaacggttcctcttatttttttcctcatcttctgcctaggttcccaaaatcccagatgcttctctccagtgccaaaagtaagtaccccacaggttttcggccgaaaattccacgtgcagcaacgtcgtgtggggtgttaaaatgtgggggcggggaaccaggacaagaggctcttgtgggagccgaatgagagcacaaagcgggcgggtgtgataagggcatttttgcccattttcccttctcctgtctctccgacggtgatggcgttgtgcgtcctctatctatttctttttatttctttttgttttatttctctgactaccgatttggcttgatttcctcaaccccacacaaataagctcgggccgaggaatatatatatacacggacacagtcgccctgtggacaacacgtcactacctctacgacgcttgagaccatccggtctctgatcatcctcgagataacttcgtataatgtatgctatacgaagttatcaggttgtgcagtatcatacatactcgatcagacaggtcgtctgaccatcatacaagctgaacaagcgctccatacttgcacgctctctatatacacagttaaatgacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcatctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacatcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagttccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgggcggcaaaggcggacttgtggacgttagctcgcgcttcgtaggagggcattttggtggtgaagaggagactgaaataaatttagtctgcacaactttttatcggaaccttatctggggcagtgaagtatatgttatggtaatagttacgagttagttgaacttatagatagactggactatacggctatcggtccaaattagaaaataacttcgtataatgtatgctatacgaagttatgtcgacgcggccgcatggagcgtgtgttctgagtcgatgttttctatggagttgtgagtgttagtagacatgatgggtttatatatgatgaatgaatagatgtgattttgatttgcacgatggaattgagaactttgtaaacgtacatgggaatgtatgaatgtgggggttttgtgactggataactgacggtcagtggacgccgttgttcaaatatccaagagatgcgagaaactttgggtcaagtgaacatgtcctctctgttcaagtaaaccatcaactatgggtagtatatttagtaaggacaagagttgagattctttggagtcctagaaacgtattttcgcgttccaagatcaaattagtagagtaatacgggcacgggaatccattcatagtctcaattttcccataggtgtgctacaaggtgttgagatgtggtacagtaccaccatgattcgaggtaaagagcccagaagtcattgatgaggtcaagaaatacacagatctacagctcaatacaatgaatatcttctttcatattcttcaggtgacaccaagggtgtctattttccccagaaatgcgtgaaaaggcgcgtgtgtagcgtggagtatgggttcggttggcgtatccttcatatatcgacgaaatagtagggcaagagatgacaaaaagtatctatatgtagacagcgtagaatatggatttgattggtataaattcatttattgcgtgtctcacaaatactctcgataagttggggttaaactggagatggaacaatgtcgatatctcgacatattttgatatttgttaattaa

SEQ ID NO: 169 - DNA, 6044 bp, synthetic construct
  misc_feature 1..6044: note = pLD111
  source 1..6044: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgaattgttcagcatttagtttttggtttgtttgtaagattattttcttctttttgtcatttaacattcaaatttcaattgcaaacccacaagaaaactttttgaagtgtttttcagaatacattccaaacaatccagctaacccaaagtttatttacacacaacatgatcaattgtacatgtcagttttgaactcaacaattcaaaacttgagatttacatcagataccacaccaaagccattggttattgttacaccatcaaacgtttcccatattcaagcatcaatcttgtgttcaaagaaggttggattgcaaattagaaccagatcaggaggacacgatgcagaaggaatgtcatacatttcacaagttccattcgttgttgttgatttgagaaacatgcactcaattaagattgatgttcattcacaaacagcatgggttgaagcaggagcaacattgggtgaagtttactactggattaacgaaaagaacgaaaacttcagttttccaggaggttactgtccaacagttggagttggaggacatttttcaggtggaggatacggagcattgatgagaaactacggattggcagcagataacattattgatgcacacttggttaacgttgatggaaaggttttggatagaaagtcaatgggagaagatttgttttgggcaattagaggaggtggtggagagaactttggaattattgcagcatggaagatcaagttggttgcagttccatcaaagtcaacaatcttttcagttaagaagaacatggaaattcatggtttggttaagttgtttaacaagtggcaaaacattgcatacaagtacgataaggatttggttttgatgacacattttattacaaagaacattacagataaccatggaaagaacaagacaacagttcacggatacttttcatcaatttttcacggaggagttgattcattggttgacttgatgaacaagtcatttccagaattgggaatcaagaagacagattgtaaggaattttcatggattgatacaacaattttctactcaggagttgttaactttaacacagcaaactttaagaaggaaattttgttggacagatcagcaggaaagaagaccgcattttccattaagttggattacgttaagaaaccaattccagaaacagcaatggttaagattttggaaaagttgtacgaagaagatgttggtgttggaatgtacgttttgtacccatacggaggaattatggaagaaatctcagaatcagcaattccatttccacatagagcaggtattatgtacgaattgtggtacacagcatcatgggaaaagcaagaagataatgaaaagcatattaactgggttagatcagtttacaactttacaacaccatacgtttcacaaaacccaagattggcatacttgaactacagagatttggatttgggaaagacaaacccagaatcaccaaacaactatacacaagctagaatttggggagaaaagtactttggtaagaacttcaacagattggttaaagttaagacaaaggcagatccaaataacttctttagaaacgaacaatcaattccaccattgccaccacatcatcattaataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 170 - DNA, 6044 bp, synthetic construct
  misc_feature 1..6044: note = pLD112
  source 1..6044: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgaattgtagcactttctcattctggtttgtttgtaagattattttctttttcttgtcatttaacattcaaatttcaattgcaaacccacaagagaactttttgaagtgtttctcagaatacattccaaacaacccagctaacccaaagtttatttacacccaacacgatcaattgtacatgtcagttttgaactcaacaattcaaaacttgagatttacatcagatacaacaccaaagccattggttattgttacaccatcaaacgttagtcatattcaagcatcaatcttgtgttcaaagaaggttggattgcaaattagaactagatcaggaggacatgatgcagaaggattgtcatacatttcacaagttccatttgcaattgttgatttgagaaacatgcacacagttaaggttgatattcattcacaaacagcatgggttgaagcaggagcaacattgggtgaagtttactactggattaacgaaatgaacgaaaacttctcatttccaggaggatactgtccaacagttggtgttggaggacacttttcaggtggtggatacggagcattgatgagaaactacggattggcagcagataacattattgatgcacatttggttaacgttgatggaaaggttttggatagaaagtcaatgggagaagatttgttttgggcaattagaggaggtggaggagaaaactttggaatcattgcagcatgtaagatcaagttggttgttgttccatcaaaggcaacaatcttttcagttaagaagaacatggaaatccatggattggttaagttgtttaacaagtggcaaaacattgcatacaagtacgataaggatttgatgttgacaacacattttagaacaagaaacattacagataaccacggaaagaataagacaacagttcatggatacttttcatcaattttcttgggaggagttgattcattggttgacttgatgaacaagagttttccagaattgggaatcaagaagacagattgtaaggaattgtcatggatcgatacaaccattttctactcaggagttgttaactacaacacagctaactttaagaaggaaattttgttggacagatcagcaggtaaaaagacagcattttcaattaagttggattacgttaagaaattgattccagaaacagcaatggttaagattttggaaaagttgtacgaagaagaagttggagttggaatgtacgttttgtacccatacggaggaattatggatgaaatttcagaatcagcaattccatttccacatagagcaggtattatgtacgaattgtggtacacagcaacatgggaaaagcaagaagataacgaaaagcatattaactgggttagatcagtttacaactttacaaccccatacgtttcacaaaacccaagattggcatacttgaactacagagatttggatttgggaaagacaaacccagaatcaccaaataactacacacaagctagaatttggggagaaaagtactttggtaagaactttaacagattggtgaaggttaagacaaaggcagacccaaacaatttctttagaaacgaacaatcaattccaccattgccaccaagacatcattaataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 171 - DNA, 7167 bp, synthetic construct
  misc_feature 1..7167: note = pLD113
  source 1..7167: mol_type = other DNA
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctgatcccaatattacacccaagtagcatgcataagctaaaagtaactcgcagcgcacaccgtgcagattcataagtctatgattaattgaacgccaataacccggcttactacaagtacaagtaggtatacatagcggtaatgaatcattagaaaaataaaaaacaaaaaaaaacaaaacaaactgttgtggatgcatcaacagtagtacatagttgtacgatgtacttgtacttgtaaaagcaaaaatgtacaatatctcagggagcgcaacttttacgttcgaagaacaatgtaccgcataccgcattctagattctgcggaacgtctaacctggaaatacgattttttatttctttcattttttttgcttcttcaaaagtatggtaatttcctaccattacagttgacactgaacgaggggggattgaatttaagcaaaaaattaaatcaaaatacctttatgtatccagcccatgtaataaacaaaaggattatataacaagaaataaatatatacctttaatggatcattagaataaaaataaatacgagaagcacaccagagaagctttttgattgccactataccgctactttggtatatcttattataattgttgaatttgcaagatagaatgtcattcattggagagaaatccaaggaatatgtgggatgaaatgactagaagtatgaacaatgagaatagtacatacttgtacctgtatttctagaagagagaaagacagttgagtgtgtgattctcgtccaataataatctcaatagtaacgtgtgaatagctgttctttgatagttgatatttctcgatgactatttatgttgtacaagggatttttttcgttgctgttgatttcgaattaggcaatgcagatatcatttatgctatccatatttaagatttcccatacgcatttataacatttattctacataaattgttaaatgaacgaactgccattataaattgtttcctaaataggaagtgtttttcataaagcaagtaagttgtctaataatactaagtaataaaaataagttcatacaatatattttgagaacatcatttggaggcggtagatggagtctgtttattattaaacaatgcgagatgaccccttaaatattgagaacatcagttggaggcggcagatggagtctgtctatttagcaatgggacatgactgtcagtatcatcatatgtatatatataatacatataatattatataacacgatttttttaaattattggcccgaaaattaatcagtgtagactggatcctcgagaaccatttaattaacaagtcgagaacgtaccactgtcctccactacaaacacacccaatctgcttcttctagtcaaggttgctacaccggtaaattataaatcatcatttcattagcagggctgggccctttttatagagtcttatacactagcggaccctgccggtagaccaacccgcaggcgcgtcagtttgctccttccatcaatgcgtcgtagaaacgacttactccttcttgagcagctccttgaccttgttggcaacaaagtctccgacctcggaggtggaggaggagcctccgatatcggcggtagtgataccagcctcgacggactccttgacggcagcctcaacagcgtcaccggcgggcttcatgttaagagagaacttgagcatcatggcggcagacagaatggtggcaatggggttgaccttctgcttgccgagatcgggggcagatccgtgacagggctcgtacagaccgaacgcctcgttggtgtcgggcagagaagccagagaggcggagggcagcagacccagagaaccggggatgacggaggcctcgtcggagatgatatcgccaaacatgttggtggtgatgatgataccattcatcttggagggctgcttgatgaggatcatggcggccgagtcgatcagctggtggttgagctccagctgggggaattcgtccttgaggactcgggtgacggtctttcgccaaagtcgagaggaggccagcacgttggccttgtcaagggaccacacgggaagaggggggttgtgctgaagggccaggaaggcggccattcgggcaattcgctcaacctcaggaacggagtaagtctcagtgtcggaagcgacgccagatccgtcatcctcctttcgctctccaaagtagatacctccgacgagctctcggacaatgatgaagtcggtgccctcaacgtttcggatgggggagagatcggcgagcttgggcgacagcagctggcagggtcgcaggttggcgtacaggttcaggtcctttcgcagcttgagaagaccctgctcgggtcgcacgtcggttcgtccgtcgggagtggtccatacggtgttggcagcgcctccgacagcaccgagcataatagagtcagcctttcggcagatgtcgagagtagcgtcggtgatgggctcgccctccttctcaatggcagctcctccaatgagtcggtcctcaaacacaaactcggtgccggaggcctcagcaacagacttgagcaccttgacggcctcggcaatcacctcggggccacagaagtcgccgccgagaagaacaatcttcttggagtcagtcttggtcttcttagtttcgggttccattgtggatgtgtgtggttgtatgtgtgatgtggtgtgtggagtgaaaatctgtggctggcaaacgctcttgtatatatacgcacttttgcccgtgctatgtggaagactaaacctccgaagattgtgactcaggtagtgcggtatcggctagggacccaaaccttgtcgatgccgatagcgctatcgaacgtaccccagccggccgggagtatgtcggaggggacatacgagatcgtcaagggtttgtggccaactggtaaataaatgatgactcaggcgacgacggaattcgacagcaactactcctttcaccaaccatgtgcattttagctcgaataacattcacaggcttggtgatctacatccatggtgtctggccgattaccgtggtgttttggcagtaacgagaatattgagtgaactcttcccatcaccaataaagactcatactacaatcacgagcgcttcagctgccactatagtgttggtgacacaatacccctcgatgctgggcattactgtagcaagagatattatttcatggcgcattttccagtctacctgactttttagtgtgatttcttctccacattttatgctcagtgtgaaaagttggagtgcacacttaattatcgccggttttcggaaagtactatgtgctcaaggttgcaccccacgttacgtatgcagcacattgagcagcctttggaccgtggagataacggtgtggagatagcaacgggtagtcttcgtattaattcaatgcattgttagttttatatgatatggtgtcgagcggccgcgaccgggttggcggcgcatttgtgtcccaaaaaacagccccaattgccccaattgaccccaaattgacccagtagcgggcccaaccccggcgagagcccccttctccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaatgtccaacctcctcaccgtccaccagaaccttcccgcccttcccgtggacgccacctccgacgaggtccgaaagaacctcatggacatgttccgagatcgacaggccttctccgagcacacttggaagatgctcctctccgtctgccgatcctgggccgcctggtgcaagctcaacaaccgaaagtggttccccgccgagcccgaggacgtccgagactacctcctttacctccaggcccgaggcctcgccgtcaagaccatccagcagcacctcggccagctcaacatgctccaccgacgatccggcctcccccgaccctccgactccaacgccgtgtccctcgtcatgcgacgaatccgaaaggagaacgtggacgccggcgagcgagccaagcaggcccttgccttcgagcgaaccgacttcgaccaggtccgatccctcatggagaactccgaccgatgccaggacatccgaaacctcgccttccttggcatcgcctacaacaccctccttcgaatcgccgagatcgcccgaatccgagtcaaggacatctcccgaaccgacggcggccgaatgctcatccacatcggccgaaccaagaccctcgtgtccaccgccggcgtcgagaaggccctctccctcggcgtcaccaagctcgtcgagcgatggatctccgtgtccggcgtcgctgacgaccccaacaactacctcttctgccgagtccgaaagaacggcgtcgctgctccctccgccacctcccagctctccacccgagcccttgagggcatcttcgaggccacccaccgactcatctacggcgccaaggacgactccggccagcgatacctcgcctggtccggccactctgctcgagtcggtgccgcccgagacatggcccgagccggtgtctccatccccgagatcatgcaggccggcggctggaccaacgtcaacatcgtcatgaactacatccgaaacctcgactccgagactggcgccatggtccgacttcttgaggacggcgactgatgatcatatgattacattaatagctaattacgtgtatccgatatatatactaattacaatagtacatattagaacatacaatagttttaattaa

SEQ ID NO: 172 - DNA, 6125 bp, synthetic construct
  misc_feature 1..6125: note = pLD125
  source 1..6125: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgttcttgaaacacatttttgttgctctcgcttttgccttgttagctgacgctaccccagcccagaagagatctcccggcttcgttgctttagactttgacatcgtcaaggttcaaaagaacgtgactgccaacgacgacgccgctgccattgttgccaagagacagaccaacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacactgagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 173 - DNA, 6230 bp, synthetic construct
  misc_feature 1..6230: note = pLD127
  source 1..6230: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgcaattgtccttgtcggttttatcaaccgttgccacggccttgttgtccctaaccaccgccgtcgatgctaagtcccacaacatcaagttgtccaagttgtccaacgaagaaacattggacgcctccacattccaagaatacacgagctccttggccaacaagtacatgaacttgttcaacgccgctcacggtaacccaaccagctttggcttgcaacacgtcttgtccaaccaagaagctgaagtcccattcgttaccccacaaaagggtggcaacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacactgagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 174 - DNA, 8110 bp, synthetic construct
  misc_feature 1..8110: note = pLD131
  source 1..8110: mol_type = other DNA
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctgatcccaatattacacccaagtagcatgcataagctaaaagtaactcgcagcgcacaccgtgcagattcataagtctatgattaattgaacgccaataacccggcttactacaagtacaagtaggtatacatagcggtaatgaatcattagaaaaataaaaaacaaaaaaaaacaaaacaaactgttgtggatgcatcaacagtagtacatagttgtacgatgtacttgtacttgtaaaagcaaaaatgtacaatatctcagggagcgcaacttttacgttcgaagaacaatgtaccgcataccgcattctagattctgcggaacgtctaacctggaaatacgattttttatttctttcattttttttgcttcttcaaaagtatggtaatttcctaccattacagttgacactgaacgaggggggattgaatttaagcaaaaaattaaatcaaaatacctttatgtatccagcccatgtaataaacaaaaggattatataacaagaaataaatatatacctttaatggatcattagaataaaaataaatacgagaagcacaccagagaagctttttgattgccactataccgctactttggtatatcttattataattgttgaatttgcaagatagaatgtcattcattggagagaaatccaaggaatatgtgggatgaaatgactagaagtatgaacaatgagaatagtacatacttgtacctgtatttctagaagagagaaagacagttgagtgtgtgattctcgtccaataataatctcaatagtaacgtgtgaatagctgttctttgatagttgatatttctcgatgactatttatgttgtacaagggatttttttcgttgctgttgatttcgaattaggcaatgcagatatcatttatgctatccatatttaagatttcccatacgcatttataacatttattctacataaattgttaaatgaacgaactgccattataaattgtttcctaaataggaagtgtttttcataaagcaagtaagttgtctaataatactaagtaataaaaataagttcatacaatatattttgagaacatcatttggaggcggtagatggagtctgtttattattaaacaatgcgagatgaccccttaaatattgagaacatcagttggaggcggcagatggagtctgtctatttagcaatgggacatgactgtcagtatcatcatatgtatatatataatacatataatattatataacacgatttttttaaattattggcccgaaaattaatcagtgtagactggatcctcgagaaccatttaattaagatcgtcgacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgcttgagtatcgaagtcttgtacctccctgtcaatcatggcaccactggtcttgacttgtctattcatactggacaagcgccagagttaagcttgtagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggaggcctaattttgacgtgcaaggggcgaattggggcgagaaacacgtcgtggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacgacgccatgggcctctctctagtatgtaccttctctttccagaccaactatcacactctactgaacccccataacaagaaccctaaaaattctcttctcagttaccagcaccccaagacgcctatcattaagtcctcctacgacaactttccctctaagtactgcctgaccaaaaacttccatctcctgggactgaactctcataacagaattagtagccagtcccgatctatccgagctggctctgaccagattgagggctcccctcaccatgaatccgacaacagcatcgctaccaagattttgaattttggtcacacatgctggaagctccagcgaccgtacgtcgtgaagggtatgatctcgattgcctgtggactgttcggacgtgagctttttaataatcgacacttgttttcatggggcctcatgtggaaggcttttttcgccctcgtgcccattctgtctttcaacttctttgccgctattatgaaccaaatctacgacgttgatattgataggatcaacaagcctgacctgccgctcgtctcgggggagatgtctatcgagacagcgtggattctttcgattatcgtcgcgctgactggccttatcgttaccataaagttgaagtctgcacccctcttcgtgtttatctacattttcggtatttttgctggattcgcgtactccgttccccctatcagatggaagcagtacccctttactaactttctgattactatcagcagccacgtcggtttagcctttacctcatattcggccaccaccagtgcactgggcctccccttcgtctggcgacctgcattttcattcatcatcgccttcatgactgtgatgggtatgaccatcgctttcgctaaggacatctccgacatcgagggtgatgctaaatatggagtgtccaccgtggccactaagctgggagcccggaacatgacgttcgtcgtctctggtgttctgctccttaactacttggtttcgatctccattggcattatctggccacaagtcttcaagtccaacattatgattctgtcccacgccattcttgccttttgcctgatcttccagacacgcgaactcgctctcgctaactacgcctccgccccatcgcgacagttcttcgagttcatctggctgctttactacgccgagtacttcgtttacgtgttcatctaataagagtaggcaattaacagatagtttgccggtgataattctcttaacctcccacactcctttgacataacgatttatgtaacgaaactgaaatttgaccagatattgttgtaaatagaaaatctggcttgtaggtggcaaaatgcggcgtctttgttcatcaattccctctgtgactactcgtcatccctttatgttcgactgtcgtatttcttattttccatacatatgcaagtgagatgcccgtgtccgaattcgctatggatccatagcgtcgacaccatatcatataaaactaacaatgcattgaattaatacgaagactacccgttgctatctccacaccgttatctccacggtccaaaggctgctcaatgtgctgcatacgtaacgtggggtgcaaccttgagcacatagtactttccgaaaaccggcgataattaagtgtgcactccaacttttcacactgagcataaaatgtggagaagaaatcacactaaaaagtcaggtagactggaaaatgcgccatgaaataatatctcttgctacagtaatgcccagcatcgaggggtattgtgtcaccaacactatagtggcagctgaagcgctcgtgattgtagtatgagtctttattggtgatgggaagagttcactcaatattctcgttactgccaaaacaccacggtaatcggccagacaccatggatgtagatcaccaagcctgtgaatgttattcgagctaaaatgcacatggttggtgaaaggagtagttgctgtcgaattccgtcgtcgcctgagtcatcatttatttaccagttggccacaaacccttgacgatctcgtatgtcccctccgacatactcccggccggctggggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgtttgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtcttctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacactgagacttactccgttcctgaggttgagcgaattgcccgaatggccgccttcctggcccttcagcacaacccccctcttcccgtgtggtcccttgacaaggccaacgtgctggcctcctctcgactttggcgaaagaccgtcacccgagtcctcaaggacgaattcccccagctggagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcctcctccacctccgaggtcggagactttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggcccagccctgctaatgaaatgatgatttataatttaccggtgtagcaaccttgactagaagaagcagattgggtgtgtttgtagtggaggacagtggtacgttttaattaa

SEQ ID NO: 175 - DNA, 7557 bp, synthetic construct
  misc_feature 1..7557: note = pLD132
  source 1..7557: mol_type = other DNA
ttaattaagatcgtcgacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgcttgagtatcgaagtcttgtacctccctgtcaatcatggcaccactggtcttgacttgtctattcatactggacaagcgccagagttaagcttgtagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggaggcctaattttgacgtgcaaggggcgaattggggcgagaaacacgtcgtggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacgacgccatgaagtgttcgacgttttctttttggtttgtttgtaaaatcattttctttttcttttctttcaacatccaaacgtcgatcgcaaaccctagagagaactttcttaagtgcttctcgcagtacatccctaataacgctaccaaccttaagctggtgtacacccagaacaaccctctttacatgtctgttctaaacagcaccatccacaatcttagattcacatcagacaccactcccaagccgctcgtcatcgtgaccccgagtcatgtgtcccatatccaaggcactatcctgtgctctaaaaaggtcggtctgcagattcggactcgctccggtggacatgattcggagggcatgtcctacattagccaggtcccctttgtgatcgtggacctgaggaacatgcggtctattaagattgatgtgcactcacagaccgcttgggtcgaggctggtgcgacattgggtgaggtgtactactgggtgaacgagaagaacgagaacctgagcctcgccgctggctactgtcccaccgtttgtgccggtggacacttcggcggaggcggatacggtccacttatgcgaaactacgggctcgcagctgataatatcatcgacgcacaccttgttaacgttcacggcaaggtgctggaccgaaaaagcatgggtgaggacctattttgggccttgcgaggcggtggtgccgaatccttcggaattatcgtggcctggaagatccgactggtcgctgtgccaaagtccactatgttctccgtcaagaaaattatggagatccacgaactcgtaaagctcgtcaataagtggcagaacatcgcctacaagtatgacaaggatctgctgctcatgactcacttcatcacgcgaaacattacagacaaccagggaaagaacaagaccgctatccatacctacttctcctctgtcttccttgggggtgtcgattccctcgttgatctcatgaacaaatcttttccagagctcggaatcaagaagaccgactgccgacagctctcttggatcgacaccattattttctactcaggagtcgtaaactacgatactgacaactttaacaaggagattctgttagatcgatcggccggccagaacggtgccttcaagatcaagctcgactatgtcaaaaagcccattcctgaatccgtcttcgttcaaattcttgaaaagttgtacgaggaggatatcggcgccggaatgtacgcgctgtacccctacggtggcattatggacgagatttctgaaagtgctattcccttcccccaccgtgctggcattctgtatgagctgtggtacatttgctcctgggaaaagcaggaggacaacgagaagcacttgaactggatacgaaacatttacaatttcatgaccccctatgtttcgaagaaccctcgactggcctacctgaattaccgcgacctcgacatcggaattaacgaccctaagaaccccaataactatactcaggccagaatctggggcgagaagtacttcggcaagaactttgaccgtctggttaaggtcaagaccctcgtggaccctaacaacttcttccgaaacgagcagtctatcccccctctgccccgacaccggcattaataagagtaggcaattaacagatagtttgccggtgataattctcttaacctcccacactcctttgacataacgatttatgtaacgaaactgaaatttgaccagatattgttgtaaatagaaaatctggcttgtaggtggcaaaatgcggcgtctttgttcatcaattccctctgtgactactcgtcatccctttatgttcgactgtcgtatttcttattttccatacatatgcaagtgagatgcccgtgtccgaattcgctatggatccatagcgcaggttgtgcagtatcatacatactcgatcagacaggtcgtctgaccatcatacaagctgaacaagcgctccatacttgcacgctctctatatacacagttaaatgacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcatctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacatcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagttccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgggcggcaaaggcggacttgtggacgttagctcgcgcttcgtaggagggcattttggtggtgaagaggagactgaaataaatttagtctgcacaactttttatcggaaccttatctggggcagtgaagtatatgttatggtaatagttacgagttagttgaacttatagatagactggactatacggctatcggtccaaattagaaattaattaaatggttctcgaggatccagtctacactgattaattttcgggccaataatttaaaaaaatcgtgttatataatattatatgtattatatatatacatatgatgatactgacagtcatgtcccattgctaaatagacagactccatctgccgcctccaactgatgttctcaatatttaaggggtcatctcgcattgtttaataataaacagactccatctaccgcctccaaatgatgttctcaaaatatattgtatgaacttatttttattacttagtattattagacaacttacttgctttatgaaaaacacttcctatttaggaaacaatttataatggcagttcgttcatttaacaatttatgtagaataaatgttataaatgcgtatgggaaatcttaaatatggatagcataaatgatatctgcattgcctaattcgaaatcaacagcaacgaaaaaaatcccttgtacaacataaatagtcatcgagaaatatcaactatcaaagaacagctattcacacgttactattgagattattattggacgagaatcacacactcaactgtctttctctcttctagaaatacaggtacaagtatgtactattctcattgttcatacttctagtcatttcatcccacatattccttggatttctctccaatgaatgacattctatcttgcaaattcaacaattataataagatataccaaagtagcggtatagtggcaatcaaaaagcttctctggtgtgcttctcgtatttatttttattctaatgatccattaaaggtatatatttatttcttgttatataatccttttgtttattacatgggctggatacataaaggtattttgatttaattttttgcttaaattcaatcccccctcgttcagtgtcaactgtaatggtaggaaattaccatacttttgaagaagcaaaaaaaatgaaagaaataaaaaatcgtatttccaggttagacgttccgcagaatctagaatgcggtatgcggtacattgttcttcgaacgtaaaagttgcgctccctgagatattgtacatttttgcttttacaagtacaagtacatcgtacaactatgtactactgttgatgcatccacaacagtttgttttgtttttttttgttttttatttttctaatgattcattaccgctatgtatacctacttgtacttgtagtaagccgggttattggcgttcaattaatcatagacttatgaatctgcacggtgtgcgctgcgagttacttttagcttatgcatgctacttgggtgtaatattgggatcaggtcaggcggaatggcacttccttattccagatgcgtgcggattatgcatgactggccgtcgttttacaacgtcgtgactgggaaaaccctggcgcttgtggtggatcacagttattggagacttccttggccgtacgatgggttttgtttcaggtatcccaacctggacgtctcgtataatgcgacaaacaacagataaaacgaaaggcccagtctttcgactgagcctttcgttttatttggatccactagttctagagcggcgggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaatttgttaattggttgtaacactggcagagcggacgatttgaagccccctacccatttacgctgacttgacgggacggcggctttgttgaatagagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttccagcgacatatgccatacgccgaactgaactccacctaatgtcttggagattgtcggatagcgcggaataggattgtgccctctgagcggataacaatttcacacaggaaacagctatgaccatgacacatgcatccaccatcgcagacttatcatcactgagcctccacctagcct

SEQ ID NO: 176 - DNA, 8500 bp, synthetic construct
  misc_feature 1..8500: note = pLD135
  source 1..8500: mol_type = other DNA
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctttaattaattggcaaattttactgtggccttcagaacggtaaaaatagaccaatcagaattctgaaaagcacatcttgatctcctcattgcggggagtccaacggtggtcttattcccccgaatttcccgctcaatctcgttccagaccgacccggacacagtgcttaacgccgttccgaaactctaccgcagatatgctccaacggactgggctgcatagatgtgatcctcggcttggagaaatggataaaagccggccaaaaaaaaagcggaaaaaagcggaaaaaaagagaaaaaaaatcgcaaaatttgaaaaatagggggaaaagacgcaaaaacgcaaggaggggggagtatatgacactgataagcaagctcacaacggttcctcttatttttttcctcatcttctgcctaggttcccaaaatcccagatgcttctctccagtgccaaaagtaagtaccccacaggttttcggccgaaaattccacgtgcagcaacgtcgtgtggggtgttaaaatgtgggggcggggaaccaggacaagaggctcttgtgggagccgaatgagagcacaaagcgggcgggtgtgataagggcatttttgcccattttcccttctcctgtctctccgacggtgatggcgttgtgcgtcctctatctatttctttttatttctttttgttttatttctctgactaccgatttggcttgatttcctcaaccccacacaaataagctcgggccgaggaatatatatatacacggacacagtcgccctgtggacaacacgtcactacctctacgacgctatggatccatagcgaattcggacacgggcatctcacttgcatatgtatggaaaataagaaatacgacagtcgaacataaagggatgacgagtagtcacagagggaattgatgaacaaagacgccgcattttgccacctacaagccagattttctatttacaacaatatctggtcaaatttcagtttcgttacataaatcgttatgtcaaaggagtgtgggaggttaagagaattatcaccggcaaactatctgttaattgcctactcttattacagtttggctcgtcgtccccgagacttgccggccttgatagcgaggagtgtcttctcgtcagcaggggcgctaccggaggctcgttgctgtctgaagtaggttcgggcctcgtcctgtcgggtggcctcagcgccggtggcgatagcggcccgaaggtgctcggctccgtagccgaaggcatcaaccagatcaagtgcgtgtggtcggatcttaacgagaagacgattaatgtaagtgccgaccgttcggcctctctgcatcgagagcctaccgttcatcagataccagctgaggtgcttctcgataagggacagaccaaaaaggtcacgcaatcgggtcaacacttccttagtgccagcgtcgtccaccttggccaaagcctcagtaaaggcctcccactgtaaaagctcagcgtgggcctgggcagcctcgatcagttcgttttgatgctgattgaacagtgctgccgcctggtgctgggggagcttgcccgcgcctttgagagcagcgccaacctctgcaaccatggactggactctgtcggtcagcagagttcgctgaccttcttcgtctcggagggcaagagcagatttctggacagatccactgtcggccacgaactgagcgacctgtcgcagtccggttcggtgtagcgcgactccggcagcctggtccacgacgtaccgcgcgagcacgccgaagttggcacctcggaactccttagcatagtcagcgagcagcctcttggccaccaactgaagaagcacggtgttgtctccttcgaaggtcacgtagacgtcgagatcggcccggagagaagcgaatcggttctcaatcagaaaacctgcacccccacacgcttctcggcactcttgtagggtatcgagagcatgccaggtagaaaggggcttcagagcagcagccagtgtctcaaggtcctgacggtcggcgtcagtatcatgagcacccgagaacacgtcgtcgaatttctgcagcagttgctcatgggcgaacgatgcggcgtaggtggtggcgagtcgggtaaagaggcgccgctggtgtcgctggtaatccaggagcacctcctcctctgtgggcgaagtggcgttgaactgtcttcgctcagcggcgtagtggatggcggattgcagagcaaccttcgatgcagccactgcagcaccgtccaggctgactcggccctggaccagcgtacctagcattgtaaagaatcttcgccccggagattcgatggttgagctgtaggtgccatcgacggcaacatcgccgtaacggttaagcaggttggttcggggaatgcgaacattggtgaaatggagacgtccgttatcaattccgtttagtccccccttaataccgtcgtcctctccaccaataccagggagaaagtctccggtggcgggatctctcagatcgacgtagaaggcgtgcactccatggttaacctttcgagtaatcagttgggcaaacacgacagcggccaaaccgtcgttagcggcgtttccaatgtagtccttccaggcggctcggaacggggtgtcaataacgaactcctgggtttcctcatcataggtggccgttgtagcaatggaagcgacatcggagccatggccagtctcagtcatggcaaagcaaccagggatttccagagacatgatgcctgggagccacttgtcgtggtgctccctagttcccaagtgcataacagcggagccgaacagtccccactggacgccggccttgatctggagggaggggtccgccgtgacaagctcctcaaaaccagcgatgtttccgccgtggtcgtcactaccacccagtcgagaaggaaaggctcggtgaactgcgttgttatcgaccaagtacttgagctggccaaagacgcgagagcggtgctctgtatgagtcagaccctccaccttctgaactacctctcgtcccgcaaggtcccgggcgtggagacggatatcagcccatcggcccagcagctgctctcccagcgcagccacatctactcggggctcgacggccaccttagcaccgtctgcggcggccgtagttgagccaggggatgcgggggatgaggctctgtcaactacttcggtcatggcgtcgttgcttgtgtgatttttgaggacccatccctttggtatataagtatactctggggttaaggttgcccgtgtagtctaggttatagttttcatgtgaaataccgagagccgagggagaataaacgggggtatttggacttgtttttttcgcggaaaagcgtcgaatcaaccctgcgggccttgcaccatgtccacgacgtgtttctcgccccaattcgccccttgcacgtcaaaattaggcctccatctagacccctccataacatgtgactgtggggaaaagtataagggaaaccatgcaaccatagacgacgtgaaagacggggaggaaccaatggaggccaaagaaatggggtagcaacagtccaggagacagacaaggagacaaggagagggcgcccgaaagatcggaaaaacaaacatgtccaattggggcagtgacggaaacgacacggacacttcagtacaatggaccgaccatctccaagccagggttattccggtatcaccttggccgtaacctcccgctggtacctgatattgtacacgttcacattcaatatactttcagctacaataagagaggctgtttgtcgggcatgtgtgtccgtcgtatggggtgatgtccgagggcgaaattcgctacaagcttaactctggcgcttgtccagtatgaatagacaagtcaagaccagtggtgccatgattgacagggaggtacaagacttcgatactcaagcattactcggacttgtggcgattgaacagacgggcgatcgcttctcccccgtattgccccgtttctacgagtgccgtacgagtacgagtacaggaggtacagtacattccgatacttgcactcccaccattgctgtatatctctgctacttgtggtatcaaatccgctactcgtagtggtttttccacttgtacaaattctatcccccatgaccaccaacccttctcccgatcgccagtctcacttggcggaatcgtcgacgatcatcctcgagatgcggccgcgtcgacataacttcgtatagcatacattatacgaagttattttctaatttggaccgatagccgtatagtccagtctatctataagttcaactaactcgtaactattaccataacatatacttcactgccccagataaggttccgataaaaagttgtgcagactaaatttatttcagtctcctcttcaccaccaaaatgccctcctacgaagcgcgagctaacgtccacaagtccgcctttgccgcccgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggaactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggatgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagatgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtcatttaactgtgtatatagagagcgtgcaagtatggagcgcttgttcagcttgtatgatggtcagacgacctgtctgatcgagtatgtatgatactgcacaacctgataacttcgtatagcatacattatacgaagttatctcgaggcggccgcatggagcgtgtgttctgagtcgatgttttctatggagttgtgagtgttagtagacatgatgggtttatatatgatgaatgaatagatgtgattttgatttgcacgatggaattgagaactttgtaaacgtacatgggaatgtatgaatgtgggggttttgtgactggataactgacggtcagtggacgccgttgttcaaatatccaagagatgcgagaaactttgggtcaagtgaacatgtcctctctgttcaagtaaaccatcaactatgggtagtatatttagtaaggacaagagttgagattctttggagtcctagaaacgtattttcgcgttccaagatcaaattagtagagtaatacgggcacgggaatccattcatagtctcaattttcccataggtgtgctacaaggtgttgagatgtggtacagtaccaccatgattcgaggtaaagagcccagaagtcattgatgaggtcaagaaatacacagatctacagctcaatacaatgaatatcttctttcatattcttcaggtgacaccaagggtgtctattttccccagaaatgcgtgaaaaggcgcgtgtgtagcgtggagtatgggttcggttggcgtatccttcatatatcgacgaaatagtagggcaagagatgacaaaaagtatctatatgtagacagcgtagaatatggatttgattggtataaattcatttattgcgtgtctcacaaatactctcgataagttggggttaaactggagatggaacaatgtcgatatctcgacatattttgatatttgttaattaa

SEQ ID NO: 177 - DNA, 5655 bp, synthetic construct
  misc_feature 1..5655: note = pLD137
  source 1..5655: mol_type = other DNA
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctgatcccaatattacacccaagtagcatgcataagctaaaagtaactcgcagcgcacaccgtgcagattcataagtctatgattaattgaacgccaataacccggcttactacaagtacaagtaggtatacatagcggtaatgaatcattagaaaaataaaaaacaaaaaaaaacaaaacaaactgttgtggatgcatcaacagtagtacatagttgtacgatgtacttgtacttgtaaaagcaaaaatgtacaatatctcagggagcgcaacttttacgttcgaagaacaatgtaccgcataccgcattctagattctgcggaacgtctaacctggaaatacgattttttatttctttcattttttttgcttcttcaaaagtatggtaatttcctaccattacagttgacactgaacgaggggggattgaatttaagcaaaaaattaaatcaaaatacctttatgtatccagcccatgtaataaacaaaaggattatataacaagaaataaatatatacctttaatggatcattagaataaaaataaatacgagaagcacaccagagaagctttttgattgccactataccgctactttggtatatcttattataattgttgaatttgcaagatagaatgtcattcattggagagaaatccaaggaatatgtgggatgaaatgactagaagtatgaacaatgagaatagtacatacttgtacctgtatttctagaagagagaaagacagttgagtgtgtgattctcgtccaataataatctcaatagtaacgtgtgaatagctgttctttgatagttgatatttctcgatgactatttatgttgtacaagggatttttttcgttgctgttgatttcgaattaggcaatgcagatatcatttatgctatccatatttaagatttcccatacgcatttataacatttattctacataaattgttaaatgaacgaactgccattataaattgtttcctaaataggaagtgtttttcataaagcaagtaagttgtctaataatactaagtaataaaaataagttcatacaatatattttgagaacatcatttggaggcggtagatggagtctgtttattattaaacaatgcgagatgaccccttaaatattgagaacatcagttggaggcggcagatggagtctgtctatttagcaatgggacatgactgtcagtatcatcatatgtatatatataatacatataatattatataacacgatttttttaaattattggcccgaaaattaatcagtgtagactggatcctcgagaaccatttaattaagatcagagaccggatggtctcaagcgtcgacaccatatcatataaaactaacaatgcattgaattaatacgaagactacccgttgctatctccacaccgttatctccacggtccaaaggctgctcaatgtgctgcatacgtaacgtggggtgcaaccttgagcacatagtactttccgaaaaccggcgataattaagtgtgcactccaacttttcacactgagcataaaatgtggagaagaaatcacactaaaaagtcaggtagactggaaaatgcgccatgaaataatatctcttgctacagtaatgcccagcatcgaggggtattgtgtcaccaacactatagtggcagctgaagcgctcgtgattgtagtatgagtctttattggtgatgggaagagttcactcaatattctcgttactgccaaaacaccacggtaatcggccagacaccatggatgtagatcaccaagcctgtgaatgttattcgagctaaaatgcacatggttggtgaaaggagtagttgctgtcgaattccgtcgtcgcctgagtcatcatttatttaccagttggccacaaacccttgacgatctcgtatgtcccctccgacatactcccggccggctggggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgtttgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtcttctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacactgagacttactccgttcctgaggttgagcgaattgcccgaatggccgccttcctggcccttcagcacaacccccctcttcccgtgtggtcccttgacaaggccaacgtgctggcctcctctcgactttggcgaaagaccgtcacccgagtcctcaaggacgaattcccccagctggagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcctcctccacctccgaggtcggagactttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggcccagccctgctaatgaaatgatgatttataatttaccggtgtagcaaccttgactagaagaagcagattgggtgtgtttgtagtggaggacagtggtacgttttaattaa

SEQ ID NO: 178 - DNA, 4664 bp, synthetic construct
  misc_feature 1..4664: note = pLD138
  source 1..4664: mol_type = other DNA
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctgatcccaatattacacccaagtagcatgcataagctaaaagtaactcgcagcgcacaccgtgcagattcataagtctatgattaattgaacgccaataacccggcttactacaagtacaagtaggtatacatagcggtaatgaatcattagaaaaataaaaaacaaaaaaaaacaaaacaaactgttgtggatgcatcaacagtagtacatagttgtacgatgtacttgtacttgtaaaagcaaaaatgtacaatatctcagggagcgcaacttttacgttcgaagaacaatgtaccgcataccgcattctagattctgcggaacgtctaacctggaaatacgattttttatttctttcattttttttgcttcttcaaaagtatggtaatttcctaccattacagttgacactgaacgaggggggattgaatttaagcaaaaaattaaatcaaaatacctttatgtatccagcccatgtaataaacaaaaggattatataacaagaaataaatatatacctttaatggatcattagaataaaaataaatacgagaagcacaccagagaagctttttgattgccactataccgctactttggtatatcttattataattgttgaatttgcaagatagaatgtcattcattggagagaaatccaaggaatatgtgggatgaaatgactagaagtatgaacaatgagaatagtacatacttgtacctgtatttctagaagagagaaagacagttgagtgtgtgattctcgtccaataataatctcaatagtaacgtgtgaatagctgttctttgatagttgatatttctcgatgactatttatgttgtacaagggatttttttcgttgctgttgatttcgaattaggcaatgcagatatcatttatgctatccatatttaagatttcccatacgcatttataacatttattctacataaattgttaaatgaacgaactgccattataaattgtttcctaaataggaagtgtttttcataaagcaagtaagttgtctaataatactaagtaataaaaataagttcatacaatatattttgagaacatcatttggaggcggtagatggagtctgtttattattaaacaatgcgagatgaccccttaaatattgagaacatcagttggaggcggcagatggagtctgtctatttagcaatgggacatgactgtcagtatcatcatatgtatatatataatacatataatattatataacacgatttttttaaattattggcccgaaaattaatcagtgtagactggatcctcgagaaccatttaattaatttctaatttggaccgatagccgtatagtccagtctatctataagttcaactaactcgtaactattaccataacatatacttcactgccccagataaggttccgataaaaagttgtgcagactaaatttatttcagtctcctcttcaccaccaaaatgccctcctacgaagcgcgagctaacgtccacaagtccgcctttgccgcccgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggaactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggatgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagatgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtcatttaactgtgtatatagagagcgtgcaagtatggagcgcttgttcagcttgtatgatggtcagacgacctgtctgatcgagtatgtatgatactgcacaacctgcgcttgagaccatccggtctctgatcttaattaa

SEQ ID NO: 179 - DNA, 5975 bp, synthetic construct
  misc_feature 1..5975: note = pLD139
  source 1..5975: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgaacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacacggaagaagggcaaagttgtaagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 180 - DNA, 5594 bp, synthetic construct
  misc_feature 1..5594: note = pLD19
  source 1..5594: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgggtttatcgtcagtgtgcactttttcttttcaaacaaactaccacaccctcctaaaccctcacaataataacccaaaaacctccttgctatgttacagacatccaaagacaccgatcaagtattcatacaacaattttcccagtaaacattgctcaacgaagtccttccacttgcaaaacaaatgcagcgaatcattgtcgatagctaaaaactcgatacgtgcggcaaccactaaccaaactgagccaccagagagcgataatcattcagtcgccaccaagattttgaactttggaaaagcctgttggaaacttcaaaggccttacaccattatcgcatttaccagttgcgcatgtggtttgttcgggaaggaattattacacaacacaaatttgatcagctggagcctaatgtttaaggcatttttcttcttagttgcaattttgtgtatagcttcgtttacaacgaccattaatcagatttacgaccttcacatcgatcggatcaataaaccagacttgccccttgcctctggggaaatctctgtaaatactgcatggatcatgctgataatcgtggctttgtttggattgattattacaattaagatgaaggggggtccattatatatattcgggtactgcttcggcattttcggtggtatcgtttactccgtcccaccctttagatggaaacagaaccccagtacggcctttctactcaatttcttggctcatatcatcacaaacttcacattctattatgcaagccgagcggcgcttggtttgccgttcgaactcagaccgagttttacatttctccttgccttcatgaaactgatgggactggcccttgcattgatcaaggatgcgtcagatgtcgaaggcgacactaagttcggcattctgacgcttgcttccaagtatggaagtagaaatctaacgcttttttgttcaggaatagtgctacttagttatgttgctgctatactcgctggcattatttggcctcaggccttcaactctaacgtaatgttgttatcccatgctattttggcgttctggttgatcttgcaaacgcgagattttgcactcactaactacgacccagaggcaggaaggcgcttttacgagtttatgtggaagttgtattatgccgaatacttggtttatgttttcatttgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 181 - DNA, 5372 bp, synthetic construct
  misc_feature 1..5372: note = pLD26
  source 1..5372: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatggcggcaaccactaaccaaactgagccaccagagagcgataatcattcagtcgccaccaagattttgaactttggaaaagcctgttggaaacttcaaaggccttacaccattatcgcatttaccagttgcgcatgtggtttgttcgggaaggaattattacacaacacaaatttgatcagctggagcctaatgtttaaggcatttttcttcttagttgcaattttgtgtatagcttcgtttacaacgaccattaatcagatttacgaccttcacatcgatcggatcaataaaccagacttgccccttgcctctggggaaatctctgtaaatactgcatggatcatgctgataatcgtggctttgtttggattgattattacaattaagatgaaggggggtccattatatatattcgggtactgcttcggcattttcggtggtatcgtttactccgtcccaccctttagatggaaacagaaccccagtacggcctttctactcaatttcttggctcatatcatcacaaacttcacattctattatgcaagccgagcggcgcttggtttgccgttcgaactcagaccgagttttacatttctccttgccttcatgaaactgatgggactggcccttgcattgatcaaggatgcgtcagatgtcgaaggcgacactaagttcggcattctgacgcttgcttccaagtatggaagtagaaatctaacgcttttttgttcaggaatagtgctacttagttatgttgctgctatactcgctggcattatttggcctcaggccttcaactctaacgtaatgttgttatcccatgctattttggcgttctggttgatcttgcaaacgcgagattttgcactcactaactacgacccagaggcaggaaggcgcttttacgagtttatgtggaagttgtattatgccgaatacttggtttatgttttcatttgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 182 - protein, 11 aa, synthetic construct
  REGION 1..11: note = VSV-G
YTDIEMNRLGK